(19) United States
(12) Patent Application Publication
(10) Pub. No.: US 2023/0265160 A1
(43) Pub. Date: Aug. 24, 2023

(54) THERAPEUTIC FUSION PROTEINS

(71) Applicant: NOVARTIS AG, Basel (CH)

(72) Inventors: Sebastien IRIGARAY, Zimrnersheim (FR); Laurent KLEIN, Zaessingue (FR); Darko SKEGRO, Riehen (CN); Marco VILLANI, Saint-Louis (FR); Karl WELZENBACH, Huningue (FR)

(21) Appl. No.: 17/640,293

(22) PCT Filed: Sep. 4, 2020
(86) PCT No.: PCT/IB2020/058251

§ 371 Date: Mar. 3, 2022

(30) Foreign Application Priority Data
Sep. 6, 2019 (EP) 19196045.9

(51) Int. Cl. C07K 14/705; C07K 14/765; A61P 29/00

(52) U.S. Cl. CPC C07K 14/70546; A61P 29/00; C07K 14/765; C07K 2319/21

(57) ABSTRACT

The present invention relates to fusion proteins suitable for use as a medicament or research tool. Therapeutic uses of the fusion proteins may include the prevention or treatment of acute or chronic inflammatory and immune system-driven organ and micro-vascular disorders, for example, acute kidney injury, acute myocardial infarction, acute respiratory distress or chronic obstructive pulmonary disease fibrosis and other organ injuries resulting from tissue trauma and acute and chronic injury.

### SEQUENCE LISTING

The instant application contains a Sequence Listing which has been submitted electronically in ASCII format and is hereby incorporated by reference in its entirety. Said ASCII copy, created on Aug. 31, 2020, is named PAT058332_SL.txt and is 653,193 bytes in size.

### FIELD OF THE INVENTION

The present invention relates to fusion proteins comprising both integrin binding and phosphatidylserine binding capabilities. The fusion proteins can be used as therapeutics, in particular for the prevention or treatment of acute or chronic inflammatory disorders and immune system- or coagulation-driven organ and micro-vascular disorders.

### BACKGROUND

Acute inflammatory organ injuries (AOIs) are historically challenging diseases with high morbidity, mortality and significant unmet medical need. Typical AOIs include myocardial infarction (MI) and stroke which occur in 32.4 million patients worldwide every year. Patients with previous MI and stroke are considered by the World Health Organization as the highest risk group for further coronary and cerebral events, which rank amongst the top causes of morbidity in the developed world. Another AOI is acute kidney injury (AKI), which occurs in about 13.3 million people per year. In high income countries, AKI incidence is 3-5/1000 and is associated with high mortality (14-46%) (Metha et al., (2015) Lancet, 385(9987): 2616-43). Similar to MI and stroke, AKI survivors often fail to recover completely and are at increased risk of developing chronic kidney disease or end-stage renal disease. There is to date no FDA-approved drug available to prevent or treat AKI. Developing new treatments for AKI has proven challenging, with no successful outcomes from clinical trials so far. This is likely due to the multifactorial and multifaceted pathophysiology of AKI including inflammatory, microvascular dysfunction and nephrotoxic pathomechanisms elicited by septic, ischemic/reperfusion and/or nephrotoxic insults. These drivers can act simultaneously or consecutively to cause mostly tubular but also glomerular cell damage, loss of renal functional reserve and eventually kidney failure.

One common denominator of AOIs is increased cell death due to tissue injury, increased generation of cell fragments and prothrombotic/proinflammatory microparticles which can enter the circulation and injured tissue. After tissue infiltration of neutrophils to defend against infection, neutrophils undergo apoptosis or other forms of cell death in the affected tissue. Neutrophils contain harmful substances, including proteolytic enzymes and danger-associated molecular patterns (DAMPs) that can promote host tissue damage and propagate inflammation. Efficient uptake of dying cells triggers signaling events that lead to the reprogramming of macrophages (MΦ) towards a non-inflammatory, pro-resolving phenotype and the release of key mediators for successful resolution and repair of the affected tissue. This reprograming has been recently attributed to a metabolic signaling which activates phagocytic anti-inflammatory responses in macrophages (Zhang et al., (2019) Cell Metabolism, 29(2): 443-56). This removal of debris, or aged or dying cells in a non-inflammatory manner is termed ‘efferocytosis’.

However, in the case where efferocytosis is delayed, necrotic cells can accumulate and cause, for example, inflammatory responses triggering of pro-inflammatory cytokines (TNF-α) or immunosuppressive IL-10 by macrophages (Greenlee-Wacker (2016) Immunol. Reviews, 273: 357-370). Furthermore, if cell debris and particulates are not removed efficiently, they can cause cell clumps and aggregates, such as neutrophil-platelet fragment clusters, micro-thrombi and/or release danger-associated molecular patterns (DAMPS) such as ATP, DNA, histones or HMGB1. The consequences can include microvasculature occlusion, dysfunction and pronounced sterile inflammation resulting in progression of tissue injury, primary and secondary organ failure or maladaptive repair.

In the acute phase of AOIs, efferocytotic pathways appear significantly downregulated. Inflammation or acute response to injury (mechanical cues, hypoxia, oxidative stress, radiation, inflammation, and infection) suppress effective efferocytosis or phagocytosis by downregulation of dedicated phosphatidylserine (PS) binding proteins which include bridging proteins and cell surface efferocytosis/clearance receptors. An example for defunctionalization of an efferocytosis receptor is the proteolytic shedding of TAM family receptors such as Mer tyrosine kinase (MerTK). MerTK is an integral membrane protein preferentially expressed on phagocytic cells, where it acts as signaling protein but also promotes efferocytosis (via proteins such as Gas6 or Protein S) and inhibits inflammatory signaling. Proteolytic cleavage and release of the soluble ectodomain of MerTK is induced by the metalloproteinase ADAM17. The shedding process can reduce efferocytosis of phagocytic cells by deprivation of surface MerTK. In addition, the released ectodomain can also inhibit efferocytosis in vitro (Zhang et al., (2015) J Mol Cell Cardiol., 87:171-9; Miller et al., (2017) Clin Cancer Res., 23(3):623-629). Increased serum/ plasma soluble Mer amounts are typically observed in inflammatory, malignant or autoimmune diseases such as diabetic nephropathy or systemic lupus erythematosus (SLE) and can mark disease severity (Ochodnicky P (2017) Am J Pathol., 187(9):1971-1983; Wu et al., (2011) Arthritis Res Ther. 13:R88). In addition, bridging proteins such as milk fat globule-EGF factor 8 protein (MFG-E8) are also downregulated during the most acute and chronic inflammatory diseases. Similar to soluble Mer, reduced serum/plasma concentration of MFG-E8 can be found in patients with MI or stable angina patients (Dai et al., (2016) World J Cardiol., 8(1): 1-23) and can mark disease severity as described for chronic obstructive pulmonary disease (COPD; Zhang et al., (2015) supra).

Phosphatidylserine (PS) exposure on dying cells is an evolutionarily conserved anti-inflammatory and immunosuppressive signal to immune cells. A vast number of major mammalian pathogens utilize PS mediated uptake as part of virulent cellular infection (Birge et al., (2016) Cell Death Diff., 23(6): 962-78). Viruses for instance can bind to PS binding-receptors directly or via proteins such as Gas6 (Morizono & Chen (2014) J Virol., 88(8):4275-90). It is possible that inactivation of endogenous clearance pathways in response to injury presents an evolutionary developed response to reduce the efficiency of an infectious agent to enter and hijack cells after injury and thereby eluding the hosts immune response and defense. In consequence, down-modulation of clearance pathways would improve the efficacy of innate and adaptive immune effectors to fight infection. As a “friendly fire” consequence, efferocytosis can be temporarily impacted during acute organ injury and the above mentioned complications in AOIs may occur. An accumulation of dying cells, debris and proinflammatory and prothrombotic MPs are hallmarks of AOIs and represent major triggers of inflammation and microvascular damage. It is noteworthy, that such accumulation of proinflammatory and prothrombotic microparticles is common in severe diseases with high medical need and may contribute to their morbidity. Examples for such indications are sepsis and cancer (Yang et al., (2016) Tumour Biol., 37(6): 7881-91; Zhao et al., (2016) J Exp Clin Cancer Res., 35: 54; Muhsin-Sharafaldine et al., (2017) Biochim Biophys Acta Gen Subj., 1861(2): 286-295; Ma et al., (2017) Sci Rep., 7(1): 4978; Souza et al., (2015) Kidney Int. 87(6): 1100-8). Previous drug discovery efforts in this area have focused on PS binding proteins, which can serve as basis for a drug candidate design as reviewed by (Li et al., (2013) Exp Opin Ther Targets, 17(11): 1275-1285).

A subset of PS binding proteins also recognize and bind to integrins, such as αvβ3 and αvβ5, which are expressed on many cell types including phagocytes. These proteins act to bridge the PS exposing apoptotic/dying cells to integrins, resulting in efferocytosis (also termed phagocytosis) by macrophages and non-professional phagocytes. Some bridging proteins are also downregulated during the most acute and chronic inflammatory diseases. Therapeutic uses for such bridging proteins or truncated versions thereof have been previously suggested (WO2006122327 (sepsis), WO2009064448 (organ injury after ischemia/reperfusion), WO2012149254 (cerebral ischemia) The Feinstein Institute for Medical Research; WO2015025959 (myocardial infarction) Kyushu University & Tokyo Medical University; WO20150175512 (bone resorption) University of Pennsylvania; WO2017018698 (tissue fibrosis) Korea University Research and Business Foundation and US20180334486 (tissue fibrosis) Nexel Co., Ltd.); WO2020084344; however use of the wild-type or naturally occurring proteins is limited by a number of problems. For example, the wild-type MFG-E8 (wtMFG-E8) is considered to have poor developability, low solubility and to express at a very low yield when cultured in cell expression systems. Work by Castellanos et al., (2016) has shown that MFG-E8 expressed in insect or CHO cells as Fc-IgG fusion is completely aggregated and could only be purified efficiently by the addition of detergents such as Triton X-100 or CHAPS (Castellanos et al., (2016) Protein Exp. Pur., 124: 10-22).

Major functions of MFG-E8 reported so far are to enhance efferocytosis (Hanayama 2004 Science), to modulate lipid uptake/processing (Nat Med. 2014). rMFG-E8 regulates enterocyte-specific lipid storage by promoting enterocyte triglyceride hydrolase (TG) activity (JCI 2016). Intracellular MFG-E8 was shown as suppressor of hepatic lipid accumulation and inflammation acting through inhibition of the ASK1-JNK/p38 signaling cascade. (Zhang et al 2020). In addition, antiinflammatory properties, promotion of angiogenesis, atherosclerosis, tissue remodeling, and hemostasis regulation have been described for MFG-E8. Furthermore, MFG-E8 has been reported to remove excessive collagen in lung tissues, by binding of collagen through its C1 domain. Interestingly, MFG-E8-/- macrophages exhibited defective collagen uptake that could be rescued by recombinant MFG-E8 containing at least one discoidin domain (Atabai et al 2009)

In preclinical studies recombinant MFG-E8 has shown convincing protection in various, mostly rodent models of acute inflammatory and organ diseases as well in disease models with aberrant healing. Recombinant MFG-E8 has shown to accelerate wound healing of diabetic and I/R-induced wounds/ulcers (Uchiyama et al 2015/2017); accelerated repair of intestinal epithelium after colitis (Bu et al 2007) and acceleration of tendon repair after injury (Shi et al 2019); Recombinant MFG-E8 reduced kidney damage and fibrosis in ureteral obstruction (UUO) model (Brisette et al 2016). Besides, efficacy was attested in typical models of fibrosis where recombinant MFG-E8 accelerated resolution of TAA and CCI4-induced liver fibrosis (An SY, Gastroenterology 2016) and protected in a bleomycin-induced lung fibrosis model (Atabai et al 2009). Recently, a C2 depleted truncated version was published to exert similar or even better efficacy in several preclinical fibrosis models including the TAA liver fibrosis model. (WO2020084344).

EDIL3 (EGF-like repeat and discoidin l-like domain-containing protein 3) was recently reviewed by Hajishengallis and Chavakis 2019. EDIL3 (alias DEL-1) was shown to mediate efferocytosis, regulate neutrophil recruitment and inflammation, can trigger as part of the hematopoietic stem cell niche emergency myelopoiesis (avb3-integrin dependent), restrains osteoclastogenesis and inhibits inflammatory bone loss in rodents and non-human primates. EDIL3 was found as to be an integral component of the immune privilege of the central nervous system. The potential of EDIL3 as therapeutic protein was tested as an fusion protein with the Fc fragment of human IgG (DEL-1-Fc). DEL-Fc administration inhibited neutrophil infiltration, blocked IL-17 driven inflammatory bone loss in a mouse model of periodontitis (Eskan et al 2012 doi:10.1038/ni.2260;). In addition, DEL-1-Fc improved periodontal inflammation, tissue destruction and bone loss in a non human primate periodontitis model (Shin et al 2015 DOI: 10.1126/scitranslmed.aac5380). Besides, DEL-1-Fc ameliorated relapsing-remitting experimental autoimmune encephalomyelitis (EAE), a translational multiple sclerosis model (Choi et al 2014 doi:10.1038/mp.2014.146); DEL-1-Fc furthermore decreased the incidence and severity of postoperative peritoneal adhesions in a mouse model Fu et al 2018.

The removal of dying cells, debris and microparticles by the bridging proteins, for example, MFG-E8, EDIL3, Gas6, could eliminate major causes of sterile inflammation and microvascular dysfunction and thus prevent progression of tissue injury and enable the resolution of inflammation. Therefore, a therapeutic approach to promote the clearance of dying cells during the course of AOIs could be used to reduce or at least alleviate the pathology of AOIs and could be meaningful in other disease settings where dying cells or PS exposing microparticles are insufficiently cleared. As such, there is a need for a therapeutic agent that can be used to reduce tissue injury and inflammation and which has desirable manufacturing properties to address the unmet medical need in AOls.

### SUMMARY OF THE DISCLOSURE

In the present disclosure, the applicants have generated recombinant, therapeutic fusion proteins based on the structure of the naturally occurring bridging proteins (e.g. MFG-E8) without the aforementioned undesirable properties and production issues of the wild-type protein. The fusion proteins of the present disclosure comprise an integrin binding domain (for example EGF-like domain), a solubilizing domain and a phosphatidylserine binding domain (for example C1 domain from MFG-E-8 or its paralogue EDIL3). The proteins of the invention are suitable for prevention or treatment of acute or chronic inflammatory, immune system- or fibrosis-driven organ disorders. The proteins of the invention may also find its application to enable, accelerate and promote repair and regeneration.

The fusion proteins maintain the major biologic functions of the wild-type MFG-E8 or EDIL3 protein, for example, by functioning to bridge PS-exposing dying cells, debris and microparticles to phagocytes and therefore triggering efferocytosis. In addition, the therapeutic fusion proteins of the present disclosure have improved developability, in particular reduced stickiness and improved solubility compared to the wild-type MFG-E8 protein (SEQ ID NO: 1), or to recombinant MFG-E8 and C2-truncated MFG-E8 (EGF_C1). Furthermore, these therapeutic fusion proteins have a longer plasma exposure and have a higher yield when expressed in cell expression systems when compared to the wild-type MFG-E8 protein. The therapeutic fusion proteins according to the invention have increased macrophage-selective activity (enhancement of efferocytosis). Moreover, the therapeutic fusion proteins according to the invention have improved safety compared to full length, wild-type MFG-E8 or other full length functional variants.

Provided herein are therapeutic fusion proteins for enhancing efferocytosis comprising an integrin binding domain, a phosphatidylserine (PS) binding domain and a solubilizing domain, wherein the PS binding domain is a truncated variant of at least one PS binding domain listed in Table 2.

In some embodiments, the therapeutic fusion protein comprises the C-terminus of an integrin binding domain linked to the N-terminus of a solubilizing domain, and the C-terminus of the solubilizing domain linked to a PS binding domain. In some embodiments, the therapeutic fusion protein comprises the general structure EGF-S-C wherein EGF represents the integrin binding domain, e.g. EGF-like domain of MFG-E8, of EDIL3 or of any other protein comprising an integrin binding domain as listed in Table 1; S represents a solubilizing domain; and C represents a truncated PS binding domain, e.g. a truncated variant of the PS binding domain found in MFG-E8, EDIL3 or in any other protein comprising any of C1 and/or C2 of a PS binding domain as listed in Table 2. Examples of proteins comprising both an integrin binding domain and a PS binding domain, for example, MFG-E8 (SEQ ID NO: 1) and EDIL3 (SEQ ID NO: 11), are listed in Table 3.

In some embodiments, the PS binding domain comprises one of the two discoidin C1-C2 sub-domains, or a functional variant thereof. For example, the PS binding domain of human MFG-E8 having an amino acid sequence as set forth in SEQ ID NO: 3 or an amino acid of at least 90%, 95%, 96%, 97%, 98% or 99% sequence identity thereto, or truncated variants thereof. In one embodiment, the truncated PS binding domain comprises a truncated PS binding domain of human MFG-E8 or a functional variant thereof comprising one, two, three, four, five, up to 10 amino acid modifications. In one embodiment, the PS binding domain comprises a truncated PS binding domain of human EDIL3 or a functional variant thereof comprising one, two, three, four, five, up to 10 amino acid modifications.

In certain aspects, provided herein is a fusion protein comprising an epidermal growth factor (EGF)-like domain, a solubilizing domain, a C1 domain, but lacking a functional C2 domain. In some embodiments, the fusion protein comprises an epidermal growth factor (EGF)-like domain, a solubilizing domain, a C1 domain, but lacking a medin polypeptide or a fragment thereof.

In some embodiments, the solubilizing domain of the fusion protein is linked to the integrin binding domain. In some embodiments, the solubilizing domain is linked to the PS binding domain. In some embodiments, the solubilizing domain is linked to both the integrin binding domain and the PS binding domain, i.e. is located between the integrin binding domain and the PS binding domain. In some embodiments, the solubilizing domain is inserted within the integrin binding domain or is inserted within the PS binding domain. In one embodiment, the therapeutic fusion protein has the structure from N- to C-terminal: integrin binding domain-solubilizing domain-PS binding domain.

In some embodiments, the integrin binding domain of the therapeutic fusion protein comprises an Arginine-Glycine-Aspartic acid (RGD) binding motif and binds to αvβ3 and/or αvβ5 or α8β1 integrin(s).

In some embodiments, the solubilizing domain of the therapeutic fusion protein is linked directly to the integrin binding domain and/or linked to the PS binding domain i.e. is inserted between said domains. In an alternative embodiment, the solubilizing domain is linked indirectly to the integrin binding domain and/or the PS binding domain by a linker, such as an external linker. In some embodiments, the solubilizing domain comprises human serum albumin (HSA), domain 3 of HSA (HSA D3) or the Fc region of an IgG (Fc-lgG), or a functional variant thereof.

In some embodiments, the integrin binding domain is an EGF-like domain, for example, having an amino acid sequence as set forth in SEQ ID NO: 2 or an amino acid of at least 90%, 95%, 96%, 97%, 98% or 99% sequence identity thereto, or truncated variants thereof. In one embodiment, the EGF-like domain comprises the EGF-like domain of human MFG-E8 or a functional variant thereof comprising one, two, three, four, five, up to 10 amino acid modifications. In one embodiment, the EGF-like domain comprises the EGF-like domain of human EDIL3 or a functional variant thereof comprising one, two, three, four, five, up to 10 amino acid modifications.

In some embodiments, the solubilizing domain is HSA or a functional variant thereof, for example, having an amino acid sequence as set forth in SEQ ID NO: 4 or an amino acid of at least 90%, 95%, 96%, 97%, 98% or 99% sequence identity thereto, or truncated variants thereof. In one embodiment the HSA comprises the amino acid substitution C34S that functions to lower the propensity of the protein to aggregation, and has the amino acid sequence as set forth in SEQ ID NO: 5. In some embodiments, the solubilizing domain comprises human serum albumin (HSA) or a functional variant thereof comprising one, two, three, four, five, up to 10 amino acid modifications, for example, HSA C34S, or a truncated variant of HSA, for example, domain 3 of HSA (HSA D3) or a functional variant thereof. In a preferred embodiment, the solubilizing domain is HSA C34S.

In an alternative embodiment, the solubilizing domain comprises the Fc region of an IgG (Fc-IgG), for example the Fc region of a human IgG1, IgG2, IgG3 or IgG4 or a functional variant thereof. In one embodiment the solubilizing domain comprises the Fc region of a human Fc-lgG1 having an amino acid sequence as set forth in SEQ ID NO: 7 or an amino acid of at least 90%, 95%, 96%, 97%, 98% or 99% sequence identity thereto, or truncated variants thereof. In one embodiment, the Fc-lgG1 comprises the amino acid substitutions D265A and P329A to reduce Fc effector function, and has the amino acid sequence as set forth in SEQ ID NO: 8. In another embodiment, the Fc-lgG1 comprises the amino acid substitution T366Wto create a ‘knob’ or it may comprise the amino acid substitutions T366S, L368A, Y407V to create a ‘hole’. In addition, the Fc-lgG1 knob may comprise the amino acid substitution S354C and the Fc-lgG1 hole may comprise the amino acid substitution Y349C so that on pairing a cysteine bridge is formed. In addition to the knob in hole modifications, the Fc-lgG1 may also comprise the D265A and P329A substitutions to reduce Fc effector function. In one embodiment, the Fc-lgG1 has the amino acid sequence as set forth in SEQ ID NO: 9 or 10.

In a preferred embodiment, the therapeutic fusion protein comprises milk fat globule-EGF factor 8 protein (MFG-E8) and a solubilizing domain, wherein MFG-E8 comprises an integrin binding EGF-like domain (SEQ ID NO: 2) and a functional variant of the phosphatidylserine binding C1-C2 domains (SEQ ID NO: 3 or SEQ ID NO: 76). The MFG-E8 may comprise naturally occurring or wild-type human MFG-E8 (SEQ ID NO: 1), or MFGE-8 with SEQ ID NO: 75, or a functional variant thereof. In one embodiment, the solubilizing domain is linked to the N or C-terminal of MFG-E8. In one embodiment, the solubilizing domain is inserted between the EGF-like domain and C1 domain or between the EGF-like domain and the C2 domain. In a preferred embodiment, the solubilizing domain is linked to the C-terminus of the EGF-like domain and linked to the N-terminus of the C1 domain. The solubilizing domain may be linked directly or indirectly to the C-terminal of the EGF-like domain and linked directly or indirectly to the N-terminus of the C1 domain. In some embodiments, the indirect linkage is by means of an external linker, for example a glycine-serine based linker.

In some embodiments, and as described in the Examples section, the therapeutic fusion proteins of the present disclosure function to promote efferocytosis by endothelial cells in a human endothelial cell-Jurkat cell efferocytosis assay and restore impaired and boost basal efferocytosis by macrophages in a human macrophage-neutrophil efferocytosis assay; the fusion proteins function to reduce numbers of plasma microparticles by clearance in a human endothelial-microparticle efferocytosis assay; and/or the fusion proteins provide protection against multi-organ injury in an acute kidney ischaemia model.

Also disclosed herein are methods, uses, diagnostic reagents, pharmaceutical compositions and kits utilizing or comprising these therapeutic fusion proteins. Also provided herein are nucleic acids encoding the disclosed fusion proteins, cloning and expression vectors comprising such nucleic acids, host cells comprising such nucleic acids, and processes of producing the disclosed fusion proteins by culturing such host cells.

### BRIEF DESCRIPTION OF THE FIGURES

FIG. 1 shows a schematic representation of examples of therapeutic fusion proteins of the present disclosure. A solubilizing domain (labelled ‘SD’) was linked at either the C-terminus, the N-terminus, or between the EGF, C1 or C2 domains of MFG-E8.

FIGS. 2 shows a number of SDS-PAGE protein gels of the fusion proteins expressed in HEK cells. FIG. 2A: EGF-HSA-C1-C2 protein (FP330; SEQ ID NO: 42); FIG. 2B: EGF-HSA-C1-C2 of EDIL3 protein (FP050; SEQ ID NO: 12); FIG. 2C: EGF-Fc(KiH) C1-C2 protein non-reduced and reduced (this protein is a heterodimer of FP071 (EGF-Fc(knob)-C1-C2; SEQ ID NO: 18) with Fc-IgG1 hole (SEQ ID NO: 10)); FIG. 2D: EGF-HSA-C1 protein (FP260; SEQ ID NO: 34). For each of FIGS. 2A, 2C and 2D, the first column shows a Precision Plus protein unstained standards marker and the second column shows the respective fusion protein. For FIG. 2B, the first column shows the fusion protein and the second column shows a Precision Plus protein unstained standards marker. FIG. 2E shows further recombinant proteins which have been produced and purified.

FIGS. 3 exemplifies the effect of loss of wild type (wt) MFG-E8 versus the fusion protein FP278 (EGF-HSA-C1-C2-His tag; SEQ ID NO: 44) protein during practical handling. FIG. 3A shows a loss of efficacy for wtMFG-E8 in the L-α-phosphatidylserine competition assay when protein dilutions were made in polypropylene plates (symbol: □) in comparison to dilutions made in non-binding plates (symbol: •). In contrast, FIG. 3B shows virtually no loss of efficacy for the fusion protein FP278 (EGF-HSA-C1-C2-His tag; SEQ ID NO: 44) in the PS competition assay when protein dilutions were made in polypropylene plates (symbol: □) versus non-binding plates (symbol: •).

FIGS. 4 shows binding of fusion proteins to L-α-phosphatidylserine. FIG. 4A shows binding of FP278 (EGF-HSA-C1-C2-His tag; SEQ ID NO: 44) to immobilized L-α-phosphatidylserine and to a weaker extent to the phospholipid cardiolipin, in a concentration dependent manner. FIG. 4B shows binding of human wtMFG-E8 and a number of therapeutic fusion proteins: FP278 (EGF-HSA-C1-C2-His tag; SEQ ID NO: 44), FP250 (EGF-HSA; SEQ ID NO: 32), FP260 (EGF-HSA-C1; SEQ ID NO: 34), and FP270 (EGF-HSA-C2; SEQ ID NO: 36), to immobilized L-α-phosphatidylserine in a concentration dependent manner in a competition assay format (competition against binding of biotinylated mouse wtMFG-E8 to L-α-phosphatidylserine).

FIGS. 5 shows αv-integrin-dependent cell adhesion to fusion proteins. FIG. 5A shows that cell adhesion to FP330 (EGF-HSA-C1-C2; SEQ ID NO: 42) is completely blocked by the αv integrin inhibitor cilengitide or 10 mM EDTA. A single point mutation in the integrin binding motif RGD (RGD > RGE) of the EGF-like domain (FP280; SEQ ID NO: 38) results in complete abrogation of cell adhesion as shown in FIG. 5B. FIG. 5C shows that immobilized EGF-HSA protein (FP250; SEQ ID NO: 32) does not or only moderately supports adhesion of BW5147.G.1.4 cells despite an EGF-like domain. As shown in FIG. 5D, a fusion protein of this disclosure (FP330; SEQ ID NO: 42) promotes αv-integrin-dependent cell adhesion similar to wtMFG-E8 when expressed in CHO cells or in HEK cells.

FIG. 6 shows the effect of the therapeutic fusion protein FP278 (EGF-HSA-C1-C2-His tag; SEQ ID NO: 44) on the promotion of efferocytosis of dying neutrophils by human macrophages. Concentration of the fusion protein is shown on the x-axis and efferocytosis [%] is shown on the y-axis.

FIGS. 7 shows that the therapeutic fusion protein FP278 (EGF-HSA-C1-C2-His tag; SEQ ID NO: 44) can rescue endotoxin (lipopolysaccharide)-impaired efferocytosis of dying neutrophils by human macrophages. FIG. 7A shows the impairment of macrophage efferocytosis of dying human neutrophils by 100 pg/ml lipopolysaccharide (LPS) in three human donors. The left panel shows the individual donor response, the right panel shows the mean impairment of efferocytosis (%) of the three donors. FIG. 7B shows the rescue of this endotoxin (LPS)- impaired efferocytosis of dying neutrophils by human macrophages in the presence of the therapeutic fusion protein FP278. Efferocytosis indices of 3 different human macrophage donors were normalized and plotted as efferocytosis (%).

FIGS. 8 shows the rescue of S. aureus particle induced impairment of efferocytosis of dying neutrophils by human macrophages with the therapeutic fusion protein FP278 (EGF-HSA-C1-C2-His tag; SEQ ID NO: 44). FIG. 8A shows the effect of a concentration of 100 nM of FP278 on promoting efferocytosis over the base level (dotted line; left-hand part of figure) as well as the effect of 100 nM FP278 in rescuing the impairment of efferocytosis caused by the administration of S. aureus (right-hand part of figure). FIG. 8B shows the effect of increasing concentrations of fusion protein FP278 (EC₅₀ 8 nM) on the rescue of impaired efferocytosis caused by the administration of S. aureus, and on the promotion of efferocytosis once the base levels of efferocytosis had been reached.

FIG. 9 shows the effect of the therapeutic fusion protein FP278 (EGF-HSA-C1-C2-His tag; SEQ ID NO: 44) on the promotion of efferocytosis of dying Jurkat cells by human endothelial cells (HUVEC). Efficiency of the fusion protein in the endothelial cell efferocytosis assay depends on the presence of a C1-C2 or C1-C1 tandem domain since, as illustrated in FIG. 9, a fusion protein of structure EGF-HSA-C2 (FP270; SEQ ID NO: 36) is ineffective in this assay.

FIG. 10 shows that the location of a HSA domain in the therapeutic fusion protein, namely in the N-or C-terminal position (FP220 (HSA-EGF-C1-C2; SEQ ID NO: 30) or FP110 (EGF-C1-C2-HSA; SEQ ID NO: 28), respectively), confers efferocytosis blocking function to the MFG-E8 HSA fusion protein in the macrophage efferocytosis assay. Concentration of fusion protein is shown on the x-axis, efferocytosis [%] is shown on the y-axis.

FIGS. 11 shows a comparison of the promotion of efferocytosis by various formats of therapeutic fusion proteins comprising a HSA or Fc moiety. Concentration of the fusion protein is shown on the x-axis (nM), efferocytosis [MFI] is shown on the y-axis. FIG. 11A shows a comparison of fusion proteins comprising HSA with the HSA positioned at the C-terminal or N-terminal or between the EGF-like and C1 domains; FP110 (EGF-C1-C2-HSA; SEQ ID NO: 28), FP220 (HSA-EGF-C1-C2; SEQ ID NO: 30) and FP278 (EGF-HSA-C1-C2-His tag; SEQ ID NO: 44), respectively. FIG. 11B shows a comparison of fusion proteins comprising a Fc moiety with the Fc positioned at the C-terminal (FP060 (EGF-C1-C2-Fc [S354C,T366W]; SEQ ID NO: 14) and FP080 (EGF-C1-C2-Fc; SEQ ID NO: 22)) or between the EGF-like and C1 domains (FP070 (EGF-Fc-C1-C2; SEQ ID NO: 16)) compared to wild-type MFG-EG (SEQ ID NO: 1). Two formats of Fc moiety are shown: wild-type Fc (FP080; SEQ ID NO: 22) and a Fc moiety with the modifications S354C and T366W (EU numbering; FP060; SEQ ID NO: 14). FIG. 11C shows a comparison of three batches of the fusion protein FP090 (Fc-EGF-C1-C2; SEQ ID NO: 24) comprising a Fc moiety positioned at the N-terminal, at three different concentrations (0.72, 7.2 and 72 nM), compared to wt-MFG-E8 control. FIG. 11D shows the promotion of efferocytosis by a fusion protein construct FP050 comprising a HSA inserted between the EGF-like domain and the C1-C2 domain of EDIL3 (EDIL3 based EGF-HSA-C1-C2; SEQ ID NO: 12). FIG. 11E shows further examples of fusion proteins of the disclosure, for example chimeric variants (FP114 or FP260; SEQ ID NO: 34, FP147 or FP1777; SEQ ID NO: 71, FP1149, FP1150, FP145; SEQ ID NO: 80, FP1145; SEQ ID NO: 103, FP146; SEQ ID NO: 82, FP1146) and combinations of the integrin binding domains of MFGE8 or EDIL3 and PS binding domains such as the IgSF V domain of TIM4 or the GLA domain of the bridging protein GAS6 (FP1147 and FP1148).

FIG. 12 shows the promotion of efferocytosis by HUVEC cells of the therapeutic fusion protein FP278 (EGF-HSA-C1-C2-His tag; SEQ ID NO: 44) tested at 3 different concentrations up to 30 nM. The promotion of efferocytosis was concentration-dependent with efferocytosis increasing as the concentration of the fusion protein FP278 increased.

FIGS. 13 shows that the therapeutic fusion proteins FP330 (EGF-HSA-C1-C2; SEQ ID NO: 42; FIG. 13A), FP278 (EGF-HSA-C1-C2-His tag; SEQ ID NO: 44; FIG. 13B) and FP776 (EGF-HSA-C1-C2; SEQ ID NO: 48; FIG. 13C) can rescue endotoxin (lipopolysaccharide)-impaired efferocytosis of dying neutrophils by human macrophages. Concentration of fusion protein is shown on the x-axis, efferocytosis [%] is shown on the y-axis.

FIGS. 14 shows the effect of the fusion proteins FP330 (EGF-HSA-C1-C2; SEQ ID NO: 42; FIG. 14A), FP278 (EGF-HSA-C1-C2-His tag; SEQ ID NO: 44; FIG. 14B) and FP776 (EGF-HSA-C1-C2; SEQ ID NO: 48; FIG. 14C) on the promotion of efferocytosis of dying Jurkat cells by human endothelial cells (HUVEC). Concentration of fusion protein is shown on the x-axis, efferocytosis [%] is shown on the y-axis.

FIGS. 15 shows that a single dose of the therapeutic fusion proteins FP278 (EGF-HSA-C1-C2-His tag; SEQ ID NO: 44), FP330 (EGF-HSA-C1-C2; SEQ ID NO: 42) or FP776 (EGF-HSA-C1-C2; SEQ ID NO: 48) protects kidney function in a model of ischemia-reperfusion injury-induced acute kidney injury (AKI). FIG. 15A shows that a raise in serum creatinine (sCr) (mg/dL; y-axis) is reduced by intraperitoneal (i.p.) administration of 0.16 mg/kg or 0.5 mg/kg of FP278 (SEQ ID NO: 44) (x-axis). As shown in FIG. 15B, intravenous (i.v.) administration of 0.5 mg/kg or 1.5 mg/kg of the fusion protein FP330 (SEQ ID NO: 42) reduced serum creatinine levels significantly. FIG. 15C shows that i.v. administration of the fusion protein FP776 (SEQ ID NO: 48) reduced serum creatinine in a dose-dependent manner.

FIG. 16 shows that a single dose of the therapeutic fusion protein FP278 (EGF-HSA-C1-C2-His tag; SEQ ID NO: 44) of either 0.16 mg/kg or 0.5 mg/kg, reduced blood urea nitrogen (BUN) levels in a murine model of acute kidney injury.

FIGS. 17 shows that a single dose of the therapeutic fusion protein FP278 (EGF-HSA-C1-C2-His tag; SEQ ID NO: 44) protects distant organs from acute phase response elicited by ischemia reperfusion-induced AKI, based on gene expression of markers of injury. FIG. 17A exemplifies such AKI-induced response of serum amyloid protein (SAA) in the murine heart and FIG. 17B exemplifies such AKI-induced response (SAA) in the murine lung, both of which were potently blocked after single i.p. injection of the MFG-E8-derived fusion protein FP278 (SEQ ID NO: 44) at 0.16 mg/kg or 0.5 mg/kg/i.p.

FIG. 18 shows the uptake of superparamagnetic iron oxide (SPIO) contrast agent (Endorem®) by the liver over time. Endorem® was injected intravenously as a bolus for 1.2 s into animals with AKI (at 24 h post disease induction) or after Sham operation (animals post 24 h\ nephrectomy). Animals with AKI showed significantly reduced uptake of the contrast agent by the liver (target = Kupffer cells) compared to Sham animals. Treatment with the fusion protein FP776 (EGF-HSA-C1-C2; SEQ ID NO: 48) dosed prophylactically -30 min before AKI induction, or dosed therapeutically +5 h post ischemia reperfusion injury induction, protected from the loss of contrast agent accumulation in the liver of AKI mice.

FIG. 19 The therapeutic fusion proteins FP114, also named herein FP260, (EGF-HSA-C1 SEQ ID No: 34) was tested in the AKI model as described in the Examples at 1.5 mg/kg/i.v. For this study FP114 was administered 30 min hours before ischemia reperfusion injury onset. Serum markers and kidney weight were assessed 24 h post induction of disease. Reduced serum creatinine and BUN as well as normal kidney weight suggest protection from AKI in this model.

FIG. 20 The therapeutic fusion protein FP135, also named herein FP261, (EGF-HSA-C1 SEQ ID No: 73) was tested in the CCL4 fibrosis model at 0.8 mg/kg/i.p. Treatment started either after 4 weeks of fibrosis induction (with CCL4) (total of 11 doses) or after 5 weeks of fibrosis induction with CCL4 (total of 8 doses) with 3 weekly doses administered. The third group of animals was dosed after 6 weeks at stop of disease induction with CCL4 (total of 4 doses). In all groups, FP135 was dosed once daily during the last 3 days. Liver stiffness was assessed at day of baseline (at start of experiment) at cessation of CCL4 and 3 days after cessation of CCL4. The data suggest that in animals which were treated with FP135 (start at after week 4 and 5 of CCI4) significant accelerated resolution of liver stiffness induced by CCL4 was achieved.

FIGS. 21. FIG. 21A The therapeutic fusion protein FP135 (EGF-HSA-C1 SEQ ID No: 73) was tested in the CCL4 fibrosis model at 0.8 mg/kg/i.p. Treatment started either after 4 weeks of fibrosis induction (with CCL4) (total of 11 doses) or after 5 weeks fibrosis induction with CCL4 (total of 8 doses) with 3 weekly doses administered or after 6 weeks at stop of disease induction with CCL4 (total of 4 doses). In all groups, FP135 was dosed once daily during the last 3 days. The reduction of serum ALT suggest that treatment with FP135 helped to accelerate the resolution of liver damage caused by CCL4 in the groups in which treatment was started at week 4 and 5 of CCI4.

FIG. 21B The therapeutic fusion protein FP135 (EGF-HSA-C1 SEQ ID No: 73) was tested in the CCL4 fibrosis model at 0.8 mg/kg/i.p. as described for FIG. 21A The collagen content in livers of sacrificed animals was quantified by hydroxyproline assay. The reduction observed in 8 and 11 times dosed animals suggest that treatment with FP135 helped to accelerate the resolution of liver fibrosis caused by CCL4

FIG. 21C The therapeutic fusion protein FP135 (EGF-HSA-C1 SEQ ID No: 73) was tested in the CCL4 fibrosis model at 0.8 mg/kg/i.p. as described for FIG. 21A. The collagen expression in livers of sacrificed animals was quantified by hydroxyproline assay. The reduction observed in 8 and 11 times dosed animals suggest that treatment with FP135 helped to accelerate the resolution of liver fibrosis caused by CCL4.

FIG. 22 shows Integrin adhesion data for section of truncated proteins FP137, FP135 and FP147.

FIG. 23 shows dynamic light scattering (DLS) of C2-truncated MFG-E8 (EGF-C1; SEQ ID NO: 115) and HSA fusion (EGF-HSA-C1; SEQ ID NO: 73).

### DETAILED DESCRIPTION

Disclosed herein are therapeutic fusion proteins comprising an integrin binding domain, a PS binding domain and a solubilizing domain. Also disclosed herein are methods of treatment using the fusion proteins of the disclosure as well as assays, such as an efferocytosis assay, useful for the characterization of the fusion proteins.

### Definitions

In order that the present disclosure may be more readily understood, certain terms are specifically defined throughout the detailed description. Unless defined otherwise, all technical and scientific terms used herein have the same meaning as commonly understood by those of ordinary skill in the art to which this disclosure pertains.

In all cases where the term ‘comprise’, ‘comprises’, ‘comprising’ or the like are used in reference to a sequence (e.g., an amino acid sequence), it shall be understood that said sequence may also be limited by the term ‘consist’, ‘consists’, ‘consisting’ or the like. As used herein, the phrase ‘consisting essentially of’ refers to the genera or species of active pharmaceutical agents included in a method or composition, as well as any excipients inactive for the intended purpose of the methods or compositions. In some aspects, the phrase ‘consisting essentially of’ expressly excludes the inclusion of one or more additional active agents other than a multi-specific binding molecule of the present disclosure. In some aspects, the phrase ‘consisting essentially of’ expressly excludes the inclusion of one or more additional active agents other than a multi-specific binding molecule of the present disclosure and a second co-administered agent.

The term ‘efferocytosis’ as used herein refers to a process in cell biology, wherein dying or dead cells, such as apoptotic or necrotic or aged cells or highly activated cells or extracellular cellular vesicles (microparticles) or cellullar debris- collectively called “prey” - are removed by phagocytosis, i.e. are engulfed by a phagocytic cell and digested. During efferocytosis, the phagocytic cells actively tether and engulf the prey, generating intracellular large fluid-filled vesicles containing the prey called an efferosome, resulting in a lysosomal compartment where degradation of prey is initiated. During apoptosis, efferocytosis ensures that the dying cells are removed before their membrane integrity is compromised and their contents could leak into the surrounding tissues preventing the exposure of the surrounding tissues to DAMPs such as toxic enzymes, oxidants and other intracellular components such as DNA, histones, and proteases. Professional phagocytic cells include cells of myeloid origin such as macrophages and dendritic cells but other, e.g. stromal cells, can also perform efferocytosis such as epithelial and endothelial cells and fibroblasts. Impaired efferocytosis has been linked to autoimmune diseases and tissue damage and has been demonstrated in diseases such as cystic fibrosis, bronchiectasis, COPD, asthma, idiopathic pulmonary fibrosis, rheumatoid arthritis, systemic lupus erythematosus, glomerulonephritis and atherosclerosis (Vandivier RW et al (2006) Chest, 129(6): 1673-82). No therapy that specifically promotes efferocytosis has entered clinics as of today.

The term ‘efferocytosis assay’ as used herein and as described in the Examples relates to an assay system developed for the profiling of fusion proteins, which utilizes human macrophages or human endothelial cells (HUVECs) as phagocytic cells. Exemplified herein are a macrophage-neutrophil efferocytosis assay, an endothelial cell-Jurkat cell efferocytosis assay or an endothelial-cell microparticle efferocytosis assay. These assays, as described in more detail in the Examples, can be used to demonstrate that MFG-E8-derived biotherapeutics such as the fusion proteins of the present disclosure, effectively promote efferocytosis of dying cells and microparticles by macrophages or endothelial cells. Furthermore, the described macrophage-neutrophil assay is suitable to demonstrate that such compounds of this invention can even rescue LPS or S. aureus impaired efferocytosis of dying cells.

The terms ‘polypeptide’ and ‘protein’ are used interchangeably herein to refer to a polymer of amino acid residues. The phrases also apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term ‘stickiness’ as used herein in relation to proteins of the present disclosure refers to a result of protein misfolding which promotes protein clumping or aggregation. These unwanted and nonfunctional effects are a result of surface hydrophobic interactions.

As used herein, ‘C-terminus’ refers to the carboxyl terminal amino acid of a polypeptide chain having a free carboxyl group (—COOH). As used herein, ‘N-terminus’ refers to the amino terminal amino acid of a polypeptide chain having a free amine group (—NH2).

As used herein, the term ‘fusion protein’ refers to a protein comprising a number of domains, which may not constitute an entire natural or wild-type protein but may be limited to an active domain of the entire protein responsible for binding to a corresponding receptor on the surface of a cell. The fusion proteins can be generated using recombinant protein design, where the term ‘recombinant protein’ refers to a protein that has been prepared, expressed, created, or isolated by recombinant DNA technology means. Tandem fusion, for example, refers to a technique whereby the proteins or protein domains of interest are simply connected end-to-end via fusion of N or C termini between the proteins. This provides a flexible bridge structure allowing enough space between fusion partners to ensure proper folding. However, the N or C terminus of the peptide are often crucial components in obtaining the desired folding pattern for the recombinant protein, with the effect that simple end-to-end conjoining of domains can be ineffective. Alternatively, the process of domain insertion involves the fusion of consecutive protein domains by encoding desired structures into a single polypeptide chain and sometimes the insertion of a domain within another domain. In both these afore mentioned processes the domains are ‘directly linked’ or ‘linked directly’. Domain insertion is often more difficult to carry out than tandem fusion due to the difficulty in finding an appropriate ligation site in the gene of interest.

In addition to the aforementioned fusion techniques of direct linkage, an external linker may be used to maintain the functionality of the protein domains in the fusion protein. Such a linker, refers to a stretch of amino acids that connects a protein domain to another protein domain and is referred to herein as an ‘indirect linker’. As such the domains are ‘indirectly linked’ or ‘linked indirectly’. For example, those of ordinary skill in the art appreciate that a polypeptide whose structure includes two or more functional or organizational domains often includes a stretch of amino acids between such domains that links them to one another. The linker permits domain interactions, reinforces stability and can reduce steric hindrance, which often makes them preferred for use in engineered protein design even when N and C termini can be fused. In some embodiments, a linker is characterized in that it tends not to adopt a rigid three-dimensional structure but rather provides flexibility to the polypeptide. Various types of naturally occurring linkers have been used in engineered proteins, for example, the immunoglobulin hinge region, which functions as a linker in many recombinant therapeutic proteins, particularly in engineered antibody constructs (Pack P et al., (1995) J. Mol. Biol.,246: 28-34). Besides natural linkers, a multitude of artificial linkers have been devised, which can be subdivided into three categories: flexible, rigid and in vivo cleavable linkers. (Yu K et al., (2015) Biotech. Advances, 33(1): 155-64; Chen X et al., (2013) Ad. Drug Delivery Reviews, 65(10): 1357-69). The most widely used flexible linker sequences are (Gly)n (Sabourin et al., (2007) Yeast, 24: 39-45) and (Gly₄Ser)n (SEQ ID NO: 64) (Huston et al., 1988, 85: 5879-83) where linker length can be adjusted by the copy number “n”. In some embodiments, a polypeptide comprising a linker element has an overall structure of the general form D1-linker-D2, wherein D1 and D2 may be the same or different and represent two domains associated with one another by the linker. In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length.

A ‘modification’ or ‘mutation’ of an amino acid residue/position, as used herein, refers to a change of a primary amino acid sequence as compared to a starting amino acid sequence, wherein the change results from a sequence alteration involving said amino acid residue/positions. For example, typical modifications include substitution of the residue (or at said position) with another amino acid (e.g., a conservative or non-conservative substitution), insertion of one or more amino acids adjacent to said residue/position, and deletion of said residue/position. An amino acid ‘substitution’ or variation thereof, refers to the replacement of an existing amino acid residue in a predetermined (starting) amino acid sequence with a different amino acid residue. Generally and preferably, the modification results in alteration in at least one physicobiochemical activity of the variant polypeptide compared to a polypeptide comprising the starting (or ‘wild-type’) amino acid sequence.

The term ‘conservatively modified variant’ applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are ‘silent variations’, which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, ‘conservatively modified variants’ include individual substitutions, deletions or additions to a polypeptide sequence which result in the substitution of an amino acid with a chemically similar amino acid. Conservative substitution tables providing functionally similar amino acids are known in the art. Such conservatively modified variants are in addition to and do not exclude polymorphic variants, interspecies homologs, and alleles. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the phrase ‘conservative sequence modifications’ are used to refer to amino acid modifications that do not significantly affect or alter the binding characteristics of the binding domains of the engineered proteins of the present disclosure.

A ‘protein variant’ or ‘variant of a protein’ as referred to herein, relates to a protein comprising a variation in which one or more, for example, 2, 3, 4, 5, 6, 7, 8, 9, 10 amino acids have been modified. A ‘functional variant’ of a protein as referred to herein, relates to a protein variant comprising a modification that results in a change to the amino acid sequence but there is no change to the overall property of the protein or to its function. A ‘truncated variant’ of a protein, or of a domain of a protein, as referred to herein, relates to a shortened version of a protein, or of the protein domain, but the shortened version of the protein retains the function of the parent protein. To determine whether a functional variant or truncated variant has no change in the overall property or function, these variant proteins can be tested against a full length or unmodified parent protein for their effect in a number os assays as described in the present disclosure. For example, promoting efferocytosis by endothelial cells in a human endothelial cell-Jurkat cell efferocytosis assay, restoring impaired efferocytosis by macrophages in a human macrophage-neutrophil efferocytosis assay, reducing the number of plasma microparticles by clearance in a human endothelial-microparticle efferocytosis assay, and/or providing protection against multi-organ injury in an acute kidney ischaemia model.

The terms ‘percentage identity’ or ‘percentage sequence identity’ in the context of two or more nucleic acids or polypeptide sequences, refers to two or more sequences or subsequences that are the same. Two sequences are ‘substantially identical’ and show ‘sequence identity’ if two sequences have a specified percentage of amino acid residues or nucleotides that are the same (i.e., at least 60% identity, optionally at least 65%, 70%, 75%, 80%, 85%, 90%, 95%, 96%, 97%, 98% or 99% identity over a specified region, or, when not specified, over the entire sequence), when compared and aligned for maximum correspondence over a comparison window, or designated region, e.g. as measured using one of the following sequence comparison algorithms or by manual alignment and visual inspection. Optionally, the identity exists over a region that is at least about 50 nucleotides (or 10 amino acids) in length, or over a region that is 100 to 500 or 1000, or 2000 or 3000 or more nucleotides in length, or alternatively, 30 to 200, or 300, or 500, or 700 or 800 or 900 or 1000 or more amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The term ‘comparison window’ as used herein includes reference to a segment of any one of the number of contiguous nucleic acid or amino acid positions selected from the group comprising of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known in the art. Optimal alignment of sequences for comparison can be conducted, e.g., by the local homology algorithm of Smith and Waterman (1970) Adv. Appl. Math. 2:482c, by the homology alignment algorithm of Needleman & Wunsch (1970) J. Mol. Biol., 48: 443, by the search for similarity method of Pearson & Lipman (1988) PNAS USA, 85: 2444, by computerized implementations of these algorithms (GAP, BESTFIT, FASTA, and TFASTA in the Wisconsin Genetics Software Package, Genetics Computer Group, 575 Science Dr., Madison, Wl), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res,. 25: 3389-3402; and Altschul et al., (1990) J. Mol. Biol., 215: 403-410, respectively. Software for performing BLAST analyses is publicly available through the National Center for Biotechnology Information.

The BLAST algorithm also performs a statistical analysis of the similarity between two sequences (see, e.g., Karlin & Altschul (1993) PNAS. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci. 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman & Wunsch (supra) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

A polypeptide is typically substantially identical to a second polypeptide, for example, where the two peptides differ only by conservative substitutions. Another indication that two nucleic acid sequences are substantially identical is that the two molecules or their complements hybridize to each other under stringent conditions.

The term ‘nucleic acid’ is used herein interchangeably with the term ‘polynucleotide’ and refers to deoxyribonucleotides or ribonucleotides and polymers thereof in either single- or doublestranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., (1991) Nucleic Acid Res., 19: 5081; Ohtsuka et al., (1985) J Biol Chem., 260: 2605-2608; and Rossolini et al., (1994) Mol Cell Probes, 8: 91-98). As used herein, the term, optimized nucleotide sequence’ means that the nucleotide sequence has been altered to encode an amino acid sequence using codons that are preferred in the production cell, e.g. a Chinese Hamster Ovary cell (CHO). The optimized nucleotide sequence is engineered to retain completely the amino acid sequence originally encoded by the starting nucleotide sequence, which is also known as the ‘parental’ sequence. In particular embodiments, the optimized sequences herein have been engineered to have codons that are preferred in CHO mammalian cells.

### THERAPEUTIC FUSION PROTEINS

### Integrin Binding Domains

Integrins are transmembrane receptors that facilitate cell-extracellular matrix (ECM) adhesion. Upon ligand binding, integrins activate signal transduction pathways that mediate cellular signals such as regulation of the cell cycle, organization of the intracellular cytoskeleton, and movement of new receptors to the cell membrane (Giancotti & Ruoslahti (1999) Science, 285 (5430): 1028-32). The presence of integrins allows rapid and flexible responses to events at the cell surface. Several types of integrins exist, and one cell may have multiple different types on its surface. Integrins have two subunits: α (alpha) and β (beta), which each penetrate the plasma membrane and possess several cytoplasmic domains (Nermut MV et al (1988). EMBO J., 7 (13): 4093-9). An acidic amino acid features in the integrin-interaction site of many ECM proteins, for example as part of the amino acid sequence Arginine-Glycine-Aspartic acid (‘RGD’ in the one-letter amino acid code). The RGD motif has been found in numerous matrix proteins such as fibronectin, fibrinogen, vitronectin and osteopontin and aids in cell adhesion. The RGD motif is found in a number of proteins in a conserved protein domain known as an EGF-like domain, which derived its name from epidermal growth factor where it was first described. The EGF-like domain is one of most common domains found in extracellular proteins (Hidai C (2018) Open Access J Trans Med Res., 2(2): 67-71) and some examples of EGF-like domains which contain an RGD binding motif are listed below in Table 1.

TABLE 1

Examples of proteins comprising EGF-like domain proteins containing an RGD integrin binding motif

| | | | |
|---|---|---|---|
| Abbreviation | UniProtKB | Name | Reference |
| EDIL3 | O43854 | EGF like repeat and discoidin domain 3 | Schürpf T et al., (2012) |
| MFG-E8 | Q08431 | Milk Fat Globule-EGF Factor 8 Protein | Taylor MR et al., (1997) |
| NRG1 | Q02297 | Neuregulin-1 | Leguchi K et al., (2010) |
| IGFBP-1 | P08833 | Insulin-like growth factor binding protein 1 | Haywood NJ et al., (2017) |
| P2Y2R | P41231 | P2Y2 nucleotide receptor | Erb L et al., (2001) |

The term ‘integrin binding domain’ as used herein refers to a stretch of amino acids, or protein domain, that has the function of binding to integrins In an embodiment of the present disclosure, ‘integrin binding domain’ as used herein refers to a stretch of amino acids, or protein domain, that has the function of binding to integrins and comprising a RGD motif. In an embodiment of the present disclosure, the integrin binding domain is an EGF-like domain from human MFG-E8 having the amino acid sequence as set forth in SEQ ID NO: 2. In an alternative embodiment of the present disclosure, the integrin binding domain is an EGF-like domain from human EDIL3 (any one of the following sequences: SEQ ID NO: 11, SEQ ID NO: 77, SEQ ID NO: 96, SEQ ID NO: 97, SEQ ID NO: 98, SEQ ID NO: 99, SEQ ID NO: 100, or SEQ ID NO: 101); e,g., where the EGF-like domains can be found within the stretch of amino acids 1-132 of SEQ ID NO: 11.

The term ‘binds to integrins’ as used herein refers to an integrin binding activity. Integrin binding activity can be determined by methods well known in the art. For example, an integrin adhesion assay is described in the Examples, section 3.2 in which the adherence of fluorescently labelled αvβ3 integrin-expressing lymphoma cells to therapeutic fusion proteins of the present disclosure was determined. An integrin binding domain is considered to have integrin binding activity if it has at least 10%, such as e.g. at least 25%, at least 50%, at least 75%, more preferably at least 80%, such as at least 90%, at least 95%, at least 96%, at least 97%, at least 98% of the integrin binding activity as observed for the human MFG-E8 protein (SEQ ID NO:1) when tested by the same method of determining the respective activity, preferably when tested using the assay described in the Examples, section 3.2.

### Phosphatidylserine Binding Domains

‘Phosphatidylserine’ (PS), as used herein, relates to the phospholipid, which is a component of the cell membrane. PS is mostly confined to the inner leaflet of the cell membrane, while phosphatidylcholine and sphingomyelin are localized largely to the outer leaflet. The asymmetric distribution of phospholipids is maintained by the action of flippases (P4-ATPases such as ATP11A and 11C) in the plasma membrane to actively translocate PS from the outer leaflet to the inner leaflet. Cell surface exposure of PS is observed not only in apoptotic cells, but also in activated lymphocytes, activated platelets, aged erythrocytes, and some cancer cells and the respective microparticles (Sakuragi et al., (2019) PNAS USA, 116(8): 2907-12). PS exposure can be a biomarker for a prothrombotic, inflammatory or ischemic disease state (Pasalic et al., (2018) J Thromb Haemost., 16(6): 1198-2010; Ma et al., (2017) supra; Zhao et al., (2016) supra. PS has a function in a multitude of cell signaling pathways and as essential phospholipid in coagulation where it can act as enhancer formation of the tenase (factors IXa, Villa and X) and prothrombinase (factors Xa, Va and prothrombin) complexes (Spronk et al., (2014) Thromb Res. 133 (Suppl 1): S54-6). Possibly the most understood function of externalized PS is still the ‘eat-me’ marker for phagocytic cells such as macrophages to engulf apoptotic cells, cell debris or PS-exposing activated cells. The term ‘phosphatidylserine binding domain’ or ‘PS binding domain’ as used herein refers to a stretch of amino acids, or protein domain, that has the function of binding to PS. Examples of endogenous proteins with PS binding domains can be found in Table 2 below.

TABLE 2

Examples of receptors/proteins with phosphatidylserine binding domains

| | | | | |
|---|---|---|---|---|
| Abbreviation | UniProt | Name | Putative PS binding domain | Reference |
| EDIL3 | O43854 | EGF like repeats and discoidin domains 3 | C1-C2 discoidin domains | Dasgupta et al., (2012) |
| MFG-E8 | Q08431 | milk fat globule-EGF factor 8 protein, lactadherin | C1-C2 discoidin domains | Andersen et al., (2000) |
| BAI1 | O14514 | Brain-specific angiogenesis inhibitor 1 | thrombospondin type 1 repeats | Park et al., (2007) |
| TIM1 | Q96D42 | T-cell immunoglobulin and mucin domain-containing protein 1 | IgSF-V domain | Kobayashi et al., (2007) |
| TIM3 | Q8TDQ0 | T-cell immunoglobulin and mucin domain-containing protein 3 | IgSF-V domain | Cao et al., (2007) |
| TIM4 | Q96H15 | T-cell immunoglobulin and mucin domain-containing protein 4 | IgSF-V domain | Kobayashi et al., (2007) |
| Stab1/Stab2 | Q9NY15/ Q8WWQ8 | Stabilin-1 and -2 | EGF-like domain repeats (EGFrps) in the extracellular region | Park SY et al., (2009) |
| TLT2 | Q5T2D2 | Triggering receptor expressed on myeloid cells-like protein 2 | IgSF domain | de Freitas et al., (2012) |
| TREM2 | Q9NZC2 | Triggering receptor expressed on myeloid cells 2 | IgSF-V domain | Takahashi et al., (2005) |
| CD300a | Q9U6N4 | CD300a molecule | IgSF-V domain | Simhadri et al., (2012) |
| RAGE | Q15109 | Receptor for advanced glycation end products | | He et al., (2011) |
| AxV | P08758 | Annexin V | | Ravanat et al., (1992) |
| PSR | | Phosphatidylserine receptor | | Mo et al., (2003) |
| CD36 | P16671 | Platelet glycoprotein 4, | | Banesh et al., (2018) |
| CD68 | P34810 | Scavenger Receptor Class D | | Chistiakov et al., (2017) |

In an embodiment of the present disclosure, the PS domain is derived from human MFG-E8 having the amino acid sequence as set forth in SEQ ID NO: 3. In an alternative embodiment of the present disclosure, the integrin binding domain is a PS binding domain from human EDIL3 (SEQ ID NO: 11), where the PS binding domain comprises amino acids 135-453 of SEQ ID NO: 11.

PS binding activity can be determined by methods well known in the art. For example, a PS binding assay is described in the Examples, section 3.1, wherein the binding of fusion proteins of the present disclosure to PS coated on microtiter plates was assessed by competing against the binding of biotinylated murine MFG-E8. In accordance with the present disclosure, a PS binding domain is considered to have PS binding activity if it has at least 10%, such as e.g. at least 25%, at least 50%, at least 75%, at least 80%, preferably at least 90%, at least 95%, at least 96%, at least 97%, at least 98% of the PS binding activity as observed for the human MFG-E8 protein shown in SEQ ID NO:1 when tested by the same method of determining the respective activity, preferably when tested using the assay described in the Examples, section 3.1.

### Bridging Proteins

There are a number of endogenous proteins that comprise both an integrin binding domain and a PS binding domain. Examples of such ‘bridging proteins’ are shown in Table 3 below.

TABLE 3

Bridging proteins containing both integrin and phosphatidylserine binding domains

| | | | | | |
|---|---|---|---|---|---|
| Abbreviation | UniProt | Name | Putative PS-binding domain | Receptor on phagocytes | Reference |
| EDIL3 (DEL-1) | O43854 | EGF like repeats and discoidin domains 3 | C1-C2 discoidin domains | integrins (αv- β2) | Dasgupta et al., (2012) |
| MFG-E8 | Q08431 | milk fat globule-EGF factor 8 protein, lactadherin | C1-C2 discoidin domains | integrins (αvb3/b5 α8b1) | Andersen et al., (2000) |
| Pros1 | P07225 | Protein S | y-carboxyglutamic acid (Gla) domain | Tyro3 and Mer “anticoagulation factor” | Stitt et al., (1995) |
| Gas6 | Q14393 | Growth arrest specific protein 6 | Gla domain | Tyro3, Mer and AXL | Stitt et al., (1995) |

To be of therapeutic value, it is useful if the bridging protein comprises an integrin binding domain that recognizes integrins on phagocytes that are typically not sensitive to proteolytic cleavage or shedding as has been observed in TAM family members or other PS binding receptors. A protein with a PS binding domain and an integrin binding domain, for example, MFG-E8 or its paralogue EDIL3/DEL1, have been shown to induce efferocytosis in vitro and therefore could be of therapeutic value as efferocytosis inductors in AOls. In contrast, the GAS6 protein for example, may not be particularly effective in promoting efferocytosis in AOIs because its receptor on phagocytes (MerTK) is proteolytically cleaved during inflammation and infection as outline above.

One example of a bridging protein, as listed in Table 3 above, is MFG-E8, which is one of the major proteins found in the milk fat globule membrane (MFGM). MFG-E8 is expressed and secreted by several different types of cells (e.g. mammary epithelial cells, vascular cells, epididymal epithelial cells, aortic smooth muscle cells, activated macrophages, stimulated endometrium, and immature dendritic cells) and tissues (e.g. Heart, lungs, mammary glands, spleen, intestines, liver, kidney, brain, blood, and endothelium). The MFG-E8 protein is also known by several different names such as, lactadherin, BP47, components 15/16, MFGM, MGP57/53, PAS-6/PAS-7glycoprotein, cell wall protein SED1, sperm surface protein SP47, breast epithelial antigen BA46, and O-acetyl GD3 ganglioside synthase (AGS). The MFG-E8 gene is located on chromosome 1 in rats, chromosome 7 in mice, and chromosome 15 in humans. Alternative splicing of the pre-mRNA of MFG-E8 results in three isoforms of the human protein and two forms of mRNA, long and short variants are expressed in mouse mammary glands. The human MFG-E8 gene (UniProtKB - Q08431) encodes a protein that is 387 residues long that is processed to form multiple protein products. The amino acid sequence of human MFG-E8, which comprises the signal peptide (residues 1-23; underlined), EGF-like domain (residues 24-67; italicized), C1 domain (residues 70-225; bold), and C2 domain (residues 230-387; bold and underlined), is provided below:

MPRPRLLAAL CGALLCAPSL LVALDICSKN PCHNGGLCEE ISQEVRGDVF PSYTCTCLKG YAGNHCETKC VEPLGLENGN IANSQIAASS VRVTFLGLQH WVPELARLNR AGMVNAWTPS SNDDNPWIQV NLLRRMWVTG VVTQGASRLA SHEYLKAFKV AYSLNGHEFD FIHDVNKKHK EFVGNWNKNA VHVNLFETPV EAQYVRLYPT SCHTACTLRF ELLGCELNGC ANPLGLKNNS IPDKQITASS SYKTWGLHLF SWNPSYARLD KQGNFNAWVA GSYGNDQWLQ VDLGSSKEVT GIITQGARNF GSVQFVASYK VAYSNDSANW TEYQDPRTGS SKIFPGNWDN HSHKKNLFET PILARYVRIL PVAWHNRIAL RLELLGC (SEQ ID NO: 1).

MFG-E8 lacks the transmembrane function that MFGM has and therefore serves as a peripheral membrane protein. Human MFG-E8 consists of one N-terminal EGF-like domain (SEQ ID NO: 2) that binds to αvβ3 and αvβ5 integrins expressed on phagocytes and a PS binding domain (SEQ ID NO: 3) comprising two F5/8-discoidin sub-domains (C1 and C2) that bind with high affinity to anionic phospholipids. The integrin-binding is a result of the RGD motif located in residues 46-48 of human MFG-E8 (SEQ ID NO: 1). Apoptotic cells, cell debris, hyperactivated cells and the majority of microparticles (MPs) expose PS and are targets of MFG-E8 that, acting as a bridging molecule, opsonizes these cells and microparticles and links them to αvβ3 and αvβ5 integrins on phagocytes. This bridging action triggers an efficient engulfment program leading to internalization of the cells, debris and microparticles. The proteins found in MFGM are highly conserved throughout species. MFG-E8 protein structure varies by species; all species currently known contain two C domains but differ on the number of EGF-like domains. For example, human MFG-E8 protein contains one EGF-like domain, whereas bovine MFG-E8 and murine MFG-E8 (SEQ ID NO: 68) have two EGF-like domains, and chicken, frog, and zebrafish have three EGF-like domains. Domains of MFG-E8, have been proposed previously as constituents of therapeutics, in particular the PS-binding domains (Kooijmans et al., (2018) Nanoscale, 10(5): 2413-2426) and fragments of MFG-E8 have been described to act in models of fibrosis (US patent application US2018/0334486).

The non-phlogistic uptake of dying cells, debris and microparticles by professional and nonprofessional phagocytes plays a critical role in homeostasis after tissue injury (Greenlee-Wacker (2016) supra). The importance of appropriate clearance became furthermore evident in genetic models where MFG-E8 knockout mice showed, for example, increased numbers of (uncleared) dying cells in tissues, exaggerated inflammatory response in disease models such as neonatal sepsis, autoimmunity, poor angiogenesis and impaired wound healing (Hanayama et al., (2004) Science, 204(5474): 1147-50; Das et al., (2016) J Immunol., 196(12): 5089-5100; Hansen et al., (2017) J Pediatr Surg., 52(9): 1520-7).

In addition, MFG-E8 has been shown to generate a tolerogenic environment by suppression of T cell activation and proliferation, inhibition of Th1, Th2, and Th17 subpopulations while increasing regulatory T cell subsets (Tregs). Interestingly, Tregs contribute in return to the resolution of inflammation by inducing efferocytosis by macrophages (Proto et al., (2018) Immunity, 49(4): 666-77). MFG-E8 has been described to promote allogeneic engraftment of embryonic stem cell-derived tissues across the MHC barrier (Tan et al., (2015) Stem Cell Reports, 5(5): 741-752). MFG-E8 also has multiple nutritional uses, which aid in promoting tissue development and protection against infectious agents. Glycoproteins such as MFG-E8 are potential health enhancing nutraceuticals for food and pharmaceutical applications. MFG-E8 can also be combined with other nutrients (e.g. probiotics, whey protein micelles, alpha-hyroxyisocaproic acid, citrulline, and branched chain fatty acids).

### Solubilizing Domain

As described herein, the therapeutic fusion proteins of the present disclosure comprise an integrin binding domain and a PS binding domain. In addition, the fusion proteins also comprise an additional domain that confers a number of desirable properties on the fusion protein. This additional domain, which has been termed a ‘solubilizing domain’ for the purposes of this application, confers improved biological properties such as increased solubility, reduced aggregation and increased bioactivity. As a result, the fusion protein shows desirable pharmacokinetic profiles. Furthermore the presence of a solubilizing domain improves the stability of the therapeutic fusion protein and results in improved expression of the fusion protein compared to wild-type protein in cell expression systems as shown by an increase in yield following purification.

The presence of a solubilizing domain may also confer an extended half-life on the therapeutic fusion protein. For example, many protein drugs are linked to polyethylene glycol (PEG), reCODE PEG, antibody scaffold, polysialic acid (PSA), hydroxyethyl starch (HES), and serum proteins, such as albumin, IgG and FcRn, to extend their plasma half-lives and to achieve enhanced therapeutic effects (Kim et al., (2010) J Pharmacol Exp Ther., 334: 682-92; Weimer et al., (2008) Thromb Haemost. 99: 659-67; Dumont et al., (2006) BioDrugs, 20: 151-60; Schellenberger et al., (2009) Nat Biotechnol., 27: 1186-90).

In some embodiments the solubilizing domain is an albumin protein such as human serum albumin (HSA; SEQ ID NO: 4) or variants thereof. For example, HSA comprising the amino acid substitution C34S to lower aggregation propensity (SEQ ID NO: 5), or domains of HSA such as HSA D3; (SEQ ID NO: 6). HSA has a very long serum half-life due to a number of factors including its relatively large size that reduces renal filtration and its neonatal Fc receptor (FcRn) binding feature thereby evading intracellular degradation. The use of N-terminal fragments of HSA for fusions to polypeptides has also been proposed (e.g. Patent application EP399666). Accordingly, genetically or chemically fusing or conjugating molecules to albumin can stabilize or extend the shelf-life, and/or retain a molecule’s activity for extended periods of time in solution, in vitro and/or in vivo. Additional methods relating to HSA fusions can be found, for example, in international patent applications WO2001/077137 and WO2003/060071.

In some embodiments, the solubilizing domain comprises an antibody Fc domain such as human Fc-immunoglobulin G1 (Fc-lgG1; SEQ ID NO: 7). The Fc domain may also be modified, for example, by using knob-into-hole (KiH) based modifications to improve heterodimerization of Fc by introducing complementary amino acid substitutions in the CH3 domain of the Fc. For example, the substitution T366W to create a ‘knob’ on one CH3 domain and the substitutions T366S, L368A and Y407V to create a ‘hole’ on the other CH3 domain (Merchant et al (1998) Nat. Biotechnol., 16(7): 677-81; EU numbering IgG1). Additional modifications that can be included in the Fc domain either alone or combined with modifications to improve heterodimerization may comprise, for example, amino acid substitutions to cysteine to create an additional cysteine bond, for example S354C and/or Y349C, and amino acid substitutions to reduce or eliminate binding to Fcy receptors and complement protein C1q, to ‘silence’ immune effector function. The so-called ‘LALA’ double mutation (L234A together with L235A; EU numbering) results in diminished effector functions (Lund et al., (1992) Mol Immunol., 29: 53-9). Alternatively, the ‘DAPA’ double mutation (D265A together with P329A; EU numbering) results in diminished effector functions. In an embodiment of the present disclosure, the Fc domain may comprise the amino acid substitutions D265A, P329A for Fc silencing and/or the KiH amino acid substitutions T366W (knob) or T366S, L368A and Y407V (hole). In one embodiment, the Fc domain is derived from human IgG1 and comprises the amino acid substitutions D265A, P329A (SEQ ID NO: 8). In another embodiment, the Fc domain is derived from human IgG1 and comprises the amino acid substitutions D265A, P329A, S354C and the amino acid substitutionT366W (Fc-lgG1-knob; SEQ ID NO: 9). In another embodiment, the Fc domain is derived from human IgG1 and comprises the amino acid substitutions D265A, P329A, Y349C and the amino acid substitutions T366S, L368A and Y407V (Fc-lgG1-hole; SEQ ID NO: 10).

In some embodiments, the the solubilizing domain comprises an antibody Fc domain derived from human IgA, IgD, IgE or IgM.

In some embodiments, the solubilizing domain comprises SUMO (Small Ubiquitin-like Modifier), Ubiquitin, GST (Glutathion S-transferase), or variants thereof.

### Linkage and Orientation of Domains of Therapeutic Fusion Proteins

The integrin binding domain, PS binding domain and solubilizing domain of the fusion proteins of the present disclosure are linked. As used herein, the term ‘linked’ or ‘linking’ refers to one domain of the fusion protein being attached, directly or indirectly, to another domain of the fusion protein. Direct attachment is a form of linkage, and is referred to herein as ‘fused’ or ‘fusion’. Using a molecule having the form A-B-C as an example: domain A is linked directly to domain B and linked directly to domain C. As such, domain A may also be described as being fused to domain B which is fused to domain C. As another example, domain A is linked directly to domain B and linked indirectly to domain C. As such, domain A may also be described as being fused to domain B which is linked indirectly by an internal linker to domain C.

In some embodiments the linkage is a direct linkage and the domains are therefore fused to each other. In some embodiments an integrin binding domain is fused to a PS binding domain that is fused to a solubilizing domain. Specifically, the PS binding domain (e.g. C1-C2 discoidin sub-domains) is fused to the C-terminus of the integrin binding domain (e.g. an EGF-like domain) and fused to the N-terminus of the solubilizing domain (e.g. HSA). In some embodiments a solubilizing domain is fused to an integrin binding domain that is fused to a PS binding domain. Specifically, the integrin binding domain (e.g. an EGF-like domain) is fused to the C-terminus of the solubilizing domain (e.g. HSA) and fused to the N-terminus of the PS binding domain (e.g. C1-C2 discoidin sub-domains). In some embodiments, an integrin binding domain is fused to a PS binding domain comprising C1-C2 discoidin sub-domains and a solubilizing domain is inserted between the C1-C2 discoidin sub-domain. Specifically, C terminus of the integrin binding domain (e.g. an EGF-like domain) is fused to the N-terminus of the C1 discoidin sub-domain and the C-terminus of the C1 discoidin sub-domain is fused to the N-terminus of the solubilizing domain (e.g. HSA) and the C-terminus of the solubilizing domain is fused to the N-terminus of the C2 discoidin sub-domain. In another embodiment, an integrin binding domain is fused to a solubilizing domain which is fused to a PS binding domain. Specifically, the solubilizing domain (e.g. HSA) is fused to the C-terminus of the integrin binding domain (e.g. EGF-like domain) and to the N-terminus of the PS binding domain (e.g. C1-C2 discoidin sub-domains). In one embodiment, HSA is fused to the C-terminus of an EGF-like domain and fused to the N-terminus of the C1 discoidin domain.

In some embodiments, the solubilizing domain (e.g. HSA) is fused between an integrin binding domain and a PS binding domain. In some embodiments, the integrin binding domain is located at the N-terminus of the fusion protein and the PS binding domain is located at the C-terminus of the fusion protein.

In some embodiments, the fusion protein comprises a first region containing an integrin binding domain, e.g. EGF-like domain, a second region containing a solubilizing domain (e.g. HSA or Fc), and a third region containing the PS binding domain, e.g. C1 and/or C2 discoidin domain. In some embodiments, the integrin binding domain is located at the N-terminus of the fusion proteinand the PS binding domain is located at the C-terminus of the fusion protein.

In some embodiments, the solubilizing domain (e.g. HSA or Fc) is HSA.

In some embodiments, the solubilizing domain is HSA, or a functional variant therefore.

In some embodiments, the solubilizing domain is the antibody Fc-immunoglobulin G1 (Fc-IgG1; SEQ ID NO: 7).

In a preferred embodiment, HSA comprising an amino acid sequence as set forth in SEQ ID NO: 5 is fused to the C-terminus of the EGF-like domain of MFG-E8 and fused to the N-terminus of the PS binding domain of MFG-E8. In one embodiment, the fusion protein comprises an amino acid sequence as set forth in SEQ ID NO: 46 (FP068). In one embodiment, the fusion protein comprises an amino acid sequence as set forth in SEQ ID NO: 48 (FP776).

In an alternative embodiment, HSA comprising an amino acid sequence as set forth in SEQ ID NO: 5 is fused to the C-terminus of the EGF-like domain of EDIL3 and fused to the N-terminus of the PS binding domain of EDIL3. In one embodiment, the fusion protein comprises an amino acid sequence as set forth in SEQ ID NO: 70 (FP1068). In one embodiment, the fusion protein comprises an amino acid sequence as set forth in SEQ ID NO: 69 (FP1776).

In some embodiments, the linkage is via a polypeptide linker and a polypeptide linker that, for example, joins an solubilizing domain to a PS binding domain in a fusion protein of the present disclosure is referred to as an ‘external linker’. These external linkers typically comprise glycine (G) and/or serine (S) and may also comprise glycine and leucine (GL) or glycine and valine (GL). In some embodiments the linker comprises multiples of G and S residues, for example, G₂S and multiples thereof such as (G₂S)₄ as set forth in SEQ ID NO: 62, (GS)₄ as set forth in SEQ ID NO: 63, G₄S as set forth in SEQ ID NO: 64 or (G₄S)₂ as set forth in SEQ ID NO: 65.

In some embodiments, an external linker is fused between the C-terminus of an integrin binding domain and the N-terminus of a solubilizing domain. Specifically, an external linker is fused to the C-terminus of an EGF-like domain and the N-terminus of HSA. In some embodiments, an external linker is fused between the C-terminus of a solubilizing domain and the N-terminus of a PS binding domain. Specifically an external linker is fused to the C-terminus of HSA and the N-terminus of the PS binding domain. In some embodiments, an external linker is fused between the C-terminus of an integrin binding domain and the N-terminus of a solubilizing domain, and an additional external linker is fused between the C-terminus of the solubilizing domain and the N-terminus of a PS binding domain. Specifically, an external linker is fused to the C-terminus of an EGF-like domain and the N-terminus of HSA, and an additional external linker is fused to the C-terminus of HSA and the N-terminus of a PS binding domain.

In some embodiments, an external linker comprising GS is fused to the C-terminus of an integrin binding domain and to the N-terminus of a solubilizing domain. In some embodiments, an external linker comprising GL is fused to the C-terminus of a solubilizing domain and to the N-terminus of a PS binding domain. In some embodiments, an external linker comprising (G₂S)₄ (SEQ ID NO: 62) is fused to the C-terminus of a solubilizing domain and to the N-terminus of a PS binding domain. In some embodiments, an external linker comprising G₄S (SEQ ID NO: 64) is fused to the C-terminus of a solubilizing domain and to the N-terminus of a PS binding domain. In some embodiments, an external linker comprising (G₄S)₂ (SEQ ID NO: 65) is fused to the C-terminus of a solubilizing domain and to the N-terminus of a PS binding domain.

In one embodiment, an external linker comprising GS is fused to the C-terminus of an EGF-like domain and to the N-terminus of HSA. A fusion protein of the present disclosure comprising this structure has an amino acid sequence as set forth in SEQ ID NO: 42 (FP330).

In one embodiment, an external linker comprising GS is fused to the C-terminus of an EGF-like domain and to the N-terminus of HSA, and a further external linker comprising (GS)₄ (SEQ ID NO: 63) is fused to the C-terminus of HSA and to the N-terminus of a PS binding domain.

In one embodiment, an external linker comprising GS is fused to the C-terminus of an EGF-like domain and to the N-terminus of HSA, and a further external linker comprising (G₂S)₄ (SEQ ID NO: 62) is fused to the C-terminus of HSA and to the N-terminus of a PS binding domain. A fusion protein of the present disclosure comprising this structure has an amino acid sequence as set forth in SEQ ID NO: 42 (FP330).

In one embodiment, an external linker comprising GS is fused to the C-terminus of an EGF-like domain and to the N-terminus of HSA. The C-terminus of HSA is directly fused to the N-terminus of a PS binding domain.

In one embodiment, an external linker comprising GS is fused to the C-terminus of an EGF-like domain and to the N-terminus of HSA, and an additional external linker comprising G₄S (SEQ ID NO: 64) is fused to the C-terminus of HSA and to the N-terminus of a PS binding domain. A fusion protein of the present disclosure comprising this structure has an amino acid sequence as set forth in SEQ ID NO: 54 (FP811).

In one embodiment, an external linker comprising GS is fused to the C-terminus of an EGF-like domain and to the N-terminus of HSA, and a further external linker comprising (G₄S)₂ (SEQ ID NO: 65) is fused to the C-terminus of HSA and to the N-terminus of a PS binding domain. A fusion protein of the present disclosure comprising this structure has an amino acid sequence as set forth in SEQ ID NO: 56 (FP010).

In some embodiments, a His tag is fused to an external linker comprising GS (GS-6xHis; SEQ ID NO: 66) which is fused to the C-terminus of a PS binding domain. In one embodiment, a fusion protein of the present disclosure comprising a His tag has an amino acid sequence as set forth in SEQ ID NO: 44 (FP278) or SEQ ID NO: 60 (FP114 or FP260).

### Functional Properties of Therapeutic Fusion Proteins

The present disclosure provides fusion proteins derived from human MFG-E8 and which are effective in promoting efferocytosis and therefore are active in eliminating the key drivers of systemic inflammation and microvascular pathology. As set out in the Examples, the fusion proteins having the general structure EGF-HSA-C1-C2 have been shown to be effective in a number of efferocytosis assays. For example, the fusion proteins have been effective in restoring lipopolysaccharide (LPS) or S.aureus impaired efferocytosis of macrophages and boosting efferocytosis of microparticles and dying cells by endothelial cells. The fusion proteins have also been effective in protecting kidney function and protecting against bodyweight loss in a mouse model of acute kidney injury.

### EXEMPLARY PROTEIN SEQUENCES

The amino acid sequences in Table 4 include examples of therapeutic fusion proteins of the present disclosure, as well as portions thereof.

Throughout the text of this application, should there be a discrepancy between the text of the specification (e.g., Table 4) and the sequence listing, the text of the specification shall prevail.

TABLE 4

Exemplary Protein Sequences

| SEQ ID NO | Description | Sequence |
|---|---|---|
| 1 | Human MFG-E8 | MPRPRLLAALCGALLCAPSLLVALDICSKNPCHNGGLCEEISQEVRGDVFPSYTC TCLKGYAGNHCETKCVEPLGLENGNIANSQIAASSVRVTFLGLQHWVPELARLN RAGMVNAWTPSSNDDNPWIQVNLLRRMWVTGVVTQGASRLASHEYLKAFKVA YSLNGHEFDFIHDVNKKHKEFVGNWNKNAVHVNLFETPVEAQYVRLYPTSCHTA CTLRFELLGCELNGCANPLGLKNNSIPDKQITASSSYKTWGLHLFSWNPSYARLD KQGNFNAWVAGSYGNDQWLQVDLGSSKEVTGIITQGARNFGSVQFVASYKVAY SNDSANWTEYQDPRTGSSKIFPGNWDNHSHKKNLFETPILARYVRILPVAWHNR IALRLELLGC |
| 2 | EGF-like domain of MFG-E8 | LDICSKNPCHNGGLCEEISQEVRGDVFPSYTCTCLKGYAGNHCETK |
| 3 | PS binding domain of MFG-E8 (C1-C2 sub-domains) | CVEPLGLENGNIANSQIAASSVRVTFLGLQHVVVPELARLNRAGMVNAWTPSSND DNPWIQVNLLRRMWVTGVVTQGASRLASHEYLKAFKVAYSLNGHEFDFIHDVNK KHKEFVGNWNKNAVHVNLFETPVEAQYVRLYPTSCHTACTLRFELLGCELNGCA NPLGLKNNSIPDKQITASSSYKTWGLHLFSWNPSYARLDKQGNFNAWVAGSYG NDQWLQVDLGSSKEVTGIITQGARNFGSVQFVASYKVAYSNDSANWTEYQDPR TGSSKIFPGNWDNHSHKKNLFETPILARYVRILPVAWHNRIALRLELLGC |
| 4 | HSA wild-type | DAHKSEVAHRFKDLGEENFKALVLIAFAQYLQQCPFEDHVKLVNEVTEFAKTCVA DESAENCDKSLHTLFGDKLCTVATLRETYGEMADCCAKQEPERNECFLQHKDD NPNLPRLVRPEVDVMCTAFHDNEETFLKKYLYEIARRHPYFYAPELLFFAKRYKA AFTECCQAADKAACLLPKLDELRDEGKASSAKQRLKCASLQKFGERAFKAWAVA RLSQRFPKAEFAEVSKLVTDLTKVHTECCHGDLLECADDRADLAKYICENQDSIS SKLKECCEKPLLEKSHCIAEVENDEMPADLPSLAADFVESKDVCKNYAEAKDVFL GMFLYEYARRHPDYSVVLLLRLAKTYETTLEKCCAAADPHECYAKVFDEFKPLVE EPQNLIKQNCELFEQLGEYKFQNALLVRYTKKVPQVSTPTLVEVSRNLGKVGSK CCKHPEAKRMPCAEDYLSVVLNQLCVLHEKTPVSDRVTKCCTESLVNRRPCFSA LEVDETYVPKEFNAETFTFHADICTLSEKERQIKKQTALVELVKHKPKATKEQLKA VMDDFAAFVEKCCKADDKETCFAEEGKKLVAASQAAL |
| 5 | HSA (C34S) | DAHKSEVAHRFKDLGEENFKALVLIAFAQYLQQSPFEDHVKLVNEVTEFAKTCVA DESAENCDKSLHTLFGDKLCTVATLRETYGEMADCCAKQEPERNECFLQHKDD NPNLPRLVRPEVDVMCTAFHDNEETFLKKYLYEIARRHPYFYAPELLFFAKRYKA AFTECCQAADKAACLLPKLDELRDEGKASSAKQRLKCASLQKFGERAFKAWAVA RLSQRFPKAEFAEVSKLVTDLTKVHTECCHGDLLECADDRADLAKYICENQDSIS SKLKECCEKPLLEKSHCIAEVENDEMPADLPSLAADFVESKDVCKNYAEAKDVFL GMFLYEYARRHPDYSVVLLLRLAKTYETTLEKCCAAADPHECYAKVFDEFKPLVE EPQNLIKQNCELFEQLGEYKFQNALLVRYTKKVPQVSTPTLVEVSRNLGKVGSK CCKHPEAKRMPCAEDYLSVVLNQLCVLHEKTPVSDRVTKCCTESLVNRRPCFSA LEVDETYVPKEFNAETFTFHADICTLSEKERQIKKQTALVELVKHKPKATKEQLKA VMDDFAAFVEKCCKADDKETCFAEEGKKLVAASQAAL |
| 6 | HSA D3 | LVEEPQNLIKQNCELFEQLGEYKFQNALLVRYTKKVPQVSTPTLVEVSRNLGKVG SKCCKHPEAKRMPCAEDCLSVFLNQLCVLHEKTPVSDRVTKCCTESLVNGRPCF SALEVDETYVPKEFNAETFTFHADICTLSEKERQIKKQTALVELVKHKPKATKEQL KAVMDDFAAFVEKCCKADDKETCFAEEGKKLVAASQAALGL |
| 7 | Fc-lgG1 wild-type | APELLGGPSVFLFPPKPKDTLMISRTPEVTCVVVDVSHEDPEVKFNVTYVDGVEV HNAKTKPREEQYNSTYRVVSVLTVLHQDWLNGKEYKCKVSNKALPAPIEKTISKA KGQPREPQVYTLPPSREEMTKNQVSLTCLVKGFYPSDIAVEWESNGQPENNYK TTPPVLDSDGSFFLYSKLTVDKSRWQQGNVFSCSVMHEALHNHYTQKSLSLSP GK |
| 8 | Fc-lgG1 silent | APELLGGPSVFLFPPKPKDTLMISRTPEVTCVVVAVSHEDPEVKFNWYVDGVEV HNAKTKPREEQYNSTYRVVSVL TVLHQDWLNGKEYKCKVSNKALAAPIEKTISKA KGQPREPQVYTLPPSREEMTKNQVSLTCLVKGFYPSDIAVEWESNGQPENNYK TTPPVLDSDGSFFLYSKLTVDKSRWQQGNVFSCSVMHEALHNHYTQKSLSLSP GK |
| 9 | Fc-lgG1 Knob | APELLGGPSVFLFPPKPKDTLMISRTPEVTCVVVAVSHEDPEVKFNWYVDGVEV HNAKTKPREEQYNSTYRVVSVL TVLHQDWLNGKEYKCKVSNKALAAPIEKTISKA KGQPREPQVYTLPPCREEMTKNQVSLWCLVKGFYPSDIAVEWESNGQPENNYK TTPPVLDSDGSFFLYSKLTVDKSRWQQGNVFSCSVMHEALHNHYTQKSLSLSP GK |
| 10 | Fc-lgG1 Hole | APELLGGPSVFLFPPKPKDTLMISRTPEVTCVVVAVSHEDPEVKFNWYVDGVEV HNAKTKPREEQYNSTYRVVSVL TVLHQDWLNGKEYKCKVSNKALAAPIEKTISKA KGQPREPQVCTLPPSREEMTKNQVSLSCAVKGFYPSDIAVEWESNGQPENNYK TTPPVLDSDGSFFLVSKLTVDKSRWQQGNVFSCSVMHEALHNHYTQKSLSLSP GK |
| 11 | Human EDIL3 | DICDPNPCENGGICLPGLADGSFSCECPDGFTDPNCSSVVEVASDEEEPTSAGP CTPNPCHNGGTCEISEAYRGDTFIGYVCKCPRGFNGIHCQHNINECEVEPCKNG GICTDLVANYSCECPGEFMGRNCQYKCSGPLGIEGGIISNQQITASSTHRALFGL QKWYPYYARLNKKGLINAWTAAENDRWPWIQINLQRKMRVTGVITQGAKRIGSP EYIKSYKIAYSNDGKTWAMYKVKGTNEDMVFRGNIDNNTPYANSFTPPIKAQYVR LYPQVCRRHCTLRMELLGCELSGCSEPLGMKSGHIQDYQITASSIFRTLNMDMF TWEPRKARLDKQGKVNAWTSGHNDQSQWLQVDLLVPTKVTGIITQGAKDFGHV QFVGSYKLAYSNDGEHWTVYQDEKQRKDKVFQGNFDNDTHRKNVIDPPIYARHI RILPWSWYGRITLRSELLGCTEEE |
| 12 | FP050 EDIL3 EGF-HSA-C1-C2 | DICDPNPCENGGICLPGLADGSFSCECPDGFTDPNCSSVVEVASDEEEPTSAGP CTPNPCHNGGTCEISEAYRGDTFIGYVCKCPRGFNGIHCQHNINECEVEPCKNG GICTDLVANYSCECPGEFMGRNCQYKGSDAHKSEVAHRFKDLGEENFKALVLIA FAQYLQQSPFEDHVKLVNEVTEFAKTCVADESAENCDKSLHTLFGDKLCTVATL RETYGEMADCCAKQEPERNECFLQHKDDNPNLPRLVRPEVDVMCTAFHDNEET FLKKYLYEIARRHPYFYAPELLFFAKRYKAAFTECCQAADKAACLLPKLDELRDE GKASSAKQRLKCASLQKFGERAFKAWAVARLSQRFPKAEFAEVSKLVTDLTKVH TECCHGDLLECADDRADLAKYICENQDSISSKLKECCEKPLLEKSHCIAEVENDE MPADLPSLAADFVESKDVCKNYAEAKDVFLGMFLYEYARRHPDYSVVLLLRLAK TYETTLEKCCAAADPHECYAKVFDEFKPLVEEPQNLIKQNCELFEQLGEYKFQNA LLVRYTKKVPQVSTPTLVEVSRNLGKVGSKCCKHPEAKRMPCAEDYLSVVLNQL CVLHEKTPVSDRVTKCCTESLVNRRPCFSALEVDETYVPKEFNAETFTFHADICT LSEKERQIKKQTALVELVKHKPKATKEQLKAVMDDFAAFVEKCCKADDKETCFAE EGKKLVAASQAALGLGGSGGSGGSGGSCSGPLGIEGGIISNQQITASSTHRALF GLQKWYPYYARLNKKGLINAWTAAENDRWPWIQINLQRKMRVTGVITQGAKRIG SPEYIKSYKIAYSNDGKTWAMYKVKGTNEDMVFRGNIDNNTPYANSFTPPIKAQY VRLYPQVCRRHCTLRMELLGCELSGCSEPLGMKSGHIQDYQITASSIFRTLNMD MFTWEPRKARLDKQGKVNAWTSGHNDQSQWLQVDLLVPTKVTGIITQGAKDFG HVQFVGSYKLAYSNDGEHWTVYQDEKQRKDKVFQGNFDNDTHRKNVIDPPIYA RHIRILPWSWYGRITLRSELLGC |
| 84 | EDIL3 EGF-like domain 1 [EDIL3]-HSA-C1-C2[EDIL3] | DICDPNPCENGGICLPGLADGSFSCECPDGFTDPNCSSVVEV ASDEEEPTGSDA HKSEVAHRFKDLGEENFKALVLIAFAQYLQQSPFEDHVKLVNEVTEFAKTCVADE SAENCDKSLHTLFGDKLCTVATLRETYGEMADCCAKQEPERNECFLQHKDDNP NLPRLVRPEVDVMCTAFHDNEETFLKKYLYEIARRHPYFYAPELLFFAKRYKAAF TECCQAADKAACLLPKLDELRDEGKASSAKQRLKCASLQKFGERAFKAWAVARL SQRFPKAEFAEVSKLVTDLTKVHTECCHGDLLECADDRADLAKYICENQDSISSK LKECCEKPLLEKSHCIAEVENDEMPADLPSLAADFVESKDVCKNYAEAKDVFLG MFLYEYARRHPDYSVVLLLRLAKTYETTLEKCCAAADPHECYAKVFDEFKPLVEE PQNLIKQNCELFEQLGEYKFQNALLVRYTKKVPQVSTPTLVEVSRNLGKVGSKC CKHPEAKRMPCAEDYLSVVLNQLCVLHEKTPVSDRVTKCCTESLVNRRPCFSAL EVDETYVPKEFNAETFTFHADICTLSEKERQIKKQTALVELVKHKPKATKEQLKAV MDDFAAFVEKCCKADDKETCFAEEGKKLVAASQAALGLGGSGGSGGSGGSCS GPLGIEGGIISNQQITASSTHRALFGLQKWYPYYARLNKKGLINAWTAAENDRWP WIQINLQRKMRVTGVITQGAKRIGSPEYIKSYKIAYSNDGKTWAMYKVKGTNEDM VFRGNIDNNTPYANSFTPPIKAQYVRLYPQVCRRHCTLRMELLGCELSGCSEPL GMKSGHIQDYQITASSIFRTLNMDMFTWEPRKARLDKQGKVNAWTSGHNDQSQ WLQVDLL VPTKVTGIITQGAKDFGHVQFVGSYKLA YSNDGEHWTVYQDEKQRK DKVFQGNFDNDTHRKNVIDPPIYARHIRILPWSWYGRITLRSELLGC |
| 85 | EDIL3 EGF-like domain 2[EDIL3]-HSA-C1-C2[EDIL3] | SAGPCTPNPCHNGGTCEISEAYRGDTFIGYVCKCPRGFNGIHCQHGSDAHKSEV AHRFKDLGEENFKALVLIAFAQYLQQSPFEDHVKLVNEVTEFAKTCVADESAENC DKSLHTLFGDKLCTVATLRETYGEMADCCAKQEPERNECFLQHKDDNPNLPRLV RPEVDVMCTAFHDNEETFLKKYLYEIARRHPYFYAPELLFFAKRYKAAFTECCQA ADKAACLLPKLDELRDEGKASSAKQRLKCASLQKFGERAFKAWAVARLSQRFPK AEFAEVSKLVTDLTKVHTECCHGDLLECADDRADLAKYICENQDSISSKLKECCE KPLLEKSHCIAEVENDEMPADLPSLAADFVESKDVCKNYAEAKDVFLGMFLYEYA RRHPDYSVVLLLRLAKTYETTLEKCCAAADPHECYAKVFDEFKPLVEEPQNLIKQ NCELFEQLGEYKFQNALLVRYTKKVPQVSTPTLVEVSRNLGKVGSKCCKHPEAK RMPCAEDYLSVVLNQLCVLHEKTPVSDRVTKCCTESLVNRRPCFSALEVDETYV PKEFNAETFTFHADICTLSEKERQIKKQTALVELVKHKPKATKEQLKAVMDDFAAF VEKCCKADDKETCFAEEGKKLVAASQAALGLGGSGGSGGSGGSCSGPLGIEGG IISNQQITASSTHRALFGLQKWYPYYARLNKKGLINAWTAAENDRWPWIQINLQR KMRVTGVITQGAKRIGSPEYIKSYKIAYSNDGKTWAMYKVKGTNEDMVFRGNID NNTPYANSFTPPIKAQYVRLYPQVCRRHCTLRMELLGCELSGCSEPLGMKSGHI QDYQITASSIFRTLNMDMFTWEPRKARLDKQGKVNAWTSGHNDQSQWLQVDLL VPTKVTGIITQGAKDFGHVQFVGSYKLAYSNDGEHWTVYQDEKQRKDKVFQGN FDNDTHRKNVIDPPIYARHIRILPWSWYGRITLRSELLGC |
| 86 | EDIL3 EGF-like domain 3[EDIL3]-HSA-C1-C2[EDIL3] | NINECEVEPCKNGGICTDLVANYSCECPGEFMGRNCQYKGSDAHKSEVAHRFK DLGEENFKALVLIAFAQYLQQSPFEDHVKLVNEVTEFAKTCVADESAENCDKSLH TLFGDKLCTVATLRETYGEMADCCAKQEPERNECFLQHKDDNPNLPRLVRPEV DVMCTAFHDNEETFLKKYLYEIARRHPYFYAPELLFFAKRYKAAFTECCQAADKA ACLLPKLDELRDEGKASSAKQRLKCASLQKFGERAFKAWAVARLSQRFPKAEFA EVSKLVTDLTKVHTECCHGDLLECADDRADLAKYICENQDSISSKLKECCEKPLL EKSHCIAEVENDEMPADLPSLAADFVESKDVCKNYAEAKDVFLGMFLYEYARRH PDYSVVLLLRLAKTYETTLEKCCAAADPHECYAKVFDEFKPLVEEPQNLIKQNCE LFEQLGEYKFQNALLVRYTKKVPQVSTPTLVEVSRNLGKVGSKCCKHPEAKRMP CAEDYLSVVLNQLCVLHEKTPVSDRVTKCCTESLVNRRPCFSALEVDETYVPKE FNAETFTFHADICTLSEKERQIKKQTALVELVKHKPKATKEQLKAVMDDFAAFVEK CCKADDKETCFAEEGKKLVAASQAALGLGGSGGSGGSGGSCSGPLGIEGGIISN QQITASSTHRALFGLQKWYPYYARLNKKGLINAWTAAENDRWPWIQINLQRKMR VTGVITQGAKRIGSPEYIKSYKIAYSNDGKTWAMYKVKGTNEDMVFRGNIDNNTP YANSFTPPIKAQYVRLYPQVCRRHCTLRMELLGCELSGCSEPLGMKSGHIQDYQ ITASSIFRTLNMDMFTWEPRKARLDKQGKVNAWTSGHNDQSQWLQVDLLVPTK VTGIITQGAKDFGHVQFVGSYKLAYSNDGEHWTVYQDEKQRKDKVFQGNFDND THRKNVIDPPIYARHIRILPWSWYGRITLRSELLGC |
| 87 | EDIL3 EGF-like domain 1-2[EDIL3]-HSA-C1-C2[EDIL3] | DICDPNPCENGGICLPGLADGSFSCECPDGFTDPNCSSVVEVASDEEEPTSAGP CTPNPCHNGGTCEISEAYRGDTFIGYVCKCPRGFNGIHCQHGSDAHKSEVAHRF KDLGEENFKALVLIAFAQYLQQSPFEDHVKLVNEVTEFAKTCVADESAENCDKSL HTLFGDKLCTVATLRETYGEMADCCAKQEPERNECFLQHKDDNPNLPRLVRPE VDVMCTAFHDNEETFLKKYLYEIARRHPYFYAPELLFFAKRYKAAFTECCQAADK AACLLPKLDELRDEGKASSAKQRLKCASLQKFGERAFKAWAVARLSQRFPKAEF AEVSKLVTDLTKVHTECCHGDLLECADDRADLAKYICENQDSISSKLKECCEKPL LEKSHCIAEVENDEMPADLPSLAADFVESKDVCKNYAEAKDVFLGMFLYEYARR HPDYSVVLLLRLAKTYETTLEKCCAAADPHECYAKVFDEFKPLVEEPQNLIKQNC ELFEQLGEYKFQNALLVRYTKKVPQVSTPTLVEVSRNLGKVGSKCCKHPEAKRM PCAEDYLSVVLNQLCVLHEKTPVSDRVTKCCTESLVNRRPCFSALEVDETYVPK EFNAETFTFHADICTLSEKERQIKKQTALVELVKHKPKATKEQLKAVMDDFAAFVE KCCKADDKETCFAEEGKKLVAASQAALGLGGSGGSGGSGGSCSGPLGIEGGIIS NQQITASSTHRALFGLQKWYPYYARLNKKGLINAWTAAENDRWPWIQINLQRKM RVTGVITQGAKRIGSPEYIKSYKIAYSNDGKTWAMYKVKGTNEDMVFRGNIDNNT PYANSFTPPIKAQYVRLYPQVCRRHCTLRMELLGCELSGCSEPLGMKSGHIQDY QITASSIFRTLNMDMFTWEPRKARLDKQGKVNAWTSGHNDQSQWLQVDLLVPT KVTGIITQGAKDFGHVQFVGSYKLAYSNDGEHWTVYQDEKQRKDKVFQGNFDN DTHRKNVIDPPIYARHIRILPWSWYGRITLRSELLGC |
| 88 | EDIL3 EGF-like domain 2-3[EDIL3]-HSA-C1-C2[EDIL3] | SAGPCTPNPCHNGGTCEISEAYRGDTFIGYVCKCPRGFNGIHCQHNINECEVEP CKNGGICTDLVANYSCECPGEFMGRNCQYKGSDAHKSEVAHRFKDLGEENFKA LVLIAFAQYLQQSPFEDHVKLVNEVTEFAKTCVADESAENCDKSLHTLFGDKLCT VATLRETYGEMADCCAKQEPERNECFLQHKDDNPNLPRLVRPEVDVMCTAFHD NEETFLKKYLYEIARRHPYFYAPELLFFAKRYKAAFTECCQAADKAACLLPKLDEL RDEGKASSAKQRLKCASLQKFGERAFKAWAVARLSQRFPKAEFAEVSKLVTDLT KVHTECCHGDLLECADDRADLAKYICENQDSISSKLKECCEKPLLEKSHCIAEVE NDEMPADLPSLAADFVESKDVCKNYAEAKDVFLGMFLYEYARRHPDYSVVLLLR LAKTYETTLEKCCAAADPHECYAKVFDEFKPLVEEPQNLIKQNCELFEQLGEYKF QNALLVRYTKKVPQVSTPTLVEVSRNLGKVGSKCCKHPEAKRMPCAEDYLSVVL NQLCVLHEKTPVSDRVTKCCTESLVNRRPCFSALEVDETYVPKEFNAETFTFHA DICTLSEKERQIKKQTALVELVKHKPKATKEQLKAVMDDFAAFVEKCCKADDKET CFAEEGKKLVAASQAALGLGGSGGSGGSGGSCSGPLGIEGGIISNQQITASSTH RALFGLQKWYPYYARLNKKGLINAWTAAENDRWPWIQINLQRKMRVTGVITQGA KRIGSPEYIKSYKIAYSNDGKTWAMYKVKGTNEDMVFRGNIDNNTPYANSFTPPI KAQYVRLYPQVCRRHCTLRMELLGCELSGCSEPLGMKSGHIQDYQITASSIFRTL NMDMFTWEPRKARLDKQGKVNAWTSGHNDQSQWLQVDLLVPTKVTGIITQGAK DFGHVQFVGSYKLAYSNDGEHWTVYQDEKQRKDKVFQGNFDNDTHRKNVIDP PIYARHIRILPWSWYGRITLRSELLGC |
| 89 | EDIL3 EGF-like domain 1-3[EDIL3]-HSA-C1-C2[EDIL3] | DICDPNPCENGGICLPGLADGSFSCECPDGFTDPNCSSVVEVASDEEEPTNINE CEVEPCKNGGICTDLVANYSCECPGEFMGRNCQYKGSDAHKSEVAHRFKDLGE ENFKALVLIAFAQYLQQSPFEDHVKLVNEVTEFAKTCVADESAENCDKSLHTLFG DKLCTVATLRETYGEMADCCAKQEPERNECFLQHKDDNPNLPRLVRPEVDVMC TAFHDNEETFLKKYLYEIARRHPYFYAPELLFFAKRYKAAFTECCQAADKAACLLP KLDELRDEGKASSAKQRLKCASLQKFGERAFKAWAVARLSQRFPKAEFAEVSKL VTDLTKVHTECCHGDLLECADDRADLAKYICENQDSISSKLKECCEKPLLEKSHCI AEVENDEMPADLPSLAADFVESKDVCKNYAEAKDVFLGMFLYEYARRHPDYSVV LLLRLAKTYETTLEKCCAAADPHECYAKVFDEFKPLVEEPQNLIKQNCELFEQLG EYKFQNALLVRYTKKVPQVSTPTLVEVSRNLGKVGSKCCKHPEAKRMPCAEDYL SVVLNQLCVLHEKTPVSDRVTKCCTESLVNRRPCFSALEVDETYVPKEFNAETFT FHADICTLSEKERQIKKQTALVELVKHKPKATKEQLKAVMDDFAAFVEKCCKADD KETCFAEEGKKLVAASQAALGLGGSGGSGGSGGSCSGPLGIEGGIISNQQITAS STHRALFGLQKWYPYYARLNKKGLINAWTAAENDRWPWIQINLQRKMRVTGVIT QGAKRIGSPEYIKSYKIAYSNDGKTWAMYKVKGTNEDMVFRGNIDNNTPYANSF TPPIKAQYVRLYPQVCRRHCTLRMELLGCELSGCSEPLGMKSGH IQDYQITASSI FRTLNMDMFTWEPRKARLDKQGKVNAWTSGHNDQSQWLQVDLLVPTKVTGIIT QGAKDFGHVQFVGSYKLAYSNDGEHWTVYQDEKQRKDKVFQGNFDNDTHRKN VIDPPIYARHIRILPWSWYGRITLRSELLGC |
| 13 | FP050 nucleic acid | gacatctgcgaccccaatccttgcgagaatggcggcatttgtctgcctggactggccgatggcagcttctcttgtga atgccccgatggcttcacagaccccaattgcagctctgtggtggaagtggccagcgacgaggaagaacctaca agcgctggcccctgcacacccaatccatgtcataatggcggaacctgcgagatcagcgaggcctacagaggcg ataccttcatcggctacgtgtgcaagtgccccagaggcttcaatggcatccactgccagcacaacatcaacgagt gcgaggtggaaccatgcaagaacggcggcatctgtaccgacctggtggccaattactcttgcgagtgccctggc gagttcatgggcagaaactgccagtacaagggatccgacgctcacaagtctgaggtggcccacagattcaagg acctgggcgaagagaacttcaaggccctggtgctgatcgccttcgctcagtatctgcagcagagccctttcgagg accacgtgaagctggtcaacgaagtgaccgagttcgccaagacctgtgtggccgatgagagcgccgagaactg tgacaagagcctgcacacactgttcggcgacaagctgtgtaccgtggccacactgagagaaacctacggcgag atggccgactgctgtgccaagcaagagcccgagagaaacgagtgcttcctgcagcacaaggacgacaacccc aacctgcctagactcgtgcgacccgaagtggatgtgatgtgcaccgcctttcacgacaacgaggaaaccttcctg aagaagtacctgtacgagatcgccagaeggcacccctacttttatgcccctgagctgctgttcttcgccaagcggta taaggccgccttcaccgaatgttgccaggccgctgataaggctgcctgtctgctgcctaagctggacgagctgag agatgagggcaaagccagctctgccaagcagagactgaagtgcgccagcctgagaagttcggcgagagag cttttaaggcctgggccgttgccagactgagccagagatttcctaaggccgagtttgccgaggtgtccaactcgtg accgatctgacaaaggtgcacaccgagtgctgtcacggcgatctgctggaatgtgccgacgatagagccgacct ggccaagtatatctgcgagaaccaggacagcatcagcagcaagctgaaagagtgctgcgagaagcccctgct ggaaaagtctcactgtatcgccgaggtcgagaacgacgagatgcctgctgatctgcctagcctggccgccgattt cgtggaaagcaaggatgtgtgcaagaactacgccgaggccaaagatgtgtttctgggcatgtttctgtatgagtac gcccgcagacaccccgactattctgtggttctgctgctgcggctggccaagacatacgagacaaccctggaaaa atgctgcgccgctgccgatcctcacgagtgttatgccaaggtgttcgacgagttcaagccactggtggaagaacc ccagaacctgatcaagcagaactgcgagctgttcgagcagctgggcgagtacaagttccagaatgccctgctcg tgcggtacaccaagaaagtgcctcaggtgtccacacctacactggttgaggtgtcccggaatctgggcaaagtgg gcagcaagtgttgcaagcaccctgaggccaagagaatgccttgcgccgaggattacctgagcgtggtgctgaat cagctgtgcgtgctgcacgagaaaacccctgtgtccgacagagtgaccaagtgctgtaccgagagcctcgtgaa cagaaggccttgctttagcgccctggaagtggacgagacatacgtgcccaaagagttcaacgccgagacattca ccttccacgccgatatctgcaccctgtccgagaaagagcggcagatcaagaagcagacagccctggtcgagct ggttaagcacaagcccaaggccaccaaagaacagctgaaggccgtgatggacgacttcgccgcctttgtcgag aagtgctgcaaggccgacgacaaagagacatgcttcgccgaagagggcaagaaactggtggctgcctctcag gctgctctcggacttggaggaagcggaggatctggcggttccggaggaagttgttctggccctcttggcatcgaag gcggcatcatcagcaatcagcagatcaccgccagcagcacccacagagcactgtttggactgcagaaatggta tccctactacgcccggctgaacaagaagggcctgattaacgcctggacagccgccgagaatgacagatggccc tggattcagatcaacctgcagcggaagatgagagtgaccggcgttatcacacagggcgccaaaagaatcggc agccccgagtacatcaagagctacaagatcgcctacagcaacgacggcaagacctgggccatgtacaaagtg aagggcaccaacgaggacatggtgttccggggcaacatcgacaacaacaccccttacgccaacagcttcacc cctcctatcaaggcccagtacgtgcggctgtaccctcaagtgtgcagaaggcactgtaccctgagaatggaactg ctgggctgcgaactgtctggctgttctgagccactgggcatgaagtccggccacatccaggattaccagatcaca gcctccagcatcttcagaaccctgaacatggatatgttcacctgggagccccggaaggccagactggataagca gggaaaagtgaatgcctggaccagcggccacaacgaccagtctcaatggctgcaagtggacctgctggtgccc accaaagtgaccggaatcattactcagggcgcaaaggacttcggccacgtgcagtttgtgggctcctacaagctg gcctactccaacgatggcgagcactggacagtgtaccaggacgagaagcagcgcaaggataaggtgttccag ggaaacttcgataacgatacccaccggaagaacgtgatcgaccctccaatctacgccagacacatcagaatcct gccttggtcttggtacggcagaatcaccctgagatccgagctgctgggatgc |
| 90 | Nucleic acid of Seq ID NO: 84 | gacatctgcgaccccaacccctgcgagaacggcggcatctgcctgcccggcctggccgacggcagcttcagct gcgagtgccccgacggcttcaccgaccccaactgcagcagcgtggtggaggtggccagcgacgaggaggag cccaccggcagcgacgcccacaagagcgaggtggcccaccggttcaaggacctgggcgaggagaacttcaa ggccctggtgctgatcgccttcgcccagtacctgcagcagagccccttcgaggaccacgtgaagctggtgaacg aggtgaccgagttcgccaagacctgcgtggccgacgagagcgccgagaactgcgacaagagcctgcacacc ctgttcggcgacaagctgtgcaccgtggccaccctgcgggagacctacggcgagatggccgactgctgcgcca agcaggagcccgagcggaacgagtgcttcctgcagcacaaggacgacaaccccaacctgccccggctggtg cggcccgaggtggacgtgatgtgcaccgccttccacgacaacgaggagaccttcctgaagaagtacctgtacg agatcgcccggcggcacccctacttctacgcccccgagctgctgttcttcgccaagcggtacaaggccgccttca ccgagtgctgccaggccgccgacaaggccgcctgcctgctgcccaagctggacgagctgcgggacgagggc aaggccagcagcgccaagcagcggctgaagtgcgccagcctgcagaagttcggcgagcgggccttcaaggc ctgggccgtggcccggctgagccagcggttccccaaggccgagttcgccgaggtgagcaagctggtgaccgac ctgaccaaggtgcacaccgagtgctgccacggcgacctgctggagtgcgccgacgaccgggccgacctggcc aagtacatctgcgagaaccaggacagcatcagcagcaagctgaaggagtgctgcgagaagcccctgctggag aagagccactgcatcgccgaggtggagaacgacgagatgcccgccgacctgcccagcctggccgccgacttc gtggagagcaaggacgtgtgcaagaactacgccgaggccaaggacgtgttcctgggcatgttcctgtacgagta cgcccggcggcaccccgactacagcgtggtgctgctgctgcggctggccaagacctacgagaccaccctggag aagtgctgcgccgccgccgacccccacgagtgctacgccaaggtgttcgacgagttcaagcccctggtggagg agccccagaacctgatcaagcagaactgcgagctgttcgagcagctgggcgagtacaagttccagaacgccct gctggtgcggtacaccaagaaggtgccccaggtgagcacccccaccctggtggaggtgagccggaacctggg caaggtgggcagcaagtgctgcaagcaccccgaggccaagcggatgccctgcgccgaggactacctgagcgt ggtgctgaaccagctgtgcgtgctgcacgagaagacccccgtgagcgaccgggtgaccaagtgctgcaccga gagcctggtgaaccggcggccctgcttcagcgccctggaggtggacgagacctacgtgcccaaggagttcaac gccgagaccttcaccttccacgccgacatctgcaccctgagcgagaaggagcggcagatcaagaagcagacc gccctggtggagctggtgaagcacaagcccaaggccaccaaggagcagctgaaggccgtgatggacgacttc gccgccttcgtggagaagtgctgcaaggccgacgacaaggagacctgcttcgccgaggagggcaagaagctg gtggccgccagccaggccgccctgggcctgggcggcagcggcggcagcggcggcagcggcggcagctgca gcggccccctgggcatcgagggcggcatcatcagcaaccagcagatcaccgccagcagcacccaccgggcc ctgttcggcctgcagaagtggtacccctactacgcccggctgaacaagaagggcctgatcaacgcctggaccgc cgccgagaacgaccggtggccctggatccagatcaacctgcagcggaagatgcgggtgaccggcgtgatcac ccagggcgccaagcggatcggcagccccgagtacatcaagagctacaagatcgcctacagcaacgacggc agacctgggccatgtacaaggtgaagggcaccaacgaggacatggtgttccggggcaacatcgacaacaaca ccccctacgccaacagcttcaccccccccatcaaggcccagtacgtgcggctgtacccccaggtgtgccggcgg cactgcaccctgcggatggagctgctgggctgcgagctgagcggctgcagcgagcccctgggcatgaagagc ggccacatccaggactaccagatcaccgccagcagcatcttccggaccctgaacatggacatgttcacctggga gccccggaaggcccggctggacaagcagggcaaggtgaacgcctggaccagcggccacaacgaccagag ccagtggctgcaggtggacctgctggtgcccaccaaggtgaccggcatcatcacccagggcgccaaggacttc ggccacgtgcagttcgtgggcagctacaagctggcctacagcaacgacggcgagcactggaccgtgtaccag gacgagaagcagcggaaggacaaggtgttccagggcaacttcgacaacgacacccaccggaagaacgtgat cgacccccccatctacgcccggcacatccggatcctgccctggagctggtacggccggatcaccctgcggagc gagctgctgggctgc |
| 91 | Nucleic acid of Seq ID NO: 85 | agcgccggcccctgcacccccaacccctgccacaacggcggcacctgcgagatcagcgaggcctaccgggg cgacaccttcatcggctacgtgtgcaagtgcccccggggcttcaacggcatccactgccagcacggcagcgacg cccacaagagcgaggtggcccaccggttcaaggacctgggcgaggagaacttcaaggccctggtgctgatcg ccttcgcccagtacctgcagcagagccccttcgaggaccacgtgaagctggtgaacgaggtgaccgagttcgcc aagacctgcgtggccgacgagagcgccgagaactgcgacaagagcctgcacaccctgttcggcgacaagctg tgcaccgtggccaccctgcgggagacctacggcgagatggccgactgctgcgccaagcaggagcccgagcg gaacgagtgcttcctgcagcacaaggacgacaaccccaacctgccccggctggtgcggcccgaggtggacgt gatgtgcaccgccttccacgacaacgaggagaccttcctgaagaagtacctgtacgagatcgcccggcggcac ccctacttctacgcccccgagctgctgttcttcgccaagcggtacaaggccgccttcaccgagtgctgccaggccg ccgacaaggccgcctgcctgctgcccaagctggacgagctgcgggacgagggcaaggccagcagcgccaa gcagcggctgaagtgcgccagcctgcagaagttcggcgagcgggccttcaaggcctgggccgtggcccggctg agccagcggttccccaaggccgagttcgccgaggtgagcaagctggtgaccgacctgaccaaggtgcacacc gagtgctgccacggcgacctgctggagtgcgccgacgaccgggccgacctggccaagtacatctgcgagaac caggacagcatcagcagcaagctgaaggagtgctgcgagaagcccctgctggagaagagccactgcatcgc cgaggtggagaacgacgagatgcccgccgacctgcccagcctggccgccgacttcgtggagagcaaggacgt gtgcaagaactacgccgaggccaaggacgtgttcctgggcatgttcctgtacgagtacgcccggcggcaccccg actacagcgtggtgctgctgctgcggctggccaagacctacgagaccaccctggagaagtgctgcgccgccgc cgacccccacgagtgctacgccaaggtgttcgacgagttcaagcccctggtggaggagccccagaacctgatc aagcagaactgcgagctgttcgagcagctgggcgagtacaagttccagaacgccctgctggtgcggtacacca agaaggtgccccaggtgagcacccccaccctggtggaggtgagccggaacctgggcaaggtgggcagcaag tgctgcaagcaccccgaggccaagcggatgccctgcgccgaggactacctgagcgtggtgctgaaccagctgt gcgtgctgcacgagaagacccccgtgagcgaccgggtgaccaagtgctgcaccgagagcctggtgaaccggc ggccctgcttcagcgccctggaggtggacgagacctacgtgcccaaggagttcaacgccgagaccttcaccttc cacgccgacatctgcaccctgagcgagaaggaqcggcagatcaagaagcagaccgccctggtggagctggt gaagcacaagcccaaggccaccaaggagcagctgaaggccgtgatggacgacttcgccgccttcgtggaga agtgctgcaaggccgacgacaaggagacctgcttcgccgaggagggcaagaagctggtggccgccagccag gccgccctgggcctgggcggcagcggcggcagcggcggcagcggcggcagctgcagcggccccctgggca tcgagggcggcatcatcagcaaccagcagatcaccgccagcagcacccaccgggccctgttcggcctgcaga agtggtacccctactacgcccggctgaacaagaagggcctgatcaacgcctggaccgccgccgagaacgacc ggtggccctggatccagatcaacctgcagcggaagatgcgggtgaccggcgtgatcacccagggcgccaagc ggatcggcagccccgagtacatcaagagctacaagatcgcctacagcaacgacggcaagacctgggccatgt acaaggtgaagggcaccaacgaggacatggtgttccggggcaacatcgacaacaacaccccctacgccaac agcttcaccccccccatcaaggcccagtacgtgcggctgtacccccaggtgtgccggcggcactgcaccctgcg gatggagctgctgggctgcgagctgagcggctgcagcgagcccctgggcatgaagagcggccacatccagga ctaccagatcaccgccagcagcatcttccggaccctgaacatggacatgttcacctgggagccccggaaggccc ggctggacaagcagggcaaggtgaacgcctggaccagcggccacaacgaccagagccagtggctgcaggt ggacctgctggtgcccaccaaggtgaccggcatcatcacccagggcgccaaggacttcggccacgtgcagttc gtgggcagctacaagctggcctacagcaacgacggcgagcactggaccgtgtaccaggacgagaagcagcg gaaggacaaggtgttccagggcaacttcgacaacgacacccaccggaagaacgtgatcgacccccccatcta cgcccggcacatccggatcctgccctggagctggtacggccggatcaccctgcggagcgagctgctgggctgc |
| 92 | Nucleic acid of Seq ID NO: 86 | aacatcaacgagtgcgaggtggagccctgcaagaacggcggcatctgcaccgacctggtggccaactacagc tgcgagtgccccggcgagttcatgggccggaactgccagtacaagggcagcgacgcccacaagagcgaggt ggcccaccggttcaaggacctgggcgaggagaacttcaaggccctggtgctgatcgccttcgcccagtacctgc agcagagccccttcgaggaccacgtgaagctggtgaacgaggtgaccgagttcgccaagacctgcgtggccg acgagagcgccgagaactgcgacaagagcctgcacaccctgttcggcgacaagctgtgcaccgtggccaccc tgcgggagacctacggcgagatggccgactgctgcgccaagcaggagcccgagcggaacgagtgcttcctgc agcacaaggacgacaaccccaacctgccccggctggtgcggcccgaggtggacgtgatgtgcaccgccttcc acgacaacgaggagaccttcctgaagaagtacctgtacgagatcgcccggcggcacccctacttctacgccccc gagctgctgttcttcgccaagcggtacaaggccgccttcaccgagtgctgccaggccgccgacaaggccgcctg cctgctgcccaagctggacgagctgcgggacgagggcaaggccagcagcgccaagcagcggctgaagtgc gccagcctgcagaagttcggcgagcgggccttcaaggcctgggccgtggcccggctgagccagcggttcccca aggccgagttcgccgaggtgagcaagctggtgaccgacctgaccaaggtgcacaccgagtgctgccacggcg acctgctggagtgcgccgacgaccgggccgacctggccaagtacatctgcgagaaccaggacagcatcagca gcaagctgaaggagtgctgcgagaagcccctgctggagaagagccactgcatcgccgaggtggagaacgac gagatgcccgccgacctgcccagcctggccgccgacttcgtggagagcaaggacgtgtgcaagaactacgcc gaggccaaggacgtgttcctgggcatgttcctgtacgagtacgcccggcggcaccccgactacagcgtggtgctg ctgctgcggctggccaagacctacgagaccaccctggagaagtgctgcgccgccgccgacccccacgagtgct acgccaaggtgttcgacgagttcaagcccctggtggaggagccccagaacctgatcaagcagaactgcgagct gttcgagcagctgggcgagtacaagttccagaacgccctgctggtgcggtacaccaagaaggtgccccaggtg agcacccccaccctggtggaggtgagccggaacctgggcaaggtgggcagcaagtgctgcaagcaccccga ggccaagcggatgccctgcgccgaggactacctgagcgtggtgctgaaccagctgtgcgtgctgcacgagaag acccccgtgagcgaccgggtgaccaagtgctgcaccgagagcctggtgaaccggcggccctgcttcagcgccc tggaggtggacgagacctacgtgcccaaggagttcaacgccgagaccttcaccttccacgccgacatctgcacc ctgagcgagaaggagcggcagatcaagaagcagaccgccctggtggagctggtgaagcacaagcccaagg ccaccaaggagcagctgaaggccgtgatggacgacttcgccgccttcgtggagaagtgctgcaaggccgacg acaaggagacctgcttcgccgaggagggcaagaagctggtggccgccagccaggccgccctgggcctgggc ggcagcggcggcagcggcggcagcggcggcagctgcagcggccccctgggcatcgagggcggcatcatca gcaaccagcagatcaccgccagcagcacccaccgggccctgttcggcctgcagaagtggtacccctactacgc ccggctgaacaagaagggcctgatcaacgcctggaccgccgccgagaacgaccggtggccctggatccagat caacctgcagcggaagatgcgggtgaccggcgtgatcacccagggcgccaagcggatcggcagccccgagt acatcaagagctacaagatcgcctacagcaacgacggcaagacctgggccatgtacaaggtgaagggcacc aacgaggacatggtgttccggggcaacatcgacaacaacaccccctacgccaacagcttcaccccccccatca aggcccagtacgtgcggctgtacccccaggtgtgccggcggcactgcaccctgcggatggagctgctgggctgc gagctgagcggctgcagcgagcccctgggcatgaagagcggccacatccaggactaccagatcaccgccag cagcatcttccggaccctgaacatggacatgttcacctgggagccccggaaggcccggctggacaagcagggc aaggtgaacgcctggaccagcggccacaacgaccagagccagtggctgcaggtggacctgctggtgcccacc aaggtgaccggcatcatcacccagggcgccaaggacttcggccacgtgcagttcgtgggcagctacaagctgg cctacagcaacgacggcgagcactggaccgtgtaccaggacgagaagcagcggaaggacaaggtgttccag ggcaacttcgacaacgacacccaccggaagaacgtgatcgacccccccatctacgcccggcacatccggatc ctgccctggagctggtacggccggatcaccctgcggagcgagctgctgggctgc |
| 93 | Nucleic acid of Seq ID NO: 87 | gacatctgcgaccccaacccctgcgagaacggcggcatctgcctgcccggcctggccgacggcagcttcagct gcgagtgccccgacggcttcaccgaccccaactgcagcagcgtggtggaggtggccagcgacgaggaggag cccaccagcgccggcccctgcacccccaacccctgccacaacggcggcacctgcgagatcagcgaggccta ccggggcgacaccttcatcggctacgtgtgcaagtgcccccggggcttcaacggcatccactgccagcacggca gcgacgcccacaagagcgaggtggcccaccggttcaaggacctgggcgaggagaacttcaaggccctggtg ctgatcgccttcgcccagtacctgcagcagagccccttcgaggaccacgtgaagctggtgaacgaggtgaccga gttcgccaagacctgcgtggccgacgagagcgccgagaactgcgacaagagcctgcacaccctgttcggcga caagctgtgcaccgtggccaccctgcgggagacctacggcgagatggccgactgctgcgccaagcaggagcc cgagcggaacgagtgcttcctgcagcacaaggacgacaaccccaacctgccccggctggtgcggcccgaggt ggacgtgatgtgcaccgccttccacgacaacgaggagaccttcctgaagaagtacctgtacgagatcgcccggc ggcacccctacttctacgcccccgagctgctgttcttcgccaagcggtacaaggccgccttcaccgagtgctgcca ggccgccgacaaggccgcctgcctgctgcccaagctggacgagctgcgggacgagggcaaggccagcagc gccaagcagcggctgaagtgcgccagcctgcagaagttcggcgagcgggccttcaaggcctgggccgtggcc cggctgagccagcggttccccaaggccgagttcgccgaggtgagcaagctggtgaccgacctgaccaaggtgc acaccgagtgctgccacggcgacctgctggagtgcgccgacgaccgggccgacctggccaagtacatctgcg agaaccaggacagcatcagcagcaagctgaaggagtgctgcgagaagcccctgctggagaagagccactgc atcgccgaggtggagaacgacgagatgcccgccgacctgcccagcctggccgccgacttcgtggagagcaag gacgtgtgcaagaactacgccgaggccaaggacgtgttcctgggcatgttcctgtacgagtacgcccggcggca ccccgactacagcgtggtgctgctgctgcggctggccaagacctacgagaccaccctggagaagtgctgcgcc gccgccgacccccacgagtgctacgccaaggtgttcgacgagttcaagcccctggtggaggagccccagaac ctgatcaagcagaactgcgagctgttcgagcagctgggcgagtacaagttccagaacgccctgctggtgcggta caccaagaaggtgccccaggtgagcacccccaccctggtggaggtgagccggaacctgggcaaggtgggca gcaagtgctgcaagcaccccgaggccaagcggatgccctgcgccgaggactacctgagcgtggtgctgaacc agctgtgcgtgctgcacgagaagacccccgtgagcgaccgggtgaccaagtgctgcaccgagagcctggtga accggcggccctgcttcagcgccctggaggtggacgagacctacgtgcccaaggagttcaacgccgagacctt caccttccacgccgacatctgcaccctgagcgagaaggagcggcagatcaagaagcagaccgccctggtgga gctggtgaagcacaagcccaaggccaccaaggagcagctgaaggccgtgatggacgacttcgccgccttcgt ggagaagtgctgcaaggccgacgacaaggagacctgcttcgccgaggagggcaagaagctggtggccgcca gccaggccgccctgggcctgggcggcagcggcggcagcggcggcagcggcggcagctgcagcggccccct gggcatcgagggcggcatcatcagcaaccagcagatcaccgccagcagcacccaccgggccctgttcggcct gcagaagtggtacccctactacgcccggctgaacaagaagggcctgatcaacgcctggaccgccgccgagaa cgaccggtggccctggatccagatcaacctgcagcggaagatgcgggtgaccggcgtgatcacccagggcgc caagcggatcggcagccccgagtacatcaagagctacaagatcgcctacagcaacgacggcaagacctggg ccatgtacaaggtgaagggcaccaacgaggacatggtgttccggggcaacatcgacaacaacaccccctacg ccaacagcttcaccccccccatcaaggcccagtacgtgcggctgtacccccaggtgtgccggcggcactgcacc ctgcggatggagctgctgggctgcgagctgagcggctgcagcgagcccctgggcatgaagagcggccacatc caggactaccagatcaccgccagcagcatcttccggaccctgaacatggacatgttcacctgggagccccgga aggcccggctggacaagcagggcaaggtgaacgcctggaccagcggccacaacgaccagagccagtggct gcaggtggacctgctggtgcccaccaaggtgaccggcatcatcacccagggcgccaaggacttcggccacgtg cagttcgtgggcagctacaagctggcctacagcaacgacggcgagcactggaccgtgtaccaggacgagaag cagcggaaggacaaggtgttccagggcaacttcgacaacgacacccaccggaagaacgtgatcgacccccc catctacgcccggcacatccggatcctgccctggagctggtacggccggatcaccctgcggagcgagctgctgg gctgc |
| 94 | Nucleic acid of Seq ID NO: 88 | agcgccggcccctgcacccccaacccctgccacaacggcggcacctgcgagatcagcgaggcctaccgggg cgacaccttcatcggctacgtgtgcaagtgcccccggggcttcaacggcatccactgccagcacaacatcaacg agtgcgaggtggagccctgcaagaacggcggcatctgcaccgacctggtggccaactacagctgcgagtgccc cggcgagttcatgggccggaactgccagtacaagggcagcgacgcccacaagagcgaggtggcccaccggt tcaaggacctgggcgaggagaacttcaaggccctggtgctgatcgccttcgcccagtacctgcagcagagcccc ttcgaggaccacgtgaagctggtgaacgaggtgaccgagttcgccaagacctgcgtggccgacgagagcgcc gagaactgcgacaagagcctgcacaccctgttcggcgacaagctgtgcaccgtggccaccctgcgggagacct acggcgagatggccgactgctgcgccaagcaggagcccgagcggaacgagtgcttcctgcagcacaaggac gacaaccccaacctgccccggctggtgcggcccgaggtggacgtgatgtgcaccgccttccacgacaacgag gagaccttcctgaagaagtacctgtacgagatcgcccggcggcacccctacttctacgcccccgagctgctgttctt cgccaagcggtacaaggccgccttcaccgagtgctgccaggccgccgacaaggccgcctgcctgctgcccaa gctggacgagctgcgggacgagggcaaggccagcagcgccaagcagcggctgaagtgcgccagcctgcag aagttcggcgagcgggccttcaaggcctgggccgtggcccggctgagccagcggttccccaaggccgagttcg ccgaggtgagcaagctggtgaccgacctgaccaaggtgcacaccgagtgctgccacggcgacctgctggagt gcgccgacgaccgggccgacctggccaagtacatctgcgagaaccaggacagcatcagcagcaagctgaag gagtgctgcgagaagcccctgctggagaagagccactgcatcgccgaggtggagaacgacgagatgcccgc cgacctgcccagcctggccgccgacttcgtggagagcaaggacgtgtgcaagaactacgccgaggccaagga cgtgttcctgggcatgttcctgtacgagtacgcccggcggcaccccgactacagcgtggtgctgctgctgcggctg gccaagacctacgagaccaccctggagaagtgctgcgccgccgccgacccccacgagtgctacgccaaggtg ttcgacgagttcaagcccctggtggaggagccccagaacctgatcaagcagaactgcgagctgttcgagcagct gggcgagtacaagttccagaacgccctgctggtgcggtacaccaagaaggtgccccaggtgagcacccccac cctggtggaggtgagccggaacctgggcaaggtgggcagcaagtgctgcaagcaccccgaggccaagcgga tgccctgcgccgaggactacctgagcgtggtgctgaaccagctgtgcgtgctgcacgagaagacccccgtgagc gaccgggtgaccaagtgctgcaccgagagcctggtgaaccggcggccctgcttcagcgccctggaggtggacg agacctacgtgcccaaggagttcaacgccgagaccttcaccttccacgccgacatctgcaccctgagcgagaa ggagcggcagatcaagaagcagaccgccctggtggagctggtgaagcacaagcccaaggccaccaaggag cagctgaaggccgtgatggacgacttcgccgccttcgtggagaagtgctgcaaggccgacgacaaggagacct gcttcgccgaggagggcaagaagctggtggccgccagccaggccgccctgggcctgggcggcagcggcggc agcggcggcagcggcggcagctgcagcggccccctgggcatcgagggcggcatcatcagcaaccagcagat caccgccagcagcacccaccgggccctgttcggcctgcagaagtggtacccctactacgcccggctgaacaag aagggcctgatcaacgcctggaccgccgccgagaacgaccggtggccctggatccagatcaacctgcagcgg aagatgcgggtgaccggcgtgatcacccagggcgccaagcggatcggcagccccgagtacatcaagagcta caagatcgcctacagcaacgacggcaagacctgggccatgtacaaggtgaagggcaccaacgaggacatgg tgttccggggcaacatcgacaacaacaccccctacgccaacagcttcaccccccccatcaaggcccagtacgt gcggctgtacccccaggtgtgccggcggcactgcaccctgcggatggagctgctgggctgcgagctgagcggct gcagcgagcccctgggcatgaagagcggccacatccaggactaccagatcaccgccagcagcatcttccgga ccctgaacatggacatgttcacctgggagccccggaaggcccggctggacaagcagggcaaggtgaacgcct ggaccagcggccacaacgaccagagccagtggctgcaggtggacctgctggtgcccaccaaggtgaccggc atcatcacccagggcgccaaggacttcggccacgtgcagttcgtgggcagctacaagctggcctacagcaacg acggcgagcactggaccgtgtaccaggacgagaagcagcggaaggacaaggtgttccagggcaacttcgac aacgacacccaccggaagaacgtgatcgacccccccatctacgcccggcacatccggatcctgccctggagct ggtacggccggatcaccctgcggagcgagctgctgggctgc |
| 95 | Nucleic acid of Seq ID NO: 89 | gacatctgcgaccccaacccctgcgagaacggcggcatctgcctgcccggcctggccgacggcagcttcagct gcgagtgccccgacggcttcaccgaccccaactgcagcagcgtggtggaggtggccagcgacgaggaggag cccaccaacatcaacgagtgcgaggtggagccctgcaagaacggcggcatctgcaccgacctggtggccaac tacagctgcgagtgccccggcgagttcatgggccggaactgccagtacaagggcagcgacgcccacaagagc gaggtggcccaccggttcaaggacctgggcgaggagaacttcaaggccctggtgctgatcgccttcgcccagta cctgcagcagagccccttcgaggaccacgtgaagctggtgaacgaggtgaccgagttcgccaagacctgcgtg gccgacgagagcgccgagaactgcgacaagagcctgcacaccctgttcggcgacaagctgtgcaccgtggcc accctgcgggagacctacggcgagatggccgactgctgcgccaagcaggagcccgagcggaacgagtgcttc ctgcagcacaaggacgacaaccccaacctgccccggctggtgcggcccgaggtggacgtgatgtgcaccgcc ttccacgacaacgaggagaccttcctgaagaagtacctgtacgagatcgcccggcggcacccctacttctacgc ccccgagctgctgttcttcgccaagcggtacaaggccgccttcaccgagtgctgccaggccgccgacaaggccg cctgcctgctgcccaagctggacgagctgcgggacgagggcaaggccagcagcgccaagcagcggctgaag tgcgccagcctgcagaagttcggcgagcgggccttcaaggcctgggccgtggcccggctgagccagcggttcc ccaaggccgagttcgccgaggtgagcaagctggtgaccgacctgaccaaggtgcacaccgagtgctgccacg gcgacctgctggagtgcgccgacgaccgggccgacctggccaagtacatctgcgagaaccaggacagcatca gcagcaagctgaaggagtgctgcgagaagcccctgctggagaagagccactgcatcgccgaggtggagaac gacgagatgcccgccgacctgcccagcctggccgccgacttcgtggagagcaaggacgtgtgcaagaactac gccgaggccaaggacgtgttcctgggcatgttcctgtacgagtacgcccggcggcaccccgactacagcgtggt gctgctgctgcggctggccaagacctacgagaccaccctggagaagtgctgcgccgccgccgacccccacga gtgctacgccaaggtgttcgacgagttcaagcccctggtggaggagccccagaacctgatcaagcagaactgc gagctgttcgagcagctgggcgagtacaagttccagaacgccctgctggtgcggtacaccaagaaggtgcccc aggtgagcacccccaccctggtggaggtgagccggaacctgggcaaggtgggcagcaagtgctgcaagcac cccgaggccaagcggatgccctgcgccgaggactacctgagcgtggtgctgaaccagctgtgcgtgctgcacg agaagacccccgtgagcgaccgggtgaccaagtgctgcaccgagagcctggtgaaccggcggccctgcttca gcgccctggaggtggacgagacctacgtgcccaaggagttcaacgccgagaccttcaccttccacgccgacat ctgcaccctgagcgagaaggagcggcagatcaagaagcagaccgccctggtggagctggtgaagcacaagc ccaaggccaccaaggagcagctgaaggccgtgatggacgacttcgccgccttcgtggagaagtgctgcaagg ccgacgacaaggagacctgcttcgccgaggagggcaagaagctggtggccgccagccaggccgccctgggc ctgggcggcagcggcggcagcggcggcagcggcggcagctgcagcggccccctgggcatcgagggcggca tcatcagcaaccagcagatcaccgccagcagcacccaccgggccctgttcggcctgcagaagtggtaccccta ctacgcccggctgaacaagaagggcctgatcaacgcctggaccgccgccgagaacgaccggtggccctggat ccagatcaacctgcagcggaagatgcgggtgaccggcgtgatcacccagggcgccaagcggatcggcagcc ccgagtacatcaagagctacaagatcgcctacagcaacgacggcaagacctgggccatgtacaaggtgaagg gcaccaacgaggacatggtgttccggggcaacatcgacaacaacaccccctacgccaacagcttcacccccc ccatcaaggcccagtacgtgcggctgtacccccaggtgtgccggcggcactgcaccctgcggatggagctgctg ggctgcgagctgagcggctgcagcgagcccctgggcatgaagagcggccacatccaggactaccagatcacc gccagcagcatcttccggaccctgaacatggacatgttcacctgggagccccggaaggcccggctggacaagc agggcaaggtgaacgcctggaccagcggccacaacgaccagagccagtggctgcaggtggacctgctggtg cccaccaaggtgaccggcatcatcacccagggcgccaaggacttcggccacgtgcagttcgtgggcagctaca agctggcctacagcaacgacggcgagcactggaccgtgtaccaggacgagaagcagcggaaggacaaggt gttccagggcaacttcgacaacgacacccaccggaagaacgtgatcgacccccccatctacgcccggcacatc cggatcctgccctggagctggtacggccggatcaccctgcggagcgagctgctgggctgc |
| 14 | FP060 EGF-C1-C2-Fc [S354C, T366W] | LDICSKNPCHNGGLCEEISQEVRGDVFPSYTCTCLKGYAGNHCETKCVEPLGME NGNIANSQIAASSVRVTFLGLQHWVPELARLNRAGMVNAWTPSSNDDNPWIQV NLLRRMWVTGVVTQGASRLASHEYLKAFKVAYSLNGHEFDFIHDVNKKHKEFVG NWNKNAVHVNLFETPVEAQYVRLYPTSCHTACTLRFELLGCELNGCANPLGLKN NSIPDKQITASSSYKTWGLHLFSWNPSYARLDKQGNFNAWVAGSYGNDQWLQV DLGSSKEVTGIITQGARNFGSVQFVASYKVAYSNDSANWTEYQDPRTGSSKIFP GNWDNHSHKKNLFETPILARYVRILPVAWHNRIALRLELLGCGGGGTDKTHTCP PCPAPELLGGPSVFLFPPKPKDTLMISRTPEVTCVVVDVSHEDPEVKFNWYVDG VEVHNAKTKPREEQYNSTYRVVSVLTVLHQDWLNGKEYKCKVSNKALPAPIEKTI SKAKGQPREPQVYTLPPCREEMTKNQVSLWCLVKGFYPSDIAVEWESNGQPEN NYKTTPPVLDSDGSFFLYSKLTVDKSRWQQGNVFSCSVMHEALHNHYTQKSLS LSPGK |
| 15 | FP060 nucleic acid | ctggacatctgcagcaagaacccctgccacaacggcggcctgtgcgaagagatcagccaggaagtgcgggg cgacgtgttccccagctacacctgtacctgcctgaagggctacgccggcaaccactgcgagactaagtgcgtgg aacccctgggcatggaaaacggcaatattgccaacagccagatcgccgccagctccgtgcgcgtgacctttctg ggactgcagcactgggtgcccgagctggccagactgaacagagccggcatggtgaacgcctggacccccagc agcaacgacgacaacccttggatccaggtgaacctgctgcggcggatgtgggtgacaggcgtggtgacacagg gcgccagcagactggccagccacgagtacctgaaggcctttaaggtggcctacagcctgaacggccacgagtt cgacttcatccacgacgtgaacaagaaacacaaagaatttgtgggcaactggaacaagaacgccgtgcacgtg aacctgttcgagacacccgtggaagcccagtacgtgcggctgtaccccaccagctgccacaccgcctgcaccct gagattcgagctgctgggctgcgagctgaacggctgcgccaaccccctgggcctgaagaacaacagcatcccc gacaagcagatcaccgcctccagcagctacaagacctggggcctgcacctgttcagctggaaccccagctacg cccggctggacaagcagggcaacttcaacgcctgggtggccggcagctacggcaacgaccagtggctgcagg tggacctgggcagcagcaaagaagtgaccggcatcatcacccagggggccagaaacttcggcagcgtgcagt tcgtggccagctacaaagtggcctactccaacgacagcgccaactggaccgagtaccaggacccccggaccg gcagctccaagatcttccccggcaactgggacaaccacagccacaagaagaatctgttcgaaacccccatcct ggccagatacgtgcggatcctgcccgtggcctggcacaaccggatcgccctgagactggaactgctgggatgtg ggggaggcggtaccgacaagacccacacctgccccccctgcccagccccagagctgctgggcggaccctcc gtgttcctgttcccccccaagcccaaggacaccctgatgatcagcaggacccccgaggtgacctgcgtggtggtg gacgtgagccacgaggacccagaggtgaagttcaactggtacgtggacggcgtggaggtgcacaacgccaa gaccaagcccagagaggagcagtacaacagcacctacagggtggtgtccgtgctgaccgtgctgcaccagga ctggctgaacggcaaggaatacaagtgcaaggtctccaacaaggccctgccagcccccatcgaaaagaccat cagcaaggccaagggccagccacgggagccccaggtgtacaccctgcccccctgccgggaggagatgacc aagaaccaggtgtccctgtggtgtctggtgaagggcttctaccccagcgacatcgccgtggagtgggagagcaa cggccagcccgagaacaactacaagaccacccccccagtgctggacagcgacggcagcttcttcctgtacagc aagctgaccgtggacaagtccaggtggcagcagggcaacgtgttcagctgcagcgtgatgcacgaggccctgc acaaccactacacccagaagagcctgagcctgtcccccggcaag |
| 16 | FP070 EGF-Fc-C1-C2 | LDICSKNPCHNGGLCEEISQEVRGDVFPSYTCTCLKGYAGNHCETKGSDKTHTC PPCPAPELLGGPSVFLFPPKPKDTLMISRTPEVTCVVVDVSHEDPEVKFNWYVD GVEVHNAKTKPREEQYNSTYRVVSVL TVLHQDWLNGKEYKCKVSNKALP API EK TISKAKGQPREPQVYTLPPSREEMTKNQVSLTCLVKGFYPSDIAVEWESNGQPE NNYKTTPPVLDSDGSFFLYSKLTVDKSRWQQGNVFSCSVMHEALHNHYTQKSL SLSPGKGGSGGSGGSGGSCVEPLGMENGNIANSQIAASSVRVTFLGLQHWVPE LARLNRAGMVNAWTPSSNDDNPWIQVNLLRRMWVTGVVTQGASRLASHEYLK AFKVAYSLNGHEFDFIHDVNKKHKEFVGNWNKNAVHVNLFETPVEAQYVRLYPT SCHTACTLRFELLGCELNGCANPLGLKNNSIPDKQITASSSYKTWGLHLFSWNPS YARLDKQGNFNAWVAGSYGNDQWLQVDLGSSKEVTGIITQGARNFGSVQFVAS YKVAYSNDSANWTEYQDPRTGSSKIFPGNWDNHSHKKNLFETPILARYVRILPVA WHNRIALRLELLGCGSHHHHHH |
| 17 | FP070 nucleic acid | ctggacatctgcagcaagaatccttgccacaacggcggcctgtgcgaagagatttctcaagaagtgcggggcga cgttttccccagctacacctgtacatgtctgaagggctacgccggcaaccactgcgagacaaaaggatccgaca agacccacacctgccccccctgcccagccccagagctgctgggcggaccctccgtgttcctgttcccccccaagc ccaaggacaccctgatgatcagcaggacccccgaggtgacctgcgtggtggtggacgtgagccacgaggacc cagaggtgaagttcaactggtacgtggacggcgtggaggtgcacaacgccaagaccaagcccagagaggag cagtacaacagcacctacagggtggtgtccgtgctgaccgtgctgcaccaggactggctgaacggcaaggaat acaagtgcaaggtctccaacaaggccctgccagcccccatcgaaaagaccatcagcaaggccaagggccag ccacgggagccccaggtgtacaccctgcccccctcccgggaggagatgaccaagaaccaggtgtccctgacct gtctggtgaagggcttctaccccagcgacatcgccgtggagtgggagagcaacggccagcccgagaacaact acaagaccacccccccagtgctggacagcgacggcagcttcttcctgtacagcaagctgaccgtggacaagtc caggtggcagcagggcaacgtgttcagctgcagcgtgatgcacgaggccctgcacaaccactacacccagaa gagcctgagcctgtcccccggcaagggaggaagcggaggatctggcggttccggaggctcttgtgtggaaccc ctcggcatggaaaacggcaatatcgccaatagccagattgccgccagcagcgtcagagtgacatttctgggact gcagcactgggtgcccgagctggctagactgaatagagccggcatggtcaacgcctggacacccagcagcaa cgacgataacccttggattcaagtgaacctgctgcggcgtatgtgggtcacaggtgttgttacacagggcgcctcta gactggccagccacgagtatctgaaggcctttaaggtggcctacagcctgaacggccacgagttcgacttcatcc acgacgtgaacaagaagcacaaagagtttgtcggcaactggaacaagaacgccgtgcacgtgaacctgttcg agacacctgtggaagcccagtacgtgcggctgtaccctacaagctgtcacaccgcctgcactctgagattcgaac tgctgggatgcgagctgaacggctgtgctaatcctctgggcctgaagaacaacagcatccccgataagcagatc accgccagctccagctataagacatggggcctgcacctgttcagctggaacccttcttacgccagactggacaag cagggcaacttcaatgcttgggtggccggcagctacggcaatgatcagtggctgcaagtggacctgggcagcag caaagaagtgacaggcatcatcacccagggcgccagaaatttcggcagcgtgcagtttgtggccagctacaaa gtggcctactccaacgacagcgccaactggaccgagtatcaggaccctagaaccggcagctccaagatcttcc ccggcaattgggacaaccacagccacaagaagaatctgttcgaaacccctatcctggccagatatgtgcgcattc tgcccgtggcctggcacaacagaattgccctgagactggaactgctcggctgtggctctcaccaccaccatcacc at |
| 18 | FP071 EGF-Fc(knob)-C1-C2 | LDICSKNPCHNGGLCEEISQEVRGDVFPSYTCTCLKGYAGNHCETKGSDKTHTC PPCPAPELLGGPSVFLFPPKPKDTLMISRTPEVTCVVVDVSHEDPEVKFNWYVD GVEVHNAKTKPREEQYNSTYRVVSVLTVLHQD\ALNGKEYKCKVSNKALPAPIEK TISKAKGQPREPQVYTLPPCREEMTKNQVSLWCLVKGFYPSDIAVEWESNGQP ENNYKTTPPVLDSDGSFFLYSKLTVDKSRWQQGNVFSCSVMHEALHNHYTQKS LSLSPGKGGSGGSGGSGGSCVEPLGMENGNIANSQIAASSVRVTFLGLQHWVP ELARLNRAGMVNAWTPSSNDDNPWIQVNLLRRMWVTGVVTQGASRLASHEYL KAFKVAYSLNGHEFDFIHDVNKKHKEFVGNWNKNAVHVNLFETPVEAQYVRLYP TSCHTACTLRFELLGCELNGCANPLGLKNNSIPDKQITASSSYKTWGLHLFSWNP SYARLDKQGNFNAWVAGSYGNDQWLQVDLGSSKEVTGIITQGARNFGSVQFVA SYKVAYSNDSANWTEYQDPRTGSSKIFPGNWDNHSHKKNLFETPILARYVRILPV AWHNRIALRLELLGCGSHHHHHH |
| 19 | FP071 nucleic acid | ctggacatctgcagcaagaatccttgccacaacggcggcctgtgcgaagagatttctcaagaagtgcggg gcgacgttttccccagctacacctgtacatgtctgaagggctacgccggcaaccactgcgagacaaaagg atccgacaagacccacacctgccccccctgcccagccccagagctgctgggcggaccctccgtgttcctgt tcccccccaagcccaaggacaccctgatgatcagcaggacccccgaggtgacctgcgtggtggtggacgt gagccacgaggacccagaggtgaagttcaactggtacgtggacggcgtggaggtgcacaacgccaaga ccaagcccagagaggagcagtacaacagcacctacagggtggtgtccgtgctgaccgtgctgcaccagg actggctgaacggcaaggaatacaagtgcaaggtctccaacaaggccctgccagcccccatcgaaaag accatcagcaaggccaagggccagccacgggagccccaggtgtacaccctgcccccctgccgggagg agatgaccaagaaccaggtgtccctgtggtgtctggtgaagggcttctaccccagcgacatcgccgtggagt gggagagcaacggccagcccgagaacaactacaagaccacccccccagtgctggacagcgacggca gcttcttcctgtacagcaagctgaccgtggacaagtccaggtggcagcagggcaacgtgttcagctgcagc gtgatgcacgaggccctgcacaaccactacacccagaagagcctgagcctgtcccccggcaagggagg aagcggaggatctggcggttccggaggctcttgtgtggaacccctcggcatggaaaacggcaatatcgcc aatagccagattgccgccagcagcgtcagagtgacatttctgggactgcagcactgggtgcccgagctggc tagactgaatagagccggcatggtcaacgcctggacacccagcagcaacgacgataacccttggattcaa gtgaacctgctgcggcgtatgtgggtcacaggtgttgttacacagggcgcctctagactggccagccacgag tatctgaaggcctttaaggtggcctacagcctgaacggccacgagttcgacttcatccacgacgtgaacaag aagcacaaagagtttgtcggcaactggaacaagaacgccgtgcacgtgaacctgttcgagacacctgtgg aagcccagtacgtgcggctgtaccctacaagctgtcacaccgcctgcactctgagattcgaactgctgggat gcgagctgaacggctgtgctaatcctctgggcctgaagaacaacagcatccccgataagcagatcaccgc cagctccagctataagacatggggcctgcacctgttcagctggaacccttcttacgccagactggacaagc agggcaacttcaatgcttgggtggccggcagctacggcaatgatcagtggctgcaagtggacctgggcag cagcaaagaagtgacaggcatcatcacccagggcgccagaaatttcggcagcgtgcagtttgtggccagc tacaaagtggcctactccaacgacagcgccaactggaccgagtatcaggaccctagaaccggcagctcc aagatcttccccggcaattgggacaaccacagccacaagaagaatctgttcgaaacccctatcctggcca gatatgtgcgcattctgcccgtggcctggcacaacagaattgccctgagactggaactgctcggctgtggctc tcaccaccaccatcaccat |
| 20 | FP072 EGF-Fc(hole)-C1-C2 | LDICSKNPCHNGGLCEEISQEVRGDVFPSYTCTCLKGYAGNHCETKGSDKT HTCPPCPAPELLGGPSVFLFPPKPKDTLMISRTPEVTCVVVDVSHEDPEVKF NWYVDGVEVHNAKTKPREEQYNSTYRVVSVLTVLHQDWLNGKEYKCKVSN KALPAPIEKTISKAKGQPREPQVCTLPPSREEMTKNQVSLSCAVKGFYPSDIA VEWESNGQPENNYKTTPPVLDSDGSFFLVSKLTVDKSRWQQGNVFSCSVM HEALHNHYTQKSLSLSPGKGGSGGSGGSGGSCVEPLGMENGNIANSQIAAS SVRVTFLGLQHWVPELARLNRAGMVNAWTPSSNDDNPWIQVNLLRRMWVT GVVTQGASRLASHEYLKAFKVAYSLNGHEFDFIHDVNKKHKEFVGNWNKNA VHVNLFETPVEAQYVRLYPTSCHTACTLRFELLGCELNGCANPLGLKNNSIP DKQITASSSYKTWGLHLFSWNPSYARLDKQGNFNAWVAGSYGNDQWLQV DLGSSKEVTGIITQGARNFGSVQFVASYKVAYSNDSANWTEYQDPRTGSSKI FPGNWDNHSHKKNLFETPILARYVRILPVAWHNRIALRLELLGCGSHHHHHH |
| 21 | FP072 nucleic acid | ctggacatctgcagcaagaatccttgccacaacggcggcctgtgcgaagagatttctcaagaagtgcggg gcgacgttttccccagctacacctgtacatgtctgaagggctacgccggcaaccactgcgagacaaaagg atccgacaagacccacacctgccccccctgcccagccccagagctgctgggcggaccctccgtgttcctgt tcccccccaagcccaaggacaccctgatgatcagcaggacccccgaggtgacctgcgtggtggtggacgt gagccacgaggacccagaggtgaagttcaactggtacgtggacggcgtggaggtgcacaacgccaaga ccaagcccagagaggagcagtacaacagcacctacagggtggtgtccgtgctgaccgtgctgcaccagg actggctgaacggcaaggaatacaagtgcaaggtctccaacaaggccctgccagcccccatcgaaaag accatcagcaaggccaagggccagccacgggagccccaggtgtgcaccctgcccccctcccgggagg agatgaccaagaaccaggtgtccctgtcctgtgcggtgaagggcttctaccccagcgacatcgccgtggag tgggagagcaacggccagcccgagaacaactacaagaccacccccccagtgctggacagcgacggca gcttcttcctggtcagcaagctgaccgtggacaagtccaggtggcagcagggcaacgtgttcagctgcagc gtgatgcacgaggccctgcacaaccactacacccagaagagcctgagcctgtcccccggcaagggagg aagcggaggatctggcggttccggaggctcttgtgtggaacccctcggcatggaaaacggcaatatcgcc aatagccagattgccgccagcagcgtcagagtgacatttctgggactgcagcactgggtgcccgagctggc tagactgaatagagccggcatggtcaacgcctggacacccagcagcaacgacgataacccttggattcaa gtgaacctgctgcggcgtatgtgggtcacaggtgttgttacacagggcgcctctagactggccagccacgag tatctgaaggcctttaaggtggcctacagcctgaacggccacgagttcgacttcatccacgacgtgaacaag aagcacaaagagtttgtcggcaactggaacaagaacgccgtgcacgtgaacctgttcgagacacctgtgg aagcccagtacgtgcggctgtaccctacaagctgtcacaccgcctgcactctgagattcgaactgctgggat gcgagctgaacggctgtgctaatcctctgggcctgaagaacaacagcatccccgataagcagatcaccgc cagctccagctataagacatggggcctgcacctgttcagctggaacccttcttacgccagactggacaagc agggcaacttcaatgcttgggtggccggcagctacggcaatgatcagtggctgcaagtggacctgggcag cagcaaagaagtgacaggcatcatcacccagggcgccagaaatttcggcagcgtgcagtttgtggccagc tacaaagtggcctactccaacgacagcgccaactggaccgagtatcaggaccctagaaccggcagctcc aagatcttccccggcaattgggacaaccacagccacaagaagaatctgttcgaaacccctatcctggcca gatatgtgcgcattctgcccgtggcctggcacaacagaattgccctgagactggaactgctcggctgtggctc tcaccaccaccatcaccat |
| 22 | FP080 EGF-C1-C2-Fc | LDICSKNPCHNGGLCEEISQEVRGDVFPSYTCTCLKGYAGNHCETKCVEPLGME NGNIANSQIAASSVRVTFLGLQHWVPELARLNRAGMVNAWTPSSNDDNPWIQV NLLRRMWVTGVVTQGASRLASHEYLKAFKVAYSLNGHEFDFIHDVNKKHKEFVG NWNKNAVHVNLFETPVEAQYVRLYPTSCHTACTLRFELLGCELNGCANPLGLKN NSIPDKQITASSSYKTWGLHLFSWNPSYARLDKQGNFNAWVAGSYGNDQWLQV DLGSSKEVTGIITQGARNFGSVQFVASYKVAYSNDSANWTEYQDPRTGSSKIFP GNWDNHSHKKNLFETPILARYVRILPVAWHNRIALRLELLGCGGGGTDKTHTCP PCPAPELLGGPSVFLFPPKPKDTLMISRTPEVTCVVVDVSHEDPEVKFNWYVDG VEVHNAKTKPREEQYNSTYRVVSVLTVLHQDWLNGKEYKCKVSNKALPAPIEKTI SKAKGQPREPQVYTLPPSREEMTKNQVSLTCLVKGFYPSDIAVEWESNGQPEN NYKTTPPVLDSDGSFFLYSKLTVDKSRWQQGNVFSCSVMHEALHNHYTQKSLS LSPGK |
| 23 | FP080 nucleic acid | ctggacatctgcagcaagaacccctgccacaacggcggcctgtgcgaagagatcagccaggaagtgcgggg cgacgtgttccccagctacacctgtacctgcctgaagggctacgccggcaaccactgcgagactaagtgcgtgg aacccctgggcatggaaaacggcaatatcgccaacagccagatcgccgccagctccgtgcgcgtgacctttctg ggactgcagcactgggtgcccgagctggccagactgaacagagccggcatggtgaacgcctggacccccagc agcaacgacgacaacccttggatccaggtgaacctgctgcggcggatgtgggtgacaggcgtggtgacacagg gcgccagcagactggccagccacgagtacctgaaggcctttaaggtggcctacagcctgaacggccacgagtt cgacttcatccacgacgtgaacaagaaacacaaagaatttgtgggcaactggaacaagaacgccgtgcacgtg aacctgttcgagacacccgtggaagcccagtacgtgcggctgtaccccaccagctgccacaccgcctgcaccct gagattcgagctgctgggctgcgagctgaacggctgcgccaaccccctgggcctgaagaacaacagcatcccc gacaagcagatcaccgcctccagcagctacaagacctggggcctgcacctgttcagctggaaccccagctacg cccggctggacaagcagggcaacttcaacgcctgggtggccggcagctacggcaacgaccagtggctgcagg tggacctgggcagcagcaaagaagtgaccggcatcatcacccagggggccagaaacttcggcagcgtgcagt tcgtggccagctacaaagtggcctactccaacgacagcgccaactggaccgagtaccaggacccccggaccg gcagctccaagatcttccccggcaactgggacaaccacagccacaagaagaatctgttcgaaacccccatcct ggccagatacgtgcggatcctgcccgtggcctggcacaaccggatcgccctgagactggaactgctgggatgtg ggggaggcggtaccgacaagacccacacctgccccccctgcccagccccagagctgctgggcggaccctcc gtgttcctgttcccccccaagcccaaggacaccctgatgatcagcaggacccccgaggtgacctgcgtggtggtg gacgtgagccacgaggacccagaggtgaagttcaactggtacgtggacggcgtggaggtgcacaacgccaa gaccaagcccagagaggagcagtacaacagcacctacagggtggtgtccgtgctgaccgtgctgcaccagga ctggctgaacggcaaggaatacaagtgcaaggtctccaacaaggccctgccagcccccatcgaaaagaccat cagcaaggccaagggccagccacgggagccccaggtgtacaccctgcccccctcccgggaggagatgacca agaaccaggtgtccctgacctgtctggtgaagggcttctaccccagcgacatcgccgtggagtgggagagcaac ggccagcccgagaacaactacaagaccacccccccagtgctggacagcgacggcagcttcttcctgtacagca agctgaccgtggacaagtccaggtggcagcagggcaacgtgttcagctgcagcgtgatgcacgaggccctgca caaccactacacccagaagagcctgagcctgtcccccggcaag |
| 24 | FP090 Fc-EGF-C1-C2 | DKTHTCPPCPAPELLGGPSVFLFPPKPKDTLMISRTPEVTCVVVDVSHEDPEVKF NWYVDGVEVHNAKTKPREEQYNSTYRVVSVLTVLHQDWLNGKEYKCKVSNKAL PAPIEKTISKAKGQPREPQVYTLPPSREEMTKNQVSLTCLVKGFYPSDIAVEWES NGQPENNYKTTPPVLDSDGSFFLYSKLTVDKSRWQQGNVFSCSVMHEALHNHY TQKSLSLSPGKGSLEVLFQGPGSSLDICSKNPCHNGGLCEEISQEVRGDVFPSY TCTCLKGYAGNHCETKCVEPLGMENGNIANSQIAASSVRVTFLGLQHWVPELAR LNRAGMVNAWTPSSNDDNPWIQVNLLRRMWVTGVVTQGASRLASHEYLKAFK VAYSLNGHEFDFIHDVNKKHKEFVGNWNKNAVHVNLFETPVEAQYVRLYPTSCH TACTLRFELLGCELNGCANPLGLKNNSIPDKQITASSSYKTWGLHLFSWNPSYAR LDKQGNFNAWVAGSYGNDQWLQVDLGSSKEVTGIITQGARNFGSVQFVASYKV AYSNDSANWTEYQDPRTGSSKIFPGNWDNHSHKKNLFETPILARYVRILPVAWH NRIALRLELLGC |
| 25 | FP090 nucleic acid | gacaagacccacacctgtcccccctgccctgctcctgagctgctgggaggacccagcgtgttcctgttccccccca agcccaaggacaccctgatgatcagccggacccccgaagtgacctgcgtggtggtggacgtgtcccacgagga ccctgaagtgaagttcaattggtacgtggacggcgtggaggtgcacaacgccaagaccaagccccgggagga acagtacaacagcacctaccgggtggtgtccgtgctgaccgtgctgcaccaggactggctgaacggcaaagaa tacaagtgcaaggtgtccaacaaggccctgcctgcccccatcgagaaaaccatcagcaaggccaagggccag cccagagaaccccaggtgtacacactcccaccaagccgggaggaaatgaccaagaaccaggtgtccctgac ctgcctggtgaagggcttctaccccagcgacattgccgtggagtgggagagcaacggccagcctgagaacaac tacaagaccacccctccagtcctcgattctgatggatctttcttcctgtactccaagctgaccgtggacaagagccg gtggcagcagggaaacgtcttttcctgttccgtcatgcatgaggctctccacaatcactacacccagaagtccctga gcctgagccccggcaagggatccctcgaggtgctgtttcagggaccaggcagcagcctggacatctgcagcaa gaacccctgccacaacggcggcctgtgcgaagagatcagccaggaagtgcggggcgacgtgttccccagcta cacctgtacctgcctgaagggctacgccggcaaccactgcgagactaagtgcgtggaacccctgggaatggaa aacggcaatatcgccaacagccagatcgccgccagctccgtcagagtgacctttctgggactccagcactgggt gcccgagctggccagactgaatagagccggcatggtcaacgcctggacccccagcagcaacgacgacaacc cctggattcaagtgaacctgctgcggcgtatgtgggtcaccggcgtcgtgacacagggcgctagcagactggcc agccacgagtacctgaaggcctttaaggtggcctacagcctgaacggccacgagttcgacttcatccacgacgtg aacaagaaacacaaagaatttgtgggcaactggaacaagaacgccgtgcacgtgaacctgttcgagacaccc gtggaagcccagtacgtgcggctgtaccctaccagctgtcacaccgcctgcaccttaagattcgagctgctgggct gcgagctgaacggctgcgctaatcctctgggcctgaagaacaacagcatccccgacaagcagatcaccgcctc cagcagctacaagacctggggactgcacctgttcagctggaaccctagctacgcccggctggacaagcagggc aacttcaatgcttgggtggccggcagctacggcaacgaccagtggctccaggtggacctgggcagcagcaaag aagtgaccggcatcatcacccagggggccagaaacttcggcagcgtgcagttcgtggcctcctacaaagtggcc tactccaacgacagcgccaactggaccgagtaccaggaccctagaaccggcagctccaagattttccccggca actgggataaccacagccacaagaagaatctgttcgaaacccccatcctggcccgctacgtgcgcattctaccg gtcgcctggcacaaccggatcgccctgagactggaactgctgggatgc |
| 26 | FP100 EGF-C2-C2 | LDICSKNPCHNGGLCEEISQEVRGDVFPSYTCTCLKGYAGNHCETKGCANP LGLKNNSIPDKQITASSSYKTWGLHLFSWNPSYARLDKQGNFNAWVAGSYG NDQWLQVDLGSSKEVTGIITQGARNFGSVQFVASYKVAYSNDSANWTEYQD PRTGSSKIFPGNWDNHSHKKNLFETPILARYVRILPVAWHNRIALRLELLGCG CANPLGLKNNSIPDKQITASSSYKTWGLHLFSWNPSYARLDKQGNFNAWVA GSYGNDQWLQVDLGSSKEVTGIITQGARNFGSVQFVASYKVAYSNDSANW TEYQDPRTGSSKIFPGNWDNHSHKKNLFETPILARYVRILPVAWHNRIALRLE LLGC |
| 27 | FP100 nucleic acid | ctggacatctgcagcaagaacccctgccacaacggcggcctgtgcgaagagatcagccaggaagtgcg gggcgacgtgttccccagctacacctgtacctgcctgaagggctacgccggcaaccactgcgagacaaag ggctgcgccaaccccctgggcctgaagaacaacagcatccccgacaagcagatcaccgccagcagca gctacaagacctggggcctgcacctgttcagctggaaccccagctacgcccggctggacaagcagggca acttcaacgcctgggtggccggcagctacggcaacgaccagtggctgcaggtggacctgggcagcagca aagaagtgaccggcatcatcacccagggcgccagaaacttcggcagcgtgcagttcgtggccagctaca aggtggcctacagcaacgacagcgccaactggaccgagtaccaggacccccggaccggcagctccaa gatcttccccggcaactgggacaaccacagccacaagaagaacctgttcgagacacccatcctggccag atacgtgcggatcctgcccgtggcctggcacaaccggatcgccctgagactggaactgctgggctgcggct gtgccaatcctctgggactgaaaaacaattccatccctgataagcagattacagcctccagctcctataaga catgggggctgcatctgttttcttggaacccctcctacgctagactggataagcagggaaatttcaatgcttgg gtggccgggtcctatggaaatgatcagtggctgcaggtggacctgggatcctccaaagaagtgacagggat tattacacagggggctcggaactttggctctgtgcagtttgtggcttcctacaaagtggcttactccaacgattcc gccaattggacagaatatcaggatcccagaaccggctccagcaagatctttcctggaaattgggataatca ctcccacaagaaaaatctgtttgaaacccctattctggctcgctatgtgcgcattctgcctgtggcttggcataat agaatcgctctgcggctggaactgctgggatgc |
| 28 | FP110 EGF-C1-C2-HSA | LDICSKNPCHNGGLCEEISQEVRGDVFPSYTCTCLKGYAGNHCETKCVEPLGME NGNIANSQIAASSVRVTFLGLQHWVPELARLNRAGMVNAWTPSSNDDNPWIQV NLLRRMWVTGVVTQGASRLASHEYLKAFKVAYSLNGHEFDFIHDVNKKHKEFVG NWNKNAVHVNLFETPVEAQYVRLYPTSCHTACTLRFELLGCELNGCANPLGLKN NSIPDKQITASSSYKTWGLHLFSWNPSYARLDKQGNFNAWVAGSYGNDQWLQV DLGSSKEVTGIITQGARNFGSVQFVASYKVAYSNDSANWTEYQDPRTGSSKIFP GNWDNHSHKKNLFETPILARYVRILPVAWHNRIALRLELLGCGGSGGSGGSGGS DAHKSEVAHRFKDLGEENFKALVLIAFAQYLQQSPFEDHVKLVNEVTEFAKTCVA DESAENCDKSLHTLFGDKLCTVATLRETYGEMADCCAKQEPERNECFLQHKDD NPNLPRLVRPEVDVMCTAFHDNEETFLKKYLYEIARRHPYFYAPELLFFAKRYKA AFTECCQAADKAACLLPKLDELRDEGKASSAKQRLKCASLQKFGERAFKAWAVA RLSQRFPKAEFAEVSKLVTDLTKVHTECCHGDLLECADDRADLAKYICENQDSIS SKLKECCEKPLLEKSHCIAEVENDEMPADLPSLAADFVESKDVCKNYAEAKDVFL GMFLYEYARRHPDYSVVLLLRLAKTYETTLEKCCAAADPHECYAKVFDEFKPLVE EPQNLIKQNCELFEQLGEYKFQNALLVRYTKKVPQVSTPTLVEVSRNLGKVGSK CCKHPEAKRMPCAEDYLSVVLNQLCVLHEKTPVSDRVTKCCTESLVNRRPCFSA LEVDETYVPKEFNAETFTFHADICTLSEKERQIKKQTALVELVKHKPKATKEQLKA VMDDFAAFVEKCCKADDKETCFAEEGKKLVAASQAALGLHHHHHH |
| 29 | FP110 nucleic acid | ctggacatctgcagcaagaacccctgccacaacggcggcctgtgcgaagagatcagccaggaagtgcgggg cgacgtgttccccagctacacctgtacctgcctgaagggctacgccggcaaccactgcgagactaagtgcgtgg aacccctgggcatggaaaacggcaatatcgccaacagccagatcgccgccagctccgtgcgcgtgacctttctg ggactgcagcactgggtgcccgagctggccagactgaacagagccggcatggtgaacgcctggacccccagc agcaacgacgacaacccttggatccaggtgaacctgctgcggcggatgtgggtgacaggcgtggtgacacagg gcgccagcagactggccagccacgagtacctgaaggcctttaaggtggcctacagcctgaacggccacgagtt cgacttcatccacgacgtgaacaagaaacacaaagaatttgtgggcaactggaacaagaacgccgtgcacgtg aacctgttcgagacacccgtggaagcccagtacgtgcggctgtaccccaccagctgccacaccgcctgcaccct gagattcgagctgctgggctgcgagctgaacggctgcgccaaccccctgggcctgaagaacaacagcatcccc gacaagcagatcaccgcctccagcagctacaagacctggggcctgcacctgttcagctggaaccccagctacg cccggctggacaagcagggcaacttcaacgcctgggtggccggcagctacggcaacgaccagtggctgcagg tggacctgggcagcagcaaagaagtgaccggcatcatcacccagggggccagaaacttcggcagcgtgcagt tcgtggccagctacaaagtggcctactccaacgacagcgccaactggaccgagtaccaggacccccggaccg gcagctccaagatcttccccggcaactgggacaaccacagccacaagaagaatctgttcgaaacccccatcct ggccagatacgtgcggatcctgcccgtggcctggcacaaccggatcgccctgagactggaactgctgggatgtg gaggaagcggaggatctggcggttccggaggctctgacgcccacaagagcgaggtggcccaccggttcaagg acctgggcgaggaaaacttcaaggccctggtgctgatcgccttcgcccagtacctgcagcagagccccttcgaa gatcacgtaaagttagtcaacgaggttacggaattcgcaaagacatgcgttgctgacgaatccgctgagaattgtg acaagagtttgcacactttattcggagataagttgtgtactgtagctactttgagagagacttacggtgaaatggctg actgctgtgcaaaacaggaaccagaacgtaacgaatgtttccttcagcataaggatgataaccctaaccttccaa ggcttgttaggccagaagtcgacgtgatgtgcaccgccttccatgataatgaagagacttttcttaaaaagtacctat acgagattgcaaggcgtcatccatatttttacgccccagagctgttgtttttcgcaaagagatacaaagctgcatttac tgagtgttgccaagctgccgacaaggccgcttgtttgctaccaaagttggacgaattgagagacgagggtaaggc atcatctgccaagcagagattaaaatgtgcatctttgcaaaaatttggagagagagcttttaaggcatgggctgttg cccgactaagccaaagattcccaaaagccgaatttgctgaagtatccaagctggtgactgatttgactaaagtaca tacagaatgttgccatggcgaccttttagaatgtgctgatgacagagcagatttggctaagtatatctgcgaaaatca agattcaatcagctctaagctgaaggaatgttgcgagaaaccactgttagaaaaatcgcattgtattgctgaagttg aaaatgatgagatgcctgctgacttgccttctcttgccgctgattttgttgagtcgaaggatgtctgtaagaattatgctg aagctaaagacgttttcctgggtatgttcttatatgagtacgcaagacgtcacccagattactctgtggttctgctactg agattggctaaaacatacgagacaacgctggagaagtgctgtgctgccgctgaccctcatgagtgctatgcaaa ggtttttgatgaattcaaaccattggttgaagagcctcaaaacttgataaagcagaactgtgagctgtttgagcaatt gggtgagtataagttccaaaatgccctgttggtgagatatacaaaaaaggtaccccaagtttcaacgcccacttta gttgaagtgtccagaaatcttggtaaagtgggtagcaaatgttgcaagcatccagaagccaagcgaatgccctgt gctgaggattatctgtccgtcgtgttgaaccaattgtgcgtattacacgaaaaaaccccagtctctgatagagtcacc aaatgttgcactgagtcactagttaatagaaggccttgtttttccgctttggaagttgatgaaacctacgtgcctaagg aatttaacgctgagacctttacctttcacgctgacatttgtactttgagtgaaaaagagcgtcaaatcaaaaagcaa accgctcttgttgaattggtgaaacacaagcctaaggctacgaaggagcagcttaaagccgtcatggacgatttc gccgcatttgttgaaaaatgctgtaaagctgatgacaaggaaacatgtttcgctgaagagggaaagaaattggttg cggccagtcaggccgcacttggtttgcaccatcatcaccatcac |
| 30 | FP220 HSA-EGF-C1-C2 | DAHKSEVAHRFKDLGEENFKALVLIAFAQYLQQSPFEDHVKLVNEVTEFAKTCVA DESAENCDKSLHTLFGDKLCTVATLRETYGEMADCCAKQEPERNECFLQHKDD NPNLPRLVRPEVDVMCTAFHDNEETFLKKYLYEIARRHPYFYAPELLFFAKRYKA AFTECCQAADKAACLLPKLDELRDEGKASSAKQRLKCASLQKFGERAFKAWAVA RLSQRFPKAEFAEVSKLVTDLTKVHTECCHGDLLECADDRADLAKYICENQDSIS SKLKECCEKPLLEKSHCIAEVENDEMPADLPSLAADFVESKDVCKNYAEAKDVFL GMFLYEYARRHPDYSVVLLLRLAKTYETTLEKCCAAADPHECYAKVFDEFKPLVE EPQNLIKQNCELFEQLGEYKFQNALLVRYTKKVPQVSTPTLVEVSRNLGKVGSK CCKHPEAKRMPCAEDYLSVVLNQLCVLHEKTPVSDRVTKCCTESLVNRRPCFSA LEVDETYVPKEFNAETFTFHADICTLSEKERQIKKQTALVELVKHKPKATKEQLKA VMDDFAAFVEKCCKADDKETCFAEEGKKLVAASQAGGSGGSGGSGGSLDICSK NPCHNGGLCEEISQEVRGDVFPSYTCTCLKGYAGNHCETKCVEPLGMENGNIA NSQIAASSVRVTFLGLQHWVPELARLNRAGMVNAWTPSSNDDNPWIQVNLLRR MVWTGVVTQGASRLASHEYLKAFKV A YSLNGH EFDFI HDVNKKHKEFVGNWNK NAVHVNLFETPVEAQYVRLYPTSCHTACTLRFELLGCELNGCANPLGLKNNSIPD KQITASSSYKTWGLHLFSWNPSYARLDKQGNFNAWVAGSYGNDQWLQVDLGS SKEVTGIITQGARNFGSVQFVASYKVAYSNDSANWTEYQDPRTGSSKIFPGNWD NHSHKKNLFETPILARYVRILPVAWHNRIALRLELLGCGSHHHHHH |
| 31 | FP220 nucleic acid | gacgcccacaagagcgaggtggcccaccggttcaaggacctgggcgaggaaaacttcaaggccctggtgctg atcgccttcgcccagtacctgcagcagagccccttcgaagatcacgtaaagttagtcaacgaggttacggaattc gcaaagacatgcgttgctgacgaatccgctgagaattgtgacaagagtttgcacactttattcggagataagttgtgt actgtagctactttgagagagacttacggtgaaatggctgactgctgtgcaaaacaggaaccagaacgtaacga atgtttccttcagcataaggatgataaccctaaccttccaaggcttgttaggccagaagtcgacgtgatgtgcaccg ccttccatgataatgaagagacttttcttaaaaagtacctatacgagattgcaaggcgtcatccatatttttacgcccc agagctgttgtttttcgcaaagagatacaaagctgcatttactgagtgttgccaagctgccgacaaggccgcttgttt gctaccaaagttggacgaattgagagacgagggtaaggcatcatctgccaagcagagattaaaatgtgcatcttt gcaaaaatttggagagagagcttttaaggcatgggctgttgcccgactaagccaaagattcccaaaagccgaatt tgctgaagtatccaagctggtgactgatttgactaaagtacatacagaatgttgccatggcgaccttttagaatgtgct gatgacagagcagatttggctaagtatatctgcgaaaatcaagattcaatcagctctaagctgaaggaatgttgcg agaaaccactgttagaaaaatcgcattgtattgctgaagttgaaaatgatgagatgcctgctgacttgccttctcttgc cgctgattttgttgagtcgaaggatgtctgtaagaattatgctgaagctaaagacgttttcctgggtatgttcttatatga gtacgcaagacgtcacccagattactctgtggttctgctactgagattggctaaaacatacgagacaacgctggag aagtgctgtgctgccgctgaccctcatgagtgctatgcaaaggtttttgatgaattcaaaccattggttgaagagcct caaaacttgataaagcagaactgtgagctgtttgagcaattgggtgagtataagttccaaaatgccctgttggtgag atatacaaaaaaggtaccccaagtttcaacgcccactttagttgaagtgtccagaaatcttggtaaagtgggtagc aaatgttgcaagcatccagaagccaagcgaatgccctgtgctgaggattatctgtccgtcgtgttgaaccaattgtg cgtattacacgaaaaaaccccagtctctgatagagtcaccaaatgttgcactgagtcactagttaatagaaggcctt gtttttccgctttggaagttgatgaaacctacgtgcctaaggaatttaacgctgagacctttacctttcacgctgacattt gtactttgagtgaaaaagagcgtcaaatcaaaaagcaaaccgctcttgttgaattggtgaaacacaagcctaagg ctacgaaggagcagcttaaagccgtcatggacgatttcgccgcatttgttgaaaaatgctgtaaagctgatgacaa ggaaacatgtttcgctgaagagggaaagaaattggttgcggccagtcaggccggaggaagcggaggatctgg cggttccggaggctctctagacatctgcagcaagaacccctgccacaacggcggcctgtgcgaagagatcagc caggaagtgcggggcgacgtgttccccagctacacctgtacctgcctgaagggctacgccggcaaccactgcg agactaagtgcgtggaacccctgggcatggaaaacggcaatatcgccaacagccagatcgccgccagctccgt gcgcgtgacctttctgggactgcagcactgggtgcccgagctggccagactgaacagagccggcatggtgaac gcctggacccccagcagcaacgacgacaacccttggatccaggtgaacctgctgcggcggatgtgggtgacag gcgtggtgacacagggcgccagcagactggccagccacgagtacctgaaggcctttaaggtggcctacagcct gaacggccacgagttcgacttcatccacgacgtgaacaagaaacacaaagaatttgtgggcaactggaacaa gaacgccgtgcacgtgaacctgttcgagacacccgtggaagcccagtacgtgcggctgtaccccaccagctgc cacaccgcctgcaccctgagattcgagctgctgggctgcgagctgaacggctgcgccaaccccctgggcctgaa gaacaacagcatccccgacaagcagatcaccgcctccagcagctacaagacctggggcctgcacctgttcagc tggaaccccagctacgcccggctggacaagcagggcaacttcaacgcctgggtggccggcagctacggcaac gaccagtggctgcaggtggacctgggcagcagcaaagaagtgaccggcatcatcacccagggggccagaaa cttcggcagcgtgcagttcgtggccagctacaaagtggcctactccaacgacagcgccaactggaccgagtacc aggacccccggaccggcagctccaagatcttccccggcaactgggacaaccacagccacaagaagaatctgt tcgaaacccccatcctggccagatacgtgcggatcctgcccgtggcctggcacaaccggatcgccctgagactg gaactgctgggatgtggctctcaccaccaccatcaccat |
| 32 | FP250 EGF-HSA | LDICSKNPCHNGGLCEEISQEVRGDVFPSYTCTCLKGYAGNHCETKGSDAH KSEVAHRFKDLGEENFKALVLIAFAQYLQQSPFEDHVKLVNEVTEFAKTCVA DESAENCDKSLHTLFGDKLCTVATLRETYGEMADCCAKQEPERNECFLQHK DDNPNLPRLVRPEVDVMCTAFHDNEETFLKKYLYEIARRHPYFYAPELLFFA KRYKAAFTECCQAADKAACLLPKLDELRDEGKASSAKQRLKCASLQKFGER AFKAWAVARLSQRFPKAEFAEVSKLVTDLTKVHTECCHGDLLECADDRADL AKYICENQDSISSKLKECCEKPLLEKSHCIAEVENDEMPADLPSLAADFVESK DVCKNYAEAKDVFLGMFLYEYARRHPDYSVVLLLRLAKTYETTLEKCCAAAD PHECYAKVFDEFKPLVEEPQNLIKQNCELFEQLGEYKFQNALLVRYTKKVPQ VSTPTLVEVSRNLGKVGSKCCKHPEAKRMPCAEDYLSVVLNQLCVLHEKTP VSDRVTKCCTESLVNRRPCFSALEVDETYVPKEFNAETFTFHADICTLSEKE RQIKKQTALVELVKHKPKATKEQLKAVMDDFAAFVEKCCKADDKETCFAEEG KKLVAASQAALGLGSHHHHHH |
| 33 | FP250 nucleic acid | ctggacatctgcagcaagaatccttgccacaacggcggcctgtgcgaagagatttctcaagaagtgcggg gcgacgttttccccagctacacctgtacatgtctgaagggctacgccggcaaccactgcgagacaaaagg atccgatgctcacaagagcgaggtggcccacagattcaaggatctgggcgaagagaacttcaaggccct ggtgctgatcgccttcgctcagtatctccagcagagccctttcgaggaccacgtgaagctggtcaacgaagt gaccgagttcgccaagacctgtgtggccgatgagagcgccgagaactgtgataagagcctgcacaccctg ttcggcgacaagctgtgtacagtggccacactgagagaaacctacggcgagatggccgactgctgtgcca agcaagagcccgagagaaacgagtgcttcctgcagcacaaggacgacaaccccaacctgcctagactc gtgcgacccgaagtggatgtgatgtgcaccgcctttcacgacaacgaggaaaccttcctgaagaagtacct gtacgagatcgccagacggcacccctacttttatgcccctgagctgctgttcttcgccaagcggtataaggcc gccttcaccgaatgttgccaggccgctgataaggctgcctgtctgctgcctaagctggacgagctgagagat gagggcaaagccagctctgccaagcagagactgaagtgcgccagcctgcagaagttcggcgagagag cttttaaggcctgggccgttgccagactgagccagagatttcctaaggccgagtttgccgaggtgtccaagct cgtgaccgatctgacaaaggtgcacaccgagtgctgtcacggcgatctgctggaatgtgccgacgataga gccgacctggccaagtacatctgcgagaaccaggacagcatcagcagcaagctgaaagagtgctgcga gaagcccctgctggaaaagtctcactgtatcgccgaggtggaaaacgacgagatgcctgccgatctgccta gcctggctgccgatttcgtggaaagcaaggacgtgtgcaagaactacgccgaggccaaggatgtgtttctg ggcatgtttctgtatgagtacgcccgcagacaccccgactattctgtggttctgctgctgcggctggccaaaac ctacgagacaaccctggaaaaatgctgcgccgctgccgatcctcacgagtgttatgccaaggtgttcgacg agttcaagcctctggtggaagaaccccagaacctgatcaagcagaactgcgagctgttcgagcagctggg cgagtacaagttccagaatgccctgctcgtgcggtacaccaagaaagtgcctcaggtgtccacacctacac tggttgaggtgtcccggaatctgggcaaagtgggcagcaagtgttgcaagcaccctgaggccaagagaat gccttgcgccgaggattacctgagcgtggtgctgaatcagctgtgcgtgctgcacgagaaaacccctgtgtc cgacagagtgaccaagtgctgtaccgagagcctcgtgaacagaaggccttgctttagcgccctggaagtg gacgagacatacgtgcccaaagagttcaacgccgagacattcaccttccacgccgacatctgtaccctgag cgagaaagagcggcagatcaagaagcagacagccctggtcgagctggttaagcacaagcccaaggcc accaaagaacagctgaaggccgtgatggacgacttcgccgcctttgtcgagaagtgctgcaaggccgacg acaaagagacatgcttcgccgaagagggcaagaaactggtggctgcctctcaggctgctctcggacttggc tctcaccaccaccatcaccat |
| 34 | FP260 EGF-HSA-C1 | LDICSKNPCHNGGLCEEISQEVRGDVFPSYTCTCLKGYAGNHCETKGSDAH KSEVAHRFKDLGEENFKALVLIAFAQYLQQSPFEDHVKLVNEVTEFAKTCVA DESAENCDKSLHTLFGDKLCTVATLRETYGEMADCCAKQEPERNECFLQHK DDNPNLPRLVRPEVDVMCTAFHDNEETFLKKYLYEIARRHPYFYAPELLFFA KRYKAAFTECCQAADKAACLLPKLDELRDEGKASSAKQRLKCASLQKFGER AFKAWAVARLSQRFPKAEFAEVSKLVTDLTKVHTECCHGDLLECADDRADL AKYICENQDSISSKLKECCEKPLLEKSHCIAEVENDEMPADLPSLAADFVESK DVCKNYAEAKDVFLGMFLYEYARRHPDYSVVLLLRLAKTYETTLEKCCAAAD PHECYAKVFDEFKPLVEEPQNLIKQNCELFEQLGEYKFQNALLVRYTKKVPQ VSTPTLVEVSRNLGKVGSKCCKHPEAKRMPCAEDYLSVVLNQLCVLHEKTP VSDRVTKCCTESLVNRRPCFSALEVDETYVPKEFNAETFTFHADICTLSEKE RQIKKQTALVELVKHKPKATKEQLKAVMDDFAAFVEKCCKADDKETCFAEEG KKLVAASQAALGLGGSGGSGGSGGSCVEPLGMENGNIANSQIAASSVRVTF LGLQHWVPELARLNRAGMVNAWTPSSNDDNPWIQVNLLRRMWVTGWTQ GASRLASHEYLKAFKVAYSLNGHEFDFIHDVNKKHKEFVGNWNKNAVHVNL FETPVEAQYVRLYPTSCHTACTLRFELLGCGSHHHHHH |
| 35 | FP260 nucleic acid | ctggacatctgcagcaagaatccttgccacaacggcggcctgtgcgaagagatttctcaagaagtgcggg gcgacgttttccccagctacacctgtacatgtctgaagggctacgccggcaaccactgcgagacaaaagg atccgatgctcacaagagcgaggtggcccacagattcaaggatctgggcgaagagaacttcaaggccct ggtgctgatcgccttcgctcagtatctccagcagagccctttcgaggaccacgtgaagctggtcaacgaagt gaccgagttcgccaagacctgtgtggccgatgagagcgccgagaactgtgataagagcctgcacaccctg ttcggcgacaagctgtgtacagtggccacactgagagaaacctacggcgagatggccgactgctgtgcca agcaagagcccgagagaaacgagtgcttcctgcagcacaaggacgacaaccccaacctgcctagactc gtgcgacccgaagtggatgtgatgtgcaccgcctttcacgacaacgaggaaaccttcctgaagaagtacct gtacgagatcgccagacggcacccctacttttatgcccctgagctgctgttcttcgccaagcggtataaggcc gccttcaccgaatgttgccaggccgctgataaggctgcctgtctgctgcctaagctggacgagctgagagat gagggcaaagccagctctgccaagcagagactgaagtgcgccagcctgcagaagttcggcgagagag cttttaaggcctgggccgttgccagactgagccagagatttcctaaggccgagtttgccgaggtgtccaagct cgtgaccgatctgacaaaggtgcacaccgagtgctgtcacggcgatctgctggaatgtgccgacgataga gccgacctggccaagtacatctgcgagaaccaggacagcatcagcagcaagctgaaagagtgctgcga gaagcccctgctggaaaagtctcactgtatcgccgaggtggaaaacgacgagatgcctgccgatctgccta gcctggctgccgatttcgtggaaagcaaggacgtgtgcaagaactacgccgaggccaaggatgtgtttctg ggcatgtttctgtatgagtacgcccgcagacaccccgactattctgtggttctgctgctgcggctggccaaaac ctacgagacaaccctggaaaaatgctgcgccgctgccgatcctcacgagtgttatgccaaggtgttcgacg agttcaagcctctggtggaagaaccccagaacctgatcaagcagaactgcgagctgttcgagcagctggg cgagtacaagttccagaatgccctgctcgtgcggtacaccaagaaagtgcctcaggtgtccacacctacac tggttgaggtgtcccggaatctgggcaaagtgggcagcaagtgttgcaagcaccctgaggccaagagaat gccttgcgccgaggattacctgagcgtggtgctgaatcagctgtgcgtgctgcacgagaaaacccctgtgtc cgacagagtgaccaagtgctgtaccgagagcctcgtgaacagaaggccttgctttagcgccctggaagtg gacgagacatacgtgcccaaagagttcaacgccgagacattcaccttccacgccgacatctgtaccctgag cgagaaagagcggcagatcaagaagcagacagccctggtcgagctggttaagcacaagcccaaggcc accaaagaacagctgaaggccgtgatggacgacttcgccgcctttgtcgagaagtgctgcaaggccgacg acaaagagacatgcttcgccgaagagggcaagaaactggtggctgcctctcaggctgctctcggacttgg aggaagcggaggatctggcggttccggaggctcttgtgtggaacccctcggcatggaaaacggcaatatc gccaatagccagattgccgccagcagcgtcagagtgacatttctgggactgcagcactgggtgcccgagct ggctagactgaatagagccggcatggtcaacgcctggacacccagcagcaacgacgataacccttggatt caagtgaacctgctgcggcgtatgtgggtcacaggtgttgttacacagggcgcctctagactggccagccac gagtatctgaaggcctttaaggtggcctacagcctgaacggccacgagttcgacttcatccacgacgtgaac aagaagcacaaagagtttgtcggcaactggaacaagaacgccgtgcacgtgaacctgttcgagacacct gtggaagcccagtacgtgcggctgtaccctacaagctgtcacaccgcctgcactctgagattcgaactgctg ggatgcggctctcaccaccaccatcaccat |
| 36 | FP270 EGF-HSA-C2 | LDICSKNPCHNGGLCEEISQEVRGDVFPSYTCTCLKGYAGNHCETKGSDAH KSEVAHRFKDLGEENFKALVLIAFAQYLQQSPFEDHVKLVNEVTEFAKTCVA DESAENCDKSLHTLFGDKLCTVATLRETYGEMADCCAKQEPERNECFLQHK DDNPNLPRLVRPEVDVMCTAFHDNEETFLKKYLYEIARRHPYFYAPELLFFA KRYKAAFTECCQAADKAACLLPKLDELRDEGKASSAKQRLKCASLQKFGER AFKAWAVARLSQRFPKAEFAEVSKLVTDLTKVHTECCHGDLLECADDRADL AKYICENQDSISSKLKECCEKPLLEKSHCIAEVENDEMPADLPSLAADFVESK DVCKNYAEAKDVFLGMFLYEYARRHPDYSVVLLLRLAKTYETTLEKCCAAAD PHECYAKVFDEFKPLVEEPQNLIKQNCELFEQLGEYKFQNALLVRYTKKVPQ VSTPTLVEVSRNLGKVGSKCCKHPEAKRMPCAEDYLSVVLNQLCVLHEKTP VSDRVTKCCTESLVNRRPCFSALEVDETYVPKEFNAETFTFHADICTLSEKE RQIKKQTALVELVKHKPKATKEQLKAVMDDFAAFVEKCCKADDKETCFAEEG KKLVAASQAALGLGGSGGSGGSGGSCANPLGLKNNSIPDKQITASSSYKTW GLHLFSWNPSYARLDKQGNFNAWVAGSYGNDQWLQVDLGSSKEVTGIITQ GARNFGSVQFVASYKVAYSNDSANWTEYQDPRTGSSKIFPGNWDNHSHKK NLFETPILARYVRILPVAWHNRIALRLELLGCGSHHHHHH |
| 37 | FP270 nucleic acid | ctggacatctgcagcaagaatccttgccacaacggcggcctgtgcgaagagatttctcaagaagtgcggg gcgacgttttccccagctacacctgtacatgtctgaagggctacgccggcaaccactgcgagacaaaagg atccgacgcccacaagagcgaggtggcccaccggttcaaggacctgggcgaggaaaacttcaaggccc tggtgctgatcgccttcgcccagtacctgcagcagagccccttcgaagatcacgtaaagttagtcaacgagg ttacggaattcgcaaagacatgcgttgctgacgaatccgctgagaattgtgacaagagtttgcacactttattc ggagataagttgtgtactgtagctactttgagagagacttacggtgaaatggctgactgctgtgcaaaacagg aaccagaacgtaacgaatgtttccttcagcataaggatgataaccctaaccttccaaggcttgttaggccag aagtcgacgtgatgtgcaccgccttccatgataatgaagagacttttcttaaaaagtacctatacgagattgca aggcgtcatccatatttttacgccccagagctgttgtttttcgcaaagagatacaaagctgcatttactgagtgtt gccaagctgccgacaaggccgcttgtttgctaccaaagttggacgaattgagagacgagggtaaggcatc atctgccaagcagagattaaaatgtgcatctttgcaaaaatttggagagagagcttttaaggcatgggctgttg cccgactaagccaaagattcccaaaagccgaatttgctgaagtatccaagctggtgactgatttgactaaag tacatacagaatgttgccatggcgaccttttagaatgtgctgatgacagagcagatttggctaagtatatctgc gaaaatcaagattcaatcagctctaagctgaaggaatgttgcgagaaaccactgttagaaaaatcgcattgt attgctgaagttgaaaatgatgagatgcctgctgacttgccttctcttgccgctgattttgttgagtcgaaggatgt ctgtaagaattatgctgaagctaaagacgttttcctgggtatgttcttatatgagtacgcaagacgtcacccaga ttactctgtggttctgctactgagattggctaaaacatacgagacaacgctggagaagtgctgtgctgccgctg accctcatgagtgctatgcaaaggtttttgatgaattcaaaccattggttgaagagcctcaaaacttgataaag cagaactgtgagctgtttgagcaattgggtgagtataagttccaaaatgccctgttggtgagatatacaaaaa aggtaccccaagtttcaacgcccactttagttgaagtgtccagaaatcttggtaaagtgggtagcaaatgttgc aagcatccagaagccaagcgaatgccctgtgctgaggattatctgtccgtcgtgttgaaccaattgtgcgtatt acacgaaaaaaccccagtctctgatagagtcaccaaatgttgcactgagtcactagttaatagaaggccttg tttttccgctttggaagttgatgaaacctacgtgcctaaggaatttaacgctgagacctttacctttcacgctgac atttgtactttgagtgaaaaagagcgtcaaatcaaaaagcaaaccgctcttgttgaattggtgaaacacaag cctaaggctacgaaggagcagcttaaagccgtcatggacgatttcgccgcatttgttgaaaaatgctgtaaa gctgatgacaaggaaacatgtttcgctgaagagggaaagaaattggttgcggccagtcaggccgcacttg gtttgggaggaagcggaggatctggcggttccggaggctcttgcgccaaccccctgggcctgaagaacaa cagcatccccgacaagcagatcaccgcctccagcagctacaagacctggggcctgcacctgttcagctgg aaccccagctacgcccggctggacaagcagggcaacttcaacgcctgggtggccggcagctacggcaa cgaccagtggctgcaggtggacctgggcagcagcaaagaagtgaccggcatcatcacccagggggcca gaaacttcggcagcgtgcagttcgtggccagctacaaagtggcctactccaacgacagcgccaactggac cgagtaccaggacccccggaccggcagctccaagatcttccccggcaactgggacaaccacagccaca agaagaatctgttcgaaacccccatcctggccagatacgtgcggatcctgcccgtggcctggcacaaccgg atcgccctgagactggaactgctgggatgtggctctcaccaccaccatcaccat |
| 38 | FP280 EGF(RGE)-HSA-C1-C2 | LDICSKNPCHNGGLCEEISQEVRGEVFPSYTCTCLKGYAGNHCETKGSDAH KSEVAHRFKDLGEENFKALVLIAFAQYLQQSPFEDHVKLVNEVTEFAKTCVA DESAENCDKSLHTLFGDKLCTVATLRETYGEMADCCAKQEPERNECFLQHK DDNPNLPRLVRPEVDVMCTAFHDNEETFLKKYLYEIARRHPYFYAPELLFFA KRYKAAFTECCQAADKAACLLPKLDELRDEGKASSAKQRLKCASLQKFGER AFKAWAVARLSQRFPKAEFAEVSKLVTDLTKVHTECCHGDLLECADDRADL AKYICENQDSISSKLKECCEKPLLEKSHCIAEVENDEMPADLPSLAADFVESK DVCKNYAEAKDVFLGMFLYEYARRHPDYSVVLLLRLAKTYETTLEKCCAAAD PHECYAKVFDEFKPLVEEPQNLIKQNCELFEQLGEYKFQNALLVRYTKKVPQ VSTPTLVEVSRNLGKVGSKCCKHPEAKRMPCAEDYLSVVLNQLCVLHEKTP VSDRVTKCCTESLVNRRPCFSALEVDETYVPKEFNAETFTFHADICTLSEKE RQIKKQTALVELVKHKPKATKEQLKAVMDDFAAFVEKCCKADDKETCFAEEG KKLVAASQAALGLGGSGGSGGSGGSCVEPLGMENGNIANSQIAASSVRVTF LGLQHWVPELARLNRAGMVNAWTPSSNDDNPWIQVNLLRRMWVTGWTQ GASRLASHEYLKAFKVAYSLNGHEFDFIHDVNKKHKEFVGNWNKNAVHVNL FETPVEAQYVRLYPTSCHTACTLRFELLGCELNGCANPLGLKNNSIPDKQITA SSSYKTWGLHLFSWNPSYARLDKQGNFNAWVAGSYGNDQWLQVDLGSSK EVTGIITQGARNFGSVQFVASYKVAYSNDSANWTEYQDPRTGSSKIFPGNW DNHSHKKNLFETPILARYVRILPVAWHNRIALRLELLGCGSHHHHHH |
| 39 | FP280 nucleic acid | ctggacatctgcagcaagaatccttgccacaacggcggcctgtgcgaagagatttctcaagaagtgcggg gcgaggttttccccagctacacctgtacatgtctgaagggctacgccggcaaccactgcgagacaaaagg atccgatgctcacaagagcgaggtggcccacagattcaaggatctgggcgaagagaacttcaaggccct ggtgctgatcgccttcgctcagtatctccagcagagccctttcgaggaccacgtgaagctggtcaacgaagt gaccgagttcgccaagacctgtgtggccgatgagagcgccgagaactgtgataagagcctgcacaccctg ttcggcgacaagctgtgtacagtggccacactgagagaaacctacggcgagatggccgactgctgtgcca agcaagagcccgagagaaacgagtgcttcctgcagcacaaggacgacaaccccaacctgcctagactc gtgcgacccgaagtggatgtgatgtgcaccgcctttcacgacaacgaggaaaccttcctgaagaagtacct gtacgagatcgccagacggcacccctacttttatgcccctgagctgctgttcttcgccaagcggtataaggcc gccttcaccgaatgttgccaggccgctgataaggctgcctgtctgctgcctaagctggacgagctgagagat gagggcaaagccagctctgccaagcagagactgaagtgcgccagcctgcagaagttcggcgagagag cttttaaggcctgggccgttgccagactgagccagagatttcctaaggccgagtttgccgaggtgtccaagct cgtgaccgatctgacaaaggtgcacaccgagtgctgtcacggcgatctgctggaatgtgccgacgataga gccgacctggccaagtacatctgcgagaaccaggacagcatcagcagcaagctgaaagagtgctgcga gaagcccctgctggaaaagtctcactgtatcgccgaggtggaaaacgacgagatgcctgccgatctgccta gcctggctgccgatttcgtggaaagcaaggacgtgtgcaagaactacgccgaggccaaggatgtgtttctg ggcatgtttctgtatgagtacgcccgcagacaccccgactattctgtggttctgctgctgcggctggccaaaac ctacgagacaaccctggaaaaatgctgcgccgctgccgatcctcacgagtgttatgccaaggtgttcgacg agttcaagcctctggtggaagaaccccagaacctgatcaagcagaactgcgagctgttcgagcagctggg cgagtacaagttccagaatgccctgctcgtgcggtacaccaagaaagtgcctcaggtgtccacacctacac tggttgaggtgtcccggaatctgggcaaagtgggcagcaagtgttgcaagcaccctgaggccaagagaat gccttgcgccgaggattacctgagcgtggtgctgaatcagctgtgcgtgctgcacgagaaaacccctgtgtc cgacagagtgaccaagtgctgtaccgagagcctcgtgaacagaaggccttgctttagcgccctggaagtg gacgagacatacgtgcccaaagagttcaacgccgagacattcaccttccacgccgacatctgtaccctgag cgagaaagagcggcagatcaagaagcagacagccctggtcgagctggttaagcacaagcccaaggcc accaaagaacagctgaaggccgtgatggacgacttcgccgcctttgtcgagaagtgctgcaaggccgacg acaaagagacatgcttcgccgaagagggcaagaaactggtggctgcctctcaggctgctctcggacttgg aggaagcggaggatctggcggttccggaggctcttgtgtggaacccctcggcatggaaaacggcaatatc gccaatagccagattgccgccagcagcgtcagagtgacatttctgggactgcagcactgggtgcccgagct ggctagactgaatagagccggcatggtcaacgcctggacacccagcagcaacgacgataacccttggatt caagtgaacctgctgcggcgtatgtgggtcacaggtgttgttacacagggcgcctctagactggccagccac gagtatctgaaggcctttaaggtggcctacagcctgaacggccacgagttcgacttcatccacgacgtgaac aagaagcacaaagagtttgtcggcaactggaacaagaacgccgtgcacgtgaacctgttcgagacacct gtggaagcccagtacgtgcggctgtaccctacaagctgtcacaccgcctgcactctgagattcgaactgctg ggatgcgagctgaacggctgtgctaatcctctgggcctgaagaacaacagcatccccgataagcagatca ccgccagctccagctataagacatggggcctgcacctgttcagctggaacccttcttacgccagactggaca agcagggcaacttcaatgcttgggtggccggcagctacggcaatgatcagtggctgcaagtggacctggg cagcagcaaagaagtgacaggcatcatcacccagggcgccagaaatttcggcagcgtgcagtttgtggcc agctacaaagtggcctactccaacgacagcgccaactggaccgagtatcaggaccctagaaccggcag ctccaagatcttccccggcaattgggacaaccacagccacaagaagaatctgttcgaaacccctatcctgg ccagatatgtgcgcattctgcccgtggcctggcacaacagaattgccctgagactggaactgctcggctgtg gctctcaccaccaccatcaccat |
| 40 | FP320 EGF-HSA D3-C1-C2-His | LDICSKNPCHNGGLCEEISQEVRGDVFPSYTCTCLKGYAGNHCETKGSLVEEPQ NLIKQNCELFEQLGEYKFQNALLVRYTKKVPQVSTPTLVEVSRNLGKVGSKCCK HPEAKRMPCAEDCLSVFLNQLCVLHEKTPVSDRVTKCCTESLVNGRPCFSALEV DETYVPKEFNAETFTFHADICTLSEKERQIKKQTALVELVKHKPKATKEQLKAVM DDFAAFVEKCCKADDKETCFAEEGKKLVAASQAALGLGGSGGSGGSGGSCVEP LGMENGNIANSQIAASSVRVTFLGLQHWVPELARLNRAGMVNAWTPSSNDDNP WIQVNLLRRMWVTGVVTQGASRLASHEYLKAFKVAYSLNGHEFDFIHDVNKKHK EFVGNWNKNAVHVNLFETPVEAQYVRLYPTSCHTACTLRFELLGCELNGCANPL GLKNNSIPDKQITASSSYKTWGLHLFSWNPSYARLDKQGNFNAWVAGSYGNDQ WLQVDLGSSKEVTGIITQGARNFGSVQFVASYKVAYSNDSANWTEYQDPRTGS SKIFPGNWDNHSHKKNLFETPILARYVRILPVAWHNRIALRLELLGCGSHHHHHH |
| 41 | FP320 nucleic acid | ctggacatctgcagcaagaatccttgccacaacggcggcctgtgcgaagagatttctcaagaagtgcggggcga cgttttccccagctacacctgtacatgtctgaagggctacgccggcaaccactgcgagacaaaaggatccctggt ggaagaaccccagaacctgatcaagcagaactgcgagctgttcgagcagctgggcgagtacaagttccagaat gccctgctcgtgcggtacaccaagaaagtgcctcaggtgtccacacctacactggtcgaggtgtccagaaacctg ggcaaagtgggcagcaagtgctgcaagcaccctgaggccaaaagaatgccttgcgccgaggattgcctgagc gtgttcctgaatcagctgtgcgtgctgcacgagaaaacccctgtgtccgacagagtgaccaagtgctgtaccgag agcctggtcaacggcagaccttgctttagcgccctggaagtggatgagacatacgtgcccaaagagttcaacgc cgagacattcaccttccacgccgacatctgtaccctgagcgagaaagagcggcagatcaagaagcagacagc cctggtcgagctggtcaagcacaagcctaaggccaccaaagaacagctgaaggccgtgatggacgacttcgc cgccttcgtggaaaagtgttgcaaggccgacgacaaagagacatgcttcgccgaagagggcaagaaactggt ggctgcctctcaggctgctctcggacttggaggaagcggaggatctggcggttccggaggctcttgtgtggaaccc ctcggcatggaaaacggcaatatcgccaatagccagatcgccgccagcagcgtcagagtgacatttctgggact gcagcactgggtgccagagctggctagactgaatagagccggcatggtcaacgcctggacacccagcagcaa cgacgacaacccctggattcaagtgaacctgctgcggcgtatgtgggtcacaggtgttgttacacagggcgcctct agactggccagccacgagtatctgaaggcctttaaggtggcctacagcctgaacggccacgagttcgacttcatc cacgacgtgaacaagaagcacaaagagtttgtcggcaactggaacaagaacgccgtgcacgtgaacctgttcg agacacctgtggaagcccagtacgtgcggctgtaccctacaagctgtcacaccgcctgcacactgagattcgaa ctgctgggatgcgagctgaacggctgtgctaatcctctgggcctgaagaacaacagcatccccgataagcagat caccgcctccagcagctataagacatggggcctgcacctgttcagctggaaccctagctacgccagactggaca agcagggcaactttaatgcctgggtggccggcagctacggcaatgatcaatggctgcaagtggacctgggcagc agcaaagaagtgaccggcatcattacccagggcgcaagaaatttcggcagcgtgcagttcgtggccagctaca aagtggcctactccaacgacagcgccaactggaccgagtatcaggaccctagaaccggcagctccaagatctt ccccggcaattgggacaaccacagccacaagaagaatctgttcgaaacccctatcctggccagatatgtgcgca ttctgcccgtggcctggcacaacagaattgccctgagactggaactgctcggctgtggctctcaccaccaccatca ccat |
| 42 | FP330 EGF-HSA-C1-C2 | LDICSKNPCHNGGLCEEISQEVRGDVFPSYTCTCLKGYAGNHCETKGSDAHKSE VAHRFKDLGEENFKALVLIAFAQYLQQSPFEDHVKLVNEVTEFAKTCVADESAEN CDKSLHTLFGDKLCTVATLRETYGEMADCCAKQEPERNECFLQHKDDNPNLPRL VRPEVDVMCTAFHDNEETFLKKYLYEIARRHPYFYAPELLFFAKRYKAAFTECCQ AADKAACLLPKLDELRDEGKASSAKQRLKCASLQKFGERAFKAWAVARLSQRFP KAEFAEVSKLVTDLTKVHTECCHGDLLECADDRADLAKYICENQDSISSKLKECC EKPLLEKSHCIAEVENDEMPADLPSLAADFVESKDVCKNYAEAKDVFLGMFLYEY ARRHPDYSVVLLLRLAKTYETTLEKCCAAADPHECYAKVFDEFKPLVEEPQNLIK QNCELFEQLGEYKFQNALLVRYTKKVPQVSTPTLVEVSRNLGKVGSKCCKHPEA KRMPCAEDYLSVVLNQLCVLHEKTPVSDRVTKCCTESLVNRRPCFSALEVDETY VPKEFNAETFTFHADICTLSEKERQIKKQTALVELVKHKPKATKEQLKAVMDDFA AFVEKCCKADDKETCFAEEGKKLVAASQAALGLGGSGGSGGSGGSCVEPLGM ENGNIANSQIAASSVRVTFLGLQHWVPELARLNRAGMVNAWTPSSNDDNPWIQ VNLLRRMWVTGVVTQGASRLASHEYLKAFKVAYSLNGHEFDFIHDVNKKHKEFV GNWNKNAVHVNLFETPVEAQYVRLYPTSCHTACTLRFELLGCELNGCANPLGLK NNSIPDKQITASSSYKTWGLHLFSWNPSYARLDKQGNFNAWVAGSYGNDQWLQ VDLGSSKEVTGIITQGARNFGSVQFVASYKVAYSNDSANWTEYQDPRTGSSKIF PGNWDNHSHKKNLFETPILARYVRILPVAWHNRIALRLELLGC |
| 43 | FP330 nucleic acid | ctggacatctgcagcaagaatccttgccacaacggcggcctgtgcgaagagatttctcaagaagtgcggggcga cgttttccccagctacacctgtacatgtctgaagggctacgccggcaaccactgcgagacaaaaggatccgatgc tcacaagagcgaggtggcccacagattcaaggatctgggcgaagagaacttcaaggccctggtgctgatcgcct tcgctcagtatctccagcagagccctttcgaggaccacgtgaagctggtcaacgaagtgaccgagttcgccaag acctgtgtggccgatgagagcgccgagaactgtgataagagcctgcacaccctgttcggcgacaagctgtgtac agtggccacactgagagaaacctacggcgagatggccgactgctgtgccaagcaagagcccgagagaaacg agtgcttcctgcagcacaaggacgacaaccccaacctgcctagactcgtgcgacccgaagtggatgtgatgtgc accgcctttcacgacaacgaggaaaccttcctgaagaagtacctgtacgagatcgccagacggcacccctacttt tatgcccctgagctgctgttcttcgccaagcggtataaggccgccttcaccgaatgttgccaggccgctgataaggc tgcctgtctgctgcctaagctggacgagctgagagatgagggcaaagccagctctgccaagcagagactgaagt gcgccagcctgcagaagttcggcgagagagcttttaaggcctgggccgttgccagactgagccagagatttccta aggccgagtttgccgaggtgtccaagctcgtgaccgatctgacaaaggtgcacaccgagtgctgtcacggcgat ctgctggaatgtgccgacgatagagccgacctggccaagtacatctgcgagaaccaggacagcatcagcagc aagctgaaagagtgctgcgagaagcccctgctggaaaagtctcactgtatcgccgaggtggaaaacgacgag atgcctgccgatctgcctagcctggctgccgatttcgtggaaagcaaggacgtgtgcaagaactacgccgaggc caaggatgtgtttctgggcatgtttctgtatgagtacgcccgcagacaccccgactattctgtggttctgctgctgcgg ctggccaaaacctacgagacaaccctggaaaaatgctgcgccgctgccgatcctcacgagtgttatgccaaggt gttcgacgagttcaagcctctggtggaagaaccccagaacctgatcaagcagaactgcgagctgttcgagcagc tgggcgagtacaagttccagaatgccctgctcgtgcggtacaccaagaaagtgcctcaggtgtccacacctacac tggttgaggtgtcccggaatctgggcaaagtgggcagcaagtgttgcaagcaccctgaggccaagagaatgcct tgcgccgaggattacctgagcgtggtgctgaatcagctgtgcgtgctgcacgagaaaacccctgtgtccgacaga gtgaccaagtgctgtaccgagagcctcgtgaacagaaggccttgctttagcgccctggaagtggacgagacata cgtgcccaaagagttcaacgccgagacattcaccttccacgccgacatctgtaccctgagcgagaaagagcgg cagatcaagaagcagacagccctggtcgagctggttaagcacaagcccaaggccaccaaagaacagctgaa ggccgtgatggacgacttcgccgcctttgtcgagaagtgctgcaaggccgacgacaaagagacatgcttcgccg aagagggcaagaaactggtggctgcctctcaggctgctctcggacttggaggaagcggaggatctggcggttcc ggaggctcttgtgtggaacccctcggcatggaaaacggcaatatcgccaatagccagattgccgccagcagcgt cagagtgacatttctgggactgcagcactgggtgcccgagctggctagactgaatagagccggcatggtcaacg cctggacacccagcagcaacgacgataacccttggattcaagtgaacctgctgcggcgtatgtgggtcacaggt gttgttacacagggcgcctctagactggccagccacgagtatctgaaggcctttaaggtggcctacagcctgaac ggccacgagttcgacttcatccacgacgtgaacaagaagcacaaagagtttgtcggcaactggaacaagaacg ccgtgcacgtgaacctgttcgagacacctgtggaagcccagtacgtgcggctgtaccctacaagctgtcacaccg cctgcactctgagattcgaactgctgggatgcgagctgaacggctgtgctaatcctctgggcctgaagaacaaca gcatccccgataagcagatcaccgccagctccagctataagacatggggcctgcacctgttcagctggaaccctt cttacgccagactggacaagcagggcaacttcaatgcttgggtggccggcagctacggcaatgatcagtggctg caagtggacctgggcagcagcaaagaagtgacaggcatcatcacccagggcgccagaaatttcggcagcgtg cagtttgtggccagctacaaagtggcctactccaacgacagcgccaactggaccgagtatcaggaccctagaac cggcagctccaagatcttccccggcaattgggacaaccacagccacaagaagaatctgttcgaaacccctatcc tggccagatatgtgcgcattctgcccgtggcctggcacaacagaattgccctgagactggaactgctcggctgt |
| 44 | FP278 EGF-HSA-C1-C2 His tag | LDICSKNPCHNGGLCEEISQEVRGDVFPSYTCTCLKGYAGNHCETKGSDAHKSE VAHRFKDLGEENFKALVLIAFAQYLQQSPFEDHVKLVNEVTEFAKTCVADESAEN CDKSLHTLFGDKLCTVATLRETYGEMADCCAKQEPERNECFLQHKDDNPNLPRL VRPEVDVMCTAFHDNEETFLKKYLYEIARRHPYFYAPELLFFAKRYKAAFTECCQ AADKAACLLPKLDELRDEGKASSAKQRLKCASLQKFGERAFKAWAVARLSQRFP KAEFAEVSKLVTDLTKVHTECCHGDLLECADDRADLAKYICENQDSISSKLKECC EKPLLEKSHCIAEVENDEMPADLPSLAADFVESKDVCKNYAEAKDVFLGMFLYEY ARRHPDYSVVLLLRLAKTYETTLEKCCAAADPHECYAKVFDEFKPLVEEPQNLIK QNCELFEQLGEYKFQNALLVRYTKKVPQVSTPTLVEVSRNLGKVGSKCCKHPEA KRMPCAEDYLSVVLNQLCVLHEKTPVSDRVTKCCTESLVNRRPCFSALEVDETY VPKEFNAETFTFHADICTLSEKERQIKKQTALVELVKHKPKATKEQLKAVMDDFA AFVEKCCKADDKETCFAEEGKKLVAASQAALGLGGSGGSGGSGGSCVEPLGM ENGNIANSQIAASSVRVTFLGLQHWVPELARLNRAGMVNAWTPSSNDDNPWIQ VNLLRRMWVTGVVTQGASRLASHEYLKAFKVAYSLNGHEFDFIHDVNKKHKEFV GNWNKNAVHVNLFETPVEAQYVRLYPTSCHTACTLRFELLGCELNGCANPLGLK NNSIPDKQITASSSYKTWGLHLFSWNPSYARLDKQGNFNAWVAGSYGNDQWLQ VDLGSSKEVTGIITQGARNFGSVQFVASYKVAYSNDSANWTEYQDPRTGSSKIF PGNWDNHSHKKNLFETPILARYVRILPVAWHNRIALRLELLGCGSHHHHHH |
| 45 | FP278 nucleic acid | ctggacatctgcagcaagaatccttgccacaacggcggcctgtgcgaagagatttctcaagaagtgcggggcga cgttttccccagctacacctgtacatgtctgaagggctacgccggcaaccactgcgagacaaaaggatccgatgc tcacaagagcgaggtggcccacagattcaaggatctgggcgaagagaacttcaaggccctggtgctgatcgcct tcgctcagtatctccagcagagccctttcgaggaccacgtgaagctggtcaacgaagtgaccgagttcgccaag acctgtgtggccgatgagagcgccgagaactgtgataagagcctgcacaccctgttcggcgacaagctgtgtac agtggccacactgagagaaacctacggcgagatggccgactgctgtgccaagcaagagcccgagagaaacg agtgcttcctgcagcacaaggacgacaaccccaacctgcctagactcgtgcgacccgaagtggatgtgatgtgc accgcctttcacgacaacgaggaaaccttcctgaagaagtacctgtacgagatcgccagacggcacccctacttt tatgcccctgagctgctgttcttcgccaagcggtataaggccgccttcaccgaatgttgccaggccgctgataaggc tgcctgtctgctgcctaagctggacgagctgagagatgagggcaaagccagctctgccaagcagagactgaagt gcgccagcctgcagaagttcggcgagagagcttttaaggcctgggccgttgccagactgagccagagatttccta aggccgagtttgccgaggtgtccaagctcgtgaccgatctgacaaaggtgcacaccgagtgctgtcacggcgat ctgctggaatgtgccgacgatagagccgacctggccaagtacatctgcgagaaccaggacagcatcagcagc aagctgaaagagtgctgcgagaagcccctgctggaaaagtctcactgtatcgccgaggtggaaaacgacgag atgcctgccgatctgcctagcctggctgccgatttcgtggaaagcaaggacgtgtgcaagaactacgccgaggc caaggatgtgtttctgggcatgtttctgtatgagtacgcccgcagacaccccgactattctgtggttctgctgctgcgg ctggccaaaacctacgagacaaccctggaaaaatgctgcgccgctgccgatcctcacgagtgttatgccaaggt gttcgacgagttcaagcctctggtggaagaaccccagaacctgatcaagcagaactgcgagctgttcgagcagc tgggcgagtacaagttccagaatgccctgctcgtgcggtacaccaagaaagtgcctcaggtgtccacacctacac tggttgaggtgtcccggaatctgggcaaagtgggcagcaagtgttgcaagcaccctgaggccaagagaatgcct tgcgccgaggattacctgagcgtggtgctgaatcagctgtgcgtgctgcacgagaaaacccctgtgtccgacaga gtgaccaagtgctgtaccgagagcctcgtgaacagaaggccttgctttagcgccctggaagtggacgagacata cgtgcccaaagagttcaacgccgagacattcaccttccacgccgacatctgtaccctgagcgagaaagagcgg cagatcaagaagcagacagccctggtcgagctggttaagcacaagcccaaggccaccaaagaacagctgaa ggccgtgatggacgacttcgccgcctttgtcgagaagtgctgcaaggccgacgacaaagagacatgcttcgccg aagagggcaagaaactggtggctgcctctcaggctgctctcggacttggaggaagcggaggatctggcggttcc ggaggctcttgtgtggaacccctcggcatggaaaacggcaatatcgccaatagccagattgccgccagcagcgt cagagtgacatttctgggactgcagcactgggtgcccgagctggctagactgaatagagccggcatggtcaacg cctggacacccagcagcaacgacgataacccttggattcaagtgaacctgctgcggcgtatgtgggtcacaggt gttgttacacagggcgcctctagactggccagccacgagtatctgaaggcctttaaggtggcctacagcctgaac ggccacgagttcgacttcatccacgacgtgaacaagaagcacaaagagtttgtcggcaactggaacaagaacg ccgtgcacgtgaacctgttcgagacacctgtggaagcccagtacgtgcggctgtaccctacaagctgtcacaccg cctgcactctgagattcgaactgctgggatgcgagctgaacggctgtgctaatcctctgggcctgaagaacaaca gcatccccgataagcagatcaccgccagctccagctataagacatggggcctgcacctgttcagctggaaccctt cttacgccagactggacaagcagggcaacttcaatgcttgggtggccggcagctacggcaatgatcagtggctg caagtggacctgggcagcagcaaagaagtgacaggcatcatcacccagggcgccagaaatttcggcagcgtg cagtttgtggccagctacaaagtggcctactccaacgacagcgccaactggaccgagtatcaggaccctagaac cggcagctccaagatcttccccggcaattgggacaaccacagccacaagaagaatctgttcgaaacccctatcc tggccagatatgtgcgcattctgcccgtggcctggcacaacagaattgccctgagactggaactgctcggctgtgg ctctcaccaccaccatcaccat |
| 46 | FP068 | LDICSKNPCHNGGLCEEISQEVRGDVFPSYTCTCLKGYAGNHCETKDAHKSEVA HRFKDLGEENFKALVLIAFAQYLQQSPFEDHVKLVNEVTEFAKTCVADESAENCD KSLHTLFGDKLCTVATLRETYGEMADCCAKQEPERNECFLQHKDDNPNLPRLVR PEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKA EFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVAASQAALCVEPLGMENGNIANSQIAASSVRVTFL GLQHWVPELARLNRAGMVNAWTPSSNDDNPWIQVNLLRRMWVTGVVTQGASR LASHEYLKAFKVAYSLNGHEFDFIHDVNKKHKEFVGNWNKNAVHVNLFETPVEA QYVRLYPTSCHTACTLRFELLGCELNGCANPLGLKNNSIPDKQITASSSYKTWGL HLFSWNPSYARLDKQGNFNAWVAGSYGNDQWLQVDLGSSKEVTGIITQGARNF GSVQFVASYKVAYSNDSANWTEYQDPRTGSSKIFPGNWDNHSHKKNLFETPILA RYVRILPVAWHNRIALRLELLGC |
| 47 | FP068 nucleic acid | ctggacatctgtagcaagaacccttgccacaacggcggcctgtgcgaagagatttctcaagaagtgcggggcga cgttttccccagctacacctgtacatgtctgaagggctacgccggcaaccactgcgagacaaaggatgcccaca agagcgaggtggcccacagattcaaggatctgggcgaagagaacttcaaggccctggtgctgatcgccttcgct cagtatctccagcagagccctttcgaggaccacgtgaagctggtcaacgaagtgaccgagttcgccaagacctg tgtggccgatgagagcgccgagaactgtgataagagcctgcacaccctgttcggcgacaagctgtgtacagtgg ccacactgagagaaacctacggcgagatggccgactgctgtgccaagcaagagcccgagagaaacgagtgc ttcctccagcacaaggacgacaaccccaacctgcctagactcgtgcgacccgaagtggatgtgatgtgcaccgc ctttcacgacaacgaggaaaccttcctgaagaagtacctgtacgagatcgccagacggcacccctacttttatgcc cctgagctgctgttcttcgccaagcggtataaggccgccttcaccgaatgttgccaggccgctgataaggctgcctg tctgctgcctaagctggacgagctgagagatgagggcaaagccagctctgccaagcagagactgaaatgcgcc agcctccagaagttcggcgagagagcttttaaggcctgggccgttgccagactgagccagagatttcctaaggcc gagtttgccgaggtgtccaagctcgtgaccgatctgacaaaggtgcacaccgagtgctgtcacggcgatctgctg gaatgtgccgacgatagagccgacctggccaagtacatctgcgagaaccaggacagcatcagcagcaagctg aaagagtgctgcgagaagcccctgctggaaaagtctcactgtatcgccgaggtggaaaacgacgagatgcctg ccgatctgcctagcctggctgccgatttcgtggaaagcaaggacgtgtgcaagaactacgccgaggccaaggat gtgtttctgggcatgtttctgtatgagtacgcccgcagacaccccgactattctgtggttctgctgctgcggctggcca aaacctacgagacaaccctggaaaaatgctgcgccgctgccgatcctcacgagtgttatgccaaggtgttcgac gagttcaagcctctggtggaagaaccccagaacctgatcaagcagaactgcgagctgttcgagcagctgggcg agtacaagttccagaatgccctgctcgtgcggtacaccaagaaagtgcctcaggtgtccacacctacactggttg aggtgtcccggaatctgggcaaagtgggcagcaagtgttgcaagcaccctgaggccaagagaatgccttgcgc cgaggattacctgagcgtggtgctgaatcagctgtgcgtgctgcacgagaaaacccctgtgtccgacagagtgac caagtgctgtaccgagagcctcgtgaacagaaggccttgctttagcgccctggaagtggacgagacatacgtgc ccaaagagttcaacgccgagacattcaccttccacgccgacatctgcaccctgtccgagaaagagcggcagat caagaagcagacagccctggtcgagctggttaagcacaagcccaaggccaccaaagaacagctgaaggcc gtgatggacgacttcgccgcctttgtcgagaagtgctgcaaggccgacgacaaagagacatgcttcgccgaaga gggcaagaaactggtggctgcttctcaggccgctctgtgtgtggaacccctcggcatggaaaacggcaatatcgc caatagccagattgccgccagcagcgtcagagtgacatttctgggactgcaacactgggtgcccgagctggcta gactgaatagagccggcatggtcaacgcctggacacccagcagcaacgacgataatccctggattcaagtgaa cctgctgcggcgtatgtgggtcacaggtgttgttacacagggcgcaagcagactggccagccacgagtatctgaa ggcctttaaggtggcctacagcctgaacggccacgagttcgacttcatccacgacgtgaacaagaagcacaaa gagtttgtcggcaactggaacaagaacgccgtgcacgtgaacctgttcgagacacctgtggaagcccagtacgt gcggctgtaccctacaagctgtcacaccgcctgcactctgagattcgaactgctgggatgcgagctgaacggctgt gctaatcctctgggcctgaagaacaacagcatccccgataagcagatcaccgccagctccagctataagacatg gggcctgcacctgttcagctggaacccttcttacgccagactggacaagcagggcaacttcaatgcttgggtggcc ggcagctacggcaatgatcagtggctgcaagtggacctgggcagcagcaaagaagtgacaggcatcatcacc caaggggccagaaatttcggcagcgtgcagttcgtggccagctacaaagtggcctactccaacgacagcgcca actggaccgagtatcaggaccctagaaccggcagctccaagatcttccccggcaattgggacaaccacagcca caagaagaatctgttcgaaacccctatcctggccagatatgtgcgcattctgcccgtggcctggcacaacagaatt gccctgagactggaactgctcggctgc |
| 48 | FP776 | LDICSKNPCHNGGLCEEISQEVRGDVFPSYTCTCLKGYAGNHCETKDAHKSEVA HRFKDLGEENFKALVLIAFAQYLQQSPFEDHVKLVNEVTEFAKTCVADESAENCD KSLHTLFGDKLCTVATLRETYGEMADCCAKQEPERNECFLQHKDDNPNLPRLVR PEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKA EFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVACVEPLGMENGNIANSQIAASSVRVTFLGLQHW VPELARLNRAGMVNAWTPSSNDDNPWIQVNLLRRMWVTGVVTQGASRLASHE YLKAFKVAYSLNGHEFDFIHDVNKKHKEFVGNWNKNAVHVNLFETPVEAQYVRL YPTSCHTACTLRFELLGCELNGCANPLGLKNNSIPDKQITASSSYKTWGLHLFSW NPSYARLDKQGNFNAWVAGSYGNDQWLQVDLGSSKEVTGIITQGARNFGSVQF VASYKVAYSNDSANWTEYQDPRTGSSKIFPGNWDNHSHKKNLFETPILARYVRIL PVAWHNRIALRLELLGC |
| 49 | FP776 nucleic acid | ctggacatctgtagcaagaacccttgccacaacggcggcctgtgcgaagagatttctcaagaagtgcggggcga cgttttccccagctacacctgtacatgtctgaagggctacgccggcaaccactgcgagacaaaggatgcccaca agagcgaggtggcccacagattcaaggatctgggcgaagagaacttcaaggccctggtgctgatcgccttcgct cagtatctccagcagagccctttcgaggaccacgtgaagctggtcaacgaagtgaccgagttcgccaagacctg tgtggccgatgagagcgccgagaactgtgataagagcctgcacaccctgttcggcgacaagctgtgtacagtgg ccacactgagagaaacctacggcgagatggccgactgctgtgccaagcaagagcccgagagaaacgagtgc ttcctccagcacaaggacgacaaccccaacctgcctagactcgtgcgacccgaagtggatgtgatgtgcaccgc ctttcacgacaacgaggaaaccttcctgaagaagtacctgtacgagatcgccagacggcacccctacttttatgcc cctgagctgctgttcttcgccaagcggtataaggccgccttcaccgaatgttgccaggccgctgataaggctgcctg tctgctgcctaagctggacgagctgagagatgagggcaaagccagctctgccaagcagagactgaaatgcgcc agcctccagaagttcggcgagagagcttttaaggcctgggccgttgccagactgagccagagatttcctaaggcc gagtttgccgaggtgtccaagctcgtgaccgatctgacaaaggtgcacaccgagtgctgtcacggcgatctgctg gaatgtgccgacgatagagccgacctggccaagtacatctgcgagaaccaggacagcatcagcagcaagctg aaagagtgctgcgagaagcccctgctggaaaagtctcactgtatcgccgaggtggaaaacgacgagatgcctg ccgatctgcctagcctggctgccgatttcgtggaaagcaaggacgtgtgcaagaactacgccgaggccaaggat gtgtttctgggcatgtttctgtatgagtacgcccgcagacaccccgactattctgtggttctgctgctgcggctggcca aaacctacgagacaaccctggaaaaatgctgcgccgctgccgatcctcacgagtgttatgccaaggtgttcgac gagttcaagcctctggtggaagaaccccagaacctgatcaagcagaactgcgagctgttcgagcagctgggcg agtacaagttccagaatgccctgctcgtgcggtacaccaagaaagtgcctcaggtgtccacacctacactggttg aggtgtcccggaatctgggcaaagtgggcagcaagtgttgcaagcaccctgaggccaagagaatgccttgcgc cgaggattacctgagcgtggtgctgaatcagctgtgcgtgctgcacgagaaaacccctgtgtccgacagagtgac caagtgctgtaccgagagcctcgtgaacagaaggccttgctttagcgccctggaagtggacgagacatacgtgc ccaaagagttcaacgccgagacattcaccttccacgccgacatctgcaccctgtccgagaaagagcggcagat caagaagcagacagccctggtcgagctggttaagcacaagcccaaggccaccaaagaacagctgaaggcc gtgatggacgacttcgccgcctttgtcgagaagtgctgcaaggccgacgacaaagagacatgcttcgccgaaga gggcaagaaactggtggcctgtgtggaacccctcggcatggaaaacggcaatatcgccaatagccagattgcc gccagcagcgtcagagtgacatttctgggactgcaacactgggtgcccgagctggctagactgaatagagccgg catggtcaacgcctggacacccagcagcaacgacgataatccctggattcaagtgaacctgctgcggcgtatgt gggtcacaggtgttgttacacagggcgcaagcagactggccagccacgagtatctgaaggcctttaaggtggcct acagcctgaacggccacgagttcgacttcatccacgacgtgaacaagaagcacaaagagtttgtcggcaactg gaacaagaacgccgtgcacgtgaacctgttcgagacacctgtggaagcccagtacgtgcggctgtaccctaca agctgtcacaccgcctgcactctgagattcgaactgctgggatgcgagctgaacggctgtgctaatcctctgggcct gaagaacaacagcatccccgataagcagatcaccgccagctccagctataagacatggggcctgcacctgttc agctggaacccttcttacgccagactggacaagcagggcaacttcaatgcttgggtggccggcagctacggcaa tgatcagtggctgcaagtggacctgggcagcagcaaagaagtgacaggcatcatcacccaaggggccagaa atttcggcagcgtgcagttcgtggccagctacaaagtggcctactccaacgacagcgccaactggaccgagtatc aggaccctagaaccggcagctccaagatcttccccggcaattgggacaaccacagccacaagaagaatctgtt cgaaacccctatcctggccagatatgtgcgcattctgcccgtggcctggcacaacagaattgccctgagactgga actgctcggctgc |
| 50 | FP284 | LDICSKNPCHNGGLCEEISQEVRGDVFPSYTCTCLKGYAGNHCETKGSDAHKSE VAHRFKDLGEENFKALVLIAFAQYLQQSPFEDHVKLVNEVTEFAKTCVADESAEN CDKSLHTLFGDKLCTVATLRETYGEMADCCAKQEPERNECFLQHKDDNPNLPRL VRPEVDVMCTAFHDNEETFLKKYLYEIARRHPYFYAPELLFFAKRYKAAFTECCQ AADKAACLLPKLDELRDEGKASSAKQRLKCASLQKFGERAFKAWAVARLSQRFP KAEFAEVSKLVTDLTKVHTECCHGDLLECADDRADLAKYICENQDSISSKLKECC EKPLLEKSHCIAEVENDEMPADLPSLAADFVESKDVCKNYAEAKDVFLGMFLYEY ARRHPDYSVVLLLRLAKTYETTLEKCCAAADPHECYAKVFDEFKPLVEEPQNLIK QNCELFEQLGEYKFQNALLVRYTKKVPQVSTPTLVEVSRNLGKVGSKCCKHPEA KRMPCAEDYLSVVLNQLCVLHEKTPVSDRVTKCCTESLVNRRPCFSALEVDETY VPKEFNAETFTFHADICTLSEKERQIKKQTALVELVKHKPKATKEQLKAVMDDFA AFVEKCCKADDKETCFAEEGKKLVAASQAALGVGGSGGSGGSGGSCVEPLGM ENGNIANSQIAASSVRVTFLGLQHWVPELARLNRAGMVNAWTPSSNDDNPWIQ VNLLRRMWVTGVVTQGASRLASHEYLKAFKVAYSLNGHEFDFIHDVNKKHKEFV GNWNKNAVHVNLFETPVEAQYVRLYPTSCHTACTLRFELLGCELNGCANPLGLK NNSIPDKQITASSSYKTWGLHLFSWNPSYARLDKQGNFNAWVAGSYGNDQWLQ VDLGSSKEVTGIITQGARNFGSVQFVASYKVAYSNDSANWTEYQDPRTGSSKIF PGNWDNHSHKKNLFETPILARYVRILPVAWHNRIALRLELLGC |
| 51 | FP284 nucleic acid | ctggacatctgcagcaagaatccttgccacaacggcggcctgtgcgaagagatttctcaagaagtgcggggcga cgttttccccagctacacctgtacatgtctgaagggctacgccggcaaccactgcgagacaaaaggatccgatgc tcacaagagcgaggtggcccacagattcaaggatctgggcgaagagaacttcaaggccctggtgctgatcgcct tcgctcagtatctccagcagagccctttcgaggaccacgtgaagctggtcaacgaagtgaccgagttcgccaag acctgtgtggccgatgagagcgccgagaactgtgataagagcctgcacaccctgttcggcgacaagctgtgtac agtggccacactgagagaaacctacggcgagatggccgactgctgtgccaagcaagagcccgagagaaacg agtgcttcctgcagcacaaggacgacaaccccaacctgcctagactcgtgcgacccgaagtggatgtgatgtgc accgcctttcacgacaacgaggaaaccttcctgaagaagtacctgtacgagatcgccagacggcacccctacttt tatgcccctgagctgctgttcttcgccaagcggtataaggccgccttcaccgaatgttgccaggccgctgataaggc tgcctgtctgctgcctaagctggacgagctgagagatgagggcaaagccagctctgccaagcagagactgaagt gcgccagcctgcagaagttcggcgagagagcttttaaggcctgggccgttgccagactgagccagagatttccta aggccgagtttgccgaggtgtccaagctcgtgaccgatctgacaaaggtgcacaccgagtgctgtcacggcgat ctgctggaatgtgccgacgatagagccgacctggccaagtacatctgcgagaaccaggacagcatcagcagc aagctgaaagagtgctgcgagaagcccctgctggaaaagtctcactgtatcgccgaggtggaaaacgacgag atgcctgccgatctgcctagcctggctgccgatttcgtggaaagcaaggacgtgtgcaagaactacgccgaggc caaggatgtgtttctgggcatgtttctgtatgagtacgcccgcagacaccccgactattctgtggttctgctgctgcgg ctggccaaaacctacgagacaaccctggaaaaatgctgcgccgctgccgatcctcacgagtgttatgccaaggt gttcgacgagttcaagcctctggtggaagaaccccagaacctgatcaagcagaactgcgagctgttcgagcagc tgggcgagtacaagttccagaatgccctgctcgtgcggtacaccaagaaagtgcctcaggtgtccacacctacac tggttgaggtgtcccggaatctgggcaaagtgggcagcaagtgttgcaagcaccctgaggccaagagaatgcct tgcgccgaggattacctgagcgtggtgctgaatcagctgtgcgtgctgcacgagaaaacccctgtgtccgacaga gtgaccaagtgctgtaccgagagcctcgtgaacagaaggccttgctttagcgccctggaagtggacgagacata cgtgcccaaagagttcaacgccgagacattcaccttccacgccgacatctgtaccctgagcgagaaagagcgg cagatcaagaagcagacagccctggtcgagctggttaagcacaagcccaaggccaccaaagaacagctgaa ggccgtgatggacgacttcgccgcctttgtcgagaagtgctgcaaggccgacgacaaagagacatgcttcgccg aagagggcaagaaactggtggctgcctctcaggctgctctcggagtgggaggaagcggaggatctggcggttc cggaggctcttgtgtggaacccctcggcatggaaaacggcaatatcgccaatagccagattgccgccagcagc gtcagagtgacatttctgggactgcagcactgggtgcccgagctggctagactgaatagagccggcatggtcaac gcctggacacccagcagcaacgacgataacccttggattcaagtgaacctgctgcggcgtatgtgggtcacagg tgttgttacacagggcgcctctagactggccagccacgagtatctgaaggcctttaaggtggcctacagcctgaac ggccacgagttcgacttcatccacgacgtgaacaagaagcacaaagagtttgtcggcaactggaacaagaacg ccgtgcacgtgaacctgttcgagacacctgtggaagcccagtacgtgcggctgtaccctacaagctgtcacaccg cctgcactctgagattcgaactgctgggatgcgagctgaacggctgtgctaatcctctgggcctgaagaacaaca gcatccccgataagcagatcaccgccagctccagctataagacatggggcctgcacctgttcagctggaaccctt cttacgccagactggacaagcagggcaacttcaatgcttgggtggccggcagctacggcaatgatcagtggctg caagtggacctgggcagcagcaaagaagtgacaggcatcatcacccagggcgccagaaatttcggcagcgtg cagtttgtggccagctacaaagtggcctactccaacgacagcgccaactggaccgagtatcaggaccctagaac cggcagctccaagatcttccccggcaattgggacaaccacagccacaagaagaatctgttcgaaacccctatcc tggccagatatgtgcgcattctgcccgtggcctggcacaacagaattgccctgagactggaactgctcggctgt |
| 52 | FP138 | LDICSKNPCHNGGLCEEISQEVRGDVFPSYTCTCLKGYAGNHCETKGSDAHKSE VAHRFKDLGEENFKALVLIAFAQYLQQSPFEDHVKLVNEVTEFAKTCVADESAEN CDKSLHTLFGDKLCTVATLRETYGEMADCCAKQEPERNECFLQHKDDNPNLPRL VRPEVDVMCTAFHDNEETFLKKYLYEIARRHPYFYAPELLFFAKRYKAAFTECCQ AADKAACLLPKLDELRDEGKASSAKQRLKCASLQKFGERAFKAWAVARLSQRFP KAEFAEVSKLVTDLTKVHTECCHGDLLECADDRADLAKYICENQDSISSKLKECC EKPLLEKSHCIAEVENDEMPADLPSLAADFVESKDVCKNYAEAKDVFLGMFLYEY ARRHPDYSVVLLLRLAKTYETTLEKCCAAADPHECYAKVFDEFKPLVEEPQNLIK QNCELFEQLGEYKFQNALLVRYTKKVPQVSTPTLVEVSRNLGKVGSKCCKHPEA KRMPCAEDYLSVVLNQLCVLHEKTPVSDRVTKCCTESLVNRRPCFSALEVDETY VPKEFNAETFTFHADICTLSEKERQIKKQTALVELVKHKPKATKEQLKAVMDDFA AFVEKCCKADDKETCFAEEGKKLVAASQAALGGSGGSGGSGGSCVEPLGMEN GNIANSQIAASSVRVTFLGLQHWVPELARLNRAGMVNAWTPSSNDDNPWIQVNL LRRMWVTGWTQGASRLASHEYLKAFKVAYSLNGHEFDFIHDVNKKHKEFVGN WNKNAVHVNLFETPVEAQYVRLYPTSCHTACTLRFELLGCELNGCANPLGLKNN SIPDKQITASSSYKTWGLHLFSWNPSYARLDKQGNFNAWVAGSYGNDQWLQVD LGSSKEVTGIITQGARNFGSVQFVASYKVAYSNDSANWTEYQDPRTGSSKIFPG NWDNHSHKKNLFETPILARYVRILPVAWHNRIALRLELLGC |
| 53 | FP138 nucleic acid | ctggacatctgcagcaagaatccttgccacaacggcggcctgtgcgaagagatttctcaagaagtgcggggcga cgttttccccagctacacctgtacatgtctgaagggctacgccggcaaccactgcgagacaaaaggatccgatgc tcacaagagcgaggtggcccacagattcaaggatctgggcgaagagaacttcaaggccctggtgctgatcgcct tcgctcagtatctccagcagagccctttcgaggaccacgtgaagctggtcaacgaagtgaccgagttcgccaag acctgtgtggccgatgagagcgccgagaactgtgataagagcctgcacaccctgttcggcgacaagctgtgtac agtggccacactgagagaaacctacggcgagatggccgactgctgtgccaagcaagagcccgagagaaacg agtgcttcctgcagcacaaggacgacaaccccaacctgcctagactcgtgcgacccgaagtggatgtgatgtgc accgcctttcacgacaacgaggaaaccttcctgaagaagtacctgtacgagatcgccagacggcacccctacttt tatgcccctgagctgctgttcttcgccaagcggtataaggccgccttcaccgaatgttgccaggccgctgataaggc tgcctgtctgctgcctaagctggacgagctgagagatgagggcaaagccagctctgccaagcagagactgaagt gcgccagcctgcagaagttcggcgagagagcttttaaggcctgggccgttgccagactgagccagagatttccta aggccgagtttgccgaggtgtccaagctcgtgaccgatctgacaaaggtgcacaccgagtgctgtcacggcgat ctgctggaatgtgccgacgatagagccgacctggccaagtacatctgcgagaaccaggacagcatcagcagc aagctgaaagagtgctgcgagaagcccctgctggaaaagtctcactgtatcgccgaggtggaaaacgacgag atgcctgccgatctgcctagcctggctgccgatttcgtggaaagcaaggacgtgtgcaagaactacgccgaggc caaggatgtgtttctgggcatgtttctgtatgagtacgcccgcagacaccccgactattctgtggttctgctgctgcgg ctggccaaaacctacgagacaaccctggaaaaatgctgcgccgctgccgatcctcacgagtgttatgccaaggt gttcgacgagttcaagcctctggtggaagaaccccagaacctgatcaagcagaactgcgagctgttcgagcagc tgggcgagtacaagttccagaatgccctgctcgtgcggtacaccaagaaagtgcctcaggtgtccacacctacac tggttgaggtgtcccggaatctgggcaaagtgggcagcaagtgttgcaagcaccctgaggccaagagaatgcct tgcgccgaggattacctgagcgtggtgctgaatcagctgtgcgtgctgcacgagaaaacccctgtgtccgacaga gtgaccaagtgctgtaccgagagcctcgtgaacagaaggccttgctttagcgccctggaagtggacgagacata cgtgcccaaagagttcaacgccgagacattcaccttccacgccgacatctgtaccctgagcgagaaagagcgg cagatcaagaagcagacagccctggtcgagctggttaagcacaagcccaaggccaccaaagaacagctgaa ggccgtgatggacgacttcgccgcctttgtcgagaagtgctgcaaggccgacgacaaagagacatgcttcgccg aagagggcaagaaactggtggctgcctctcaggctgctctcggaggaagcggaggatctggcggttccggagg ctcttgtgtggaacccctcggcatggaaaacggcaatatcgccaatagccagattgccgccagcagcgtcagag tgacatttctgggactgcagcactgggtgcccgagctggctagactgaatagagccggcatggtcaacgcctgga cacccagcagcaacgacgataacccttggattcaagtgaacctgctgcggcgtatgtgggtcacaggtgttgttac acagggcgcctctagactggccagccacgagtatctgaaggcctttaaggtggcctacagcctgaacggccacg agttcgacttcatccacgacgtgaacaagaagcacaaagagtttgtcggcaactggaacaagaacgccgtgca cgtgaacctgttcgagacacctgtggaagcccagtacgtgcggctgtaccctacaagctgtcacaccgcctgcac tctgagattcgaactgctgggatgcgagctgaacggctgtgctaatcctctgggcctgaagaacaacagcatccc cgataagcagatcaccgccagctccagctataagacatggggcctgcacctgttcagctggaacccttcttacgc cagactggacaagcagggcaacttcaatgcttgggtggccggcagctacggcaatgatcagtggctgcaagtg gacctgggcagcagcaaagaagtgacaggcatcatcacccagggcgccagaaatttcggcagcgtgcagtttg tggccagctacaaagtggcctactccaacgacagcgccaactggaccgagtatcaggaccctagaaccggca gctccaagatcttccccggcaattgggacaaccacagccacaagaagaatctgttcgaaacccctatcctggcc agatatgtgcgcattctgcccgtggcctggcacaacagaattgccctgagactggaactgctcggctgt |
| 54 | FP811 | LDICSKNPCHNGGLCEEISQEVRGDVFPSYTCTCLKGYAGNHCETKGSDAHKSE VAHRFKDLGEENFKALVLIAFAQYLQQSPFEDHVKLVNEVTEFAKTCVADESAEN CDKSLHTLFGDKLCTVATLRETYGEMADCCAKQEPERNECFLQHKDDNPNLPRL VRPEVDVMCTAFHDNEETFLKKYLYEIARRHPYFYAPELLFFAKRYKAAFTECCQ AADKAACLLPKLDELRDEGKASSAKQRLKCASLQKFGERAFKAWAVARLSQRFP KAEFAEVSKLVTDLTKVHTECCHGDLLECADDRADLAKYICENQDSISSKLKECC EKPLLEKSHCIAEVENDEMPADLPSLAADFVESKDVCKNYAEAKDVFLGMFLYEY ARRHPDYSVVLLLRLAKTYETTLEKCCAAADPHECYAKVFDEFKPLVEEPQNLIK QNCELFEQLGEYKFQNALLVRYTKKVPQVSTPTLVEVSRNLGKVGSKCCKHPEA KRMPCAEDYLSVVLNQLCVLHEKTPVSDRVTKCCTESLVNRRPCFSALEVDETY VPKEFNAETFTFHADICTLSEKERQIKKQTALVELVKHKPKATKEQLKAVMDDFA AFVEKCCKADDKETCFAEEGKKLVAASQAALGGGGSCVEPLGMENGNIANSQIA ASSVRVTFLGLQHWVPELARLNRAGMVNAWTPSSNDDNPWIQVNLLRRMWVT GVVTQGASRLASHEYLKAFKVAYSLNGHEFDFIHDVNKKHKEFVGNWNKNAVH VNLFETPVEAQYVRLYPTSCHTACTLRFELLGCELNGCANPLGLKNNSIPDKQIT ASSSYKTWGLHLFSWNPSYARLDKQGNFNAWVAGSYGNDQWLQVDLGSSKEV TGIITQGARNFGSVQFVASYKVAYSNDSANWTEYQDPRTGSSKIFPGNWDNHSH KKNLFETPILARYVRILPVAWHNRIALRLELLGC |
| 55 | FP811 nucleic acid | ctggacatctgcagcaagaatccttgccacaacggcggcctgtgcgaagagatttctcaagaagtgcggggcga cgttttccccagctacacctgtacatgtctgaagggctacgccggcaaccactgcgagacaaaaggatccgatgc tcacaagagcgaggtggcccacagattcaaggatctgggcgaagagaacttcaaggccctggtgctgatcgcct tcgctcagtatctccagcagagccctttcgaggaccacgtgaagctggtcaacgaagtgaccgagttcgccaag acctgtgtggccgatgagagcgccgagaactgtgataagagcctgcacaccctgttcggcgacaagctgtgtac agtggccacactgagagaaacctacggcgagatggccgactgctgtgccaagcaagagcccgagagaaacg agtgcttcctgcagcacaaggacgacaaccccaacctgcctagactcgtgcgacccgaagtggatgtgatgtgc accgcctttcacgacaacgaggaaaccttcctgaagaagtacctgtacgagatcgccagacggcacccctacttt tatgcccctgagctgctgttcttcgccaagcggtataaggccgccttcaccgaatgttgccaggccgctgataaggc tgcctgtctgctgcctaagctggacgagctgagagatgagggcaaagccagctctgccaagcagagactgaagt gcgccagcctgcagaagttcggcgagagagcttttaaggcctgggccgttgccagactgagccagagatttccta aggccgagtttgccgaggtgtccaagctcgtgaccgatctgacaaaggtgcacaccgagtgctgtcacggcgat ctgctggaatgtgccgacgatagagccgacctggccaagtacatctgcgagaaccaggacagcatcagcagc aagctgaaagagtgctgcgagaagcccctgctggaaaagtctcactgtatcgccgaggtggaaaacgacgag atgcctgccgatctgcctagcctggctgccgatttcgtggaaagcaaggacgtgtgcaagaactacgccgaggc caaggatgtgtttctgggcatgtttctgtatgagtacgcccgcagacaccccgactattctgtggttctgctgctgcgg ctggccaaaacctacgagacaaccctggaaaaatgctgcgccgctgccgatcctcacgagtgttatgccaaggt gttcgacgagttcaagcctctggtggaagaaccccagaacctgatcaagcagaactgcgagctgttcgagcagc tgggcgagtacaagttccagaatgccctgctcgtgcggtacaccaagaaagtgcctcaggtgtccacacctacac tggttgaggtgtcccggaatctgggcaaagtgggcagcaagtgttgcaagcaccctgaggccaagagaatgcct tgcgccgaggattacctgagcgtggtgctgaatcagctgtgcgtgctgcacgagaaaacccctgtgtccgacaga gtgaccaagtgctgtaccgagagcctcgtgaacagaaggccttgctttagcgccctggaagtggacgagacata cgtgcccaaagagttcaacgccgagacattcaccttccacgccgacatctgtaccctgagcgagaaagagcgg cagatcaagaagcagacagccctggtcgagctggttaagcacaagcccaaggccaccaaagaacagctgaa ggccgtgatggacgacttcgccgcctttgtcgagaagtgctgcaaggccgacgacaaagagacatgcttcgccg aagagggcaagaaactggtggctgcctctcaagctgctctcggaggcggaggatcttgtgtggaacccctcggc atggaaaacggcaatatcgccaatagccagattgccgccagcagcgtcagagtgacatttctgggactgcagca ctgggtgcccgagctggctagactgaatagagccggcatggtcaacgcctggacacccagcagcaacgacga taacccttggattcaagtgaacctgctgcggcgtatgtgggtcacaggtgttgttacacagggcgcctctagactgg ccagccacgagtatctgaaggcctttaaggtggcctacagcctgaacggccacgagttcgacttcatccacgacg tgaacaagaagcacaaagagtttgtcggcaactggaacaagaacgccgtgcacgtgaacctgttcgagacacc tgtggaagcccagtacgtgcggctgtaccctacaagctgtcacaccgcctgcactctgagattcgaactgctggga tgcgagctgaacggctgtgctaatcctctgggcctgaagaacaacagcatccccgataagcagatcaccgccag ctccagctataagacatggggcctgcacctgttcagctggaacccttcttacgccagactggacaagcagggcaa cttcaatgcttgggtggccggcagctacggcaatgatcagtggctgcaagtggacctgggcagcagcaaagaa gtgacaggcatcatcacccagggcgccagaaatttcggcagcgtgcagtttgtggccagctacaaagtggccta ctccaacgacagcgccaactggaccgagtatcaggaccctagaaccggcagctccaagatcttccccggcaat tgggacaaccacagccacaagaagaatctgttcgaaacccctatcctggccagatatgtgcgcattctgcccgtg gcctggcacaacagaattgccctgagactggaactgctcggctgc |
| 56 | FP010 | LDICSKNPCHNGGLCEEISQEVRGDVFPSYTCTCLKGYAGNHCETKGSDAHKSE VAHRFKDLGEENFKALVLIAFAQYLQQSPFEDHVKLVNEVTEFAKTCVADESAEN CDKSLHTLFGDKLCTVATLRETYGEMADCCAKQEPERNECFLQHKDDNPNLPRL VRPEVDVMCTAFHDNEETFLKKYLYEIARRHPYFYAPELLFFAKRYKAAFTECCQ AADKAACLLPKLDELRDEGKASSAKQRLKCASLQKFGERAFKAWAVARLSQRFP KAEFAEVSKLVTDLTKVHTECCHGDLLECADDRADLAKYICENQDSISSKLKECC EKPLLEKSHCIAEVENDEMPADLPSLAADFVESKDVCKNYAEAKDVFLGMFLYEY ARRHPDYSVVLLLRLAKTYETTLEKCCAAADPHECYAKVFDEFKPLVEEPQNLIK QNCELFEQLGEYKFQNALLVRYTKKVPQVSTPTLVEVSRNLGKVGSKCCKHPEA KRMPCAEDYLSVVLNQLCVLHEKTPVSDRVTKCCTESLVNRRPCFSALEVDETY VPKEFNAETFTFHADICTLSEKERQIKKQTALVELVKHKPKATKEQLKAVMDDFA AFVEKCCKADDKETCFAEEGKKLVAASQAALGGGGSGGGGSCVEPLGMENGNI ANSQIAASSVRVTFLGLQHWVPELARLNRAGMVNAWTPSSNDDNPWIQVNLLR RMWVTGWTQGASRLASHEYLKAFKVAYSLNGHEFDFIHDVNKKHKEFVGNWN KNAVHVNLFETPVEAQYVRLYPTSCHTACTLRFELLGCELNGCANPLGLKNNSIP DKQITASSSYKTWGLHLFSWNPSYARLDKQGNFNAWVAGSYGNDQWLQVDLG SSKEVTGIITQGARNFGSVQFVASYKVAYSNDSANWTEYQDPRTGSSKIFPGNW DNHSHKKNLFETPILARYVRILPVAWHNRIALRLELLGC |
| 57 | FP010 nucleic acid | ctggacatctgcagcaagaatccttgccacaacggcggcctgtgcgaagagatttctcaagaagtgcggggcga cgttttccccagctacacctgtacatgtctgaagggctacgccggcaaccactgcgagacaaaaggatccgatgc tcacaagagcgaggtggcccacagattcaaggatctgggcgaagagaacttcaaggccctggtgctgatcgcct tcgctcagtatctccagcagagccctttcgaggaccacgtgaagctggtcaacgaagtgaccgagttcgccaag acctgtgtggccgatgagagcgccgagaactgtgataagagcctgcacaccctgttcggcgacaagctgtgtac agtggccacactgagagaaacctacggcgagatggccgactgctgtgccaagcaagagcccgagagaaacg agtgcttcctgcagcacaaggacgacaaccccaacctgcctagactcgtgcgacccgaagtggatgtgatgtgc accgcctttcacgacaacgaggaaaccttcctgaagaagtacctgtacgagatcgccagacggcacccctacttt tatgcccctgagctgctgttcttcgccaagcggtataaggccgccttcaccgaatgttgccaggccgctgataaggc tgcctgtctgctgcctaagctggacgagctgagagatgagggcaaagccagctctgccaagcagagactgaagt gcgccagcctgcagaagttcggcgagagagcttttaaggcctgggccgttgccagactgagccagagatttccta aggccgagtttgccgaggtgtccaagctcgtgaccgatctgacaaaggtgcacaccgagtgctgtcacggcgat ctgctggaatgtgccgacgatagagccgacctggccaagtacatctgcgagaaccaggacagcatcagcagc aagctgaaagagtgctgcgagaagcccctgctggaaaagtctcactgtatcgccgaggtggaaaacgacgag atgcctgccgatctgcctagcctggctgccgatttcgtggaaagcaaggacgtgtgcaagaactacgccgaggc caaggatgtgtttctgggcatgtttctgtatgagtacgcccgcagacaccccgactattctgtggttctgctgctgcgg ctggccaaaacctacgagacaaccctggaaaaatgctgcgccgctgccgatcctcacgagtgttatgccaaggt gttcgacgagttcaagcctctggtggaagaaccccagaacctgatcaagcagaactgcgagctgttcgagcagc tgggcgagtacaagttccagaatgccctgctcgtgcggtacaccaagaaagtgcctcaggtgtccacacctacac tggttgaggtgtcccggaatctgggcaaagtgggcagcaagtgttgcaagcaccctgaggccaagagaatgcct tgcgccgaggattacctgagcgtggtgctgaatcagctgtgcgtgctgcacgagaaaacccctgtgtccgacaga gtgaccaagtgctgtaccgagagcctcgtgaacagaaggccttgctttagcgccctggaagtggacgagacata cgtgcccaaagagttcaacgccgagacattcaccttccacgccgacatctgtaccctgagcgagaaagagcgg cagatcaagaagcagacagccctggtcgagctggttaagcacaagcccaaggccaccaaagaacagctgaa ggccgtgatggacgacttcgccgcctttgtcgagaagtgctgcaaggccgacgacaaagagacatgcttcgccg aagagggcaagaaactggtggctgcctctcaagctgctctcggaggcggaggctccggaggcggaggatcttg tgtggaacccctcggcatggaaaacggcaatatcgccaatagccagattgccgccagcagcgtcagagtgaca tttctgggactgcagcactgggtgcccgagctggctagactgaatagagccggcatggtcaacgcctggacacc cagcagcaacgacgataacccttggattcaagtgaacctgctgcggcgtatgtgggtcacaggtgttgttacacag ggcgcctctagactggccagccacgagtatctgaaggcctttaaggtggcctacagcctgaacggccacgagttc gacttcatccacgacgtgaacaagaagcacaaagagtttgtcggcaactggaacaagaacgccgtgcacgtga acctgttcgagacacctgtggaagcccagtacgtgcggctgtaccctacaagctgtcacaccgcctgcactctga gattcgaactgctgggatgcgagctgaacggctgtgctaatcctctgggcctgaagaacaacagcatccccgata agcagatcaccgccagctccagctataagacatggggcctgcacctgttcagctggaacccttcttacgccagac tggacaagcagggcaacttcaatgcttgggtggccggcagctacggcaatgatcagtggctgcaagtggacctg ggcagcagcaaagaagtgacaggcatcatcacccagggcgccagaaatttcggcagcgtgcagtttgtggcca gctacaaagtggcctactccaacgacagcgccaactggaccgagtatcaggaccctagaaccggcagctcca agatcttccccggcaattgggacaaccacagccacaagaagaatctgttcgaaacccctatcctggccagatatg tgcgcattctgcccgtggcctggcacaacagaattgccctgagactggaactgctcggctgc |
| 58 | FP816 | LDICSKNPCHNGGLCEEISQEVRGDVFPSYTCTCLKGYAGNHCETKDAHKSEVA HRFKDLGEENFKALVLIAFAQYLQQSPFEDHVKLVNEVTEFAKTCVADESAENCD KSLHTLFGDKLCTVATLRETYGEMADCCAKQEPERNECFLQHKDDNPNLPRLVR PEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKA EFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVAASQAALGLCVEPLGMENGNIANSQIAASSVRV TFLGLQHWVPELARLNRAGMVNAWTPSSNDDNPWIQVNLLRRMWVTGVVTQG ASRLASHEYLKAFKVAYSLNGHEFDFIHDVNKKHKEFVGNWNKNAVHVNLFETP VEAQYVRLYPTSCHTACTLRFELLGCELNGCANPLGLKNNSIPDKQITASSSYKT WGLHLFSWNPSYARLDKQGNFNAWVAGSYGNDQWLQVDLGSSKEVTGIITQG ARNFGSVQFVASYKVAYSNDSANWTEYQDPRTGSSKIFPGNWDNHSHKKNLFE TPILARYVRILPVAWHNRIALRLELLGC |
| 59 | FP816 nucleic acid | ctggacatctgcagcaagaatccttgccacaacggcggcctgtgcgaagagatttctcaagaagtgcggggcga cgttttccccagctacacctgtacatgtctgaagggctacgccggcaaccactgcgagacaaaggatgcccaca agagcgaggtggcccacagattcaaggatctgggcgaagagaacttcaaggccctggtgctgatcgccttcgct cagtatctccagcagagccctttcgaggaccacgtgaagctggtcaacgaagtgaccgagttcgccaagacctg tgtggccgatgagagcgccgagaactgtgataagagcctgcacaccctgttcggcgacaagctgtgtacagtgg ccacactgagagaaacctacggcgagatggccgactgctgtgccaagcaagagcccgagagaaacgagtgc ttcctgcagcacaaggacgacaaccccaacctgcctagactcgtgcgacccgaagtggatgtgatgtgcaccgc ctttcacgacaacgaggaaaccttcctgaagaagtacctgtacgagatcgccagacggcacccctacttttatgcc cctgagctgctgttcttcgccaagcggtataaggccgccttcaccgaatgttgccaggccgctgataaggctgcctg tctgctgcctaagctggacgagctgagagatgagggcaaagccagctctgccaagcagagactgaagtgcgcc agcctgcagaagttcggcgagagagcttttaaggcctgggccgttgccagactgagccagagatttcctaaggcc gagtttgccgaggtgtccaagctcgtgaccgatctgacaaaggtgcacaccgagtgctgtcacggcgatctgctg gaatgtgccgacgatagagccgacctggccaagtacatctgcgagaaccaggacagcatcagcagcaagctg aaagagtgctgcgagaagcccctgctggaaaagtctcactgtatcgccgaggtggaaaacgacgagatgcctg ccgatctgcctagcctggctgccgatttcgtggaaagcaaggacgtgtgcaagaactacgccgaggccaaggat gtgtttctgggcatgtttctgtatgagtacgcccgcagacaccccgactattctgtggttctgctgctgcggctggcca aaacctacgagacaaccctggaaaaatgctgcgccgctgccgatcctcacgagtgttatgccaaggtgttcgac gagttcaagcctctggtggaagaaccccagaacctgatcaagcagaactgcgagctgttcgagcagctgggcg agtacaagttccagaatgccctgctcgtgcggtacaccaagaaagtgcctcaggtgtccacacctacactggttg aggtgtcccggaatctgggcaaagtgggcagcaagtgttgcaagcaccctgaggccaagagaatgccttgcgc cgaggattacctgagcgtggtgctgaatcagctgtgcgtgctgcacgagaaaacccctgtgtccgacagagtgac caagtgctgtaccgagagcctcgtgaacagaaggccttgctttagcgccctggaagtggacgagacatacgtgc ccaaagagttcaacgccgagacattcaccttccacgccgacatctgtaccctgagcgagaaagagcggcagat caagaagcagacagccctggtcgagctggttaagcacaagcccaaggccaccaaagaacagctgaaggcc gtgatggacgacttcgccgcctttgtcgagaagtgctgcaaggccgacgacaaagagacatgcttcgccgaaga gggcaagaaactggtggctgcttctcaggctgccctgggactgtgtgtggaacccctcggcatggaaaacggca atatcgccaatagccagattgccgccagcagcgtcagagtgacatttctgggactgcagcactgggtgcccgag ctggctagactgaatagagccggcatggtcaacgcctggacacccagcagcaacgacgataatccctggatcc aagtgaacctgctgcggcgtatgtgggtcacaggtgttgttacacagggcgcctctagactggccagccacgagt atctgaaggcctttaaggtggcctacagcctgaacggccacgagttcgacttcatccacgacgtgaacaagaag cacaaagagtttgtcggcaactggaacaagaacgccgtgcacgtgaacctgttcgagacacctgtggaagccc agtacgtgcggctgtaccctacaagctgtcacaccgcctgcactctgagattcgaactgctgggatgcgagctga acggctgtgctaatcctctgggcctgaagaacaacagcatccccgataagcagatcaccgccagctccagctat aagacatggggcctgcacctgttcagctggaacccttcttacgccagactggacaagcagggcaacttcaatgct tgggtggccggcagctacggcaatgatcagtggctgcaagtggacctgggcagcagcaaagaagtgacaggc atcatcacccagggcgccagaaatttcggcagcgtgcagtttgtggccagctacaaagtggcctactccaacgac agcgccaactggaccgagtaccaggatcctagaaccggcagctccaagatcttccccggcaattgggacaacc acagccacaagaagaatctgttcgaaacccctatcctggccagatatgtgcggattctgcccgtggcctggcaca acagaattgccctgagactggaactgctcggctgt |
| 62 | (G₂S)₄ linker | GGSGGSGGSGGS |
| 63 | (GS)₄ linker | GSGSGSGS |
| 64 | G₄S linker | GGGGS |
| 65 | (G₄S)₂ linker | GGGGSGGGGS |
| 66 | GS His-tag | GSHHHHHH |
| 67 | His-tag | HHHHHH |
| 68 | Murine MFG-E8 | MQVSRVLAALCGMLLCASGLFAASGDFCDSSLCLNGGTCLTGQDNDIYCLCPEG FTGLVCNETERGPCSPNPCYNDAKCLVTLDTQRGDIFTEYICQCPVGYSGIHCET ETNYYNLDGEYMFTTAVPNTAVPTPAPTPDLSNNLASRCSTQLGMEGGAIADSQ ISASSVYMGFMGLQRWGPELARLYRTGIVNAWTASNYDSKPWIQVNLLRKMRV SGVMTQGASRAGRAEYLKTFKVAYSLDGRKFEFIQDESGGDKEFLGNLDNNSLK VNMFNPTLEAQYIKLYPVSCHRGCTLRFELLGCELHGCSEPLGLKNNTIPDSQMS ASSSYKTWNLRAFGWYPHLGRLDNQGKINAWTAQSNSAKEWLQVDLGTQRQV TGIITQGARDFGHIQYVASYKVAHSDDGVQWTVYEEQGSSKVFQGNLDNNSHK KNIFEKPFMARYVRVLPVSWHNRITLRLELLGC |
| 69 | FP1776 | DICDPNPCENGGICLPGLADGSFSCECPDGFTDPNCSSWEVASDEEEPTSAGP CTPNPCHNGGTCEISEAYRGDTFIGYVCKCPRGFNGIHCQHNINECEVEPCKNG GICTDLVANYSCECPGEFMGRNCQYKDAHKSEVAHRFKDLGEENFKALVLIAFA QYLQQSPFEDHVKLVNEVTEFAKTCVADESAENCDKSLHTLFGDKLCTVATLRE TYGEMADCCAKQEPERNECFLQHKDDNPNLPRLVRPEVDVMCTAFHDNEETFL KKYLYEIARRHPYFYAPELLFFAKRYKAAFTECCQAADKAACLLPKLDELRDEGK ASSAKQRLKCASLQKFGERAFKAWAVARLSQRFPKAEFAEVSKLVTDLTKVHTE CCHGDLLECADDRADLAKYICENQDSISSKLKECCEKPLLEKSHCIAEVENDEMP ADLPSLAADFVESKDVCKNYAEAKDVFLGMFLYEYARRHPDYSVVLLLRLAKTYE TTLEKCCAAADPHECYAKVFDEFKPLVEEPQNLIKQNCELFEQLGEYKFQNALLV RYTKKVPQVSTPTLVEVSRNLGKVGSKCCKHPEAKRMPCAEDYLSVVLNQLCVL HEKTPVSDRVTKCCTESLVNRRPCFSALEVDETYVPKEFNAETFTFHADICTLSE KERQIKKQTALVELVKHKPKATKEQLKAVMDDFAAFVEKCCKADDKETCFAEEG KKLVACSGPLGIEGGIISNQQITASSTHRALFGLQKWYPYYARLNKKGLINAWTAA ENDRWPWIQINLQRKMRVTGVITQGAKRIGSPEYIKSYKIAYSNDGKTWAMYKVK GTNEDMVFRGNIDNNTPYANSFTPPIKAQYVRLYPQVCRRHCTLRMELLGCELS GCSEPLGMKSGHIQDYQITASSIFRTLNMDMFTWEPRKARLDKQGKVNAWTSG HNDQSQWLQVDLLVPTKVTGIITQGAKDFGHVQFVGSYKLAYSNDGEHWTVYQ DEKQRKDKVFQGNFDNDTHRKNVIDPPIYARHIRILPWSWYGRITLRSELLGC |
| 70 | FP1068 | DICDPNPCENGGICLPGLADGSFSCECPDGFTDPNCSSWEVASDEEEPTSAGP CTPNPCHNGGTCEISEAYRGDTFIGYVCKCPRGFNGIHCQHNINECEVEPCKNG GICTDLVANYSCECPGEFMGRNCQYKDAHKSEVAHRFKDLGEENFKALVLIAFA QYLQQSPFEDHVKLVNEVTEFAKTCVADESAENCDKSLHTLFGDKLCTVATLRE TYGEMADCCAKQEPERNECFLQHKDDNPNLPRLVRPEVDVMCTAFHDNEETFL KKYLYEIARRHPYFYAPELLFFAKRYKAAFTECCQAADKAACLLPKLDELRDEGK ASSAKQRLKCASLQKFGERAFKAWAVARLSQRFPKAEFAEVSKLVTDLTKVHTE CCHGDLLECADDRADLAKYICENQDSISSKLKECCEKPLLEKSHCIAEVENDEMP ADLPSLAADFVESKDVCKNYAEAKDVFLGMFLYEYARRHPDYSVVLLLRLAKTYE TTLEKCCAAADPHECYAKVFDEFKPLVEEPQNLIKQNCELFEQLGEYKFQNALLV RYTKKVPQVSTPTLVEVSRNLGKVGSKCCKHPEAKRMPCAEDYLSVVLNQLCVL HEKTPVSDRVTKCCTESLVNRRPCFSALEVDETYVPKEFNAETFTFHADICTLSE KERQIKKQTALVELVKHKPKATKEQLKAVMDDFAAFVEKCCKADDKETCFAEEG KKLVAASQAALCSGPLGIEGGIISNQQITASSTHRALFGLQKWYPYYARLNKKGLI NAWTAAENDRWPWIQINLQRKMRVTGVITQGAKRIGSPEYIKSYKIAYSNDGKT WAMYKVKGTNEDMVFRGNIDNNTPYANSFTPPIKAQYVRLYPQVCRRHCTLRM ELLGCELSGCSEPLGMKSGHIQDYQITASSIFRTLNMDMFTWEPRKARLDKQGK VNAWTSGHNDQSQWLQVDLLVPTKVTGIITQGAKDFGHVQFVGSYKLAYSNDG EHWTVYQDEKQRKDKVFQGNFDNDTHRKNVIDPPIYARHIRILPWSWYGRITLRS ELLGC |
| 71 =133 | FP1777 | DICDPNPCENGGICLPGLADGSFSCECPDGFTDPNCSSWEVASDEEEPTSAGP CTPNPCHNGGTCEISEAYRGDTFIGYVCKCPRGFNGIHCQHNINECEVEPCKNG GICTDLVANYSCECPGEFMGRNCQYKDAHKSEVAHRFKDLGEENFKALVLIAFA QYLQQSPFEDHVKLVNEVTEFAKTCVADESAENCDKSLHTLFGDKLCTVATLRE TYGEMADCCAKQEPERNECFLQHKDDNPNLPRLVRPEVDVMCTAFHDNEETFL KKYLYEIARRHPYFYAPELLFFAKRYKAAFTECCQAADKAACLLPKLDELRDEGK ASSAKQRLKCASLQKFGERAFKAWAVARLSQRFPKAEFAEVSKLVTDLTKVHTE CCHGDLLECADDRADLAKYICENQDSISSKLKECCEKPLLEKSHCIAEVENDEMP ADLPSLAADFVESKDVCKNYAEAKDVFLGMFLYEYARRHPDYSVVLLLRLAKTYE TTLEKCCAAADPHECYAKVFDEFKPLVEEPQNLIKQNCELFEQLGEYKFQNALLV RYTKKVPQVSTPTLVEVSRNLGKVGSKCCKHPEAKRMPCAEDYLSVVLNQLCVL HEKTPVSDRVTKCCTESLVNRRPCFSALEVDETYVPKEFNAETFTFHADICTLSE KERQIKKQTALVELVKHKPKATKEQLKAVMDDFAAFVEKCCKADDKETCFAEEG KKLVACSGPLGIEGGIISNQQITASSTHRALFGLQKWYPYYARLNKKGLINAWTAA ENDRWPWIQINLQRKMRVTGVITQGAKRIGSPEYIKSYKIAYSNDGKTWAMYKVK GTNEDMVFRGNIDNNTPYANSFTPPIKAQYVRLYPQVCRRHCTLRMELLGCELS G |
| 72 | FP1069 | DICDPNPCENGGICLPGLADGSFSCECPDGFTDPNCSSWEVASDEEEPTSAGP CTPNPCHNGGTCEISEAYRGDTFIGYVCKCPRGFNGIHCQHNINECEVEPCKNG GICTDLVANYSCECPGEFMGRNCQYKDAHKSEVAHRFKDLGEENFKALVLIAFA QYLQQSPFEDHVKLVNEVTEFAKTCVADESAENCDKSLHTLFGDKLCTVATLRE TYGEMADCCAKQEPERNECFLQHKDDNPNLPRLVRPEVDVMCTAFHDNEETFL KKYLYEIARRHPYFYAPELLFFAKRYKAAFTECCQAADKAACLLPKLDELRDEGK ASSAKQRLKCASLQKFGERAFKAWAVARLSQRFPKAEFAEVSKLVTDLTKVHTE CCHGDLLECADDRADLAKYICENQDSISSKLKECCEKPLLEKSHCIAEVENDEMP ADLPSLAADFVESKDVCKNYAEAKDVFLGMFLYEYARRHPDYSVVLLLRLAKTYE TTLEKCCAAADPHECYAKVFDEFKPLVEEPQNLIKQNCELFEQLGEYKFQNALLV RYTKKVPQVSTPTLVEVSRNLGKVGSKCCKHPEAKRMPCAEDYLSVVLNQLCVL HEKTPVSDRVTKCCTESLVNRRPCFSALEVDETYVPKEFNAETFTFHADICTLSE KERQIKKQTALVELVKHKPKATKEQLKAVMDDFAAFVEKCCKADDKETCFAEEG KKLVAASQAALCSGPLGIEGGIISNQQITASSTHRALFGLQKWYPYYARLNKKGLI NAWTAAENDRWPWIQINLQRKMRVTGVITQGAKRIGSPEYIKSYKIAYSNDGKT WAMYKVKGTNEDMVFRGNIDNNTPYANSFTPPIKAQYVRLYPQVCRRHCTLRM ELLGCELSG |
| 73 =121 | FP261 | LDICSKNPCHNGGLCEEISQEVRGDVFPSYTCTCLKGYAGNHCETKDAHKSEVA HRFKDLGEENFKALVLIAFAQYLQQSPFEDHVKLVNEVTEFAKTCVADESAENCD KSLHTLFGDKLCTVATLRETYGEMADCCAKQEPERNECFLQHKDDNPNLPRLVR PEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKA EFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVACVEPLGMENGNIANSQIAASSVRVTFLGLQHW VPELARLNRAGMVNAWTPSSNDDNPWIQVNLLRRMWVTGVVTQGASRLASHE YLKAFKVAYSLNGHEFDFIHDVNKKHKEFVGNWNKNAVHVNLFETPVEAQYVRL YPTSCHTACTLRFELLGCELNG |
| 74 =119 | FP262 | LDICSKNPCHNGGLCEEISQEVRGDVFPSYTCTCLKGYAGNHCETKDAHKSEVA HRFKDLGEENFKALVLIAFAQYLQQSPFEDHVKLVNEVTEFAKTCVADESAENCD KSLHTLFGDKLCTVATLRETYGEMADCCAKQEPERNECFLQHKDDNPNLPRLVR PEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKA EFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVAASQAALCVEPLGMENGNIANSQIAASSVRVTFL GLQHWVPELARLNRAGMVNAWTPSSNDDNPWIQVNLLRRMWVTGVVTQGASR LASHEYLKAFKVAYSLNGHEFDFIHDVNKKHKEFVGNWNKNAVHVNLFETPVEA QYVRLYPTSCHTACTLRFELLGCELNG |
| 75 | Full lenght MFG-E8 [L76M] | MPRPRLLAALCGALLCAPSLLVALDICSKNPCHNGGLCEEISQEVRGDVFPSYTC TCLKGYAGNHCETKCVEPLGMENGNIANSQIAASSVRVTFLGLQHWVPELARLN RAGMVNAWTPSSNDDNPWIQVNLLRRMWVTGVVTQGASRLASHEYLKAFKVA YSLNGHEFDFIHDVNKKHKEFVGNWNKNAVHVNLFETPVEAQYVRLYPTSCHTA CTLRFELLGCELNGCANPLGLKNNSIPDKQITASSSYKTWGLHLFSWNPSYARLD KQGNFNAWVAGSYGNDQWLQVDLGSSKEVTGIITQGARNFGSVQFVASYKVAY SNDSANWTEYQDPRTGSSKIFPGNWDNHSHKKNLFETPILARYVRILPVAWHNR IALRLELLGC |
| 76 | PS binding domain MFG-E8 with [L76M] | CVEPLGMENGNIANSQIAASSVRVTFLGLQHWVPELARLNRAGMVNAWTPSSN DDNPWIQVNLLRRMWVTGVVTQGASRLASHEYLKAFKVAYSLNGHEFDFIHDVN KKHKEFVGNWNKNAVHVNLFETPVEAQYVRLYPTSCHTACTLRFELLGCELNGC ANPLGLKNNSIPDKQITASSSYKTWGLHLFSWNPSYARLDKQGNFNAWVAGSY GNDQWLQVDLGSSKEVTGIITQGARNFGSVQFVASYKVAYSNDSANWTEYQDP RTGSSKIFPGNWDNHSHKKNLFETPILARYVRILPVAWHNRIALRLELLGC |
| 77 | EGF binding domain EDIL-3 (EGF-like domains 1-2-3) | DICDPNPCENGGICLPGLADGSFSCECPDGFTDPNCSSWEVASDEEEPTSAGP CTPNPCHNGGTCEISEAYRGDTFIGYVCKCPRGFNGIHCQHNINECEVEPCKNG GICTDLVANYSCECPGEFMGRNCQYK |
| 96 | EGF binding domain EDIL-3 (EGF-like domain 1) | DICDPNPCENGGICLPGLADGSFSCECPDGFTDPNCSSWEVASDEEEPT |
| 97 | EGF binding domain EDIL-3 (EGF-like domain 2) | SAGPCTPNPCHNGGTCEISEAYRGDTFIGYVCKCPRGFNGIHCQH |
| 98 | EGF binding domain EDIL-3 (EGF-like domain 3) | NINECEVEPCKNGGICTDLVANYSCECPGEFMGRNCQYK |
| 99 | EGF binding domain EDIL-3 (EGF-like domains 1 and 2) | DICDPNPCENGGICLPGLADGSFSCECPDGFTDPNCSSWEVASDEEEPTSAGP CTPNPCHNGGTCEISEAYRGDTFIGYVCKCPRGFNGIHCQH |
| 100 | EGF binding domain EDIL-3 (EGF-like domain 2 and 3) | SAGPCTPNPCHNGGTCEISEAYRGDTFIGYVCKCPRGFNGIHCQHNINECEVEP CKNGGICTDLVANYSCECPGEFMGRNCQYK |
| 101 | EGF binding domain EDIL-3 (EGF-like domain 1 and 3) | DICDPNPCENGGICLPGLADGSFSCECPDGFTDPNCSSVVEVASDEEEPTNINE CEVEPCKNGGICTDLVANYSCECPGEFMGRNCQYK |
| 78 | PS binding domain EDIL-3 | CSGPLGIEGGIISNQQITASSTHRALFGLQKWYPYYARLNKKGLINAWTAAENDR WPWIQINLQRKMRVTGVITQGAKRIGSPEYIKSYKIAYSNDGKTWAMYKVKGTNE DMVFRGNIDNNTPYANSFTPPIKAQYVRLYPQVCRRHCTLRMELLGCELSGCSE PLGMKSGHIQDYQITASSIFRTLNMDMFTWEPRKARLDKQGKVNAWTSGHNDQ SQWLQVDLLVPTKVTGIITQGAKDFGHVQFVGSYKLAYSNDGEHWTVYQDEKQ RKDKVFQGNFDNDTHRKNVIDPPIYARHIRILPWSWYGRITLRSELLGCTEEE |
| 79 | PS binding domain EDIL-3 TEEE truncated | CSGPLGIEGGIISNQQITASSTHRALFGLQKWYPYYARLNKKGLINAWTAAENDR WPWIQINLQRKMRVTGVITQGAKRIGSPEYIKSYKIAYSNDGKTWAMYKVKGTNE DMVFRGNIDNNTPYANSFTPPIKAQYVRLYPQVCRRHCTLRMELLGCELSGCSE PLGMKSGHIQDYQITASSIFRTLNMDMFTWEPRKARLDKQGKVNAWTSGHNDQ SQWLQVDLLVPTKVTGIITQGAKDFGHVQFVGSYKLAYSNDGEHWTVYQDEKQ RKDKVFQGNFDNDTHRKNVIDPPIYARHIRILPWSWYGRITLRSELLGC |
| 80 | EGF-like domain 1-2-3 [EDIL3]_HSA[A 626-L633]removed_ C1_C2[MFG-E8] Non-M 3163 | DICDPNPCENGGICLPGLADGSFSCECPDGFTDPNCSSWEVASDEEEPTSAGP CTPNPCHNGGTCEISEAYRGDTFIGYVCKCPRGFNGIHCQHNINECEVEPCKNG GICTDLVANYSCECPGEFMGRNCQYKDAHKSEVAHRFKDLGEENFKALVLIAFA QYLQQSPFEDHVKLVNEVTEFAKTCVADESAENCDKSLHTLFGDKLCTVATLRE TYGEMADCCAKQEPERNECFLQHKDDNPNLPRLVRPEVDVMCTAFHDNEETFL KKYLYEIARRHPYFYAPELLFFAKRYKAAFTECCQAADKAACLLPKLDELRDEGK ASSAKQRLKCASLQKFGERAFKAWAVARLSQRFPKAEFAEVSKLVTDLTKVHTE CCHGDLLECADDRADLAKYICENQDSISSKLKECCEKPLLEKSHCIAEVENDEMP ADLPSLAADFVESKDVCKNYAEAKDVFLGMFLYEYARRHPDYSVVLLLRLAKTYE TTLEKCCAAADPHECYAKVFDEFKPLVEEPQNLIKQNCELFEQLGEYKFQNALLV RYTKKVPQVSTPTLVEVSRNLGKVGSKCCKHPEAKRMPCAEDYLSVVLNQLCVL HEKTPVSDRVTKCCTESLVNRRPCFSALEVDETYVPKEFNAETFTFHADICTLSE KERQIKKQTALVELVKHKPKATKEQLKAVMDDFAAFVEKCCKADDKETCFAEEG KKLVACVEPLGMENGNIANSQIAASSVRVTFLGLQHWVPELARLNRAGMVNAWT PSSNDDNPWIQVNLLRRMWVTGVVTQGASRLASHEYLKAFKVAYSLNGHEFDFI HDVNKKHKEFVGNWNKNAVHVNLFETPVEAQYVRLYPTSCHTACTLRFELLGCE LNGCANPLGLKNNSIPDKQITASSSYKTWGLHLFSWNPSYARLDKQGNFNAWVA GSYGNDQWLQVDLGSSKEVTGIITQGARNFGSVQFVASYKVAYSNDSANWTEY QDPRTGSSKIFPGNWDNHSHKKNLFETPILARYVRILPVAWHNRIALRLELLGC |
| 102 | EGF-like domain 1[EDIL3]_HSA[ A626-L633]removed_ C1_C2[MFG-E8] | DICDPNPCENGGICLPGLADGSFSCECPDGFTDPNCSSVVEVASDEEEPTDAHK SEVAHRFKDLGEENFKALVLIAFAQYLQQSPFEDHVKLVNEVTEFAKTCVADESA ENCDKSLHTLFGDKLCTVATLRETYGEMADCCAKQEPERNECFLQHKDDNPNL PRLVRPEVDVMCTAFHDNEETFLKKYLYEIARRHPYFYAPELLFFAKRYKAAFTE CCQAADKAACLLPKLDELRDEGKASSAKQRLKCASLQKFGERAFKAWAVARLS QRFPKAEFAEVSKLVTDLTKVHTECCHGDLLECADDRADLAKYICENQDSISSKL KECCEKPLLEKSHCIAEVENDEMPADLPSLAADFVESKDVCKNYAEAKDVFLGM FLYEYARRHPDYSVVLLLRLAKTYETTLEKCCAAADPHECYAKVFDEFKPLVEEP QNLIKQNCELFEQLGEYKFQNALLVRYTKKVPQVSTPTLVEVSRNLGKVGSKCC KHPEAKRMPCAEDYLSVVLNQLCVLHEKTPVSDRVTKCCTESLVNRRPCFSALE VDETYVPKEFNAETFTFHADICTLSEKERQIKKQTALVELVKHKPKATKEQLKAVM DDFAAFVEKCCKADDKETCFAEEGKKLVACVEPLGMENGNIANSQIAASSVRVT FLGLQHWVPELARLNRAGMVNAWTPSSNDDNPWIQVNLLRRMWVTGVVTQGA SRLASHEYLKAFKVAYSLNGHEFDFIHDVNKKHKEFVGNWNKNAVHVNLFETPV EAQYVRLYPTSCHTACTLRFELLGCELNGCANPLGLKNNSIPDKQITASSSYKTW GLHLFSWNPSYARLDKQGNFNAWVAGSYGNDQWLQVDLGSSKEVTGIITQGAR NFGSVQFVASYKVAYSNDSANWTEYQDPRTGSSKIFPGNWDNHSHKKNLFETPI LARYVRILPVAWHNRIALRLELLGC |
| 103 | EGF-like domain 2[EDIL3]_HSA[ A626-L633]removed_ C1_C2[MFG-E8] | SAGPCTPNPCHNGGTCEISEAYRGDTFIGYVCKCPRGFNGIHCQHDAHKSEVAH RFKDLGEENFKALVLIAFAQYLQQSPFEDHVKLVNEVTEFAKTCVADESAENCDK SLHTLFGDKLCTVATLRETYGEMADCCAKQEPERNECFLQHKDDNPNLPRLVRP EVDVMCTAFHDNEETFLKKYLYEIARRHPYFYAPELLFFAKRYKAAFTECCQAAD KAACLLPKLDELRDEGKASSAKQRLKCASLQKFGERAFKAWAVARLSQRFPKAE FAEVSKLVTDLTKVHTECCHGDLLECADDRADLAKYICENQDSISSKLKECCEKP LLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVACVEPLGMENGNIANSQIAASSVRVTFLGLQHW VPELARLNRAGMVNAWTPSSNDDNPWIQVNLLRRMWVTGVVTQGASRLASHE YLKAFKVAYSLNGHEFDFIHDVNKKHKEFVGNWNKNAVHVNLFETPVEAQYVRL YPTSCHTACTLRFELLGCELNGCANPLGLKNNSIPDKQITASSSYKTWGLHLFSW NPSYARLDKQGNFNAWVAGSYGNDQWLQVDLGSSKEVTGIITQGARNFGSVQF VASYKVAYSNDSANWTEYQDPRTGSSKIFPGNWDNHSHKKNLFETPILARYVRIL PVAWHNRIALRLELLGC |
| 104 | EGF-like domain 3[EDIL3]_HSA[ A626-L633]removed_ C1_C2[MFG-E8] | NINECEVEPCKNGGICTDLVANYSCECPGEFMGRNCQYKDAHKSEVAHRFKDL GEENFKALVLIAFAQYLQQSPFEDHVKLVNEVTEFAKTCVADESAENCDKSLHTL FGDKLCTVATLRETYGEMADCCAKQEPERNECFLQHKDDNPNLPRLVRPEVDV MCTAFHDNEETFLKKYLYEIARRHPYFYAPELLFFAKRYKAAFTECCQAADKAAC LLPKLDELRDEGKASSAKQRLKCASLQKFGERAFKAWAVARLSQRFPKAEFAEV SKLVTDLTKVHTECCHGDLLECADDRADLAKYICENQDSISSKLKECCEKPLLEK SHCIAEVENDEMPADLPSLAADFVESKDVCKNYAEAKDVFLGMFLYEYARRHPD YSVVLLLRLAKTYETTLEKCCAAADPHECYAKVFDEFKPLVEEPQNLIKQNCELFE QLGEYKFQNALLVRYTKKVPQVSTPTLVEVSRNLGKVGSKCCKHPEAKRMPCA EDYLSVVLNQLCVLHEKTPVSDRVTKCCTESLVNRRPCFSALEVDETYVPKEFN AETFTFHADICTLSEKERQIKKQTALVELVKHKPKATKEQLKAVMDDFAAFVEKC CKADDKETCFAEEGKKLVACVEPLGMENGNlANSQlAASSVRVTFLGLQHWVPE LARLNRAGMVNAWTPSSNDDNPWIQVNLLRRMWVTGVVTQGASRLASHEYLK AFKVAYSLNGHEFDFIHDVNKKHKEFVGNWNKNAVHVNLFETPVEAQYVRLYPT SCHTACTLRFELLGCELNGCANPLGLKNNSIPDKQITASSSYKTWGLHLFSWNPS YARLDKQGNFNAWVAGSYGNDQWLQVDLGSSKEVTGIITQGARNFGSVQFVAS YKVAYSNDSANWTEYQDPRTGSSKIFPGNWDNHSHKKNLFETPILARYVRILPVA WHNRIALRLELLGC |
| 105 | EGF-like domain 1-2[EDIL3]_HSA[ A626-L633]removed_ C1_C2[MFG-E8] | DICDPNPCENGGICLPGLADGSFSCECPDGFTDPNCSSWEVASDEEEPTSAGP CTPNPCHNGGTCEISEAYRGDTFIGYVCKCPRGFNGIHCQHDAHKSEVAHRFKD LGEENFKALVLIAFAQYLQQSPFEDHVKLVNEVTEFAKTCVADESAENCDKSLHT LFGDKLCTVATLRETYGEMADCCAKQEPERNECFLQHKDDNPNLPRLVRPEVD VMCTAFHDNEETFLKKYLYEIARRHPYFYAPELLFFAKRYKAAFTECCQAADKAA CLLPKLDELRDEGKASSAKQRLKCASLQKFGERAFKAWAVARLSQRFPKAEFAE VSKLVTDLTKVHTECCHGDLLECADDRADLAKYICENQDSISSKLKECCEKPLLE KSHCIAEVENDEMPADLPSLAADFVESKDVCKNYAEAKDVFLGMFLYEYARRHP DYSVVLLLRLAKTYETTLEKCCAAADPHECYAKVFDEFKPLVEEPQNLIKQNCEL FEQLGEYKFQNALLVRYTKKVPQVSTPTLVEVSRNLGKVGSKCCKHPEAKRMPC AEDYLSVVLNQLCVLHEKTPVSDRVTKCCTESLVNRRPCFSALEVDETYVPKEF NAETFTFHADICTLSEKERQIKKQTALVELVKHKPKATKEQLKAVMDDFAAFVEK CCKADDKETCFAEEGKKLVACVEPLGMENGNIANSQIAASSVRVTFLGLQHWVP ELARLNRAGMVNAWTPSSNDDNPWIQVNLLRRMWVTGVVTQGASRLASHEYL KAFKVAYSLNGHEFDFIHDVNKKHKEFVGNWNKNAVHVNLFETPVEAQYVRLYP TSCHTACTLRFELLGCELNGCANPLGLKNNSIPDKQITASSSYKTWGLHLFSWNP SYARLDKQGNFNAWVAGSYGNDQWLQVDLGSSKEVTGIITQGARNFGSVQFVA SYKVAYSNDSANWTEYQDPRTGSSKIFPGNWDNHSHKKNLFETPILARYVRILPV AWHNRIALRLELLGC |
| 106 | EGF-like domain 2-3[EDIL3]_HSA[ A626-L633]removed_ C1_C2[MFG-E8] | SAGPCTPNPCHNGGTCEISEAYRGDTFIGYVCKCPRGFNGIHCQHNINECEVEP CKNGGICTDLVANYSCECPGEFMGRNCQYKDAHKSEVAHRFKDLGEENFKALV LIAFAQYLQQSPFEDHVKLVNEVTEFAKTCVADESAENCDKSLHTLFGDKLCTVA TLRETYGEMADCCAKQEPERNECFLQHKDDNPNLPRLVRPEVDVMCTAFHDNE ETFLKKYLYEIARRHPYFYAPELLFFAKRYKAAFTECCQAADKAACLLPKLDELRD EGKASSAKQRLKCASLQKFGERAFKAWAVARLSQRFPKAEFAEVSKLVTDLTKV HTECCHGDLLECADDRADLAKYICENQDSISSKLKECCEKPLLEKSHCIAEVEND EMPADLPSLAADFVESKDVCKNYAEAKDVFLGMFLYEYARRHPDYSVVLLLRLA KTYETTLEKCCAAADPHECYAKVFDEFKPLVEEPQNLIKQNCELFEQLGEYKFQN ALLVRYTKKVPQVSTPTLVEVSRNLGKVGSKCCKHPEAKRMPCAEDYLSVVLNQ LCVLHEKTPVSDRVTKCCTESLVNRRPCFSALEVDETYVPKEFNAETFTFHADIC TLSEKERQIKKQTALVELVKHKPKATKEQLKAVMDDFAAFVEKCCKADDKETCFA EEGKKLVACVEPLGMENGNIANSQIAASSVRVTFLGLQHWVPELARLNRAGMVN AWTPSSNDDNPWIQVNLLRRMWVTGVVTQGASRLASHEYLKAFKVAYSLNGHE FDFIHDVNKKHKEFVGNWNKNAVHVNLFETPVEAQYVRLYPTSCHTACTLRFEL LGCELNGCANPLGLKNNSIPDKQITASSSYKTWGLHLFSWNPSYARLDKQGNFN AWVAGSYGNDQWLQVDLGSSKEVTGIITQGARNFGSVQFVASYKVAYSNDSAN WTEYQDPRTGSSKIFPGNWDNHSHKKNLFETPILARYVRILPVAWHNRIALRLEL LGC |
| 107 | EGF-like domain 1-3[EDIL3]_HSA[ A626-L633]removed_ C1_C2[MFG-E8] | DICDPNPCENGGICLPGLADGSFSCECPDGFTDPNCSSVVEVASDEEEPTNINE CEVEPCKNGGICTDLVANYSCECPGEFMGRNCQYKDAHKSEVAHRFKDLGEEN FKALVLIAFAQYLQQSPFEDHVKLVNEVTEFAKTCVADESAENCDKSLHTLFGDK LCTVATLRETYGEMADCCAKQEPERNECFLQHKDDNPNLPRLVRPEVDVMCTA FHDNEETFLKKYLYEIARRHPYFYAPELLFFAKRYKAAFTECCQAADKAACLLPKL DELRDEGKASSAKQRLKCASLQKFGERAFKAWAVARLSQRFPKAEFAEVSKLVT DLTKVHTECCHGDLLECADDRADLAKYICENQDSISSKLKECCEKPLLEKSHCIAE VENDEMPADLPSLAADFVESKDVCKNYAEAKDVFLGMFLYEYARRHPDYSVVLL LRLAKTYETTLEKCCAAADPHECYAKVFDEFKPLVEEPQNLIKQNCELFEQLGEY KFQNALLVRYTKKVPQVSTPTLVEVSRNLGKVGSKCCKHPEAKRMPCAEDYLSV VLNQLCVLHEKTPVSDRVTKCCTESLVNRRPCFSALEVDETYVPKEFNAETFTFH ADICTLSEKERQIKKQTALVELVKHKPKATKEQLKAVMDDFAAFVEKCCKADDKE TCFAEEGKKLVACVEPLGMENGNIANSQIAASSVRVTFLGLQHWVPELARLNRA GMVNAWTPSSNDDNPWIQVNLLRRMWVTGVVTQGASRLASHEYLKAFKVAYSL NGHEFDFIHDVNKKHKEFVGNWNKNAVHVNLFETPVEAQYVRLYPTSCHTACTL RFELLGCELNGCANPLGLKNNSIPDKQITASSSYKTWGLHLFSWNPSYARLDKQ GNFNAWVAGSYGNDQWLQVDLGSSKEVTGIITQGARNFGSVQFVASYKVAYSN DSANWTEYQDPRTGSSKIFPGNWDNHSHKKNLFETPILARYVRILPVAWHNRIAL RLELLGC |
| 81 | Nucleic acid of Seq ID NO: 80 | gacatctgcgaccccaatccttgcgagaatggcggcatttgtctgcctggactggccgatggcagcttctcttgtga atgccccgatggcttcacagaccccaattgcagctctgtggtggaagtggccagcgacgaggaagaacctaca agcgctggcccctgcacacccaatccatgtcataatggcggaacctgcgagatcagcgaggcctacagaggcg ataccttcatcggctacgtgtgcaagtgccccagaggcttcaatggcatccactgccagcacaacatcaacgagt gcgaggtggaaccatgcaagaacggcggcatctgtaccgacctggtggccaattactcttgcgagtgccctggc gagttcatgggcagaaactgccagtacaaggacgcccacaagagcgaggtggcccacagattcaaggacctg ggcgaagagaacttcaaggccctggtgctgatcgccttcgctcagtatctccagcagagccctttcgaggaccac gtgaagctggtcaacgaagtgaccgagttcgccaagacctgtgtggccgatgagagcgccgagaactgtgaca agagcctgcacacactgttcggcgacaagctgtgtaccgtggccacactgagagaaacctacggcgagatggc cgactgctgtgccaagcaagagcccgagagaaacgagtgcttcctccagcacaaggatgacaaccccaacct gcctagactcgtgcggcctgaagtggatgtgatgtgcaccgcctttcacgacaacgaggaaaccttcctgaagaa gtacctgtacgagatcgccagacggcacccctacttttatgcccctgagctgctgttcttcgccaagcggtataagg ccgccttcaccgaatgttgccaggccgctgataaggctgcctgtctgctgcctaagctggacgagctgagagatg agggcaaagccagctctgccaagcagagactgaaatgcgccagcctccagaagttcggcgagagagcttttaa ggcctgggccgttgccagactgagccagagatttcctaaggccgagtttgccgaggtgtccaagctcgtgaccga tctgacaaaggtgcacaccgagtgctgtcacggcgatctgctggaatgtgccgacgatagagccgacctggcca agtatatctgcgagaaccaggacagcatcagcagcaagctgaaagagtgctgcgagaagcccctgctggaaa agtctcactgtatcgccgaagtggaaaacgacgagatgcccgccgatctgccttctctggctgccgatttcgtgga aagcaaggatgtgtgcaagaactacgccgaggccaaagatgtgtttctgggcatgtttctgtatgagtacgcccgc agacaccccgactattctgtggttctgctgctgcggctggccaagacatacgagacaaccctggaaaaatgctgc gccgctgccgatcctcacgagtgttatgccaaggtgttcgacgagttcaagccactggtggaagaaccccagaa cctgatcaagcagaactgcgagctgttcgagcagctgggcgagtacaagttccagaatgccctgctcgtgcggta caccaagaaagtgcctcaggtgtccacacctacactggttgaggtgtcccggaatctgggcaaagtgggcagca agtgttgcaagcaccctgaggccaagagaatgccttgcgccgaggattacctgagcgtggtgctgaatcagctgt gcgtgctgcacgagaaaacccctgtgtccgacagagtgaccaagtgctgtaccgagagcctcgtgaacagaag gccttgctttagcgccctggaagtggacgagacatacgtgcccaaagagttcaacgccgagacattcaccttcca cgccgatatctgcaccctgtccgagaaagagcggcagatcaagaagcagacagccctggtcgagctggttaag cacaagcccaaggccaccaaagaacagctgaaggccgtgatggacgacttcgccgcctttgtcgagaagtgct gcaaggccgacgacaaagagacatgcttcgccgaagagggcaagaaactggtggcctgtgtggaacccctcg gcatggaaaacggcaatatcgccaatagccagattgccgccagcagcgtcagagtgacatttctgggactgcaa cactgggtgcccgagctggctagactgaatagagccggcatggtcaacgcctggacacccagcagcaacgac gataacccctggattcaagtgaacctgctgcggcgtatgtgggtcacaggtgttgttacacagggcgcaagcaga ctggcctctcacgagtacctgaaggcctttaaggtggcctacagcctgaacggccacgagttcgacttcatccacg acgtgaacaagaagcacaaagagtttgtcggcaactggaacaagaacgccgtgcacgtgaacctgttcgaga cacctgtggaagcccagtacgtgcggctgtaccctacaagctgtcacaccgcctgcactctgagattcgaactgct gggatgcgagctgaacggctgtgctaatcctctgggcctgaagaacaacagcatccccgataagcagatcacc gccagctccagctataagacatggggcctgcacctgttcagctggaacccttcttacgccagactggacaagcag ggcaacttcaatgcttgggtggccggcagctacggcaatgatcagtggctgcaagtggatctgggcagcagcaa agaagtgacaggcatcatcacccaaggggccagaaatttcggcagcgtgcagttcgtggccagctacaaagtg gcctactccaacgacagcgccaactggaccgagtatcaggaccctagaaccggcagctccaagatcttccccg gcaattgggacaaccacagccacaagaagaatctcttcgagactcccatcctggccagatatgtgcggattctgc ctgtggcctggcacaacagaatcgccctgagactggaactgctcggctgt |
| 108 | Nucleic acid of Seq ID NO: 102 | gacatctgcgaccccaacccctgcgagaacggcggcatctgcctgcccggcctggccgacggcagcttcagct gcgagtgccccgacggcttcaccgaccccaactgcagcagcgtggtggaggtggccagcgacgaggaggag cccaccgacgcccacaagagcgaggtggcccaccggttcaaggacctgggcgaggagaacttcaaggccct ggtgctgatcgccttcgcccagtacctgcagcagagccccttcgaggaccacgtgaagctggtgaacgaggtga ccgagttcgccaagacctgcgtggccgacgagagcgccgagaactgcgacaagagcctgcacaccctgttcg gcgacaagctgtgcaccgtggccaccctgcgggagacctacggcgagatggccgactgctgcgccaagcagg agcccgagcggaacgagtgcttcctgcagcacaaggacgacaaccccaacctgccccggctggtgcggcccg aggtggacgtgatgtgcaccgccttccacgacaacgaggagaccttcctgaagaagtacctgtacgagatcgcc cggcggcacccctacttctacgcccccgagctgctgttcttcgccaagcggtacaaggccgccttcaccgagtgct gccaggccgccgacaaggccgcctgcctgctgcccaagctggacgagctgcgggacgagggcaaggccag cagcgccaagcagcggctgaagtgcgccagcctgcagaagttcggcgagcgggccttcaaggcctgggccgt ggcccggctgagccagcggttccccaaggccgagttcgccgaggtgagcaagctggtgaccgacctgaccaa ggtgcacaccgagtgctgccacggcgacctgctggagtgcgccgacgaccgggccgacctggccaagtacat ctgcgagaaccaggacagcatcagcagcaagctgaaggagtgctgcgagaagcccctgctggagaagagcc actgcatcgccgaggtggagaacgacgagatgcccgccgacctgcccagcctggccgccgacttcgtggaga gcaaggacgtgtgcaagaactacgccgaggccaaggacgtgttcctgggcatgttcctgtacgagtacgcccgg cggcaccccgactacagcgtggtgctgctgctgcggctggccaagacctacgagaccaccctggagaagtgct gcgccgccgccgacccccacgagtgctacgccaaggtgttcgacgagttcaagcccctggtggaggagcccca gaacctgatcaagcagaactgcgagctgttcgagcagctgggcgagtacaagttccagaacgccctgctggtgc ggtacaccaagaaggtgccccaggtgagcacccccaccctggtggaggtgagccggaacctgggcaaggtg ggcagcaagtgctgcaagcaccccgaggccaagcggatgccctgcgccgaggactacctgagcgtggtgctg aaccagctgtgcgtgctgcacgagaagacccccgtgagcgaccgggtgaccaagtgctgcaccgagagcctg gtgaaccggcggccctgcttcagcgccctggaggtggacgagacctacgtgcccaaggagttcaacgccgaga ccttcaccttccacgccgacatctgcaccctgagcgagaaggagcggcagatcaagaagcagaccgccctggt ggagctggtgaagcacaagcccaaggccaccaaggagcagctgaaggccgtgatggacgacttcgccgcctt cgtggagaagtgctgcaaggccgacgacaaggagacctgcttcgccgaggagggcaagaagctggtggcct gcgtggagcccctgggcatggagaacggcaacatcgccaacagccagatcgccgccagcagcgtgcgggtg accttcctgggcctgcagcactgggtgcccgagctggcccggctgaaccgggccggcatggtgaacgcctgga cccccagcagcaacgacgacaacccctggatccaggtgaacctgctgcggcggatgtgggtgaccggcgtggt gacccagggcgccagccggctggccagccacgagtacctgaaggccttcaaggtggcctacagcctgaacgg ccacgagttcgacttcatccacgacgtgaacaagaagcacaaggagttcgtgggcaactggaacaagaacgc cgtgcacgtgaacctgttcgagacccccgtggaggcccagtacgtgcggctgtaccccaccagctgccacaccg cctgcaccctgcggttcgagctgctgggctgcgagctgaacggctgcgccaaccccctgggcctgaagaacaa cagcatccccgacaagcagatcaccgccagcagcagctacaagacctggggcctgcacctgttcagctggaa ccccagctacgcccggctggacaagcagggcaacttcaacgcctgggtggccggcagctacggcaacgacc agtggctgcaggtggacctgggcagcagcaaggaggtgaccggcatcatcacccagggcgcccggaacttcg gcagcgtgcagttcgtggccagctacaaggtggcctacagcaacgacagcgccaactggaccgagtaccagg acccccggaccggcagcagcaagatcttccccggcaactgggacaaccacagccacaagaagaacctgttc gagacccccatcctggcccggtacgtgcggatcctgcccgtggcctggcacaaccggatcgccctgcggctgga gctgctgggctgc |
| 109 | Nucleic acid of Seq ID NO: 103 | agcgccggcccctgcacccccaacccctgccacaacggcggcacctgcgagatcagcgaggcctaccgggg cgacaccttcatcggctacgtgtgcaagtgcccccggggcttcaacggcatccactgccagcacgacgcccaca agagcgaggtggcccaccggttcaaggacctgggcgaggagaacttcaaggccctggtgctgatcgccttcgc ccagtacctgcagcagagccccttcgaggaccacgtgaagctggtgaacgaggtgaccgagttcgccaagacc tgcgtggccgacgagagcgccgagaactgcgacaagagcctgcacaccctgttcggcgacaagctgtgcacc gtggccaccctgcgggagacctacggcgagatggccgactgctgcgccaagcaggagcccgagcggaacga gtgcttcctgcagcacaaggacgacaaccccaacctgccccggctggtgcggcccgaggtggacgtgatgtgc accgccttccacgacaacgaggagaccttcctgaagaagtacctgtacgagatcgcccggcggcacccctactt ctacgcccccgagctgctgttcttcgccaagcggtacaaggccgccttcaccgagtgctgccaggccgccgaca aggccgcctgcctgctgcccaagctggacgagctgcgggacgagggcaaggccagcagcgccaagcagcg gctgaagtgcgccagcctgcagaagttcggcgagcgggccttcaaggcctgggccgtggcccggctgagccag cggttccccaaggccgagttcgccgaggtgagcaagctggtgaccgacctgaccaaggtgcacaccgagtgct gccacggcgacctgctggagtgcgccgacgaccgggccgacctggccaagtacatctgcgagaaccaggac agcatcagcagcaagctgaaggagtgctgcgagaagcccctgctggagaagagccactgcatcgccgaggtg gagaacgacgagatgcccgccgacctgcccagcctggccgccgacttcgtggagagcaaggacgtgtgcaag aactacgccgaggccaaggacgtgttcctgggcatgttcctgtacgagtacgcccggcggcaccccgactacag cgtggtgctgctgctgcggctggccaagacctacgagaccaccctggagaagtgctgcgccgccgccgacccc cacgagtgctacgccaaggtgttcgacgagttcaagcccctggtggaggagccccagaacctgatcaagcaga actgcgagctgttcgagcagctgggcgagtacaagttccagaacgccctgctggtgcggtacaccaagaaggtg ccccaggtgagcacccccaccctggtggaggtgagccggaacctgggcaaggtgggcagcaagtgctgcaa gcaccccgaggccaagcggatgccctgcgccgaggactacctgagcgtggtgctgaaccagctgtgcgtgctg cacgagaagacccccgtgagcgaccgggtgaccaagtgctgcaccgagagcctggtgaaccggcggccctg cttcagcgccctggaggtggacgagacctacgtgcccaaggagttcaacgccgagaccttcaccttccacgccg acatctgcaccctgagcgagaaggagcggcagatcaagaagcagaccgccctggtggagctggtgaagcac aagcccaaggccaccaaggagcagctgaaggccgtgatggacgacttcgccgccttcgtggagaagtgctgc aaggccgacgacaaggagacctgcttcgccgaggagggcaagaagctggtggcctgcgtggagcccctggg catggagaacggcaacatcgccaacagccagatcgccgccagcagcgtgcgggtgaccttcctgggcctgca gcactgggtgcccgagctggcccggctgaaccgggccggcatggtgaacgcctggacccccagcagcaacg acgacaacccctggatccaggtgaacctgctgcggcggatgtgggtgaccggcgtggtgacccagggcgcca gccggctggccagccacgagtacctgaaggccttcaaggtggcctacagcctgaacggccacgagttcgacttc atccacgacgtgaacaagaagcacaaggagttcgtgggcaactggaacaagaacgccgtgcacgtgaacctg ttcgagacccccgtggaggcccagtacgtgcggctgtaccccaccagctgccacaccgcctgcaccctgcggtt cgagctgctgggctgcgagctgaacggctgcgccaaccccctgggcctgaagaacaacagcatccccgacaa gcagatcaccgccagcagcagctacaagacctggggcctgcacctgttcagctggaaccccagctacgcccgg ctggacaagcagggcaacttcaacgcctgggtggccggcagctacggcaacgaccagtaggctgcaggtggac ctgggcagcagcaaggaggtgaccggcatcatcacccagggcgcccggaacttcggcagcgtgcagttcgtg gccagctacaaggtggcctacagcaacgacagcgccaactggaccgagtaccaggacccccggaccggca gcagcaagatcttccccggcaactgggacaaccacagccacaagaagaacctgttcgagacccccatcctgg cccggtacgtgcggatcctgcccgtggcctggcacaaccggatcgccctgcggctggagctgctgggctgc |
| 110 | Nucleic acid of Seq ID NO: 104 | aacatcaacgagtgcgaggtggagccctgcaagaacggcggcatctgcaccgacctggtggccaactacagc tgcgagtgccccggcgagttcatgggccggaactgccagtacaaggacgcccacaagagcgaggtggcccac cggttcaaggacctgggcgaggagaacttcaaggccctggtgctgatcgccttcgcccagtacctgcagcagag ccccttcgaggaccacgtgaagctggtgaacgaggtgaccgagttcgccaagacctgcgtggccgacgagag cgccgagaactgcgacaagagcctgcacaccctgttcggcgacaagctgtgcaccgtggccaccctgcggga gacctacggcgagatggccgactgctgcgccaagcaggagcccgagcggaacgagtgcttcctgcagcacaa ggacgacaaccccaacctgccccggctggtgcggcccgaggtggacgtgatgtgcaccgccttccacgacaac gaggagaccttcctgaagaagtacctgtacgagatcgcccggcggcacccctacttctacgcccccgagctgct gttcttcgccaagcggtacaaggccgccttcaccgagtgctgccaggccgccgacaaggccgcctgcctgctgc ccaagctggacgagctgcgggacgagggcaaggccagcagcgccaagcagcggctgaagtgcgccagcct gcagaagttcggcgagcgggccttcaaggcctgggccgtggcccggctgagccagcggttccccaaggccga gttcgccgaggtgagcaagctggtgaccgacctgaccaaggtgcacaccgagtgctgccacggcgacctgctg gagtgcgccgacgaccgggccgacctggccaagtacatctgcgagaaccaggacagcatcagcagcaagct gaaggagtgctgcgagaagcccctgctggagaagagccactgcatcgccgaggtggagaacgacgagatgc ccgccgacctgcccagcctggccgccgacttcgtggagagcaaggacgtgtgcaagaactacgccgaggcca aggacgtgttcctgggcatgttcctgtacgagtacgcccggcggcaccccgactacagcgtggtgctgctgctgcg gctggccaagacctacgagaccaccctggagaagtgctgcgccgccgccgacccccacgagtgctacgccaa ggtgttcgacgagttcaagcccctggtggaggagccccagaacctgatcaagcagaactgcgagctgttcgagc agctgggcgagtacaagttccagaacgccctgctggtgcggtacaccaagaaggtgccccaggtgagcacccc caccctggtggaggtgagccggaacctgggcaaggtgggcagcaagtgctgcaagcaccccgaggccaagc ggatgccctgcgccgaggactacctgagcgtggtgctgaaccagctgtgcgtgctgcacgagaagacccccgtg agcgaccgggtgaccaagtgctgcaccgagagcctggtgaaccggcggccctgcttcagcgccctggaggtgg acgagacctacgtgcccaaggagttcaacgccgagaccttcaccttccacgccgacatctgcaccctgagcgag aaggagcggcagatcaagaagcagaccgccctggtggagctggtgaagcacaagcccaaggccaccaagg agcagctgaaggccgtgatggacgacttcgccgccttcgtggagaagtgctgcaaggccgacgacaaggaga cctgcttcgccgaggagggcaagaagctggtggcctgcgtggagcccctgggcatggagaacggcaacatcg ccaacagccagatcgccgccagcagcgtgcgggtgaccttcctgggcctgcagcactgggtgcccgagctggc ccggctgaaccgggccggcatggtgaacgcctggacccccagcagcaacgacgacaacccctggatccagg tgaacctgctgcggcggatgtgggtgaccggcgtggtgacccagggcgccagccggctggccagccacgagt acctgaaggccttcaaggtggcctacagcctgaacggccacgagttcgacttcatccacgacgtgaacaagaa gcacaaggagttcgtgggcaactggaacaagaacgccgtgcacgtgaacctgttcgagacccccgtggaggc ccagtacgtgcggctgtaccccaccagctgccacaccgcctgcaccctgcggttcgagctgctgggctgcgagct gaacggctgcgccaaccccctgggcctgaagaacaacagcatccccgacaagcagatcaccgccagcagca gctacaagacctggggcctgcacctgttcagctggaaccccagctacgcccggctggacaagcagggcaactt caacgcctgggtggccggcagctacggcaacgaccagtggctgcaggtggacctgggcagcagcaaggagg tgaccggcatcatcacccagggcgcccggaacttcggcagcgtgcagttcgtggccagctacaaggtggcctac agcaacgacagcgccaactggaccgagtaccaggacccccggaccggcagcagcaagatcttccccggca actgggacaaccacagccacaagaagaacctgttcgagacccccatcctggcccggtacgtgcggatcctgcc cgtggcctggcacaaccggatcgccctgcggctggagctgctgggctgc |
| 111 | Nucleic acid of Seq ID NO: 105 | gacatctgcgaccccaacccctgcgagaacggcggcatctgcctgcccggcctggccgacggcagcttcagct gcgagtgccccgacggcttcaccgaccccaactgcagcagcgtggtggaggtggccagcgacgaggaggag cccaccagcgccggcccctgcacccccaacccctgccacaacggcggcacctgcgagatcagcgaggccta ccggggcgacaccttcatcggctacgtgtgcaagtgcccccggggcttcaacggcatccactgccagcacgacg cccacaagagcgaggtggcccaccggttcaaggacctgggcgaggagaacttcaaggccctggtgctgatcg ccttcgcccagtacctgcagcagagccccttcgaggaccacgtgaagctggtgaacgaggtgaccgagttcgcc aagacctgcgtggccgacgagagcgccgagaactgcgacaagagcctgcacaccctgttcggcgacaagctg tgcaccgtggccaccctgcgggagacctacggcgagatggccgactgctgcgccaagcaggagcccgagcg gaacgagtgcttcctgcagcacaaggacgacaaccccaacctgccccggctggtgcggcccgaggtggacgt gatgtgcaccgccttccacgacaacgaggagaccttcctgaagaagtacctgtacgagatcgcccggcggcac ccctacttctacgcccccgagctgctgttcttcgccaagcggtacaaggccgccttcaccgagtgctgccaggccg ccgacaaggccgcctgcctgctgcccaagctgg acgagctgcgggacgagggcaaggccagcagcgccaa gcagcggctgaagtgcgccagcctgcagaagttcggcgagcgggccttcaaggcctgggccgtggcccggctg agccagcggttccccaaggccgagttcgccgaggtgagcaagctggtgaccgacctgaccaaggtgcacacc gagtgctgccacggcgacctgctggagtgcgccgacgaccgggccgacctggccaagtacatctgcgagaac caggacagcatcagcagcaagctgaaggagtgctgcgagaagcccctgctggagaagagccactgcatcgc cgaggtggagaacgacgagatgcccgccgacctgcccagcctggccgccgacttcgtggagagcaaggacgt gtgcaagaactacgccgaggccaaggacgtgttcctgggcatgttcctgtacgagtacgcccggcggcaccccg actacagcgtggtgctgctgctgcggctggccaagacctacgagaccaccctggagaagtgctgcgccgccgc cgacccccacgagtgctacgccaaggtgttcgacgagttcaagcccctggtggaggagccccagaacctgatc aagcagaactgcgagctgttcgagcagctgggcgagtacaagttccagaacgccctgctggtgcggtacacca agaaggtgccccaggtgagcacccccaccctggtggaggtgagccggaacctgggcaaggtgggcagcaag tgctgcaagcaccccgaggccaagcggatgccctgcgccgaggactacctgagcgtggtgctgaaccagctgt gcgtgctgcacgagaagacccccgtgagcgaccgggtgaccaagtgctgcaccgagagcctggtgaaccggc ggccctgcttcagcgccctggaggtggacgagacctacgtgcccaaggagttcaacgccgagaccttcaccttc cacgccgacatctgcaccctgagcgagaaggagcggcagatcaagaagcagaccgccctggtggagctggt gaagcacaagcccaaggccaccaaggagcagctgaaggccgtgatggacgacttcgccgccttcgtggaga agtgctgcaaggccgacgacaaggagacctgcttcgccgaggagggcaagaagctggtggcctgcgtggagc ccctgggcatggagaacggcaacatcgccaacagccagatcgccgccagcagcgtgcgggtgaccttcctgg gcctgcagcactgggtgcccgagctggcccggctgaaccgggccggcatggtgaacgcctggacccccagca gcaacgacgacaacccctggatccaggtgaacctgctgcggcggatgtgggtgaccggcgtggtgacccagg gcgccagccggctggccagccacgagtacctgaaggccttcaaggtggcctacagcctgaacggccacgagtt cgacttcatccacgacgtgaacaagaagcacaaggagttcgtgggcaactggaacaagaacgccgtgcacgt gaacctgttcgagacccccgtggaggcccagtacgtgcggctgtaccccaccagctgccacaccgcctgcacc ctgcggttcgagctgctgggctgcgagctgaacggctgcgccaaccccctgggcctgaagaacaacagcatcc ccgacaagcagatcaccgccagcagcagctacaagacctggggcctgcacctgttcagctggaaccccagct acgcccggctggacaagcagggcaacttcaacgcctgggtggccggcagctacggcaacgaccagtggctgc aggtggacctgggcagcagcaaggaggtgaccggcatcatcacccagggcgcccggaacttcggcagcgtg cagttcgtggccagctacaaggtggcctacagcaacgacagcgccaactggaccgagtaccaggacccccgg accggcagcagcaagatcttccccggcaactgggacaaccacagccacaagaagaacctgttcgagacccc catcctggcccggtacgtgcggatcctgcccgtggcctggcacaaccggatcgccctgcggctggagctgctgg gctgc |
| 112 | Nucleic acid of Seq ID NO: 106 | agcgccggcccctgcacccccaacccctgccacaacggcggcacctgcgagatcagcgaggcctaccgggg cgacaccttcatcggctacgtgtgcaagtgcccccggggcttcaacggcatccactgccagcacaacatcaacg agtgcgaggtggagccctgcaagaacggcggcatctgcaccgacctggtggccaactacagctgcgagtgccc cggcgagttcatgggccggaactgccagtacaaggacgcccacaagagcgaggtggcccaccggttcaagg acctgggcgaggagaacttcaaggccctggtgctgatcgccttcgcccagtacctgcagcagagccccttcgag gaccacgtgaagctggtgaacgaggtgaccgagttcgccaagacctgcgtggccgacgagagcgccgagaa ctgcgacaagagcctgcacaccctgttcggcgacaagctgtgcaccgtggccaccctgcgggagacctacggc gagatggccgactgctgcgccaagcaggagcccgagcggaacgagtgcttcctgcagcacaaggacgacaa ccccaacctgccccggctggtgcggcccgaggtggacgtgatgtgcaccgccttccacgacaacgaggagacc ttcctgaagaagtacctgtacgagatcgcccggcggcacccctacttctacgcccccgagctgctgttcttcgccaa gcggtacaaggccgccttcaccgagtgctgccaggccgccgacaaggccgcctgcctgctgcccaagctggac gagctgcgggacgagggcaaggccagcagcgccaagcagcggctgaagtgcgccagcctgcagaagttcg gcgagcgggccttcaaggcctgggccgtggcccggctgagccagcggttccccaaggccgagttcgccgaggt gagcaagctggtgaccgacctgaccaaggtgcacaccgagtgctgccacggcgacctgctggagtgcgccga cgaccgggccgacctggccaagtacatctgcgagaaccaggacagcatcagcagcaagctgaaggagtgct gcgagaagcccctgctggagaagagccactgcatcgccgaggtggagaacgacgagatgcccgccgacctg cccagcctggccgccgacttcgtggagagcaaggacgtgtgcaagaactacgccgaggccaaggacgtgttcc tgggcatgttcctgtacgagtacgcccggcggcaccccgactacagcgtggtgctgctgctgcggctggccaaga cctacgagaccaccctggagaagtgctgcgccgccgccgacccccacgagtgctacgccaaggtgttcgacga gttcaagcccctggtggaggagccccagaacctgatcaagcagaactgcgagctgttcgagcagctgggcgag tacaagttccagaacgccctgctggtgcggtacaccaagaaggtgccccaggtgagcacccccaccctggtgg aggtgagccggaacctgggcaaggtgggcagcaagtgctgcaagcaccccgaggccaagcggatgccctgc gccgaggactacctgagcgtggtgctgaaccagctgtgcgtgctgcacgagaagacccccgtgagcgaccgg gtgaccaagtgctgcaccgagagcctggtgaaccggcggccctgcttcagcgccctggaggtggacgagacct acgtgcccaaggagttcaacgccgagaccttcaccttccacgccgacatctgcaccctgagcgagaaggagcg gcagatcaagaagcagaccgccctggtggagctggtgaagcacaagcccaaggccaccaaggagcagctg aaggccgtgatggacgacttcgccgccttcgtggagaagtgctgcaaggccgacgacaaggagacctgcttcg ccgaggagggcaagaagctggtggcctgcgtggagcccctgggcatggagaacggcaacatcgccaacagc cagatcgccgccagcagcgtgcgggtgaccttcctgggcctgcagcactgggtgcccgagctggcccggctga accgggccggcatggtgaacgcctggacccccagcagcaacgacgacaacccctggatccaggtgaacctg ctgcggcggatgtgggtgaccggcgtggtgacccagggcgccagccggctggccagccacgagtacctgaag gccttcaaggtggcctacagcctgaacggccacgagttcgacttcatccacgacgtgaacaagaagcacaagg agttcgtgggcaactggaacaagaacgccgtgcacgtgaacctgttcgagacccccgtggaggcccagtacgt gcggctgtaccccaccagctgccacaccgcctgcaccctgcggttcgagctgctgggctgcgagctgaacggct gcgccaaccccctgggcctgaagaacaacagcatccccgacaagcagatcaccgccagcagcagctacaag acctggggcctgcacctgttcagctggaaccccagctacgcccggctggacaagcagggcaacttcaacgcct gggtggccggcagctacggcaacgaccagtggctgcaggtggacctgggcagcagcaaggaggtgaccggc atcatcacccagggcgcccggaacttcggcagcgtgcagttcgtggccagctacaaggtggcctacagcaacg acagcgccaactggaccgagtaccaggacccccggaccggcagcagcaagatcttccccggcaactgggac aaccacagccacaagaagaacctgttcgagacccccatcctggcccggtacgtgcggatcctgcccgtggcctg gcacaaccggatcgccctgcggctggagctgctgggctgc |
| 113 | Nucleic acid of Seq ID NO: 107 | gacatctgcgaccccaacccctgcgagaacggcggcatctgcctgcccggcctggccgacggcagcttcagct gcgagtgccccgacggcttcaccgaccccaactgcagcagcgtggtggaggtggccagcgacgaggaggag cccaccaacatcaacgagtgcgaggtggagccctgcaagaacggcggcatctgcaccgacctggtggccaac tacagctgcgagtgccccggcgagttcatgggccggaactgccagtacaaggacgcccacaagagcgaggtg gcccaccggttcaaggacctgggcgaggagaacttcaaggccctggtgctgatcgccttcgcccagtacctgca gcagagccccttcgaggaccacgtgaagctggtgaacgaggtgaccgagttcgccaagacctgcgtggccga cgagagcgccgagaactgcgacaagagcctgcacaccctgttcggcgacaagctgtgcaccgtggccaccct gcgggagacctacggcgagatggccgactgctgcgccaagcaggagcccgagcggaacgagtgcttcctgc agcacaaggacgacaaccccaacctgccccggctggtgcggcccgaggtggacgtgatgtgcaccgccttcc acgacaacgaggagaccttcctgaagaagtacctgtacgagatcgcccggcggcacccctacttctacgccccc gagctgctgttcttcgccaagcggtacaaggccgccttcaccgagtgctgccaggccgccgacaaggccgcctg cctgctgcccaagctggacgagctgcgggacgagggcaaggccagcagcgccaagcagcggctgaagtgc gccagcctgcagaagttcggcgagcgggccttcaaggcctgggccgtggcccggctgagccagcggttcccca aggccgagttcgccgaggtgagcaagctggtgaccgacctgaccaaggtgcacaccgagtgctgccacggcg acctgctggagtgcgccgacgaccgggccgacctggccaagtacatctgcgagaaccaggacagcatcagca gcaagctgaaggagtgctgcgagaagcccctgctggagaagagccactgcatcgccgaggtggagaacgac gagatgcccgccgacctgcccagcctggccgccgacttcgtggagagcaaggacgtgtgcaagaactacgcc gaggccaaggacgtgttcctgggcatgttcctgtacgagtacgcccggcggcaccccgactacagcgtggtgctg ctgctgcggctggccaagacctacgagaccaccctggagaagtgctgcgccgccgccgacccccacgagtgct acgccaaggtgttcgacgagttcaagcccctggtggaggagccccagaacctgatcaagcagaactgcgagct gttcgagcagctgggcgagtacaagttccagaacgccctgctggtgcggtacaccaagaaggtgccccaggtg agcacccccaccctggtggaggtgagccggaacctgggcaaggtgggcagcaagtgctgcaagcaccccga ggccaagcggatgccctgcgccgaggactacctgagcgtggtgctgaaccagctgtgcgtgctgcacgagaag acccccgtgagcgaccgggtgaccaagtgctgcaccgagagcctggtgaaccggcggccctgcttcagcgccc tggaggtggacgagacctacgtgcccaaggagttcaacgccgagaccttcaccttccacgccgacatctgcacc ctgagcgagaaggagcggcagatcaagaagcagaccgccctggtggagctggtgaagcacaagcccaagg ccaccaaggagcagctgaaggccgtgatggacgacttcgccgccttcgtggagaagtgctgcaaggccgacg acaaggagacctgcttcgccgaggagggcaagaagctggtggcctgcgtggagcccctgggcatggagaacg gcaacatcgccaacagccagatcgccgccagcagcgtgcgggtgaccttcctgggcctgcagcactgggtgcc cgagctggcccggctgaaccgggccggcatggtgaacgcctggacccccagcagcaacgacgacaacccct ggatccaggtgaacctgctgcggcggatgtgggtgaccggcgtggtgacccagggcgccagccggctggcca gccacgagtacctgaaggccttcaaggtggcctacagcctgaacggccacgagttcgacttcatccacgacgtg aacaagaagcacaaggagttcgtgggcaactggaacaagaacgccgtgcacgtgaacctgttcgagaccccc gtggaggcccagtacgtgcggctgtaccccaccagctgccacaccgcctgcaccctgcggttcgagctgctggg ctgcgagctgaacggctgcgccaaccccctgggcctgaagaacaacagcatccccgacaagcagatcaccgc cagcagcagctacaagacctggggcctgcacctgttcagctggaaccccagctacgcccggctggacaagca gggcaacttcaacgcctgggtggccggcagctacggcaacgaccagtggctgcaggtggacctgggcagcag caaggaggtgaccggcatcatcacccagggcgcccggaacttcggcagcgtgcagttcgtggccagctacaag gtggcctacagcaacgacagcgccaactggaccgagtaccaggacccccggaccggcagcagcaagatctt ccccggcaactgggacaaccacagccacaagaagaacctgttcgagacccccatcctggcccggtacgtgcg gatcctgcccgtggcctggcacaaccggatcgccctgcggctggagctgctgggctgc |
| 82 | EGF[MFG-E8]_HSA[A626-L633]removed_ C1_C2[EDIL3] | LDICSKNPCHNGGLCEEISQEVRGDVFPSYTCTCLKGYAGNHCETKDAHKSEVA HRFKDLGEENFKALVLIAFAQYLQQSPFEDHVKLVNEVTEFAKTCVADESAENCD KSLHTLFGDKLCTVATLRETYGEMADCCAKQEPERNECFLQHKDDNPNLPRLVR PEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKA EFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVACSGPLGIEGGIISNQQITASSTHRALFGLQKWY PYYARLNKKGLINAWTAAENDRWPWIQINLQRKMRVTGVITQGAKRIGSPEYIKS YKIAYSNDGKTWAMYKVKGTNEDMVFRGNIDNNTPYANSFTPPIKAQYVRLYPQ VCRRHCTLRMELLGCELSGCSEPLGMKSGHIQDYQITASSIFRTLNMDMFTWEP RKARLDKQGKVNAWTSGHNDQSQWLQVDLLVPTKVTGIITQGAKDFGHVQFVG SYKLAYSNDGEHWTVYQDEKQRKDKVFQGNFDNDTHRKNVIDPPIYARHIRILP WSWYGRITLRSELLGC |
| 83 | Nucleic acid of Seq ID NO: 82 | ctggacatctgtagcaagaacccttgccacaacggcggcctgtgcgaagagatttctcaagaagtgcggggcga cgttttccccagctacacctgtacatgtctgaagggctacgccggcaaccactgcgagacaaaggatgcccaca agagcgaggtggcccacagattcaaggatctgggcgaagagaacttcaaggccctggtgctgatcgccttcgct cagtatctccagcagagccctttcgaggaccacgtgaagctggtcaacgaagtgaccgagttcgccaagacctg tgtggccgatgagagcgccgagaactgtgataagagcctgcacaccctgttcggcgacaagctgtgtacagtgg ccacactgagagaaacctacggcgagatggccgactgctgtgccaagcaagagcccgagagaaacgagtgc ttcctccagcacaaggacgacaaccccaacctgcctagactcgtgcgacccgaagtggatgtgatgtgcaccgc ctttcacgacaacgaggaaaccttcctgaagaagtacctgtacgagatcgccagacggcacccctacttttatgcc cctgagctgctgttcttcgccaagcggtataaggccgccttcaccgaatgttgccaggccgctgataaggctgcctg tctgctgcctaagctggacgagctgagagatgagggcaaagccagctctgccaagcagagactgaaatgcgcc agcctccagaagttcggcgagagagcttttaaggcctgggccgttgccagactgagccagagatttcctaaggcc gagtttgccgaggtgtccaagctcgtgaccgatctgacaaaggtgcacaccgagtgctgtcacggcgatctgctg gaatgtgccgacgatagagccgacctggccaagtacatctgcgagaaccaggacagcatcagcagcaagctg aaagagtgctgcgagaagcccctgctggaaaagtctcactgtatcgccgaggtggaaaacgacgagatgcctg ccgatctgcctagcctggctgccgatttcgtggaaagcaaggacgtgtgcaagaactacgccgaggccaaggat gtgtttctgggcatgtttctgtatgagtacgcccgcagacaccccgactattctgtggttctgctgctgcggctggcca aaacctacgagacaaccctggaaaaatgctgcgccgctgccgatcctcacgagtgttatgccaaggtgttcgac gagttcaagcctctggtggaagaaccccagaacctgatcaagcagaactgcgagctgttcgagcagctgggcg agtacaagttccagaatgccctgctcgtgcggtacaccaagaaagtgcctcaggtgtccacacctacactggttg aggtgtcccggaatctgggcaaagtgggcagcaagtgttgcaagcaccctgaggccaagagaatgccttgcgc cgaggattacctgagcgtggtgctgaatcagctgtgcgtgctgcacgagaaaacccctgtgtccgacagagtgac caagtgctgtaccgagagcctcgtgaacagaaggccttgctttagcgccctggaagtggacgagacatacgtgc ccaaagagttcaacgccgagacattcaccttccacgccgacatctgcaccctgtccgagaaagagcggcagat caagaagcagacagccctggtcgagctggttaagcacaagcccaaggccaccaaagaacagctgaaggcc gtgatggacgacttcgccgcctttgtcgagaagtgctgcaaggccgacgacaaagagacatgcttcgccgaaga gggcaagaaactggtggcctgttctggccctctgggcatcgaaggcggcatcatcagcaatcagcagatcaccg ccagcagcacccacagagcactgtttggcctgcaaaagtggtatccctactacgcccggctgaacaagaaggg cctgattaacgcctggacagccgccgagaatgacagatggccctggattcagatcaacctccagcggaagatg agagtgaccggcgttatcacacagggcgcaaagagaatcggctcccctgagtacatcaagagctacaagatcg cctacagcaacgacggcaagacctgggccatgtacaaagtgaagggcaccaacgaggacatggtgttccggg gcaacatcgacaacaacaccccttacgccaacagcttcacccctcctatcaaggcccagtacgtgcggctgtac cctcaagtgtgcagaaggcactgtaccctgagaatggaactgctgggctgcgaactgtctggctgttctgagccac tgggaatgaagtccggccacatccaggactaccagattaccgcctccagcatcttcagaaccctgaacatggata tgttcacctgggagccccggaaggccagactggataagcagggaaaagtgaatgcctggaccagcggccaca acgaccagtctcaatggctgcaagtggacctgctggtgcctaccaaagtgaccggaatcatcacccaaggcgct aaggatttcggccacgtgcagttcgtgggctcctacaagctggcctactccaatgatggcgagcactggaccgtgt accaggacgagaagcagcggaaggataaggtgttccagggaaacttcgataacgatacccaccggaagaac gtgatcgaccctccaatctacgccagacacatcagaatcctgccttggtcttggtacggcagaatcaccctgagat ccgagctgctgggatgc |
| 115 | EGF-C1-His6 | LDICSKNPCHNGGLCEEISQEVRGDVFPSYTCTCLKGYAGNHCETKCVEPLGME NGNIANSQIAASSVRVTFLGLQHWVPELARLNRAGMVNAWTPSSNDDNPWIQV NLLRRMWVTGVVTQGASRLASHEYLKAFKVAYSLNGHEFDFIHDVNKKHKEFVG NWNKNAVHVNLFETPVEAQYVRLYPTSCHTACTLRFELLGCELNGHHHHHH |
| 116 | Nucleic acid of 115 | ctggacatctgtagcaagaacccttgccacaacggcggcctgtgcgaagagatttctcaagaagtgcggggcga cgttttccccagctacacctgtacatgtctgaagggctacgccggcaaccactgcgagacaaaatgtgtggaacc cctcggcatggaaaacggcaatatcgccaatagccagatcgccgccagcagcgtcagagtgacatttctggga ctgcaacactgggtgccagagctggccagactgaatagagccggcatggttaacgcctggacacccagcagc aacgacgacaacccctggattcaagtgaacctgctgcggcgtatgtgggtcacaggtgttgttacacagggcgca agcagactggccagccacgagtatctgaaggcctttaaggtggcctacagcctgaacggccacgagttcgactt catccacgacgtgaacaagaagcacaaagagttcgtcggcaactggaacaagaacgccgtgcacgtgaacct gttcgagacacctgtggaagcccagtacgtgcggctgtaccctacaagctgtcacaccgcctgcacactgagatt cgagctgctgggctgtgaactgaatggccaccaccaccatcaccac |
| 117 =147 | EGF-HSA-C1 C1 | LDICSKNPCHNGGLCEEISQEVRGDVFPSYTCTCLKGYAGNHCETKGSDAHKSE VAHRFKDLGEENFKALVLIAFAQYLQQSPFEDHVKLVNEVTEFAKTCVADESAEN CDKSLHTLFGDKLCTVATLRETYGEMADCCAKQEPERNECFLQHKDDNPNLPRL VRPEVDVMCTAFHDNEETFLKKYLYEIARRHPYFYAPELLFFAKRYKAAFTECCQ AADKAACLLPKLDELRDEGKASSAKQRLKCASLQKFGERAFKAWAVARLSQRFP KAEFAEVSKLVTDLTKVHTECCHGDLLECADDRADLAKYICENQDSISSKLKECC EKPLLEKSHCIAEVENDEMPADLPSLAADFVESKDVCKNYAEAKDVFLGMFLYEY ARRHPDYSVVLLLRLAKTYETTLEKCCAAADPHECYAKVFDEFKPLVEEPQNLIK QNCELFEQLGEYKFQNALLVRYTKKVPQVSTPTLVEVSRNLGKVGSKCCKHPEA KRMPCAEDYLSVVLNQLCVLHEKTPVSDRVTKCCTESLVNRRPCFSALEVDETY VPKEFNAETFTFHADICTLSEKERQIKKQTALVELVKHKPKATKEQLKAVMDDFA AFVEKCCKADDKETCFAEEGKKLVAASQAALGLGGSGGSGGSGGSCVEPLGM ENGNIANSQIAASSVRVTFLGLQHWVPELARLNRAGMVNAWTPSSNDDNPWIQ VNLLRRMVVVTGVVTQGASRLASHEYLKAFKVAYSLNGHEFDFIHDVNKKHKEFV GNWNKNAVHVNLFETPVEAQYVRLYPTSCHTACTLRFELLGCELNG |
| 118 | Nucleic acid of 117 | ctggacatctgtagcaagaacccttgccacaacggcggcctgtgcgaagagatttctcaagaagtgcggggcga cgttttccccagctacacctgtacatgtctgaagggctacgccggcaaccactgcgagacaaaaggatctgatgc ccacaagagcgaggtggcccacagattcaaggatctgggcgaagagaacttcaaggccctggtgctgatcgcc ttcgctcagtatctccagcagagccctttcgaggaccacgtgaagctggtcaacgaagtgaccgagttcgccaag acctgtgtggccgatgagagcgccgagaactgtgataagagcctgcacaccctgttcggcgacaagctgtgtac agtggccacactgagagaaacctacggcgagatggccgactgctgtgccaagcaagagcccgagagaaacg agtgcttcctccagcacaaggacgacaaccccaacctgcctagactcgtgcgacccgaagtggatgtgatgtgc accgcctttcacgacaacgaggaaaccttcctgaagaagtacctgtacgagatcgccagacggcacccctacttt tatgcccctgagctgctgttcttcgccaagcggtataaggccgccttcaccgaatgttgccaggccgctgataaggc tgcctgtctgctgcctaagctggacgagctgagagatgagggcaaagccagctctgccaagcagagactgaaat gcgccagcctccagaagttcggcgagagagcttttaaggcctgggccgttgccagactgagccagagatttccta aggccgagtttgccgaggtgtccaagctcgtgaccgatctgacaaaggtgcacaccgagtgctgtcacggcgat ctgctggaatgtgccgacgatagagccgacctggccaagtacatctgcgagaaccaggacagcatcagcagc aagctgaaagagtgctgcgagaagcccctgctggaaaagtctcactgtatcgccgaggtggaaaacgacgag atgcctgccgatctgcctagcctggctgccgatttcgtggaaagcaaggacgtgtgcaagaactacgccgaggc caaggatgtgtttctgggcatgtttctgtatgagtacgcccgcagacaccccgactattctgtggttctgctgctgcgg ctggccaaaacctacgagacaaccctggaaaaatgctgcgccgctgccgatcctcacgagtgttatgccaaggt gttcgacgagttcaagcctctggtggaagaaccccagaacctgatcaagcagaactgcgagctgttcgagcagc tgggcgagtacaagttccagaatgccctgctcgtgcggtacaccaagaaagtgcctcaggtgtccacacctacac tggttgaggtgtcccggaatctgggcaaagtgggcagcaagtgttgcaagcaccctgaggccaagagaatgcct tgcgccgaggattacctgagcgtggtgctgaatcagctgtgcgtgctgcacgagaaaacccctgtgtccgacaga gtgaccaagtgctgtaccgagagcctcgtgaacagaaggccttgctttagcgccctggaagtggacgagacata cgtgcccaaagagttcaacgccgagacattcaccttccacgccgacatctgcaccctgtccgagaaagagcgg cagatcaagaagcagacagccctggtcgagctggttaagcacaagcccaaggccaccaaagaacagctgaa ggccgtgatggacgacttcgccgcctttgtcgagaagtgctgcaaggccgacgacaaagagacatgcttcgccg aagagggcaagaaactggtggctgcctctcaggctgctctcggacttggtggaagcggaggaagtggtggatct ggcggatcttgtgtggaacccctcggcatggaaaacggcaatatcgccaatagccagattgccgccagcagcgt cagagtgacatttctgggactgcaacactgggtgcccgagctggctagactgaatagagccggcatggtcaacg cctggacacccagcagcaacgacgataatccctggattcaagtgaacctgctgcggcgtatgtgggtcacaggt gttgttacacagggcgcaagcagactggccagccacgagtatctgaaggcctttaaggtggcctacagcctgaa cggccacgagttcgacttcatccacgacgtgaacaagaagcacaaagagtttgtcggcaactggaacaagaac gccgtgcacgtgaacctgttcgagacacctgtggaagcccagtacgtgcggctgtaccctacaagctgtcacacc gcctgcactctgagattcgaactgctgggatgcgagctgaacggc |
| 119 =74 | EGF-HSA-C1 | LDICSKNPCHNGGLCEEISQEVRGDVFPSYTCTCLKGYAGNHCETKDAHKSEVA HRFKDLGEENFKALVLIAFAQYLQQSPFEDHVKLVNEVTEFAKTCVADESAENCD KSLHTLFGDKLCTVATLRETYGEMADCCAKQEPERNECFLQHKDDNPNLPRLVR PEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKA EFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVAASQAALCVEPLGMENGNIANSQIAASSVRVTFL GLQHWVPELARLNRAGMVNAWTPSSNDDNPWIQVNLLRRMWVTGVVTQGASR LASHEYLKAFKVAYSLNGHEFDFIHDVNKKHKEFVGNWNKNAVHVNLFETPVEA QYVRLYPTSCHTACTLRFELLGCELNG |
| 120 | Nucleic acid of 119 | ctggacatctgtagcaagaacccttgccacaacggcggcctgtgcgaagagatttctcaagaagtgcggggcga cgttttccccagctacacctgtacatgtctgaagggctacgccggcaaccactgcgagacaaaggatgcccaca agagcgaggtggcccacagattcaaggatctgggcgaagagaacttcaaggccctggtgctgatcgccttcgct cagtatctccagcagagccctttcgaggaccacgtgaagctggtcaacgaagtgaccgagttcgccaagacctg tgtggccgatgagagcgccgagaactgtgataagagcctgcacaccctgttcggcgacaagctgtgtacagtgg ccacactgagagaaacctacggcgagatggccgactgctgtgccaagcaagagcccgagagaaacgagtgc ttcctccagcacaaggacgacaaccccaacctgcctagactcgtgcgacccgaagtggatgtgatgtgcaccgc ctttcacgacaacgaggaaaccttcctgaagaagtacctgtacgagatcgccagacggcacccctacttttatgcc cctgagctgctgttcttcgccaagcggtataaggccgccttcaccgaatgttgccaggccgctgataaggctgcctg tctgctgcctaagctggacgagctgagagatgagggcaaagccagctctgccaagcagagactgaaatgcgcc agcctccagaagttcggcgagagagcttttaaggcctgggccgttgccagactgagccagagatttcctaaggcc gagtttgccgaggtgtccaagctcgtgaccgatctgacaaaggtgcacaccgagtgctgtcacggcgatctgctg gaatgtgccgacgatagagccgacctggccaagtacatctgcgagaaccaggacagcatcagcagcaagctg aaagagtgctgcgagaagcccctgctggaaaagtctcactgtatcgccgaggtggaaaacgacgagatgcctg ccgatctgcctagcctggctgccgatttcgtggaaagcaaggacgtgtgcaagaactacgccgaggccaaggat gtgtttctgggcatgtttctgtatgagtacgcccgcagacaccccgactattctgtggttctgctgctgcggctggcca aaacctacgagacaaccctggaaaaatgctgcgccgctgccgatcctcacgagtgttatgccaaggtgttcgac gagttcaagcctctggtggaagaaccccagaacctgatcaagcagaactgcgagctgttcgagcagctgggcg agtacaagttccagaatgccctgctcgtgcggtacaccaagaaagtgcctcaggtgtccacacctacactggttg aggtgtcccggaatctgggcaaagtgggcagcaagtgttgcaagcaccctgaggccaagagaatgccttgcgc cgaggattacctgagcgtggtgctgaatcagctgtgcgtgctgcacgagaaaacccctgtgtccgacagagtgac caagtgctgtaccgagagcctcgtgaacagaaggccttgctttagcgccctggaagtggacgagacatacgtgc ccaaagagttcaacgccgagacattcaccttccacgccgacatctgcaccctgtccgagaaagagcggcagat caagaagcagacagccctggtcgagctggttaagcacaagcccaaggccaccaaagaacagctgaaggcc gtgatggacgacttcgccgcctttgtcgagaagtgctgcaaggccgacgacaaagagacatgcttcgccgaaga gggcaagaaactggtggctgcttctcaggccgctctgtgtgtggaacccctcggcatggaaaacggcaatatcgc caatagccagattgccgccagcagcgtcagagtgacatttctgggactgcaacactgggtgcccgagctggcta gactgaatagagccggcatggtcaacgcctggacacccagcagcaacgacgataatccctggattcaagtgaa cctgctgcggcgtatgtgggtcacaggtgttgttacacagggcgcaagcagactggccagccacgagtatctgaa ggcctttaaggtggcctacagcctgaacggccacgagttcgacttcatccacgacgtgaacaagaagcacaaa gagtttgtcggcaactggaacaagaacgccgtgcacgtgaacctgttcgagacacctgtggaagcccagtacgt gcggctgtaccctacaagctgtcacaccgcctgcactctgagattcgaactgctgggatgcgagctgaacggc |
| 121 =73 | FP135 | LDICSKNPCHNGGLCEEISQEVRGDVFPSYTCTCLKGYAGNHCETKDAHKSEVA HRFKDLGEENFKALVLIAFAQYLQQSPFEDHVKLVNEVTEFAKTCVADESAENCD KSLHTLFGDKLCTVATLRETYGEMADCCAKQEPERNECFLQHKDDNPNLPRLVR PEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKA EFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVACVEPLGMENGNIANSQIAASSVRVTFLGLQHW VPELARLNRAGMVNAWTPSSNDDNPWIQVNLLRRMWVTGVVTQGASRLASHE YLKAFKVAYSLNGHEFDFIHDVNKKHKEFVGNWNKNAVHVNLFETPVEAQYVRL YPTSCHTACTLRFELLGCELNG |
| 122 | Nucleic acid of 121 | ctggacatctgtagcaagaacccttgccacaacggcggcctgtgcgaagagatttctcaagaagtgcggggcga cgttttccccagctacacctgtacatgtctgaagggctacgccggcaaccactgcgagacaaaggatgcccaca agagcgaggtggcccacagattcaaggatctgggcgaagagaacttcaaggccctggtgctgatcgccttcgct cagtatctccagcagagccctttcgaggaccacgtgaagctggtcaacgaagtgaccgagttcgccaagacctg tgtggccgatgagagcgccgagaactgtgataagagcctgcacaccctgttcggcgacaagctgtgtacagtgg ccacactgagagaaacctacggcgagatggccgactgctgtgccaagcaagagcccgagagaaacgagtgc ttcctccagcacaaggacgacaaccccaacctgcctagactcgtgcgacccgaagtggatgtgatgtgcaccgc ctttcacgacaacgaggaaaccttcctgaagaagtacctgtacgagatcgccagacggcacccctacttttatgcc cctgagctgctgttcttcgccaagcggtataaggccgccttcaccgaatgttgccaggccgctgataaggctgcctg tctgctgcctaagctggacgagctgagagatgagggcaaagccagctctgccaagcagagactgaaatgcgcc agcctccagaagttcggcgagagagcttttaaggcctgggccgttgccagactgagccagagatttcctaaggcc gagtttgccgaggtgtccaagctcgtgaccgatctgacaaaggtgcacaccgagtgctgtcacggcgatctgctg gaatgtgccgacgatagagccgacctggccaagtacatctgcgagaaccaggacagcatcagcagcaagctg aaagagtgctgcgagaagcccctgctggaaaagtctcactgtatcgccgaggtggaaaacgacgagatgcctg ccgatctgcctagcctggctgccgatttcgtggaaagcaaggacgtgtgcaagaactacgccgaggccaaggat gtgtttctgggcatgtttctgtatgagtacgcccgcagacaccccgactattctgtggttctgctgctgcggctggcca aaacctacgagacaaccctggaaaaatgctgcgccgctgccgatcctcacgagtgttatgccaaggtgttcgac gagttcaagcctctggtggaagaaccccagaacctgatcaagcagaactgcgagctgttcgagcagctgggcg agtacaagttccagaatgccctgctcgtgcggtacaccaagaaagtgcctcaggtgtccacacctacactggttg aggtgtcccggaatctgggcaaagtgggcagcaagtgttgcaagcaccctgaggccaagagaatgccttgcgc cgaggattacctgagcgtggtgctgaatcagctgtgcgtgctgcacgagaaaacccctgtgtccgacagagtgac caagtgctgtaccgagagcctcgtgaacagaaggccttgctttagcgccctggaagtggacgagacatacgtgc ccaaagagttcaacgccgagacattcaccttccacgccgacatctgcaccctgtccgagaaagagcggcagat caagaagcagacagccctggtcgagctggttaagcacaagcccaaggccaccaaagaacagctgaaggcc gtgatggacgacttcgccgcctttgtcgagaagtgctgcaaggccgacgacaaagagacatgcttcgccgaaga gggcaagaaactggtggcctgtgtggaacccctcggcatggaaaacggcaatatcgccaatagccagattgcc gccagcagcgtcagagtgacatttctgggactgcaacactgggtgcccgagctggctagactgaatagagccgg catggtcaacgcctggacacccagcagcaacgacgataatccctggattcaagtgaacctgctgcggcgtatgt gggtcacaggtgttgttacacagggcgcaagcagactggccagccacgagtatctgaaggcctttaaggtggcct acagcctgaacggccacgagttcgacttcatccacgacgtgaacaagaagcacaaagagtttgtcggcaactg gaacaagaacgccgtgcacgtgaacctgttcgagacacctgtggaagcccagtacgtgcggctgtaccctaca agctgtcacaccgcctgcactctgagattcgaactgctgggatgcgagctgaacggc |
| 123 | hlgG1_FC_DAP A_Hole | DKTHTCPPCPAPELLGGPSVFLFPPKPKDTLMISRTPEVTCVVVAVSHEDPEVKF NWYVDGVEVHNAKTKPREEQYNSTYRWSVLTVLHQDWLNGKEYKCKVSNKAL AAPIEKTISKAKGQPREPQVCTLPPSRDELTKNQVSLSCAVKGFYPSDIAVEWES NGQPENNYKTTPPVLDSDGSFFLVSKLTVDKSRWQQGNVFSCSVMHEALHNHY TQKSLSLSPGK |
| 124 | Nucleic acid of 123 | gataagacccacacctgtcctccatgtcctgctccagaactgctcggcggaccctccgttttcctgtttccacctaag cctaaggacaccctgatgatcagcagaacccctgaagtgacctgtgtggtggtggccgtgtctcacgaagatccc gaagtgaagttcaattggtacgtggacggcgtggaagtgcacaacgccaagaccaagcctagagaggaacag tacaacagcacctacagagtggtgtccgtgctgaccgtgctgcaccaggattggctgaacggcaaagagtacaa gtgcaaggtgtccaacaaggccctggccgctcctatcgagaaaaccatctctaaggccaagggccagcctcgg gaacctcaagtctgtacactgcctcctagccgggacgagctgaccaaaaatcaggtgtccctgagctgcgccgtg aagggcttttacccttccgatatcgccgtggaatgggagagcaatggccagcctgagaacaactacaagaccac acctcctgtgctggacagcgacggctcattctttctggtgtccaagctgacagtggacaagagcagatggcagca gggcaacgtgttcagctgttctgtgatgcacgaggccctgcacaaccactacacccagaagtctctgtctctgagc cccqqcaaa |
| 125 | EGF_hlgG1_FC _DAPA_Knob_ C1 | LDICSKNPCHNGGLCEEISQEVRGDVFPSYTCTCLKGYAGNHCETKGSDKTHTC PPCPAPELLGGPSVFLFPPKPKDTLMISRTPEVTCWVAVSHEDPEVKFNWYVD GVEVHNAKTKPREEQYNSTYRWSVLTVLHQDWLNGKEYKCKVSNKALAAPIEK TISKAKGQPREPQVYTLPPCREEMTKNQVSLWCLVKGFYPSDIAVEWESNGQP ENNYKTTPPVLDSDGSFFLYSKLTVDKSRWQQGNVFSCSVMHEALHNHYTQKS LSLSPGKGGSGGSGGSGGSCVEPLGMENGNIANSQIAASSVRVTFLGLQHWVP ELARLNRAGMVNAWTPSSNDDNPWIQVNLLRRMWVTGVVTQGASRLASHEYL KAFKVAYSLNGHEFDFIHDVNKKHKEFVGNWNKNAVHVNLFETPVEAQYVRLYP TSCHTACTLRFELLGCELNGCANPLGLKNNSIPDKQITASSSYKTWGLHLFSWNP SYARLDKQGNFNAWVAGSYGNDQWLQVDLGSSKEVTGIITQGARNFGSVQFVA SYKVAYSNDSANWTEYQDPRTGSSKIFPGNWDNHSHKKNLFETPILARYVRILPV AWHNRIALRLELLGC |
| 126 | Nucleic acid of 125 | ctggacatctgtagcaagaacccttgccacaacggcggcctgtgcgaagagatttctcaagaagtgcggggcga cgttttccccagctacacctgtacatgtctgaagggctacgccggcaaccactgcgagacaaagggcagcgata agacccacacctgtcctccatgtcctgctccagaactgctcggcggaccctccgttttcctgtttccacctaagccta aggacaccctgatgatcagcagaacccctgaagtgacctgtgtggtggtggccgtgtctcacgaagatcccgaa gtgaagttcaattggtacgtggacggcgtggaagtgcacaacgccaagaccaagcctagagaggaacagtac aacagcacctacagagtggtgtccgtgctgaccgtgctgcaccaggattggctgaacggcaaagagtacaagtg caaggtgtccaacaaggccctggccgctcctatcgagaaaaccatctctaaggccaagggccagcctcgggaa cctcaggtttacaccctgcctccatgccgggaagagatgaccaagaatcaggtgtccctgtggtgcctggtcaag ggcttctacccttccgatatcgccgtggaatgggagagcaatggccagcctgagaacaactacaagaccacacc tcctgtgctggacagcgacggctcattcttcctgtacagcaagctgacagtggacaagagcagatggcagcagg gcaacgtgttcagctgttctgtgatgcacgaggccctgcacaaccactacacccagaagtctctgtctctgagccct ggcaaaggcggaagcggtggaagcggaggatctggcggatcttgtgtggaacccctcggcatggaaaacggc aatatcgccaatagccagatcgccgccagcagcgtcagagtgacatttctgggactgcaacactgggtgccaga gctggccagactgaatagagccggcatggttaacgcctggacacccagcagcaacgacgacaacccctggatt caagtgaacctgctgcggcgtatgtgggtcacaggtgttgttacacagggcgcaagcagactggccagccacga gtatctgaaggcctttaaggtggcctacagcctgaacggccacgagttcgacttcatccacgacgtgaacaagaa gcacaaagagttcgtcggcaactggaacaagaacgccgtgcacgtgaacctgttcgagacacctgtggaagcc cagtacgtgcggctgtaccctacaagctgtcacaccgcctgcacactgagattcgagctgctgggctgcgagctg aatggctgtgctaatcctctgggcctgaagaacaatagcatccccgacaagcagatcaccgcctccagcagctat aagacatggggcctgcacctgtttagctggaaccctagctacgccagactggacaagcagggaaacttcaatgc ctgggtggccggcagctacggcaatgatcaatggctgcaagtggacctgggcagcagcaaagaagtgaccgg catcattacccagggcgctagaaatttcggcagcgtgcagttcgtggccagctacaaagtggcctactccaacga cagcgccaactggaccgagtatcaggaccctagaaccggcagctccaagatcttccccggcaattgggacaac cacagccacaagaagaatctgttcgaaacccctatcctggccagatatgtgcgcattctgcccgtggcctggcac aacagaattgccctgagactggaactgctgggatgc |
| 127 | hlgG1_FC_DAP A_Knob | DKTHTCPPCPAPELLGGPSVFLFPPKPKDTLMISRTPEVTCVVVAVSHEDPEVKF NWYVDGVEVHNAKTKPREEQYNSTYRWSVLTVLHQDWLNGKEYKCKVSNKAL AAPIEKTISKAKGQPREPQVYTLPPCREEMTKNQVSLWCLVKGFYPSDIAVEWE SNGQPENNYKTTPPVLDSDGSFFLYSKLTVDKSRWQQGNVFSCSVMHEALHNH YTQKSLSLSPGK |
| 128 | Nucleic acid of 127 | gataagacccacacctgtcctccatgtcctgctccagaactgctcggcggaccctccgttttcctgtttccacctaag cctaaggacaccctgatgatcagcagaacccctgaagtgacctgtgtggtggtggccgtgtctcacgaagatccc gaagtgaagttcaattggtacgtggacggcgtggaagtgcacaacgccaagaccaagcctagagaggaacag tacaacagcacctacagagtggtgtccgtgctgaccgtgctgcaccaggattggctgaacggcaaagagtacaa gtgcaaggtgtccaacaaggccctggccgctcctatcgagaaaaccatctctaaggccaagggccagcctcgg gaacctcaggtttacaccctgcctccatgccgggaagagatgaccaagaatcaggtgtccctgtggtgcctggtc aagggcttctacccttccgatatcgccgtggaatgggagagcaatggccagcctgagaacaactacaagacca cacctccttgctggacagcgacggctcaftcttcctgtacagcaagctgacagtggacaagagcagatggcag cagggcaacgtgttcagctgttctgtgatgcacgaggccctgcacaaccactacacccagaagtctctgtctctga gccccggcaaa |
| 129 | EGF_hlgG1_FC _DAPA_Hole_C 1 | LDICSKNPCHNGGLCEEISQEVRGDVFPSYTCTCLKGYAGNHCETKGSDKTHTC PPCPAPELLGGPSVFLFPPKPKDTLMISRTPEVTCWVAVSHEDPEVKFNWYVD GVEVHNAKTKPREEQYNSTYRWSVL TVLHQDWLNGKEYKCKVSNKALAAPI EK TISKAKGQPREPQVCTLPPSRDELTKNQVSLSCAVKGFYPSDIAVEWESNGQPE NNYKTTPPVLDSDGSFFLVSKLTVDKSRWQQGNVFSCSVMHEALHNHYTQKSL SLSPGKGGSGGSGGSGGSCVEPLGMENGNIANSQIAASSVRVTFLGLQHWVPE LARLNRAGMVNAWTPSSNDDNPWIQVNLLRRMWVTGVVTQGASRLASHEYLK AFKVAYSLNGHEFDFIHDVNKKHKEFVGNWNKNAVHVNLFETPVEAQYVRLYPT SCHTACTLRFELLGCELNGCANPLGLKNNSIPDKQITASSSYKTWGLHLFSWNPS YARLDKQGNFNAWVAGSYGNDQWLQVDLGSSKEVTGIITQGARNFGSVQFVAS YKVAYSNDSANWTEYQDPRTGSSKIFPGNWDNHSHKKNLFETPILARYVRILPVA WHNRIALRLELLGC |
| 130 | Nucleic acid of 129 | ctggacatctgtagcaagaacccttgccacaacggcggcctgtgcgaagagatttctcaagaagtgcggggcga cgttttccccagctacacctgtacatgtctgaagggctacgccggcaaccactgcgagacaaagggcagcgata agacccacacctgtcctccatgtcctgctccagaactgctcggcggaccctccgttttcctgtttccacctaagccta aggacaccctgatgatcagcagaacccctgaagtgacctgtgtggtggtggccgtgtctcacgaagatcccgaa gtgaagttcaattggtacgtggacggcgtggaagtgcacaacgccaagaccaagcctagagaggaacagtac aacagcacctacagagtggtgtccgtgctgaccgtgctgcaccaggattggctgaacggcaaagagtacaagtg caaggtgtccaacaaggccctggccgctcctatcgagaaaaccatctctaaggccaagggccagcctcgggaa cctcaagtctgtacactgcctcctagccgggacgagctgaccaaaaatcaggtgtccctgagctgcgccgtgaag ggcttttacccttccgatatcgccgtggaatgggagagcaatggccagcctgagaacaactacaagaccacacc tcctgtgctggacagcgacggctcattctttctggtgtccaagctgacagtggacaagagcagatggcagcaggg caacgtgttcagctgttctgtgatgcacgaggccctgcacaaccactacacccagaagtctctgtctctgagccctg gcaaaggcggaagcggtggaagcggaggatctggcggatcttgtgtggaacccctcggcatggaaaacggca atatcgccaatagccagatcgccgccagcagcgtcagagtgacatttctgggactgcaacactgggtgccagag ctggccagactgaatagagccggcatggttaacgcctggacacccagcagcaacgacgacaacccctggattc aagtgaacctgctgcggcgtatgtgggtcacaggtgttgttacacagggcgcaagcagactggccagccacgag tatctgaaggcctttaaggtggcctacagcctgaacggccacgagttcgacttcatccacgacgtgaacaagaag cacaaagagttcgtcggcaactggaacaagaacgccgtgcacgtgaacctgttcgagacacctgtggaagccc agtacgtgcggctgtaccctacaagctgtcacaccgcctgcacactgagattcgagctgctgggctgcgagctga atggctgtgctaatcctctgggcctgaagaacaatagcatccccgacaagcagatcaccgcctccagcagctata agacatggggcctgcacctgtttagctggaaccctagctacgccagactggacaagcagggaaacttcaatgcc tgggtggccggcagctacggcaatgatcaatggctgcaagtggacctgggcagcagcaaagaagtgaccggc atcattacccagggcgctagaaatttcggcagcgtgcagttcgtggccagctacaaagtggcctactccaacgac agcgccaactggaccgagtatcaggaccctagaaccggcagctccaagatcttccccggcaattgggacaacc acagccacaagaagaatctgttcgaaacccctatcctggccagatatgtgcgcattctgcccgtggcctggcaca acagaattgccctgagactggaactgctgggatgc |
| 131 | EGF(RGE)_HS A[A626-L633]removed_ C1 | LDICSKNPCHNGGLCEEISQEVRGEVFPSYTCTCLKGYAGNHCETKDAHKSEVA HRFKDLGEENFKALVLIAFAQYLQQSPFEDHVKLVNEVTEFAKTCVADESAENCD KSLHTLFGDKLCTVATLRETYGEMADCCAKQEPERNECFLQHKDDNPNLPRLVR PEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKA EFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVACVEPLGMENGNIANSQIAASSVRVTFLGLQHW VPELARLNRAGMVNAWTPSSNDDNPWIQVNLLRRMWVTGVVTQGASRLASHE YLKAFKVAYSLNGHEFDFIHDVNKKHKEFVGNWNKNAVHVNLFETPVEAQYVRL YPTSCHTACTLRFELLGCELNG |
| 132 | Nucleic acid of 131 | ctggacatctgtagcaagaacccttgccacaacggcggcctgtgcgaagagatcagtcaagaagtgcggggcg aagtctttcccagctacacctgtacctgtctgaagggctatgccggcaaccactgcgagacaaaggatgcccaca agagcgaggtggcccacagattcaaggatctgggcgaagagaacttcaaggccctggtgctgatcgccttcgct cagtatctccagcagagccctttcgaggaccacgtgaagctggtcaacgaagtgaccgagttcgccaagacctg tgtggccgatgagagcgccgagaactgtgataagagcctgcacaccctgttcggcgacaagctgtgtacagtgg ccacactgagagaaacctacggcgagatggccgactgctgtgccaagcaagagcccgagagaaacgagtgc ttcctccagcacaaggacgacaaccccaacctgcctagactcgtgcgacccgaagtggatgtgatgtgcaccgc ctttcacgacaacgaggaaaccttcctgaagaagtacctgtacgagatcgccagacggcacccctacttttatgcc cctgagctgctgttcttcgccaagcggtataaggccgccttcaccgaatgttgccaggccgctgataaggctgcctg tctgctgcctaagctggacgagctgagagatgagggcaaagccagctctgccaagcagagactgaaatgcgcc agcctccagaagttcggcgagagagcttttaaggcctgggccgttgccagactgagccagagatttcctaaggcc gagtttgccgaggtgtccaagctcgtgaccgatctgacaaaggtgcacaccgagtgctgtcacggcgatctgctg gaatgtgccgacgatagagccgacctggccaagtacatctgcgagaaccaggacagcatcagcagcaagctg aaagagtgctgcgagaagcccctgctggaaaagtctcactgtatcgccgaggtggaaaacgacgagatgcctg ccgatctgcctagcctggctgccgatttcgtggaaagcaaggacgtgtgcaagaactacgccgaggccaaggat gtgtttctgggcatgtttctgtatgagtacgcccgcagacaccccgactattctgtggttctgctgctgcggctggcca aaacctacgagacaaccctggaaaaatgctgcgccgctgccgatcctcacgagtgttatgccaaggtgttcgac gagttcaagcctctggtggaagaaccccagaacctgatcaagcagaactgcgagctgttcgagcagctgggcg agtacaagttccagaatgccctgctcgtgcggtacaccaagaaagtgcctcaggtgtccacacctacactggttg aggtgtcccggaatctgggcaaagtgggcagcaagtgttgcaagcaccctgaggccaagagaatgccttgcgc cgaggattatctgagcgtggtgctgaatcagctgtgcgtgctgcacgagaaaacccctgtgtccgacagagtgac caagtgctgtaccgagagcctcgtgaacagaaggccttgctttagcgccctggaagtggacgagacatacgtgc ccaaagagttcaacgccgagacattcaccttccacgccgacatctgcaccctgtccgagaaagagcggcagat caagaagcagacagccctggtcgagctggttaagcacaagcccaaggccaccaaagaacagctgaaggcc gtgatggacgacttcgccgcctttgtcgagaagtgctgcaaggccgacgacaaagagacatgcttcgccgaaga gggcaagaaactggtggcctgtgtggaacccctcggcatggaaaacggcaatatcgccaatagccagattgcc gccagcagcgtcagagtgacatttctgggactgcaacactgggtgcccgagctggctagactgaatagagccgg catggtcaacgcctggacacccagcagcaacgacgataatccctggattcaagtgaacctgctgcggcgtatgt gggtcacaggtgttgttacacagggcgcaagcagactggccagccacgagtatctgaaggcctttaaggtggcct acagcctgaacggccacgagttcgacttcatccacgacgtgaacaagaagcacaaagagtttgtcggcaactg gaacaagaacgccgtgcacgtgaacctgttcgagacacctgtggaagcccagtacgtgcggctgtaccctaca agctgtcacaccgcctgcactctgagattcgaactgctgggatgcgagctgaacggc |
| 133 =71 | EGF[EDIL3]_HS A[A626-L633]removed_ C1 [EDIL3] | DICDPNPCENGGICLPGLADGSFSCECPDGFTDPNCSVVEVASDEEEPTSAGP CTPNPCHNGGTCEISEAYRGDTFIGYVCKCPRGFNGIHCQHNINECEVEPCKNG GICTDLVANYSCECPGEFMGRNCQYKDAHKSEVAHRFKDLGEENFKALVLIAFA QYLQQSPFEDHVKLVNEVTEFAKTCVADESAENCDKSLHTLFGDKLCTVATLRE TYGEMADCCAKQEPERNECFLQHKDDNPNLPRLVRPEVDVMCTAFHDNEETFL KKYLYEIARRHPYFYAPELLFFAKRYKAAFTECCQAADKAACLLPKLDELRDEGK ASSAKQRLKCASLQKFGERAFKAWAVARLSQRFPKAEFAEVSKLVTDLTKVHTE CCHGDLLECADDRADLAKYICENQDSISSKLKECCEKPLLEKSHCIAEVENDEMP ADLPSLAADFVESKDVCKNYAEAKDVFLGMFLYEYARRHPDYSVVLLLRLAKTYE TTLEKCCAAADPHECYAKVFDEFKPLVEEPQNLIKQNCELFEQLGEYKFQNALLV RYTKKVPQVSTPTLVEVSRNLGKVGSKCCKHPEAKRMPCAEDYLSVVLNQLCVL HEKTPVSDRVTKCCTESLVNRRPCFSALEVDETYVPKEFNAETFTFHADICTLSE KERQIKKQTALVELVKHKPKATKEQLKAVMDDFAAFVEKCCKADDKETCFAEEG KKLVACSGPLGIEGGIISNQQITASSTHRALFGLQKWYPYYARLNKKGLINAWTAA ENDRWPWIQINLQRKMRVTGVITQGAKRIGSPEYIKSYKIAYSNDGKTWAMYKVK GTNEDMVFRGNIDNNTPYANSFTPPIKAQYVRLYPQVCRRHCTLRMELLGCELS G |
| 134 | Nucleic acid of 133 | gacatctgcgaccccaatccttgcgagaatggcggcatttgtctgcctggactggccgatggcagcttctcttgtga atgccccgatggcttcacagaccccaattgcagctctgtggtggaagtggccagcgacgaggaagaacctaca agcgctggcccctgcacacccaatccatgtcataatggcggaacctgcgagatcagcgaggcctacagaggcg ataccttcatcggctacgtgtgcaagtgccccagaggcttcaatggcatccactgccagcacaacatcaacgagt gcgaggtggaaccatgcaagaacggcggcatctgtaccgacctggtggccaattactcttgcgagtgccctggc gagttcatgggcagaaactgccagtacaaggacgcccacaagagcgaggtggcccacagattcaaggacctg ggcgaagagaacttcaaggccctggtgctgatcgccttcgctcagtatctccagcagagccctttcgaggaccac gtgaagctggtcaacgaagtgaccgagttcgccaagacctgtgtggccgatgagagcgccgagaactgtgaca agagcctgcacacactgttcggcgacaagctgtgtaccgtggccacactgagagaaacctacggcgagatggc cgactgctgtgccaagcaagagcccgagagaaacgagtgcttcctccagcacaaggatgacaaccccaacct gcctagactcgtgcggcctgaagtggatgtgatgtgcaccgcctttcacgacaacgaggaaaccttcctgaagaa gtacctgtacgagatcgccagacggcacccctacttttatgcccctgagctgctgttcttcgccaagcggtataagg ccgccttcaccgaatgttgccaggccgctgataaggctgcctgtctgctgcctaagctggacgagctgagagatg agggcaaagccagctctgccaagcagagactgaaatgcgccagcctccagaagttcggcgagagagcttttaa ggcctgggccgttgccagactgagccagagatttcctaaggccgagtttgccgaggtgtccaagctcgtgaccga tctgacaaaggtgcacaccgagtgctgtcacggcgatctgctggaatgtgccgacgatagagccgacctggcca agtatatctgcgagaaccaggacagcatcagcagcaagctgaaagagtgctgcgagaagcccctgctggaaa agtctcactgtatcgccgaagtggaaaacgacgagatgcccgccgatctgccttctctggctgccgatttcgtgga aagcaaggatgtgtgcaagaactacgccgaggccaaagatgtgtttctgggcatgtttctgtatgagtacgcccgc agacaccccgactattctgtggttctgctgctgcggctggccaagacatacgagacaaccctggaaaaatgctgc gccgctgccgatcctcacgagtgttatgccaaggtgttcgacgagttcaagccactggtggaagaaccccagaa cctgatcaagcagaactgcgagctgttcgagcagctgggcgagtacaagttccagaatgccctgctcgtgcggta caccaagaaagtgcctcaggtgtccacacctacactggttgaggtgtcccggaatctgggcaaagtgggcagca agtgttgcaagcaccctgaggccaagagaatgccttgcgccgaggattacctgagcgtggtgctgaatcagctgt gcgtgctgcacgagaaaacccctgtgtccgacagagtgaccaagtgctgtaccgagagcctcgtgaacagaag gccttgctttagcgccctggaagtggacgagacatacgtgcccaaagagttcaacgccgagacattcaccttcca cgccgatatctgcaccctgtccgagaaagagcggcagatcaagaagcagacagccctggtcgagctggttaag cacaagcccaaggccaccaaagaacagctgaaggccgtgatggacgacttcgccgcctttgtcgagaagtgct gcaaggccgacgacaaagagacatgcttcgccgaagagggcaagaaactggtggcctgttctggccctctggg catcgaaggcggcatcatcagcaatcagcagatcaccgccagcagcacccacagagcactgtttggcctgcaa aagtggtatccctactacgcccggctgaacaagaagggcctgattaacgcctggacagccgccgagaatgaca gatggccctggattcagatcaacctccagcggaagatgagagtgaccggcgttatcacacagggcgcaaagag aatcggctcccctgagtacatcaagagctacaagatcgcctacagcaacgacggcaagacctgggccatgtac aaagtgaagggcaccaacgaggacatggtgttccggggcaacatcgacaacaacaccccttacgccaacag cttcacccctcctatcaaggcccagtacgtgcggctgtaccctcaagtgtgcagaaggcactgtaccctgagaatg gaactgctgggctgcgaactgtctggc |
| 135 | EGF[EDIL3]_HS A[A626-L633]removed_ C2[EDIL3] | DICDPNPCENGGICLPGLADGSFSCECPDGFTDPNCSSVVEVASDEEEPTSAGP CTPNPCHNGGTCEISEAYRGDTFIGYVCKCPRGFNGIHCQHNINECEVEPCKNG GICTDLVANYSCECPGEFMGRNCQYKDAHKSEVAHRFKDLGEENFKALVLIAFA QYLQQSPFEDHVKLVNEVTEFAKTCVADESAENCDKSLHTLFGDKLCTVATLRE TYGEMADCCAKQEPERNECFLQHKDDNPNLPRLVRPEVDVMCTAFHDNEETFL KKYLYEIARRHPYFYAPELLFFAKRYKAAFTECCQAADKAACLLPKLDELRDEGK ASSAKQRLKCASLQKFGERAFKAWAVARLSQRFPKAEFAEVSKLVTDLTKVHTE CCHGDLLECADDRADLAKYICENQDSISSKLKECCEKPLLEKSHCIAEVENDEMP ADLPSLAADFVESKDVCKNYAEAKDVFLGMFLYEYARRHPDYSVVLLLRLAKTYE TTLEKCCAAADPHECYAKVFDEFKPLVEEPQNLIKQNCELFEQLGEYKFQNALLV RYTKKVPQVSTPTLVEVSRNLGKVGSKCCKHPEAKRMPCAEDYLSVVLNQLCVL HEKTPVSDRVTKCCTESLVNRRPCFSALEVDETYVPKEFNAETFTFHADICTLSE KERQIKKQTALVELVKHKPKATKEQLKAVMDDFAAFVEKCCKADDKETCFAEEG KKLVACSEPLGMKSGHIQDYQITASSIFRTLNMDMFTWEPRKARLDKQGKVNAW TSGHNDQSQWLQVDLLVPTKVTGIITQGAKDFGHVQFVGSYKLAYSNDGEHWT VYQDEKQRKDKVFQGNFDNDTHRKNVIDPPIYARHIRILPWSWYGRITLRSELLG C |
| 136 | Nucleic acid of 135 | gacatctgcgaccccaatccttgcgagaatggcggcatttgtctgcctggactggccgatggcagcttctcttgtga atgccccgatggcttcacagaccccaattgcagctctgtggtggaagtggccagcgacgaggaagaacctaca agcgctggcccctgcacacccaatccatgtcataatggcggaacctgcgagatcagcgaggcctacagaggcg ataccttcatcggctacgtgtgcaagtgccccagaggcttcaatggcatccactgccagcacaacatcaacgagt gcgaggtggaaccatgcaagaacggcggcatctgtaccgacctggtggccaattactcttgcgagtgccctggc gagttcatgggcagaaactgccagtacaaggacgcccacaagagcgaggtggcccacagattcaaggacctg ggcgaagagaacttcaaggccctggtgctgatcgccttcgctcagtatctccagcagagccctttcgaggaccac gtgaagctggtcaacgaagtgaccgagttcgccaagacctgtgtggccgatgagagcgccgagaactgtgaca agagcctgcacacactgttcggcgacaagctgtgtaccgtggccacactgagagaaacctacggcgagatggc cgactgctgtgccaagcaagagcccgagagaaacgagtgcttcctccagcacaaggatgacaaccccaacct gcctagactcgtgcggcctgaagtggatgtgatgtgcaccgcctttcacgacaacgaggaaaccttcctgaagaa gtacctgtacgagatcgccagacggcacccctacttttatgcccctgagctgctgttcttcgccaagcggtataagg ccgccttcaccgaatgttgccaggccgctgataaggctgcctgtctgctgcctaagctggacgagctgagagatg agggcaaagccagctctgccaagcagagactgaaatgcgccagcctccagaagttcggcgagagagcttttaa ggcctgggccgttgccagactgagccagagatttcctaaggccgagtttgccgaggtgtccaagctcgtgaccga tctgacaaaggtgcacaccgagtgctgtcacggcgatctgctggaatgtgccgacgatagagccgacctggcca agtatatctgcgagaaccaggacagcatcagcagcaagctgaaagagtgctgcgagaagcccctgctggaaa agtctcactgtatcgccgaagtggaaaacgacgagatgcccgccgatctgccttctctggctgccgatttcgtgga aagcaaggatgtgtgcaagaactacgccgaggccaaagatgtgtttctgggcatgtttctgtatgagtacgcccgc agacaccccgactattctgtggttctgctgctgcggctggccaagacatacgagacaaccctggaaaaatgctgc gccgctgccgatcctcacgagtgttatgccaaggtgttcgacgagttcaagccactggtggaagaaccccagaa cctgatcaagcagaactgcgagctgttcgagcagctgggcgagtacaagttccagaatgccctgctcgtgcggta caccaagaaagtgcctcaggtgtccacacctacactggttgaggtgtcccggaatctgggcaaagtgggcagca agtgttgcaagcaccctgaggccaagagaatgccttgcgccgaggattacctgagcgtggtgctgaatcagctgt gcgtgctgcacgagaaaacccctgtgtccgacagagtgaccaagtgctgtaccgagagcctcgtgaacagaag gccttgctttagcgccctggaagtggacgagacatacgtgcccaaagagttcaacgccgagacattcaccttcca cgccgatatctgcaccctgtccgagaaagagcggcagatcaagaagcagacagccctggtcgagctggttaag cacaagcccaaggccaccaaagaacagctgaaggccgtgatggacgacttcgccgcctttgtcgagaagtgct gcaaggccgacgacaaagagacatgcttcgccgaagagggcaagaaactggtggcctgttctgagccactgg gcatgaagtctggccacatccaggattaccagatcaccgccagcagcatcttcagaaccctgaacatggatatgt tcacctgggagccccggaaggccagactggataagcagggaaaagtgaacgcctggaccagcggccacaat gaccagtctcagtggctgcaagtggacctgctggtgcctaccaaagtgaccggcatcatcacacagggcgcaa aggatttcggccacgtgcagtttgtgggcagctacaagctggcctacagcaacgatggcgagcactggacagtgt accaggacgagaagcagcggaaggataaggtgttccagggcaacttcgacaacgacacccaccggaagaa cgtgatcgaccctcctatctacgcccggcacatcagaatcctgccttggtcttggtacggccggatcaccctgaga agcgagctgcttggatgt |
| 137 | EGF_HSA[A626 L633]removed_ C2 | LDICSKNPCHNGGLCEEISQEVRGDVFPSYTCTCLKGYAGNHCETKDAHKSEVA HRFKDLGEENFKALVLIAFAQYLQQSPFEDHVKLVNEVTEFAKTCVADESAENCD KSLHTLFGDKLCTVATLRETYGEMADCCAKQEPERNECFLQHKDDNPNLPRLVR PEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKA EFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVACANPLGLKNNSIPDKQITASSSYKTWGLHLFS WNPSY ARLDKQGN FNA VW AGSYGNDQWLQVDLGSSKEVTG IITQGARNFGSV QFVASYKVAYSNDSANWTEYQDPRTGSSKIFPGNWDNHSHKKNLFETPILARYV RILPVAWHNRIALRLELLGC |
| 138 | Nucleic acid of 137 | ctggacatctgtagcaagaacccttgccacaacggcggcctgtgcgaagagatttctcaagaagtgcggggcga cgttttccccagctacacctgtacatgtctgaagggctacgccggcaaccactgcgagacaaaggatgcccaca agagcgaggtggcccacagattcaaggatctgggcgaagagaacttcaaggccctggtgctgatcgccttcgct cagtatctccagcagagccctttcgaggaccacgtgaagctggtcaacgaagtgaccgagttcgccaagacctg tgtggccgatgagagcgccgagaactgtgataagagcctgcacaccctgttcggcgacaagctgtgtacagtgg ccacactgagagaaacctacggcgagatggccgactgctgtgccaagcaagagcccgagagaaacgagtgc ttcctccagcacaaggacgacaaccccaacctgcctagactcgtgcgacccgaagtggatgtgatgtgcaccgc ctttcacgacaacgaggaaaccttcctgaagaagtacctgtacgagatcgccagacggcacccctacttttatgcc cctgagctgctgttcttcgccaagcggtataaggccgccttcaccgaatgttgccaggccgctgataaggctgcctg tctgctgcctaagctggacgagctgagagatgagggcaaagccagctctgccaagcagagactgaaatgcgcc agcctccagaagttcggcgagagagcttttaaggcctgggccgttgccagactgagccagagatttcctaaggcc gagtttgccgaggtgtccaagctcgtgaccgatctgacaaaggtgcacaccgagtgctgtcacggcgatctgctg gaatgtgccgacgatagagccgacctggccaagtacatctgcgagaaccaggacagcatcagcagcaagctg aaagagtgctgcgagaagcccctgctggaaaagtctcactgtatcgccgaggtggaaaacgacgagatgcctg ccgatctgcctagcctggctgccgatttcgtggaaagcaaggacgtgtgcaagaactacgccgaggccaaggat gtgtttctgggcatgtttctgtatgagtacgcccgcagacaccccgactattctgtggttctgctgctgcggctggcca aaacctacgagacaaccctggaaaaatgctgcgccgctgccgatcctcacgagtgttatgccaaggtgttcgac gagttcaagcctctggtggaagaaccccagaacctgatcaagcagaactgcgagctgttcgagcagctgggcg agtacaagttccagaatgccctgctcgtgcggtacaccaagaaagtgcctcaggtgtccacacctacactggttg aggtgtcccggaatctgggcaaagtgggcagcaagtgttgcaagcaccctgaggccaagagaatgccttgcgc cgaggattacctgagcgtggtgctgaatcagctgtgcgtgctgcacgagaaaacccctgtgtccgacagagtgac caagtgctgtaccgagagcctcgtgaacagaaggccttgctttagcgccctggaagtggacgagacatacgtgc ccaaagagttcaacgccgagacattcaccttccacgccgacatctgcaccctgtccgagaaagagcggcagat caagaagcagacagccctggtcgagctggttaagcacaagcccaaggccaccaaagaacagctgaaggcc gtgatggacgacttcgccgcctttgtcgagaagtgctgcaaggccgacgacaaagagacatgcttcgccgaaga gggcaagaaactggtggcctgtgctaaccctctgggcctgaagaacaacagcatccccgataagcagatcacc gccagcagcagctataagacatggggcctgcacctgttcagctggaacccttcttacgccagactggacaagca gggcaacttcaatgcttgggtggccggcagctacggcaatgatcagtggctgcaagtggacctgggcagcagca aagaagtgacaggcatcatcacccagggcgcaagaaatttcggcagcgtgcagttcgtggccagctacaaggt ggcctacagcaacgatagcgccaactggaccgagtatcaggaccctagaaccggcagctccaagatcttcccc ggcaactgggacaaccacagccacaagaagaatctgttcgagacacccatcctggccagatacgtgcggattc tgcctgtggcctggcacaacagaatcgccctgagactggaactgctgggctgt |
| 139 | EGF_HSA[A626 -L633] | LDICSKNPCHNGGLCEEISQEVRGDVFPSYTCTCLKGYAGNHCETKDAHKSEVA HRFKDLGEENFKALVLIAFAQYLQQSPFEDHVKLVNEVTEFAKTCVADESAENCD KSLHTLFGDKLCTVATLRETYGEMADCCAKQEPERNECFLQHKDDNPNLPRLVR PEVDVMCTAFHDNEETFLKKYLYEIARRHPYFYAPELLFFAKRYKAAFTECCQAA DKAACLLPKLDELRDEGKASSAKQRLKCASLQKFGERAFKAWAVARLSQRFPKA EFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYAR RHPDYSVVLLLRLAKTYETTLEKCCAAADPHECYAKVFDEFKPLVEEPQNLIKQN CELFEQLGEYKFQNALLVRYTKKVPQVSTPTLVEVSRNLGKVGSKCCKHPEAKR MPCAEDYLSVVLNQLCVLHEKTPVSDRVTKCCTESLVNRRPCFSALEVDETYVP KEFNAETFTFHADICTLSEKERQIKKQTALVELVKHKPKATKEQLKAVMDDFAAFV EKCCKADDKETCFAEEGKKLVA |
| 140 | Nucleic acid of 139 | ctggacatctgtagcaagaacccttgccacaacggcggcctgtgcgaagagatttctcaagaagtgcggggcga cgttttccccagctacacctgtacatgtctgaagggctacgccggcaaccactgcgagacaaaggatgcccaca agagcgaggtggcccacagattcaaggatctgggcgaagagaacttcaaggccctggtgctgatcgccttcgct cagtatctccagcagagccctttcgaggaccacgtgaagctggtcaacgaagtgaccgagttcgccaagacctg tgtggccgatgagagcgccgagaactgtgataagagcctgcacaccctgttcggcgacaagctgtgtacagtgg ccacactgagagaaacctacggcgagatggccgactgctgtgccaagcaagagcccgagagaaacgagtgc ttcctccagcacaaggacgacaaccccaacctgcctagactcgtgcgacccgaagtggatgtgatgtgcaccgc ctttcacgacaacgaggaaaccttcctgaagaagtacctgtacgagatcgccagacggcacccctacttttatgcc cctgagctgctgttcttcgccaagcggtataaggccgccttcaccgaatgttgccaggccgctgataaggctgcctg tctgctgcctaagctggacgagctgagagatgagggcaaagccagctctgccaagcagagactgaaatgcgcc agcctccagaagttcggcgagagagcttttaaggcctgggccgttgccagactgagccagagatttcctaaggcc gagtttgccgaggtgtccaagctcgtgaccgatctgacaaaggtgcacaccgagtgctgtcacggcgatctgctg gaatgtgccgacgatagagccgacctggccaagtacatctgcgagaaccaggacagcatcagcagcaagctg aaagagtgctgcgagaagcccctgctggaaaagtctcactgtatcgccgaggtggaaaacgacgagatgcctg ccgatctgcctagcctggctgccgatttcgtggaaagcaaggacgtgtgcaagaactacgccgaggccaaggat gtgtttctgggcatgtttctgtatgagtacgcccgcagacaccccgactattctgtggttctgctgctgcggctggcca aaacctacgagacaaccctggaaaaatgctgcgccgctgccgatcctcacgagtgttatgccaaggtgttcgac gagttcaagcctctggtggaagaaccccagaacctgatcaagcagaactgcgagctgttcgagcagctgggcg agtacaagttccagaatgccctgctcgtgcggtacaccaagaaagtgcctcaggtgtccacacctacactggttg aggtgtcccggaatctgggcaaagtgggcagcaagtgttgcaagcaccctgaggccaagagaatgccttgcgc cgaggattacctgagcgtggtgctgaatcagctgtgcgtgctgcacgagaaaacccctgtgtccgacagagtgac caagtgctgtaccgagagcctcgtgaacagaaggccttgctttagcgccctggaagtggacgagacatacgtgc ccaaagagttcaacgccgagacattcaccttccacgccgacatctgcaccctgtccgagaaagagcggcagat caagaagcagacagccctggtcgagctggttaagcacaagcccaaggccaccaaagaacagctgaaggcc gtgatggacgacttcgccgcctttgtcgagaagtgctgcaaggccgacgacaaagagacatgcttcgccgaaga gggcaagaaactggtggct |
| 141 | PS binding MFG-E8 C1 | CVEPLGLENGNIANSQIAASSVRVTFLGLQHWVPELARLNRAGMVNAWTPSSND DNPWIQVNLLRRMWVTGVVTQGASRLASHEYLKAFKVAYSLNGHEFDFIHDVNK KHKEFVGNWNKNAVHVNLFETPVEAQYVRLYPTSCHTACTLRFELLGCELNG |
| 142 | PS binding MFG-E8 [L76M] C1 | CVEPLGMENGNIANSQIAASSVRVTFLGLQHWVPELARLNRAGMVNAWTPSSN DDNPWIQVNLLRRMWVTGVVTQGASRLASHEYLKAFKVAYSLNGHEFDFIHDVN KKHKEFVGNWNKNAVHVNLFETPVEAQYVRLYPTSCHTACTLRFELLGCELNG |
| 143 | PS binding MFG-E8 C2 | CANPLGLKNNSIPDKQITASSSYKTWGLHLFSWNPSYARLDKQGNFNAWVAGS YGNDQWLQVDLGSSKEVTGIITQGARNFGSVQFVASYKVAYSNDSANWTEYQD PRTGSSKIFPGNWDNHSHKKNLFETPILARYVRILPVAWHNRIALRLELLGC |
| 144 | PS binding EDIL-3 C1 | CSGPLGIEGGIISNQQITASSTHRALFGLQKWYPYYARLNKKGLINAWTAAENDR WPWIQINLQRKMRVTGVITQGAKRIGSPEYIKSYKIAYSNDGKTWAMYKVKGTNE DMVFRGNIDNNTPYANSFTPPIKAQYVRLYPQVCRRHCTLRMELLGCELSG |
| 145 | PS binding EDIL-3 C2 | CSEPLGMKSGHIQDYQITASSIFRTLNMDMFTWEPRKARLDKQGKVNAWTSGH NDQSQWLQVDLLVPTKVTGIITQGAKDFGHVQFVGSYKLAYSNDGEHWTVYQD EKQRKDKVFQGNFDNDTHRKNVIDPPIYARHIRILPWSWYGRITLRSELLGCTEE E |
| 146 | PS binding EDIL-3 C2 TEEE truncated | CSEPLGMKSGHIQDYQITASSIFRTLNMDMFTWEPRKARLDKQGKVNAWTSGH NDQSQWLQVDLLVPTKVTGIITQGAKDFGHVQFVGSYKLAYSNDGEHWTVYQD EKQRKDKVFQGNFDNDTHRKNVIDPPIYARHIRILPWSWYGRITLRSELLGC |
| 147 =117 | FP133 protein sequence | LDICSKNPCHNGGLCEEISQEVRGDVFPSYTCTCLKGYAGNHCETKGSDAHKSE VAHRFKDLGEENFKALVLIAFAQYLQQSPFEDHVKLVNEVTEFAKTCVADESAEN CDKSLHTLFGDKLCTVATLRETYGEMADCCAKQEPERNECFLQHKDDNPNLPRL VRPEVDVMCTAFHDNEETFLKKYLYEIARRHPYFYAPELLFFAKRYKAAFTECCQ AADKAACLLPKLDELRDEGKASSAKQRLKCASLQKFGERAFKAWAVARLSQRFP KAEFAEVSKLVTDLTKVHTECCHGDLLECADDRADLAKYICENQDSISSKLKECC EKPLLEKSHCIAEVENDEMPADLPSLAADFVESKDVCKNYAEAKDVFLGMFLYEY ARRHPDYSVVLLLRLAKTYETTLEKCCAAADPHECYAKVFDEFKPLVEEPQNLIK QNCELFEQLGEYKFQNALLVRYTKKVPQVSTPTLVEVSRNLGKVGSKCCKHPEA KRMPCAEDYLSVVLNQLCVLHEKTPVSDRVTKCCTESLVNRRPCFSALEVDETY VPKEFNAETFTFHADICTLSEKERQIKKQTALVELVKHKPKATKEQLKAVMDDFA AFVEKCCKADDKETCFAEEGKKLVAASQAALGLGGSGGSGGSGGSCVEPLGM ENGNIANSQIAASSVRVTFLGLQHWVPELARLNRAGMVNAWTPSSNDDNPWIQ VNLLRRMWVTGVVTQGASRLASHEYLKAFKVAYSLNGHEFDFIHDVNKKHKEFV GNWNKNAVHVNLFETPVEAQYVRLYPTSCHTACTLRFELLGCELNG |
| 148 | FP133 Nucleic acid sequence | ctggacatctgtagcaagaacccttgccacaacggcggcctgtgcgaagagatttctcaagaagtgcggggcga cgttttccccagctacacctgtacatgtctgaagggctacgccggcaaccactgcgagacaaaaggatctgatgc ccacaagagcgaggtggcccacagattcaaggatctgggcgaagagaacttcaaggccctggtgctgatcgcc ttcgctcagtatctccagcagagccctttcgaggaccacgtgaagctggtcaacgaagtgaccgagttcgccaag acctgtgtggccgatgagagcgccgagaactgtgataagagcctgcacaccctgttcggcgacaagctgtgtac agtggccacactgagagaaacctacggcgagatggccgactgctgtgccaagcaagagcccgagagaaacg agtgcttcctccagcacaaggacgacaaccccaacctgcctagactcgtgcgacccgaagtggatgtgatgtgc accgcctttcacgacaacgaggaaaccttcctgaagaagtacctgtacgagatcgccagacggcacccctacttt tatgcccctgagctgctgttcttcgccaagcggtataaggccgccttcaccgaatgttgccaggccgctgataaggc tgcctgtctgctgcctaagctggacgagctgagagatgagggcaaagccagctctgccaagcagagactgaaat gcgccagcctccagaagttcggcgagagagcttttaaggcctgggccgttgccagactgagccagagatttccta aggccgagtttgccgaggtgtccaagctcgtgaccgatctgacaaaggtgcacaccgagtgctgtcacggcgat ctgctggaatgtgccgacgatagagccgacctggccaagtacatctgcgagaaccaggacagcatcagcagc aagctgaaagagtgctgcgagaagcccctgctggaaaagtctcactgtatcgccgaggtggaaaacgacgag atgcctgccgatctgcctagcctggctgccgatttcgtggaaagcaaggacgtgtgcaagaactacgccgaggc caaggatgtgtttctgggcatgtttctgtatgagtacgcccgcagacaccccgactattctgtggttctgctgctgcgg ctggccaaaacctacgagacaaccctggaaaaatgctgcgccgctgccgatcctcacgagtgttatgccaaggt gttcgacgagttcaagcctctggtggaagaaccccagaacctgatcaagcagaactgcgagctgttcgagcagc tgggcgagtacaagttccagaatgccctgctcgtgcggtacaccaagaaagtgcctcaggtgtccacacctacac tggttgaggtgtcccggaatctgggcaaagtgggcagcaagtgttgcaagcaccctgaggccaagagaatgcct tgcgccgaggattacctgagcgtggtgctgaatcagctgtgcgtgctgcacgagaaaacccctgtgtccgacaga gtgaccaagtgctgtaccgagagcctcgtgaacagaaggccttgctttagcgccctggaagtggacgagacata cgtgcccaaagagttcaacgccgagacattcaccttccacgccgacatctgcaccctgtccgagaaagagcgg cagatcaagaagcagacagccctggtcgagctggttaagcacaagcccaaggccaccaaagaacagctgaa ggccgtgatggacgacttcgccgcctttgtcgagaagtgctgcaaggccgacgacaaagagacatgcttcgccg aagagggcaagaaactggtggctgcctctcaggctgctctcggacttggtggaagcggaggaagtggtggatct ggcggatcttgtgtggaacccctcggcatggaaaacggcaatatcgccaatagccagattgccgccagcagcgt cagagtgacatttctgggactgcaacactgggtgcccgagctggctagactgaatagagccggcatggtcaacg cctggacacccagcagcaacgacgataatccctggattcaagtgaacctgctgcggcgtatgtgggtcacaggt gttgttacacagggcgcaagcagactggccagccacgagtatctgaaggcctttaaggtggcctacagcctgaa cggccacgagttcgacttcatccacgacgtgaacaagaagcacaaagagtttgtcggcaactggaacaagaac gccgtgcacgtgaacctgttcgagacacctgtggaagcccagtacgtgcggctgtaccctacaagctgtcacacc gcctgcactctgagattcgaactgctgggatgcgagctgaacggc |

The present application also includes variants of each of SEQ ID NOs: 69, 70 and 72, wherein the EGF-like domain of EDIL3 sequence included therein corresponds to any one of the following sequences: SEQ ID NO: 96, SEQ ID NO: 97, SEQ ID NO: 98, SEQ ID NO: 99, SEQ ID NO: 100, or SEQ ID NO: 101.

The present application also includes therapeutic fusion protein comprising the integrin binding domains of MFGE8 or EDIL3, and a truncated PS binding domains such as a truncated variant of IgSF V domain of TIM4 or a truncated variant of the GLA domain of the bridging protein GAS6 variants.

### MODIFICATION OF THE PROTEINS OF THE PRESENT DISCLOSURE

The present application includes variants of the proteins described herein and/or fragments thereof having various modifications in domains as well as fusions and conjugates of the disclosed molecules. For example, a domain of the therapeutic fusion protein may have conservative modification of amino acid residues, and wherein the modified proteins retain or have enhanced properties as compared to a fusion protein comprising the parent domain. Alternatively, a domain of the therapeutic fusion protein may have a deletion(s) of amino acid residues, wherein the modified fusion proteins retain or have enhanced properties as compared to the protein comprising the parent domain. Alternatively, the therapeutic fusion proteins may have an insertion(s) of amino acid residues, wherein the modified proteins retain or have enhanced properties as compared to the unmodified protein. In one embodiment, such an amino acid insertion includes glycine or serine residues in a number of combinations to function as a linker between domains of the parent protein.

Site-directed mutagenesis or PCR-mediated mutagenesis can be performed to introduce the mutation(s) and the effect on integrin and/or PS binding, or other functional property of interest, can be evaluated in in vitro or in vivo assays. Conservative modifications (as discussed above) can be introduced and/or the mutations may be amino acid substitutions, additions or deletions. Moreover, typically no more than one, two, three, four or five residues within a binding domain are altered.

Amino acid sequence variants of the therapeutic fusion proteins, which have essentially similar properties as unmodified variants, can be prepared by introducing appropriate nucleotide changes into the encoding DNAs, or by synthesis of the desired variants. Such variants include, for example, deletions from, or insertions or substitutions of, residues within the amino acid sequences of present molecules. In some embodiments, variants may include additional linker sequences, reduced linker sequences or removal of linker sequences, and/or amino acid mutations or substitutions and deletion of one or more amino acids. Any combination of deletion, insertion and substitution is made to arrive at the final construct, provided that the final construct possesses the desired characteristics. The amino acid changes also may alter post-translational processes of the molecules, such as changing the number or position of possible glycosylation sites.

### METHODS OF PRODUCING RECOMBINANT MOLECULES

### Nucleic Acids and Expression Systems

In one embodiment, the present application provides a method of producing one or more polypeptide chains of the therapeutic fusion protein recombinantly, comprising: 1) producing one or more DNA constructs comprising a nucleic acid molecule encoding a polypeptide chain of the multi-specific binding molecule; 2) introducing said DNA construct(s) into one or more expression vectors; 3) co-transfecting said expression vector(s) in one or more host cells; and 4) expressing and assembling the molecule in a host cell or in solution.

In this respect, the disclosure provides isolated nucleic acids, e.g., one or more polynucleotides, encoding the therapeutic fusion proteins described herein. Nucleic acid molecules include DNA and RNA in both single-stranded and double-stranded form, as well as the corresponding complementary sequences. The nucleic acid molecules of the invention include full-length genes or cDNA molecules as well as a combination of fragments thereof. The nucleic acids of the invention are derived from human sources but the invention includes those derived from non-human species.

An ‘isolated nucleic acid’ is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, NY (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

The present invention also provides expression systems and constructs in the form of plasmids, expression vectors, transcription or expression cassettes, which comprise at least one polynucleotide as described above. In addition, the invention provides host cells comprising such expression systems or constructs.

In one embodiment, the present disclosure provides a method of preparing a therapeutic fusion protein comprising the steps of: (a) culturing a host cell comprising a nucleic acid encoding the fusion protein, wherein the cultured host cell expresses the fusion protein; and (b) recovering the fusion protein from the host cell culture.

Also provided in the disclosure are expression vectors and host cells for producing the therapeutic fusion proteins described above. The term “vector” means any molecule or entity (e.g. nucleic acid, plasmid, bacteriophage or virus) that is suitable for transformation or transfection of a host cell and contains nucleic acid sequences that direct and/or control (in conjunction with the host cell) expression of one or more heterologous coding regions operatively linked thereto. Various expression vectors can be employed to express the polynucleotides encoding chains or binding domains of the molecule. Both viral-based and non-viral expression vectors can be used to produce the therapeutic fusion protein in a mammalian host cell. Non-viral vectors and systems include plasmids, episomal vectors, typically with an expression cassette for expressing a protein or RNA, and human artificial chromosomes (see, e.g., Harrington et al., (1997) Nat Genet 15: 345). For example, non-viral vectors useful for expression of the polynucleotides and polypeptides in mammalian (e.g., human) cells include pThioHis A, B & C, pcDNA3.1/His, pEBVHis A, B & C, (Invitrogen, San Diego, CA), MPSV vectors, and numerous other vectors known in the art for expressing other proteins. Useful viral vectors include vectors based on retroviruses, adenoviruses, adeno associated viruses, herpes viruses, vectors based on SV40, papilloma virus, HBP Epstein Barr virus, vaccinia virus vectors and Semliki Forest virus (SFV). See, Brent et al., (1995) supra; Smith, Annu. Rev. Microbiol. 49: 807; and Rosenfeld et al., (1992) Cell 68: 143.

The choice of expression vector depends on the intended host cells in which the vector is to be expressed. Typically, the expression vectors contain a promoter and other regulatory sequences (e.g., enhancers) that are operably linked to the polynucleotides encoding a therapeutic fusion protein. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements may also be required or desired for efficient expression of the therapeutic fusion proteins. These elements typically include an ATG initiation codon and adjacent ribosome binding site or other sequences. In addition, the efficiency of expression may be enhanced by the inclusion of enhancers appropriate to the cell system in use (see, e.g., Scharf et al., (1994) Results Probl. Cell Differ. 20: 125; and Bittner et al., (1987) Meth. Enzymol., 153 :516). For example, the SV40 enhancer or CMV enhancer may be used to increase expression in mammalian host cells.

The expression vectors may also provide a secretion signal sequence position to form a fusion protein with polypeptides encoded by inserting the above-described sequences of binding domains and/or solubilizing domains. More often, the inserted sequences are linked to signal sequences before inclusion in the vector. Vectors that allow expression of the binding domains and solubilizing domain as fusion proteins thereby lead to production of intact engineered proteins. A host cell, when cultured under appropriate conditions, can be used to express an engineered protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule. A host cell may be eukaryotic or prokaryotic.

Mammalian cell lines available as hosts for expression are known in the art and include, but are not limited to, immortalized cell lines available from the American Type Culture Collection (ATCC) and any cell lines used in an expression system known in the art can be used to make the recombinant fusion proteins of the invention. In general, host cells are transformed with a recombinant expression vector that comprises DNA encoding a desired fusion protein. Among the host cells that may be employed are prokaryotes, yeast or higher eukaryotic cells. Prokaryotes include gram negative or gram positive organisms, for example E. coli or bacilli. Higher eukaryotic cells include insect cells and established cell lines of mammalian origin. Examples of suitable mammalian host cell lines include the COS-7 cells, L cells, CI27 cells, 3T3 cells, Chinese hamster ovary (CHO) cells, or their derivatives and related cell lines which grow in serum free media, HeLa cells, BHK cell lines, the CV-1 EBNA cell line, human embryonic kidney (HEK) cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepGB, KB, NIH 3T3 or S49, for example, can be used for expression of the polypeptide when it is desirable to use the polypeptide in various signal transduction or reporter assays. Alternatively, it is possible to produce the polypeptide in lower eukaryotes such as yeast or in prokaryotes such as bacteria. Suitable yeasts include P. pastoris, S. cerevisiae, S. pombe, Kluyveromyces strains, Candida, or any yeast strain capable of expressing heterologous polypeptides. Suitable bacterial strains include E. coli, B. subtilis, S. typhimurium, or any bacterial strain capable of expressing heterologous polypeptides. If the fusion protein is made in yeast or bacteria, it may be desirable to modify the product produced therein, for example by phosphorylation or glycosylation of the appropriate sites, in order to obtain a functional product. Such covalent attachments can be accomplished using known chemical or enzymatic methods.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22, agent-enhanced uptake of DNA, and ex vivo transduction. For long-term, high-yield production of recombinant proteins, stable expression will often be desired. For example, cell lines which stably express engineered proteins can be prepared using expression vectors of the disclosure which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

The fusion proteins are typically recovered from the culture medium as a secreted polypeptide, although they may also be recovered from host cell lysate when directly produced without a secretory signal. If the polypeptide is membrane-bound, it can be released from the membrane using a suitable detergent solution (e.g., Triton-X 100).

When the fusion protein is produced in a recombinant cell other than one of human origin, it is completely free of proteins or polypeptides of human origin. However, it is necessary to purify the fusion protein from recombinant cell proteins or polypeptides. As a first step, the culture medium or lysate is normally centrifuged to remove particulate cell debris. The produced molecules can be conveniently purified by hydroxylapatite chromatography, gel electrophoresis, dialysis, or affinity chromatography, with affinity chromatography being the preferred purification technique. Other techniques for protein purification such as fractionation on an ion-exchange column, ethanol precipitation, reverse phase HPLC, chromatography on silica, chromatography on heparin Sepharose, chromatography on an anion or cation exchange resin (such as a polyaspartic acid column), chromatofocusing, SDS-PAGE, and ammonium sulfate precipitation are also available.

In certain aspects, provided herein is a viral vector comprising a polynucleotide encoding a therapeutic fusion protein of the present invention. In some embodiments, the viral vector is derived from AAV. In certain some embodiments, the viral vector is administered to a subject, e.g., a human, wherein the therapeutic fusion protein is expressed, and can be used for the treatment of and/or prevention of the diseases as listed herein.

### Pharmaceutical Compositions

In another aspect, the present disclosure provides a composition, e.g., a pharmaceutical composition, containing a therapeutic fusion protein of the present invention, in combination with one or more pharmaceutically acceptable excipient, diluent or carrier. Such compositions may include one or a combination of (e.g., two or more different) therapeutic fusion proteins of the disclosure.

Pharmaceutical compositions as described herein can also be administered in combination therapy, i.e., combined with other agents. For example, the combination therapy can include a fusion protein of the present disclosure combined with, for example, at least one anti-inflammatory, anti-infective agent or immunosuppressant agent. Examples of therapeutic agents that can be used in combination therapy are described in greater detail below in the section on uses of the therapeutic fusion proteins of the disclosure.

To prepare pharmaceutical or sterile compositions including a fusion protein of the present disclosure, the fusion protein is mixed with a pharmaceutically acceptable carrier or excipient.

The phrase ‘pharmaceutically acceptable’ means approved by a regulatory agency of a federal or a state government, or listed in the U.S. Pharmacopeia or other generally recognized pharmacopeia for use in animals, and more particularly, in humans.

The term ‘pharmaceutical composition’ refers to a mixture of at least one active ingredient (e.g., an engineered protein) and at least one pharmaceutically acceptable excipient, diluent or carrier.

A ‘medicament’ refers to a substance used for medical treatment.

As used herein, ‘pharmaceutically acceptable carrier’ includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). In one embodiment, the carrier should be suitable for subcutaneous route. Depending on the route of administration, the active compound, i.e. fusion protein, may be coated in a material to protect the compound from the action of acids and other natural conditions that may inactivate the compound.

The pharmaceutical compositions as described herein may include one or more pharmaceutically acceptable salts. A pharmaceutical composition as described herein may also include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions as described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition.

Reviews on the development of stable protein formulations may be found in Cleland et al., (1993) Crit Reviews Ther Drug Carrier Systems, 10(4): 307-377 and Wei W (1999) Int J Pharmaceutics, 185: 129-88.

Solutions or suspensions used for intradermal or subcutaneous application typically include one or more of the following components: a sterile diluent such as water for injection, saline solution, fixed oils, polyethylene glycols, glycerin, propylene glycol or other synthetic solvents, antibacterial agents such as benzyl alcohol or methyl parabens, antioxidants such as ascorbic acid or sodium bisulfite, chelating agents such ethylenediaminetetraacetic acid, buffers such as acetates, citrates or phosphates, and agents for the adjustment of tonicity such as sodium chloride or dextrose. The pH can be adjusted with acids or bases, such as hydrochloric acid or sodium hydroxide. Such preparations may be enclosed in ampoules, disposables syringes or multiple dose vials made of glass or plastic.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the fusion proteins of the invention into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 per cent to about ninety-nine percent of active ingredient, from about 0.1 per cent to about 70 per cent, or from about 1 percent to about 30 percent of active ingredient in combination with a pharmaceutically acceptable carrier.

Selecting an administration regimen for a therapeutic engineered protein depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of protein delivered depends in part on the particular entity and the severity of the condition being treated. Guidance in selecting appropriate doses of biologic and small molecules are available (see, e.g., Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

Dosage regimens are adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

For administration of the therapeutic fusion protein, the dosage ranges from about 0.0001 to 150 mg/kg, such as 5, 15, and 50 mg/kg subcutaneous administration, and more usually 0.01 to 5 mg/kg, of the host body weight. An exemplary treatment regime entails administration once per week, once every two weeks, once every three weeks, once every four weeks, once per month, once every 3 months or once every three to 6 months.

Therapeutic fusion proteins of the invention may be administered on multiple occasions. Intervals between single dosages can be, for example, weekly, monthly, every three months or yearly. Intervals can also be irregular as indicated by measuring blood levels of engineered protein in the patient. In some methods, dosage is adjusted to achieve a plasma protein concentration of about 1-1000 µg/ml and in some methods about 25-300 µg/ml.

Alternatively, the therapeutic fusion protein can be administered as a sustained release formulation, in which case less frequent administration is required. Dosage and frequency vary depending on the half-life of the protein in the patient and can vary depending on whether the treatment is prophylactic or therapeutic. In prophylactic applications, a relatively low dosage is administered at relatively infrequent intervals over a long period of time. Some patients may continue to receive treatment for the rest of their lives. In therapeutic applications, a relatively high dosage at relatively short intervals is sometimes required until progression of the condition or disease is reduced or terminated or until the patient shows partial or complete amelioration of symptoms of the condition or disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A ‘therapeutically effective dosage’ of a fusion protein of the invention can result in a decrease in severity of a condition or symptoms or a disease and/or a prevention of impairment or disability due to the condition.

A composition of the present disclosure can be administered by one or more routes of administration using one or more of a variety of methods known in the art. As will be appreciated by the skilled artisan, the route and/or mode of administration will vary depending upon the desired results. Routes of administration for engineered proteins of the invention include intravenous, intramuscular, intradermal, intraperitoneal, subcutaneous, spinal or other parenteral routes of administration, for example by injection or infusion. The phrase ‘parenteral administration’ as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Alternatively, a therapeutic fusion protein of the invention can be administered by a non-parenteral route, such as a topical, epidermal or mucosal route of administration.

The therapeutic fusion proteins of the disclosure can be prepared with carriers that will protect the proteins against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are patented or generally known to those skilled in the art. See, e.g., Sustained and Controlled Release Drug Delivery Systems, J.R. Robinson, ed., Marcel Dekker, Inc., New York, 1978.

In certain embodiments, the therapeutic fusion proteins of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. 4,522,811; 5,374,548; and 5,399,331. The liposomes may comprise one or more moieties which are selectively transported into specific cells or organs, thus enhance targeted drug delivery (see, e.g., Ranade VV (1989) J. Clin. Pharmacol., 29:685).

### Therapeutic Uses and Methods of the Invention

The therapeutic fusion proteins of the present invention have in vitro and in vivo diagnostic and therapeutic utilities. For example, these molecules can be administered to cells in culture, e.g. in vitro, or in a subject, e.g., in vivo, to treat, prevent or diagnose a variety of disorders. The methods are particularly suitable for treating, preventing or diagnosing acute or chronic inflammatory and immune system-driven organ and micro-vascular disorders.

The therapeutic fusion proteins of the invention, whilst not being limited to, are useful for the treatment, prevention, or amelioration of acute and chronic inflammatory organ injuries, in particular inflammatory injuries where endogenous homeostatic clearance mechanisms or efferocytosis pathways for the removal of dying cells, cell fragments and prothrombotic/ proinflammatory microparticles are significantly downregulated. Examples of acute inflammatory organ injuries include myocardial infarction, acute kidney injury (AKI), acute stroke and inflammation and organ injuries resulting from ischemia/ reperfusion such as ischemia/ reperfusion of the gastrointestinal tract, liver, spleen, lung, kidney, pancreas, heart, brain, spinal cord and/or crushed limb.

The therapeutic fusion proteins of the disclosure may also be useful for the diagnosis, treatment, prevention, or amelioration of inhibiting or slowing blood coagulation, microbiome treatment, Inflammatory bowel disease (IBD), fatty acid uptake and/or decreasing gastric motility, microthrombi-dependent disorders, atherosclerosis, cardiac remodeling, tissue fibrosis, acute liver injury, chronic liver diseases, non-alcoholic steatohepatitis (NASH), vascular diseases, age-related vascular disorders, intestinal diseases, sepsis, bone disorders, cancer, Thalassemia, pancreatitis, hepatitis, endocarditis, pneumonia, acute lung injury, osteoarthritis, periodontitis, tissue trauma-induced inflammation, colitis, diabetes, hemorrhagic shock, transplant rejection, radiation-induced damage, splenomegaly, sepsis-induced AKI or multi-organ failure, acute burns, adult respiratory distress syndrome, wound healing, tendon repair and neurological diseases.

In one embodiment, neurological diseases may be selected from conditions having a neuro-psychiatric, neuroinflammatory and/or neurodegenerative component including symptoms such as sickness syndromes, nausea, passive avoidance, suppression of behavioral agility, memory disturbance and memory dysfunction. Examples of neurological diseases include amyloid-beta related neurological diseases such as Alzheimer’s disease, Parkinson’s disease, and depression.

In one embodiment, bone disorders may be selected from conditions including osteoporosis, osteomalacia, ostersclerosis and osteopetrosis. More particularly, administration of a fusion protein of the present disclosure may inhibit expression of at least one osteoclast marker, such as NFATc1, cathepsin K and αvβ3 integrin. In one embodiment, the administration inhibits osteoclastogenesis. In another embodiment, the administration inhibits RANKL-induced osteoclastogenesis. In yet another embodiment, the administration inhibits bone resorption. In still another embodiment, the administration inhibits expression of at least one bone resorption stimulator, such as a bone resorption stimulator comprising TNF, IL-6, IL-17A, MMP-9, Ptgs2, RANKL, Tnfsf11, CXCL1, CXCL2, CXCL3, CXCL5, and combinations thereof. In another embodiment, the administration inhibits expression of at least one pro inflammatory cytokine selected from the group consisting of IL-8 and CCL2/MCP-1.

In one embodiment, tissue fibrosis may be fibrosis in the liver, lung, diaphragm, kidney, brain, heart in which the fusion protein of the invention reduces collagen expression. In one embodiment, the lung fibrosis is interstitial pulmonary fibrosis (IPF). In one embodiment the liver fibrosis is liver cirrhosis, which may or may not be attributable to NASH.

Multiple respiratory diseases feature accumulation of apoptotic cells. Furthermore, defective efferocytosis and phagocytosis by macrophages in Chronic Obstructive Pulmonary Disorder (COPD) are associated with exacerbations and severity. The therapeutic fusion proteins of the disclosure may also be useful for the diagnosis, treatment, prevention, or amelioration of respiratory diseases, such as Acute Respiratory Distress Syndrome, or COPD. The therapeutic fusion proteins of the disclosure may also be useful for the diagnosis, treatment, prevention, or amelioration of Acute Lung Injury (ALI), e.g. lung injury induced by inhalation or aspiration of toxic exogenous or endogenous compounds or drugs; lung injury caused by lung edema, shock, pancreatitis, burns, traumata of thorax or polytraumata, radiation, sepsis, pathogens (bacteria, viruses or parasites such as plasmodia); Chronic pulmonary insufficiency diseases leading to hypoxemia.

The therapeutic fusion proteins of the disclosure may also be useful for the diagnosis, treatment, prevention, or amelioration of severity of lung injury caused by viruses of the Cornona type, e.g. SARS-CoV, SARS-CoV-2, or MERS-CoV. In one embodiment, the therapeutic fusion proteins of the disclosure are provided for the use in treatment of SARS-CoV-2 infection in COVID 19 patients.

The therapeutic fusion proteins of the disclosure may also be useful for the diagnosis, treatment, prevention, or amelioration of severity of transfusion associated lung insufficiency (TRALI).

The therapeutic fusion proteins of the disclosure may also be useful for the diagnosis, treatment, prevention, or amelioration of severity of chronic pulmonary insufficiency diseases leading to hypoxemia.

The therapeutic fusion proteins of the disclosure, e.g. the therapeutic fusion proteins contains a domain of EDIL3 of the disclosure, may also be useful for the diagnosis, treatment, prevention, or amelioration of severity of postoperative peritoneal adhesions.

The therapeutic fusion proteins of the disclosure may also be useful for the diagnosis, treatment, prevention, or amelioration of severity of heart failure.

The therapeutic fusion proteins of the disclosure may also be useful for the diagnosis, treatment, prevention, or amelioration of severity of hemodialysis.

The therapeutic fusion proteins of the disclosure may also be useful for the diagnosis, treatment, prevention, or amelioration of severity of delayed graft function or of graft versus host disease.

The therapeutic fusion proteins of the disclosure may also be useful for the diagnosis, treatment, prevention, or amelioration of severity of severe frostbites, trench foot, pyoderma gangraenosum/gangrene.

The therapeutic fusion proteins of the disclosure may also be useful for the diagnosis, treatment, prevention, or amelioration of severity of pathologies induced by bacteria, fungi, viruses or parasits (for example, sepsis or other pathologies directly induced by the pathogens such as in anthrax, plague, Necrotizing soft-tissue infections (NSTIs such as necrotizing fasciitis,) osteomyelitis, malaria).

The therapeutic fusion proteins of the disclosure may also be useful for the diagnosis, treatment, prevention, or amelioration of severity of trauma/polytraumata caused by injury-causing accidents, such as work accidents, falls, traffic accidents, ballistic and combat injury or other injury mechanisms.

The therapeutic fusion proteins of the disclosure may also be useful for the diagnosis, treatment, prevention, or amelioration of severity of osteoclast mediated pathology.

The therapeutic fusion proteins of the disclosure may be administered as the sole active ingredient or in conjunction with, e.g. as an adjuvant to or in combination to, other drugs e.g. immunosuppressive or immunomodulating agents or other anti-inflammatory agents or e.g. cytotoxic or anti-cancer agents, e.g. for the treatment or prevention of diseases mentioned above.

Administered ‘in combination’, in reference to an additional therapeutic agent, means that two (or more) different treatments are delivered to the subject during the course of the subject’s affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The term ‘concurrently’ is not limited to the administration of therapies (e.g., prophylactic or therapeutic agents) at exactly the same time, but rather it is meant that a pharmaceutical composition comprising therapeutic fusion proteins thereof of the present disclosure are administered to a subject in a sequence and within a time interval such that the fusion proteins can act together with the additional therapeutic agent(s) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route.

A therapeutic fusion protein as described herein, and the additional therapeutic agent(s) can be administered simultaneously, in the same or in separate pharmaceutical composition as the disclosed fusion protein, or sequentially. For sequential administration, the fusion protein as described herein, can be administered first, and the additional agent can be administered second, or the order of administration can be reversed. The additional therapeutic agent(s) may be administered to a subject by the same or different routes of administration compared to the fusion protein.

The therapeutic fusion protein as described herein, and/or additional therapeutic agent(s), procedures or modalities can be administered during periods of active disorder, or during a period of remission or less active disease. The therapeutic fusion protein as described herein, can be administered before the other treatment, concurrently with the treatment, post-treatment, or during remission of the disorder.

When administered in combination, the therapeutic fusion protein as described herein, and the additional therapeutic agent (e.g., second or third agent), or all, can be administered in an amount or dose that is higher, lower or the same than the amount or dosage of each agent used individually, e.g., as a monotherapy. In certain embodiments, the therapeutic fusion protein as described herein, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the therapeutic fusion protein as described herein, the additional agent (e.g., second or third agent), or all, that results in a desired effect (e.g., treatment of an inflammatory disease or condition) is lower (e.g., at least 20%, at least 30%, at least 40%, or at least 50% lower) than the amount or dosage of each agent used individually, e.g., as a monotherapy, required to achieve the same therapeutic effect.

For example, the therapeutic fusion proteins of the disclosure may be used in combination with DMARD, e.g. Gold salts, sulphasalazine, anti-malarias, methotrexate, D-penicillamine, azathioprine, mycophenolic acid, tacrolimus, sirolimus, minocycline, leflunomide, glucocorticoids; a calcineurin inhibitor, e.g. cyclosporin A or FK 506; a modulator of lymphocyte recirculation, e.g. FTY720 and FTY720 analogs; a mTOR inhibitor, e.g. rapamycin, 40-O-(2-hydroxyethyl)-rapamycin, CCI779, ABT578, AP23573 or TAFA-93; an ascomycin having immuno-suppressive properties, e.g. ABT-281, ASM981, etc.; corticosteroids; cyclophosphamide; azathioprine; leflunomide; mizoribine; mycophenolate mofetil; 15-deoxyspergualine or an immunosuppressive homologue, analogue or derivative thereof; immunosuppressive monoclonal antibodies, e.g., monoclonal antibodies to leukocyte receptors, e.g., MHC, CD2, CD3, CD4, CD7, CD8, CD25, CD28, CD40. CD45, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4lg (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent, e.g. paclitaxel, gemcitabine, cisplatinum, doxorubicin or 5-fluorouracil; anti TNF agents, e.g. monoclonal antibodies to TNF, e.g. infliximab, adalimumab, CDP870, or receptor constructs to TNF-RI or TNF-RII, e.g. Etanercept, PEG-TNF-RI; blockers of proinflammatory cytokines, IL-1 blockers, e.g. Anakinra or IL-1 trap, canakinumab, IL-13 blockers, IL-4 blockers, IL-6 blockers; chemokines blockers, e.g inhibitors or activators of proteases, e.g. metalloproteases, anti-IL-15 antibodies, anti-IL-6 antibodies, anti-IL-4 antibodies, anti-IL-13 antibodies, anti-CD20 antibodies, NSAIDs, such as aspirin or an anti-infectious agent; damage-associated molecular pattern (DAMP) or pathogen-associated molecular pattern (PAMP) antagonists, e.g. converters, detoxifiers, removers, e.g. ATP converters, HMGB-1 modulators, histone-detoxifiers; inhibitors of superantigen induced immune-responses; complement inhibitors and extracorporal plasmapheresis devices.

### Kits

Also within the scope of the invention are kits consisting of the compositions e.g., therapeutic fusion proteins of the disclosure, and instructions for use. Such kits comprise a therapeutically effective amount of a fusion protein according to the disclosure. Additionally, such kits may comprise means for administering the therapeutic fusion protein (e.g., an auto injector, a syringe and vial, a prefilled syringe, a prefilled pen) and instructions for use. These kits may contain additional therapeutic agents (described infra) for treating a patient having an autoimmune disease or an inflammatory disorder or AOI. Such kits may also comprise instructions for administration of the therapeutic fusion protein to treat the patient. Such instructions may provide the dose, route of administration, regimen, and total treatment duration for use with the enclosed fusion protein. Kits typically include a label indicating the intended use of the contents of the kit. The term label includes any writing, or recorded material supplied on or with the kit, or which otherwise accompanies the kit. The kit may further comprise tools for diagnosing whether a patient belongs to a group that will respond to treatment with a therapeutic fusion protein of the present invention, as defined above.

### EMBODIMENTS

The present disclosure provides the following embodiments:
1. A therapeutic fusion protein for enhancing efferocytosis comprising an integrin binding domain, a phosphatidylserine (PS) binding domain and a solubilizing domain, wherein the solubilizing domain is inserted between the integrin binding domain and the PS binding domain, and wherein the PS binding domain is a truncated variant.
2. The fusion protein of embodiment 1, wherein the PS binding domain is a truncated variant of at least one PS binding domain listed in Table 2.
3. The fusion protein of embodiment 1 or embodiment 2, wherein the PS binding domain is a truncated variant of the PS binding motif of MFG-E8 or of EDIL3.
4. The fusion protein of embodiment 3, wherein the PS binding domain is a truncated variant of the PS binding motif of MFG-E8.
5. The fusion protein of embodiment 4, wherein the PS binding domain is a discoidin domain.
6. The fusion protein of any one of the preceding embodiments, wherein the PS binding domain is a C1 domain.
7. The fusion protein of any one of the preceding embodiments, wherein the PS binding domain does not comprise a C2 domain.
8. A fusion protein for enhancing efferocytosis comprising an integrin binding domain, a phosphatidylserine (PS) binding domain and a solubilizing domain, wherein the solubilizing domain is inserted between the integrin binding domain and the PS binding domain, and wherein the PS binding domain is a C1 domain.
9. The fusion protein of any one of the preceding embodiments, wherein the integrin binding domain binds to one or more intergins.
10. The fusion protein of embodiment 9, wherein the integrin binding domain binds to αvβ3 and/or αvβ5 and/or α8β1 integrin.
11. The fusion protein of any one of the preceding embodiments, wherein the integrin binding domain comprises a Arginine-Glycine-Aspartic acid (RGD) motif.
12. A therapeutic fusion protein of formula EGF - S - C (Formula I), wherein
   (i) EGF is an integrin binding domain, wherein the integrin binding domain binds to one or more intergins,
   (ii) S is a solubilizing domain, and
   (iii) C is a truncated PS binding domain.
13. The therapeutic fusion protein of embodiment 12, wherein the integrin binding domain binds to αvβ3 and/or avβ5 and/or α8β1 integrin.
14. The therapeutic fusion protein of embodiment 12 or embodiment 13, wherein the integrin binding domain comprises a Arginine-Glycine-Aspartic acid (RGD) motif.
15. The therapeutic fusion protein of any one of embodiment 12 to 14, wherein the integrin binding domain is an EGF-like domain of MFG-E8, EDIL3 or a protein comprising an integrin binding domain listed in Table 1.
16. The therapeutic fusion protein of any one of embodiments 12 to 15, wherein the truncated PS binding domain is a truncated variant of a PS binding domain listed in Table 2.
17. The therapeutic fusion protein of any one of embodiments 12 to 16, wherein the PS binding domain is a truncated variant of the PS binding motif of MFG-E8 or of EDIL3.
18. The fusion protein of any one of embodiments 12 to 16, wherein the PS binding domain is a truncated variant of the PS binding motif of MFG-E8.
19. The fusion protein of any one of embodiments 12 to 18, wherein the PS binding domain is a discoidin domain.
20. The therapeutic fusion protein of any one of embodiments 11 to 16, wherein the truncated PS binging domain comprises any of C1 domain and/or C2 domain of a PS binding domain listed in Table 2.
21. The therapeutic fusion protein of any one of embodiments 11 to 18, wherein the truncated PS binding domain is a C1 domain.
22. The therapeutic fusion protein of any one of embodiments 11 to 21, wherein the truncated PS binding domain does not comprise a C2 domain.
23. The fusion protein of any one of the preceding embodiments, wherein the solubilizing domain is linked directly to the integrin binding domain, to the PS binding domain or to both domains.
24. The fusion protein of any one of the preceding embodiments, wherein the solubilizing domain is linked indirectly to the integrin binding domain and/or the PS binding domain by a linker.
25. The fusion protein of any one of the preceding embodiments, wherein the integrin binding domain has an amino acid sequence of SEQ ID NO: 2, or at least 90% sequence identity thereto.
26. The fusion protein of any one of the preceding embodiments, wherein the integrin binding domain has an amino acid sequence of SEQ ID NO: 77 or at least 90% sequence identity thereto.
27. The fusion protein of any one of the preceding embodiments, wherein the integrin binding domain has an amino acid sequence selected from: SEQ ID NO: 96, SEQ ID NO: 97, SEQ ID NO: 98, SEQ ID NO: 99, SEQ ID NO: 100, or SEQ ID NO: 101; or at least 90% sequence identity thereto.
28. The fusion protein of any one of the preceding embodiments, wherein the PS binding domain has an amino acid sequence of SEQ ID NO: 141 or SEQ ID NO: 142; or at least 90% sequence identity thereto.
29. The fusion protein of any one of the preceding embodiments, wherein the PS binding domain has an amino acid sequence of SEQ ID NO: 144, or at least 90% sequence identity thereto.
30. The fusion protein of any one of the preceding embodiments, wherein the solubilizing domain is HSA and has an amino acid sequence of SEQ ID NO: 4, or at least 90% sequence identity thereto.
31. The fusion protein of any one of the preceding embodiments comprising in sequence: an integrin binding domain-HSA-PS binding domain.
32. A therapeutic fusion protein comprising MFG-E8 and a solubilizing domain, wherein the MFG-E8 comprises from N-terminal to C-terminal: an EGF-like domain, a C1 domain or a C2 domain, and comprises a sequence from wild-type human MFG-E8 (SEQ ID NO: 1) or a functional variant thereof.
33. The fusion protein of embodiment 32, wherein the solubilizing domain is inserted between the EGF-like domain and the C1 or C2 domain.
34. The fusion protein of any one of the preceding embodiments, wherein the solubilizing domain is HSA, HSA D3 or Fc-IgG, or a functional variant thereof.
35. The fusion protein of any one of the preceding embodiments wherein the solubilizing domain comprises human serum albumin (HSA), or a functional variant thereof.
36. A fusion protein, wherein the fusion protein has an amino acid sequence of SEQ ID NO: 34, or at least 90% sequence identity thereto.
37. A fusion protein, wherein the fusion protein has an amino acid sequence of SEQ ID NO: 36, or at least 90% sequence identity thereto.
38. A fusion protein, wherein the fusion protein has an amino acid sequence selected from: SEQ ID NO: 119, SEQ ID NO: 121, SEQ ID NO: 125, SEQ ID NO: 129, SEQ ID NO: 131, SEQ ID NO: 133, SEQ ID NO: 135, SEQ ID NO: 137, or SEQ ID NO: 147; or at least 90% sequence identity thereto.
39. A fusion protein, wherein the fusion protein has an amino acid sequence of SEQ ID NO: 147, or at least 90% sequence identity thereto.
40. The fusion protein of any one of the preceding embodiments, wherein said fusion protein:
   a. restores impaired efferocytosis of macrophages in a human macrophage-neutrophil efferocytosis assay;
   b. reduces the number of plasma microparticles by clearance in a human endothelial-microparticle efferocytosis assay; and/or
   c. protects against multi-organ injury in a model of acute kidney ischaemia.
   d. ameliorates disease burden in a model of liver fibrosis
41. An isolated nucleic acid encoding the amino acid sequence of any one of embodiments 36 to 39.
42. A cloning or expression vector comprising the nucleic acid according to embodiment 41.
43. A viral vector comprising the isolated nucleic acid according to embodiment 41, preferably the viral vector comprising the isolated nucleic acid according to embodiment 41 is derived from AAV.
44. The viral vector according to embodiment 43, wherein the vector is administered to a subject, e.g., a human subject, in need therefor.
45. The viral vector according to embodiment 43, for use in the treatment and/or prevention of the diseases as listed herein.
46. A recombinant host cell suitable for the production of a therapeutic fusion protein, comprising one or more cloning or expression vectors according to embodiment 42 and optionally, secretion signals.
47. The recombinant host cell of embodiment 46, wherein the host cell is e.g. a prokaryotic, yeast, insect or mammalian cell.
48. The fusion protein of any one of the preceding embodiments, wherein expression of the protein in a host cell results in a yield of at least 10 mg/L.
49. The fusion protein of any one of the preceding embodiments, wherein expression of the protein in a mammalian cell results in an increase in yield of at least 100 fold over wild-type MFG-E8 (SEQ ID NO: 1).
50. A pharmaceutical composition comprising the fusion protein of any one of embodiments 1 to 40 and at least one pharmaceutically acceptable carrier.
51. A method of treatment or prevention of an inflammatory disorder or inflammatory organ injury in an individual in need thereof, comprising administering to the individual a therapeutically effective amount of the fusion protein of any one of embodiments 1 to 40.
52. The fusion protein of any one of embodiments 1 to 40 for use in the treatment or prevention of an inflammatory disorder or inflammatory organ injury in an individual in need thereof.
53. The method of embodiment 51 or the use of embodiment 52, wherein the inflammatory disorder or inflammatory organ injury is acute kidney injury, sepsis, myocardial infarction, acute stroke, burns, traumatic injury, and inflammatory and organ injuries resulting from ischemia/ reperfusion.
54. The method of embodiment 51 or the use of embodiment 52, wherein the fusion protein is administered in combination with another therapeutic agent.
55. The method or use of embodiment 54, wherein the another therapeutic agent is an immunosuppressive agent, an immunomodulating agent, an anti-inflammatory agent, an anti-oxidant, an anti-infective agent, a cytotoxic agent or an anti-cancer agent.
56. A therapeutic fusion protein comprising MFG-E8 and a solubilizing domain, wherein the MFG-E8 comprises from N-terminal to C-terminal: an EGF-like domain, a C1 domain or a C2 domain, and comprises a functional variant of the sequence from wild-type human MFG-E8 (SEQ ID NO: 1).
57. The fusion protein of embodiment 56, wherein the solubilizing domain is inserted between the EGF-like domain and the C1 domain.
58. The fusion protein of embodiment 56, wherein the solubilizing domain is inserted between the EGF-like domain and the C2 domain.
59. The fusion protein of any one of embodiments 56 to 58, wherein the solubilizing domain is HSA, HSA D3 or Fc-IgG, or a functional variant thereof.
60. The fusion protein of any one of embodiments 56 to 59, wherein the fusion protein has an amino acid sequence selected from the SEQ ID listed in Table 4.
61. An isolated nucleic acid encoding the fusion protein of any one of embodiments 56 to 60.
62. A viral vector comprising the isolated nucleic acid according to embodiment 61, preferably the viral vector comprising the isolated nucleic acid according to embodiment 60 is derived from AAV.
63. The viral vector according to embodiment 62, wherein the vector is administered to a subject, e.g., a human subject, in need therefor.
64. The viral vector according to embodiment 62, for use in the treatment and/or prevention of the diseases as listed herein.
65. A cloning or expression vector comprising the nucleic acid according to embodiment 61.
66. A recombinant host cell suitable for the production of a therapeutic fusion protein, comprising one or more cloning or expression vectors according to embodiment 65 and optionally, secretion signals.
67. The recombinant host cell of embodiment 66, wherein the host cell is e.g. a prokaryotic, yeast, insect or mammalian cell.
67. The fusion protein of any one of embodiments 56 to 60, wherein expression of the protein in a host cell results in a yield of at least 10 mg/L.
69. The fusion protein of any one of embodiments 56 to 60, wherein expression of the protein in a mammalian cell results in an increase in yield of at least 100 fold over wild-type MFG-E8.

It is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

All references cited herein, including patents, patent applications, papers, publications, text books, and the like, and the references cited therein, to the extent that they are not already, are hereby incorporated herein by reference in their entirety.

### EXAMPLES

The following examples are provided to further illustrate the disclosure but not to limit its scope. Other variants of the disclosure will be readily apparent to one of ordinary skill in the art and are encompassed by the appended claims.

### Example 1: Generation of Fusion Proteins

MFG-E8 is a multi-domain protein consisting of a N-terminal epidermal growth factor (EGF-like) domain and two C-terminal lectin-type C domains (C1 and C2). Attempts to produce recombinant full-length human protein, as documented in the literature, have shown that the protein aggregates and expression rates are very low (Castellanos et al., (2016) Protein Expression Purification 1124: 10-22). Therefore, in order to try to solubilize the protein and boost its expression, we investigated the effect of fusing a number of proteins to MFG-E8.

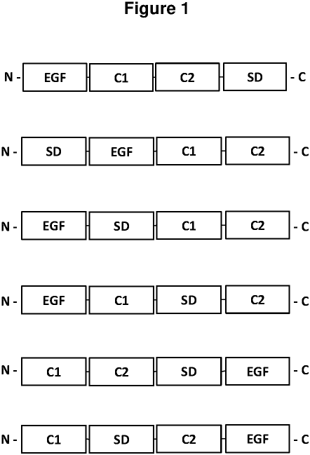

A solubilizing domain (SD) derived from human Fc-lgG1, human serum albumin (HSA) and domain 3 of HSA (HSA D3) were fused in different positions to MFG-E8; at the N- or C-terminus, or in between the EGF and C1 or C1 and C2 domains, as shown schematically in FIG. 1. Furthermore, fusions to Fc-lgG1 or HSA have the potential to extend the half-life of the molecule in vivo, since these proteins bind to FcRn. Fusion of MFG-E8 to Fc-lgG1 or HSA can also enhance the production and solubility (Castellanos et al., (2016) supra) of the fusion protein as is shown in the following examples.

Table 5 shows the binding of fusion protein FP330 (EGF-HSA-C1-C2; SEQ ID NO: 42) comprising a HSA insert, to human neonatal Fc-receptor (See also Example 5.1).

TABLE 5

Binding affinity of fusion protein FP330 to human FcRn

| Sample | human FcRn (pH 5.8) | | human FcRn (pH 7.4) | |
|---|---|---|---|---|
| | K_{D} (nM) | n | K_{D} (nM) | n |
| FP330 (SEQ ID NO: 42) | 1380 ± 95 | 2 | no binding | 1 |

### Example 2: Generation of wtMFG-E8 and MFG-E8 HSA Fusions; Expression and Purification

Methods for generation of fusion proteins are described below; in brief, MFG-E8 and MFG-E8 fusions and EDIL fusions, in particular fusions to HSA, were generated according to the following method.

DNA was synthesized at GeneArt (Regensburg, Germany) and cloned into a mammalian expression vector using restriction enzyme-ligation based cloning techniques. The resulting plasmid was transfected into HEK293T cells. For transient expression of proteins, vectors for wild-type or engineered chains were transfected into suspension-adapted HEK293T cells using Polyethylenimine (PEI; Cat# 24765 Polysciences, Inc.). Typically, 100 ml of cells in suspension at a density of 1-2 Mio cells per ml was transfected with DNA containing 100 µg of expression vectors encoding the engineered chains. The recombinant expression vectors were then introduced into the host cells and the construct produced by further culturing of the cells for a period of 7 days to allow for secretion into the culture medium (HEK, serum-fee medium) supplemented with 0.1% pluronic acid, 4 mM glutamine, and 0.25 µg/ml antibiotic.

The produced constructs were then purified from cell-free supernatant, using immobilized metal ion affinity chromatography (IMAC), or Protein A capture, or anti-HSA capture chromatography.

When his-tagged protein was captured by IMAC, filtered conditioned media was mixed with IMAC resin (GE Healthcare), equilibrated with 1% triton and 20 mM NaPO4, 0.5Mn NaCl, 20 mM Imidazole, pH7.0. The resin was washed three times with 15 column volumes of 20 mM NaPO4, 0.5Mn NaCl, 20 mM Imidazole, pH7.0 before the protein was eluted with 10 column volumes elution buffer (20 mM NaPO4, 0.5Mn NaCl, 500 mM Imidazole, pH7.0).

When protein was captured by Protein A or anti-HSA chromatography, filtered conditioned media was mixed with Protein A resin (CaptivA PriMab™, Repligen) or anti-HSA resin (Capture Select Human Albumin affinity matrix, Thermo), equilibrated with PBS, pH7.4. The resin was washed three times with 15 column volumes of PBS, pH7.4 before the protein was eluted with 10 column volumes elution buffer (50 mM citrate, 90 mM NaCl, pH 2.5) and pH neutralized using 1 M TRIS pH10.0.

Finally, eluted fractions were polished by using size exclusion chromatography (HiPrep Superdex 200, 16/60, GE Healthcare Life Sciences) and analyzed by SDS-PAGE against a Precision Plus Protein Unstained Standards marker (Biorad, ref#161-0363).

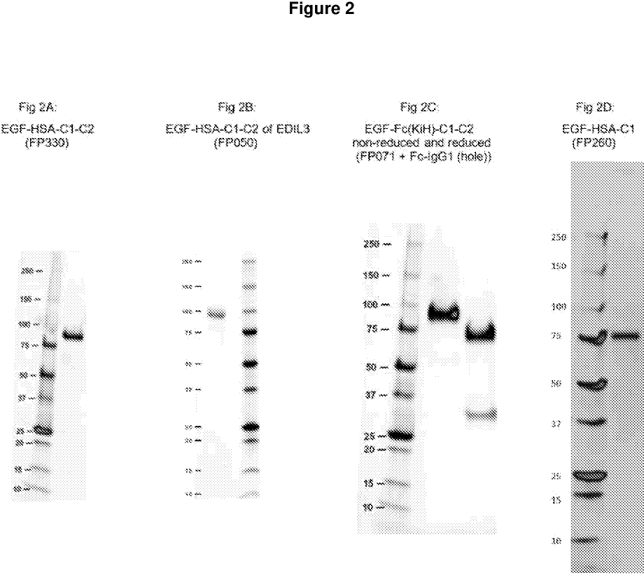

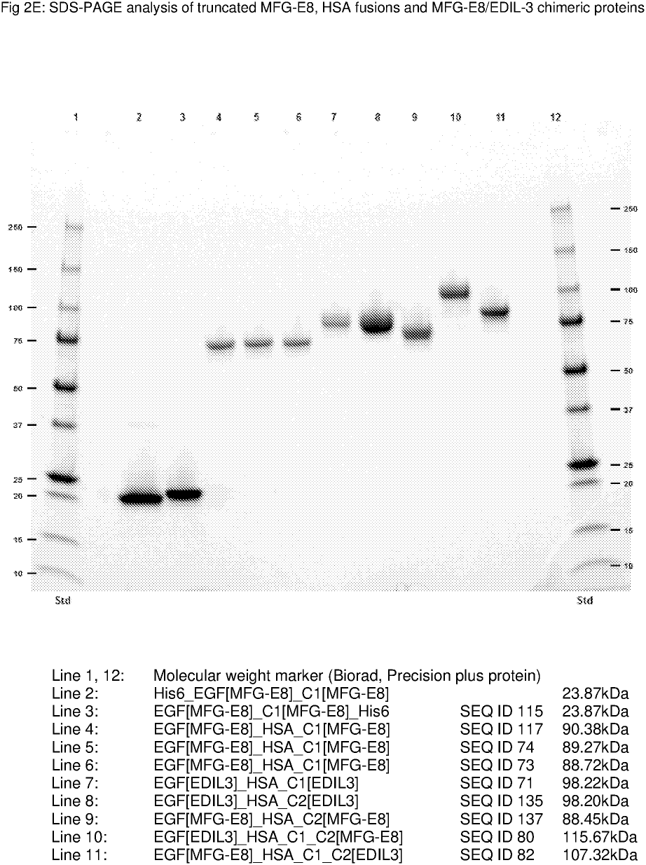

Representative expression gels for the fusion proteins are shown in FIGS. 2: FIG. 2A: EGF-HSA-C1-C2 protein (FP330; SEQ ID NO: 42); FIG. 2B: EGF-HSA-C1-C2 of EDIL3 protein (FP050; SEQ ID NO: 12); FIG. 2C: EGF-Fc(KiH) C1-C2 protein non-reduced and reduced. This protein is a heterodimer of FP071 (EGF-Fc(knob)-C1-C2; SEQ ID NO: 18) with Fc-lgG1 hole (SEQ ID NO: 10); FIG. 2D: EGF-HSA-C1 protein (FP260; SEQ ID NO: 34). Protein under reduced and non-reduced conditions is shown in FIG. 2C because heterodimers tend to fall apart under reducing conditions therefore both conditions were tested. Results of expression and the yield following purification for a further set of fusion proteins are shown in Table 6; As can be seen from the expression data, HSA fusions of MFG-E8, even with HSA in different positions, show at least a 100-fold improvement in expression over wtMFG-E8. As is shown in the right hand column of Table 6, HSA fusions of MFG-E8 also show an increase in yield of at least 100-fold over wtMFG-E8.

TABLE 6

Expression and yield of fusion proteins expressed in a HEK cell line

| Protein | Expression post His capture (mg/l) | Final yield after His and SEC (mg/l) |
|---|---|---|
| wtMFG-E8 | 0.2 | 0.04 |
| FP220 (HSA-EGF-C1-C2) | 23 | 5.5 |
| FP110 (EGF-C1-C2-HSA) | 34 | 7.8 |
| FP330 (EGF-HSA-C1-C2) | 23 | 4.0 |

Other examples of therapeutic fusion proteins of the disclosure were generated according to the above method and further analyzed by SDS-PAGE (Sodium dodecyl sulfate polyacrylamide gel electrophoresis), were proteins are separated based on their molecular weight. Each protein was mixed with Laemmli buffer before loading on polyacrylamide gel (Biorad, 4-20% Mini-PROTEAN TGX Stain free). After 30 min migration at 200 V in TRIS-Glycine-SDS running buffer, proteins contained in the gel were revealed in a stain-free enabled imager (Biorad, Gel Doc EZ). As described FIG. 2E, SDS-PAGE shows recombinant proteins which have been produced and purified:
Line 1, 12: Molecular weight marker (Biorad, Precision plus protein)
Line 2: His6_EGF[MFG-E8]_C1[MFG-E8] 23.87 kDa
Line 3: EGF[MFG-E8]_C1[MFG-E8]_His6 SEQ ID 115 23.87 kDa
Line 4: EGF[MFG-E8]_HSA_C1[MFG-E8] SEQ ID 117 90.38 kDa
Line 5: EGF[MFG-E8]_HSA_C1[MFG-E8] SEQ ID 74 89.27 kDa
Line 6: EGF[MFG-E8]_HSA_C1[MFG-E8] SEQ ID 73 88.72 kDa
Line 7: EGF[EDIL3]_HSA_C1[EDIL3] SEQ ID 71 98.22 kDa
Line 8: EGF[EDIL3]_HSA_C2[EDIL3] SEQ ID 135 98.20 kDa
Line 9: EGF[MFG-E8]_HSA_C2[MFG-E8] SEQ ID 137 88.45 kDa
Line 10: EGF[EDIL3]_HSA_C1_C2[MFG-E8] SEQ ID 80 115.67 kDa
Line 11: EGF[MFG-E8]_HSA_C1_C2[EDIL3] SEQ ID 82 107.32 kDa

### Example 3: Characterization of MFG-E8-HSA Engineered Proteins

### 3.1 Phosphatidylserine Binding (Biochemical)

L-α-phosphatidylserine (brain, porcine, Avanti 840032, Alabama, US) was dissolved in chloroform, diluted in methanol and coated onto 384-well microtiter plates (Corning™ 3653, Kennebunk ME, US) at 1 µg/mL. After overnight incubation at 4° C., the solvent was evaporated using a SpeedVac™ System (Thermo Scientific™). The plates were treated with phosphate buffered saline (PBS) containing 3% fatty acid-free bovine serum albumin (BSA) at RT for 1.5h.

Binding of fusion proteins to L-α-phosphatidylserine was assessed by competing against binding of biotinylated murine MFG-E8/lactadherin (produced in-house, mMFG-E8:biotin). The proteins were diluted in PBS containing 3% fatty acid free BSA, pH 7.4 and incubated with L-α-phosphatidylserine -coated microtiter plates for 30 min. mMFG-E8:biotin in PBS containing 3% fatty acid free BSA, pH 7.4 was added at 1 nM and incubated for additional 30 min. Unbound mMFG-E8:biotin was removed by three washing steps with dissociation-enhanced lanthanide fluorescence immunoassay (DELFIA™) wash buffer (Perkin Elmer 1244-114 MA, US). Europium-labelled streptavidin (Perkin Elmer 1244-360, Wallac Oy, Finland) was added in DELFIA™ Assay buffer (Perkin Elmer 1244-111 MA, US) at RT for 20 min. This was followed by three washing steps with DELFIA™ Assay buffer. Europium was revealed as instructed by manufacturer (Perkin Elmer 1244-105, Boston MA, US). Time resolved-fluorescence of Europium was quantified with an Envision™2103 multi-label plate reader, Perkin Elmer, CT,US). Data analysis was performed using MS Excel and GraphPad Prism software.

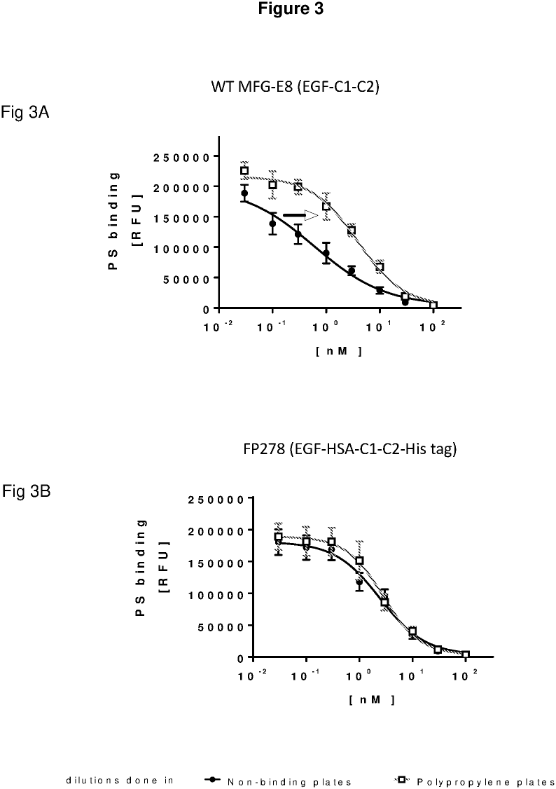

Polypropylene plates are low-protein binding microtiter plates that are typically used in laboratories for serial dilutions. Compared to polystyrene, these plates have the advantage of reducing protein loss during dilutions and are typically classified as “low-protein binding” plates. When dilutions of wtMFG-E8 were made in polypropylene plates, compared to dilutions made in non-binding plates, wtMFG-E8 lost potency in the L-α-phosphatidylserine competition assay. These data, as shown in FIGS. 3, suggest that wtMFG-E8 is partially lost during liquid handling and dilution steps when using polypropylene plates which have already been optimized for low protein binding (FIG. 3A). These results indicate that the inherent stickiness of wtMFG-E8 poses a challenge in handling in the laboratory and most likely during drug manufacturing and production, where capture and polish steps are required to produce drug substance with high yield and very high purity. In contrast, the stickiness of the engineered protein FP278 (EGF-HSA-C1-C2-His tag; SEQ ID NO: 44) was drastically reduced compared to wtMFG-E8 and virtually no difference between dilutions performed in non-binding plates versus polypropylene plates was observed (FIG. 3B). These data suggest that inserting a solubilizing domain into the proteins of the present disclosure can improve their technical handling to improve step yield and thus the overall yield during the manufacturing process.

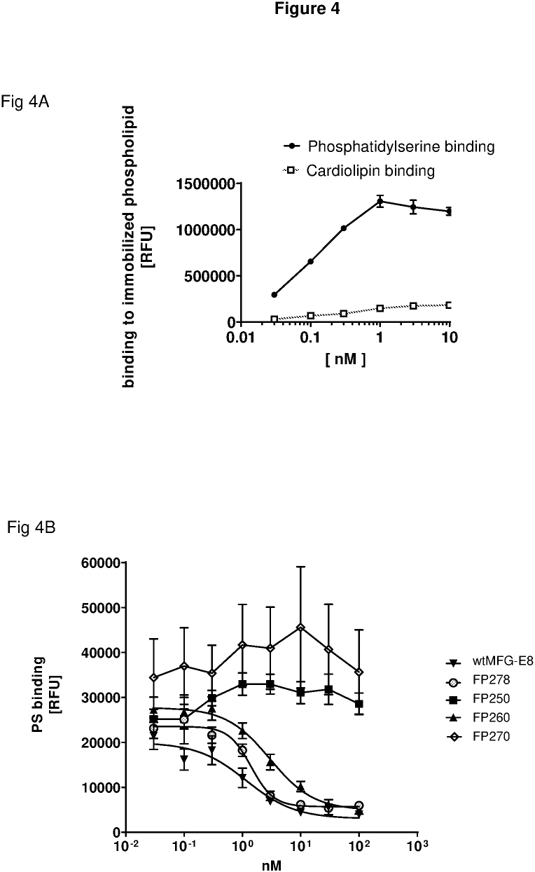

The assessment of binding of the fusion proteins to L-α-phosphatidylserine is shown in FIGS. 4. The engineered MFG-E8-derived protein FP278 (EGF-HSA-C1-C2-His tag; SEQ ID NO: 44) bound to immobilized PS and to a lesser extent to the phospholipid cardiolipin in a concentration dependent manner (FIG. 4A). The binding of FP278 to immobilized L-α-phosphatidylserine or binding to cardiolipin (1,3-bis(sn-3′-phosphatidyl)-sn-glycerol) was detected using an antibody against the EGF-L domain of wtMFG-E8. The binding strength of several recombinant fusion proteins to immobilized L-α-phosphatidylserine is shown in FIG. 4B. Human wtMFG-E8, and the fusion proteins FP278 (EGF-HSA-C1-C2-His tag; SEQ ID NO: 44) and FP260 (EGF-HSA-C1; SEQ ID NO: 34) efficiently competed with binding of 1 nM biotinylated mouse MFG-E8 to immobilized L-α-phosphatidylserine in a concentration-dependent manner.

The IC₅₀ values obtained for the fusion proteins signify highly similar L-α-phosphatidylserine -binding strengths of the C1-C2 domains of the engineered protein FP278 (EGF-HSA-C1-C2-His tag; SEQ ID NO: 44) compared to human wtMFG-E8. Surprisingly, these data also suggest that the human C2 domain does not, or only weakly interacts with L-α-phosphatidylserine as shown by the result for FP270 (EGF-HSA-C2; SEQ ID NO: 36), which along with FP250 (EGF-HSA; SEQ ID NO: 32) did not compete in this assay format. FP100, an EGF-C2-C2 protein (SEQ ID NO: 26) was tested and did not compete in this assay format (not shown), leaving the C1 domain as the major PS-binding moiety in human MFG-E8. This finding was surprising as a major body of literature suggests that the C2 domain of MFG-E8 is the major domain responsible for PS binding (Andersen et al., (2000) Biochemistry, 39(20): 6200-6; Shi & Gilbert (2003) Blood, 101: 2628-2636; Shao et al., (2008) J Biol Chem., 283(11): 7230-41). In conclusion, these findings demonstrate that the C1 domain is the major integral PS binding domain of the MFG-E8 engineered proteins and is important for PS-binding dependent functions. As such, the C1 domain may be useful for substitution into heterologous proteins to confer PS binding; however, the highest PS binding was shown for fusion proteins containing a C1-C2 or C1-C1 tandem domain (latter not shown).

### 3.2 αV Integrin Adhesion Assay

Fusion proteins were diluted in phosphate buffered saline (PBS) pH 7.4 and 50 µL of a 24 nM solution was immobilized by adsorption (96 well plate, Nunc Maxisorb) overnight (1.2 nM /well). The plates were subsequently treated with PBS containing 3% fatty acid free bovine serum albumin (BSA) at RT for 1.5 h. αvβ3 integrin- expressing lymphoma cells (ATCC-TIB-48 BW5147.G.1.4, ATCC, US) were cultivated in RPMI 1640 supplemented with GlutaMax, 25 mM HEPES, 10% FBS, Pen/Strep, 1 mM NaPyruvate, 50 µM β-Mercaptoethanol. The cells were split the day before the adhesion experiment. Cells were labelled with 3 µg/mL 2′,7′-bis-(2-carboxyethyl)-5-(and-6)-carboxyfluorescein, acetoxymethyl ester (BCECF AM) (Thermo Fisher Scientific Inc, US) for 30 min. BW5147.G.1.4 cells were resuspended in adhesion buffer (TBS, 0.5% BSA, 1 mM MnCl₂, pH 7.4) and 50000 cells/well were allowed to adhere at RT for 40 min. Non-adherent cells were removed by repeated washing with adhesion buffer. Fluorescence of adherent cells was quantified using an Envision™2103 multilabel plate reader, Perkin Elmer, US. Data analysis was performed using MS Excel and GraphPad Prism software.

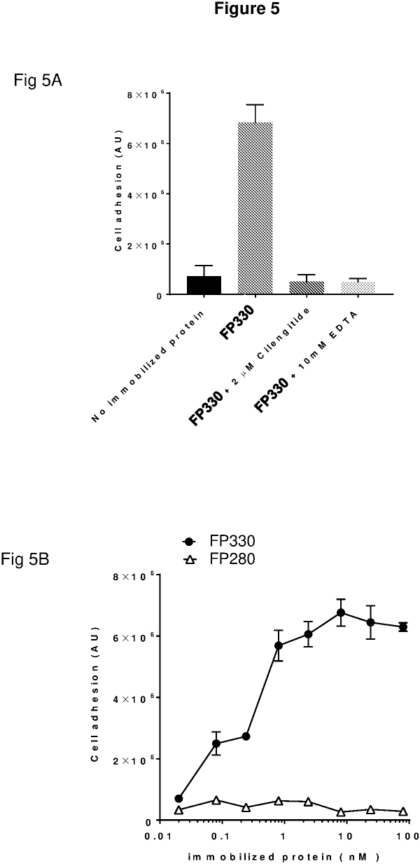

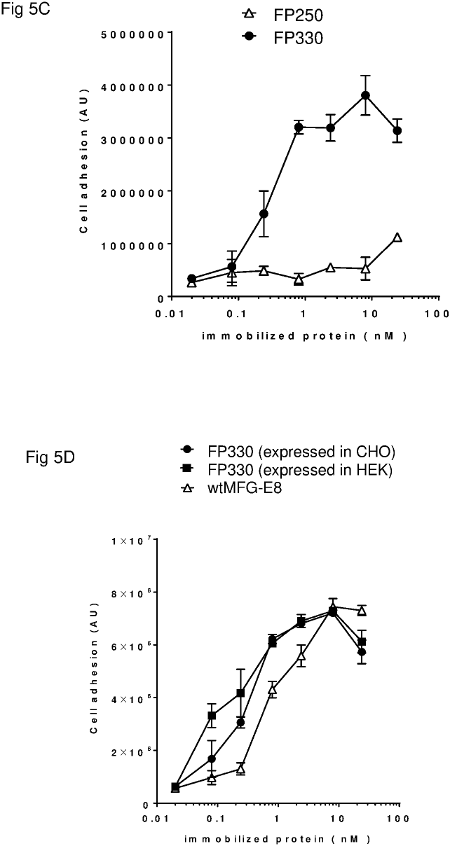

Cell adhesion to the immobilized fusion protein FP330 (EGF-HSA-C1-C2; SEQ ID NO: 42) was completely blocked by the αv integrin inhibitor cilengitide or 10 mM EDTA demonstrating integrin-dependent cell adhesion to immobilized engineered protein (FIG. 5A). A single point mutation in the integrin binding motif RGD (RGD > RGE) of the EGF-like domain (FP280; SEQ ID NO: 38) resulted in complete abrogation of cell adhesion demonstrating that a functional and accessible RGD binding motif in the fusion protein is essential for αv integrin-dependent adhesion (FIG. 5B). An immobilized EGF-HSA protein lacking the C1-C2 domains, FP250 (SEQ ID NO: 32) did not, or only marginally, support adhesion of BW5147.G.1.4 cells despite an EGF-like domain (FIG. 5C). This finding suggests that under the tested experimental conditions, the RGD loop in EGF-like domain fused to HSA may be insufficiently accessible to cell surface integrins possibly due to steric reasons. This disturbance was not apparent once C1, C2 or C1-C2 were fused to the EGF-HSA in the C-terminal position. Recombinant proteins of this disclosure, for example, FP330 promote αv-integrin-dependent cell adhesion similar to wtMFG-E8 if expressed in CHO cells or HEK cells (FIG. 5D).

Taken together, these data demonstrate that fusion proteins of the present disclosure bind to cellular integrins, support integrin-dependent cell adhesion and indicate that in proteins with a HSA domain insert, the C-terminal EGF-like domain may functionally profit from a C-terminally fused protein domain to support integrin binding.

### 3.3 Human Macrophage-Neutrophil Efferocytosis Assay

Human peripheral blood mononuclear cells (PBMCs) were isolated from buffy coat by means of Ficoll gradient centrifugation (Ficoll®-Paque PLUS, GE Healthcare, Sweden) followed by negative selection of monocytes using a Stemcell isolation kit (Stemcell 19059, Vancouver, Canada). Monocytes were differentiated to “M0” macrophages using recombinant human M-CSF 40 ng/mL (Macrophage Colony Stimulating Factor, R&D Systems, US) in RPMI 1640 containing 25 mM HEPES, 10 % FBS, Pen/Strep, 1 mM NaPyr, 50 µM β-Merc for 5 days. One day prior to efferocytosis, macrophages were labeled with PKH26 using the Red Fluorescent Dye Linker kit (Sigma MINI26, US). Cells were resuspended in RPMI 1640 containing 25 mM HEPES, 10% FBS, Pen/Strep, 1 mM NaPyr, 50 µM β-Merc and seeded into black 96-well plates (Corning,US) at 40000 cells/well and allowed to adhere for 20h.

Neutrophils: Human neutrophils were isolated from buffy coats by dextran sedimentation in combination with a Ficoll™ density gradient as follows: Plasma of the buffy coat was removed by centrifugation of the diluted buffy coat. Cellular harvest was diluted in 1% dextran (from Leuconostoc spp. MW 450.000-650.000; Sigma, US) and allowed to sediment on ice for 20-30 min.

Leukocytes from supernatant were harvested and on a Ficoll™-Paque layer (GE Healthcare Sweden). After centrifugation the pellet was harvested and remaining erythrocytes were lysed using red blood cell (RBC) lysis buffer (BioConcept, Switzerland). Neutrophils were washed once in medium (RPMI 1640+GlutaMax containing 25 mM HEPES, 10% FBS, Pen/Strep, 0.1 mM NaPyr, 50 uM b-Merc) and kept overnight at 15° C. Apoptosis/cell death was induced by treatment of neutrophils with 1 µg/mL Superfas Ligand (Enzo Life Sciences, Lausanne, Switzerland) at 37° C. for 3h. Neutrophils were stained with both Hoechst 33342 (Life technologies, US) for 25 min and with DRAQ5 (eBioscience, UK, diluted 1:2000) at 37° C. in the dark for 5 min.

### Efferocytosis Assay

M0 macrophages were incubated with the fusion proteins for 30 min. Apoptotic labelled neutrophils were added at a ratio of M0/neutrophil 1:4. Efferocytosis of apoptotic neutrophils by macrophages was visualized taking advantage of the fluorescence intensity increase of DRAQ5 upon localization of neutrophils in the pH-low lysosomal compartment of M0 macrophages.

Efferocytosis was quantified using an ImageXpress Micro XLS wide field high-content analysis system (Molecular DEVICES. CA, US). Macrophages were identified via PKH26 fluorescence. The efferocytosis index (El, displayed as %) was calculated as the ratio of macrophages containing at least one ingested apoptotic neutrophil (DRAQ5high) event to the total number of macrophages. Data analysis was performed using MS Excel and GraphPad Prism software.

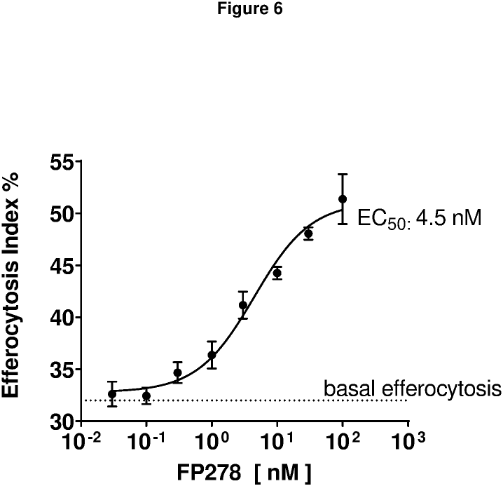

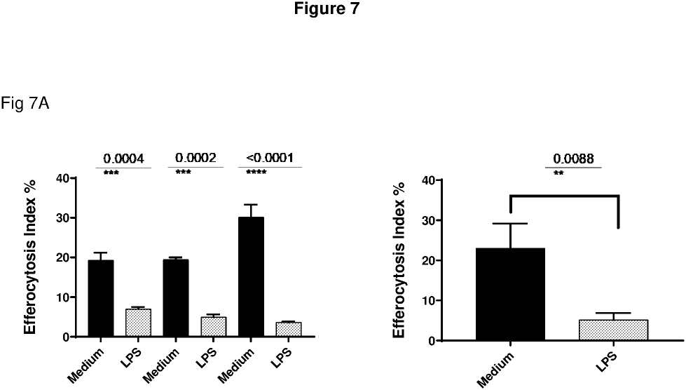

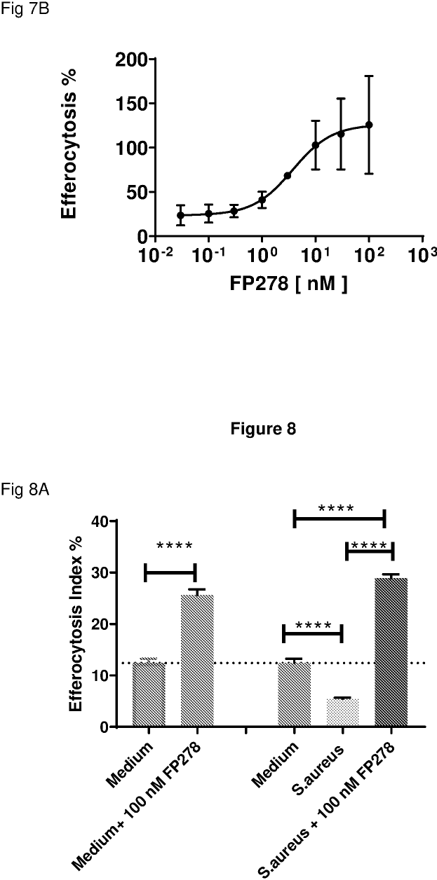

The effect of the fusion protein FP278 (EGF-HSA-C1-C2-His tag; SEQ ID NO: 44) on the promotion of efferocytosis of dying neutrophils by human macrophages is shown in FIG. 6. The fusion proteins increase internalization of pHrodo-labelled dying human neutrophils into macrophages over the already high efferocytosis capacity of M0 macrophages, shown as the basal level. In FIGS. 7 it is shown that recombinant fusion protein FP278 can rescue endotoxin (lipopolysaccharide)-impaired efferocytosis of dying neutrophils by human macrophages. FIG. 7A shows the impairment of macrophage efferocytosis of dying human neutrophils by 100 pg/ml lipopolysaccharide (LPS) in three human donors. The left panel shows the individual donor response, the right panel shows the mean impairment of efferocytosis (%) of the three donors. FIG. 7B shows the rescue of this endotoxin (LPS)- impaired efferocytosis of dying neutrophils by human macrophages with the fusion protein FP278.

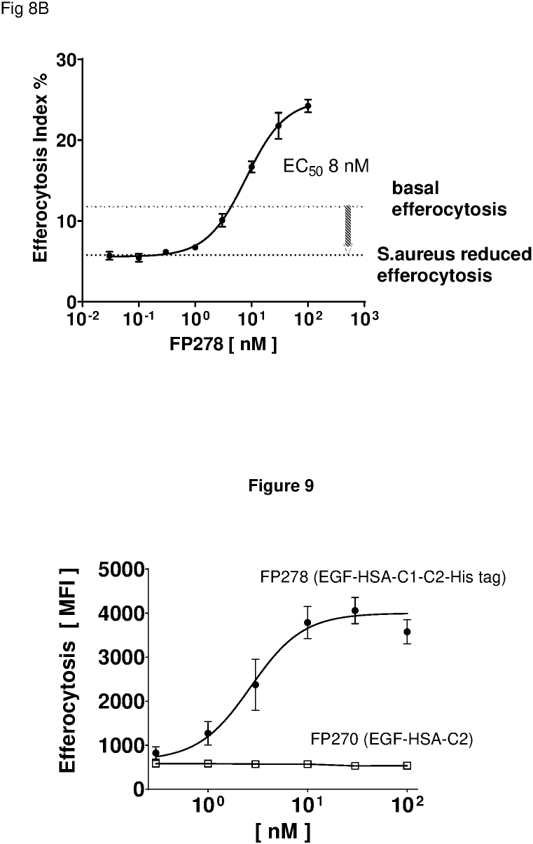

The rescue of S. aureus particle impaired efferocytosis of dying neutrophils by human macrophages with the fusion protein FP330 is shown in FIGS. 8. FIG. 8A shows the effect of a concentration of 100 nM of fusion protein on promoting efferocytosis over the base level (dotted line; left-hand part of figure) as well as the effect of 100 nM fusion protein in rescuing the impairment of efferocytosis caused by the addition of S. aureus (right-hand part of figure). FIG. 8B shows the effect of increasing concentrations of fusion protein FP278 (EC₅₀ 8 nM) on the rescue of impaired efferocytosis caused by the addition of S. aureus, and on the promotion of efferocytosis once the base levels of efferocytosis had been reached.

### 3.4 Human Endothelial - Jurkat Efferocytosis Assay Cell Culture

Human umbilical vein endothelial cells (HUVECs) were obtained from Lonza (Basel, Switzerland). Cells were cultivated in flasks coated with gelatin (from bovine skin, 0.2% final concentration in PBS, dilution of 2% stock solution, Sigma, Germany). Cells were grown with culture medium 199 (Thermo Fischer Scientific, US) supplemented with 10% FBS (GE Healthcare, United Kingdom), 1% Pen/Strep (Thermo Fischer Scientific, US), 1% Glutamax (Thermo Fischer Scientific, US) and 1 ng/mL recombinant Fibroblast Growth Factor-basic (Peprotech, UK). Cells were detached for harvesting or passaging using Accutase™ (Thermo Fischer Scientific, US).

Jurkat E6-1 cells were obtained from ATCC (American Type Culture Collection, US) and grown in culture medium RPMI 1640 (Thermo Fischer Scientific, US) supplemented with 10% FBS (GE Healthcare, UK), 1% Pen/Strep (Thermo Fischer Scientific, US), 10 mM Sodium Pyruvate (Thermo Fischer Scientific, US) and 10 mM HEPES (4-(2-hydroxyethyl)-1-piperazineethanesulfonic acid, Thermo Fischer Scientific, US).

Apoptosis of Jurkat E6-1 cells was induced using recombinant human TRAIL (R&D Systems, US). Apoptotic cells were labeled with pHrodo™ Green STP ester dye (Thermo Fischer Scientific, US). Flow cytometry buffer was prepared with PBS (Thermo Fischer Scientific, US) supplemented with 1 % FBS (GE Healthcare, United Kingdom), 0.05% w/v sodium azide (Merck, Germany) and 0.5 mM EDTA (Ethylenediaminetetraacetic acid, Thermo Fischer Scientific, US).

### Efferocytosis Assay

At day 1, HUVECs (confluence 70-90%) were harvested by detachment with Accutase™ for 5 minutes washed with PBS and re-suspended in cell culture medium. Cell numbers and viability were assessed using a Guava EasyCyte flow cytometer (Merck, Germany) and the Guava ViaCount reagent (Merck, Germany) according to manufacturer’s instructions. Required amount of cells were centrifuged at 300xg for 5 min at RT and re-suspended in culture medium to allow a cell number of 6.6×10⁴ cells/mL. 150 µL/well of this cell suspension was added to 96-well tissue culture plates (Corning™, US). HUVECs were incubated in incubator at 37° C. / 5% CO₂ / 95% humidity for additional 16-20 hours.

Jurkat E6-1 cell numbers and viability/cell death status were assessed using a Guava EasyCyte flow cytometer (Merck, Germany) and the Guava ViaCount reagent (Merck, Germany) according to manufacturer’s instructions. Required amount of cells were centrifuged at 300xg for 5 min at RT and re-suspended at a density of 1×10⁶ cells/mL in culture medium supplemented with recombinant human TRAIL at a final concentration of 50 ng/mL. Cell death was induced at 37° C. / 5% CO₂ / 95% humidity over-night.

At day 2, medium was removed from HUVECs by aspiration and 25 µL of fresh pre-warmed (37° C.) culture medium added, followed by the addition of 25 µL fusion protein or controls diluted in pre-warmed (37° C.) culture medium. For dilution non-binding surface (NBS) treated 96-well plates (Corning™, US) were used. The fusion proteins were allowed to interact with HUVECs for 30 min at 37° C. / 5% CO₂ / 95% humidity before addition of dying Jurkat cells.

Apoptotic/dying Jurkat E6-1 cell numbers were counted using a Guava EasyCyte flow cytometer (Merck, Germany) and the Guava ViaCount reagent (Merck, Germany). The required amount of apoptotic cells were centrifuged at 400xg at RT for 5 min and re-suspended at a density of 5×10⁶ cells/mL in RPMI 1640 medium (no FBS) supplemented with pHrodo™ Green STP ester dye at a final concentration of 5 µg/mL (Staining medium). After staining for 10 min at 37° C. remaining reactive pHrodo™ Green STP ester was inactivated with staining medium supplemented with 10% FBS for additional 5 min at 37° C. pHrodo™ Green labelled cells were washed once and cell number was adjusted to 3×10⁶ cells/mL in HUVEC culture medium. 1.5×10⁶ /well pHrodo™ Green labeled Jurkat cells were added to HUVECs and incubated at 37° C. / 5% CO₂ / 95% humidity for 5 h. Medium was removed, HUVECs were washed once in PBS and detached by 40 µL/well of Accutase™ solution. Cells were harvested by addition of 80 µL of ice-cold flow cytometry buffer, transferred to a 1.5 mL polypropylene 96-well block, washed with an excess of ice-cold flow cytometry buffer and centrifuged at 400xg (4° C.) for 5 min. Supernatants were removed by aspiration and pellets were re-suspended in 80 µL ice-cold flow cytometry buffer and transferred in 96- well V-bottom microtiter plate (BD Biosciences, US). Samples were then measured on a BD LSRFortessa™ flow cytometer (BD Biosciences, US). pHrodo™ Green fluorescence intensity, as an indicator of lysosomal localization of engulfed Jurkat cells, was recorded. Flow cytometry data analysis was performed on using FlowJo™ software. The median fluorescence intensity (MFI) values of pHrodo™ Green signal from singlet-gated HUVECs was used as readout. Data analysis was performed using MS Excel and GraphPad Prism software for EC₅₀ calculation.

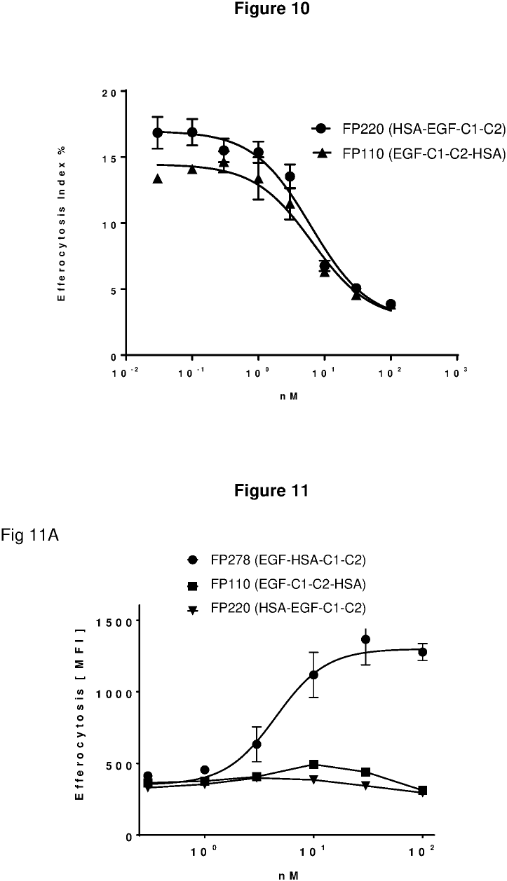

The effect of the fusion proteins FP278 (EGF-HSA-C1-C2-His tag; SEQ ID NO: 44) and FP270 (EGF-HSA-C2; SEQ ID NO: 36) on the promotion of efferocytosis of dying Jurkat cells by HUVEC endothelial cells is shown in FIG. 9. The internalization of pHrodo-labelled dying human Jurkat T cells by HUVECs is potently promoted by the fusion protein FP278. Results demonstrate that endothelial cells are armed by the fusion protein to become efficient phagocytes of dying cells. Surprisingly, the efficacy of the fusion proteins in this assay clearly depends on the presence of a C1-C2 or C1-C1 tandem domain. A fusion protein consisting of EGF-HSA-C2 (FP270), for example is inactive in this experimental setting, as shown in FIG. 9. FIG. 10 demonstrates our highly surprising finding that the location of an HSA domain in the engineered proteins, namely in the N-or C-terminal position (HSA-EGF-C1-C2 (FP220; SEQ ID NO: 30) or EGF-C1-C2-HSA (FP110; SEQ ID NO: 28), respectively), confers efferocytosis blocking ability in the macrophage efferocytosis assay to the MFG-E8 HSA engineered proteins. These data clearly demonstrate the importance to position the HSA domain between the integrin binding and the PS-binding domains for efficient promotion of efferocytosis by the fusion proteins of the present disclosure.

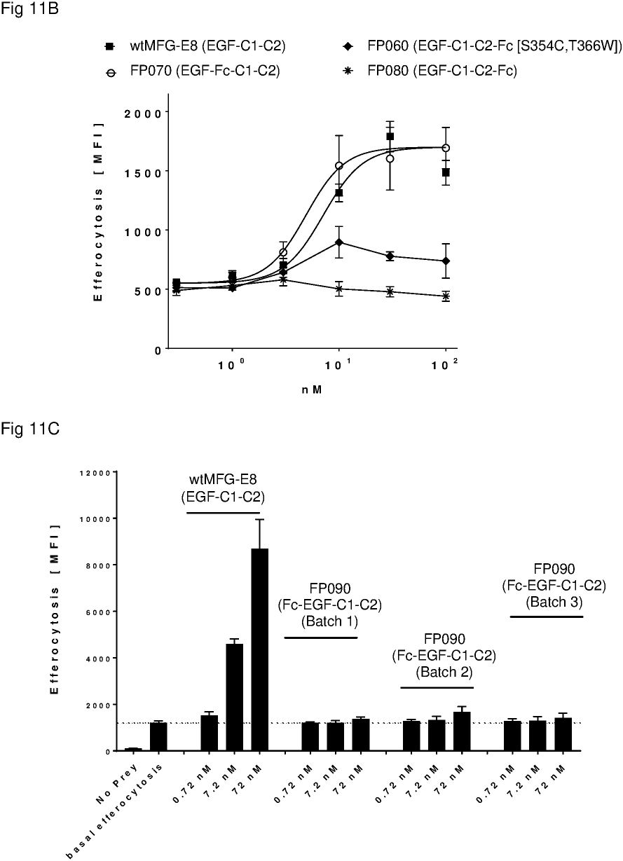

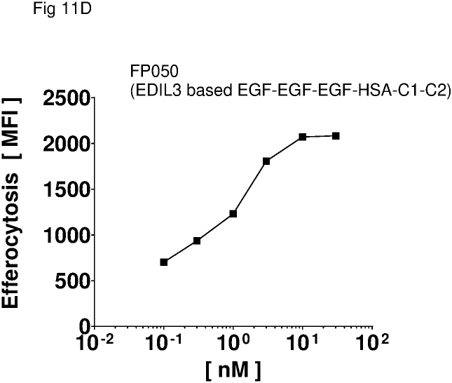

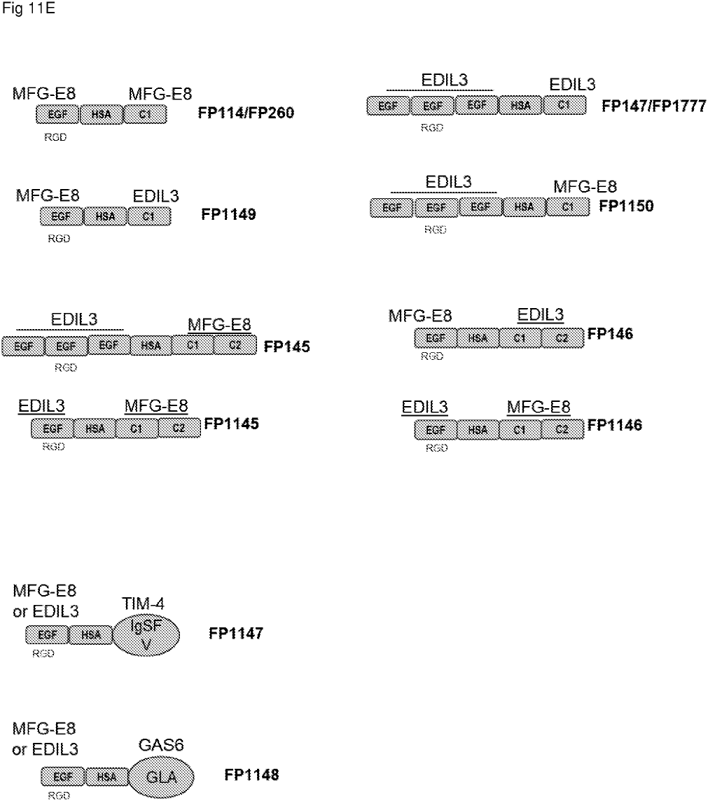

FIGS. 11 shows a comparison of the promotion of endothelial efferocytosis by various formats of fusion proteins comprising combinations of an EGF domain, a C1-C2 domain, HSA or a Fc domain. FIG. 11A shows a comparison of fusion proteins comprising HSA with the HSA positioned at the C-terminal or N-terminal or between the EGF-like and C1-C2 domains; EGF-C1-C2-HSA (FP110; SEQ ID NO: 28), HSA-EGF-C1-C2 (FP220; SEQ ID NO: 30) and EGF-HSA-C1-C2-His tag (FP278; SEQ ID NO: 44), respectively. FIG. 11B shows a comparison of fusion proteins comprising a Fc domain with the Fc positioned at the C-terminal or between the EGF-like and C1 domains. Two formats of Fc moiety are shown: wild type Fc (SEQ ID NO: 7) as found in FP070 (EGF-Fc-C1-C2; SEQ ID NO: 17) and FP080 (EGF-C1-C2-Fc; SEQ ID NO: 22) and Fc moieties with the KiH modifications S354C and T366W on one arm of the Fc (FP060; EGF-C1-C2-Fc [S354C, T366W]; SEQ ID NO: 14) EU numbering (Merchant et al (1998) supra). FIG. 11C shows a comparison of the fusion proteins FP090 (Fc-EGF-C1-C2; SEQ Id NO: 24) comprising a Fc moiety positioned at the N-terminal, for three batches of FP090 at three different concentrations (0.72, 7.2 and 72 nM) compared to wtMFG-E8 control. Efferocytosis of dying Jurkat cells by HUVECs was only promoted by engineered proteins with a HSA or Fc moiety inserted after the EGF-like domain. FIG. 11D shows that the insert of a solubilizing domain can lead to a novel bioactive fusion protein based on the endogenous bridging protein EDIL3, a paralogue of MFG-E8. As shown in FIG. 11D, HSA was inserted between the EGF-like domain and the C1-C2 domain of EDIL3, the paralogue of MFG-E8. This EDIL3 construct (FP050 (EDIL3 based EGF-HSA-C1-C2; SEQ ID NO: 12) has only one (RGD loop-containing) of the 3 EGF-like domains that are found in wtEDIL3. In this construct we surprisingly found a similar toleration of the HSA domain insert with regards to expression of a novel recombinant engineered protein with very high purity (FIG. 2B). In addition it was found surprisingly, that the EDIL3-derived recombinant engineered protein FP050 promoted efferocytosis of dying Jurkat cells by endothelial cells (HUVECS) demonstrating core functionality of a bridging protein and exemplifying that the domains of bridging proteins are useful to design functional novel recombinant engineered proteins.

### Example 4: Efferocytosis of Prothrombotic Plasma Microparticles

### 4.1 Human Endothelial-Microparticle Efferocytosis Assay Cell Culture

HUVEC cells were obtained from Lonza (Basel, Switzerland). Cells were cultured in flasks coated with gelatin (from bovine skin, 0.2% final concentration in PBS, dilution of 2 % stock solution, Sigma Aldrich/Merck, Germany). Cells were grown with culture medium 199 (Thermo Fischer Scientific, US) supplemented with 10% FBS (GE Healthcare, United Kingdom), 1% Pen/Strep (Thermo Fischer Scientific, US), 1% Glutamax (Thermo Fischer Scientific, US) and 1 ng/mL recombinant Fibroblast Growth Factor-basic (Peprotech, United Kingdom). Cells were detached for harvesting or passaging using Accutase™ (Thermo Fischer Scientific, US).

Platelet-derived microparticles were prepared according to following procedure: citrated venous blood was collected (Coagulation 9NC Citrate Monovette, Sarstedt, Germany) from healthy adult volunteers after granted written informed consent. Platelet rich plasma (PRP) was prepared by centrifugation (200xg, 15 minutes, no brake, room temperature). Platelet-derived microparticles/debris were generated by subjecting the PRP to three snap / freeze cycles using liquid nitrogen and thaws at 37° C. Platelet fragments/ microparticles were pelleted by centrifugation at 20′000xg for 15 min RT. The pellet was re-suspended in PBS, aliquots were prepared and stored at -80° C. Microparticle preparations were 85-100% PS positive as determined by flow cytometry using Alexa Fluor™ 488-labeled murine MFG-E8/lactadherin (Novartis in-house). Numbers of microparticles were determined using dedicated counting beads (BioCytex / Stago, France). Flow cytometry buffer was prepared with PBS (Thermo Fischer Scientific, US) supplemented with 1 % FBS (GE Healthcare, United Kingdom), 0.05% w/v sodium azide (Merck, Germany) and 0.5 mM EDTA (Ethylenediaminetetraacetic acid, Thermo Fischer Scientific, US).

### 4.2 Efferocytosis Assay

At day 1, HUVEC cells (confluence 70-90%) were harvested by detachment with Accutase™ for 5 min washed with PBS and re-suspended in cell culture medium. Cell numbers and viability were assessed using a Guava EasyCyte flow cytometer (Merck, Germany) and the Guava ViaCount reagent (Merck, Germany) according to manufacturer’s instructions. Required amount of cells were centrifuged at 300xg for 5 min at RT and re-suspended in culture medium to allow a cell number of 6.6×10⁴ cells/mL. 150 µL/well of this cell suspension was added to 96-well tissue culture plates (Corning™, US). HUVEC cells were incubated in incubator at 37° C. / 5% CO₂ / 95% humidity for additional 16-20 hours.

At day 2, medium was removed from HUVEC cells by aspiration and 25 µL of fresh pre-warmed (37° C.) culture medium added, followed by the addition of 25 µL of the fusion protein FP278 (EGF-HSA-C1-C2-His tag; SEQ ID NO: 44) at three different concentrations: 0.3 nM, 3 nM or 30 nM or control, diluted in pre-warmed (37° C.) culture medium. For dilution non-binding surface (NBS) treated 96-well plates (Corning™, US) were used. The test proteins were allowed to interact with HUVEC cells at 37° C. / 5% CO₂ / 95% humidity for 30 min before addition of platelet-derived microparticles.

Required amount of microparticles were centrifuged for at 20′000xg at 4° C. for 15 min and re-suspended at density of 2×10⁸ particles/mL in RPMI 1640 medium (no FBS) supplemented with pHrodo™ Green STP Ester dye at a final concentration of 5 µg/mL (Staining medium). After staining for 10 min at 37° C. remaining reactive pHrodo™ Green STP ester was inactivated with staining medium supplemented with 10% FBS for additional 5 min at 37° C. pHrodo™ Green labelled microparticles were washed once by centrifugation at 20′000xg at 4° C. for 15 min and number was adjusted to 1×10⁸ particles /mL in HUVEC cell culture medium. 5×10⁶ particles/well pHrodo™ Green labeled microparticles were added to HUVEC cells and incubated at 37° C. / 5% CO₂ / 95% humidity for 5 h. Medium was removed, HUVEC cells were washed once in PBS and detached by 40 µL/well of Accutase™ solution. Cells were harvested by addition 80 µL of ice-cold flow cytometry buffer, transferred to a 1.5 mL polypropylene 96-well block, washed with an excess of ice-cold flow cytometry buffer and centrifuged at 400xg (4° C.) for 5 min. Supernatants were removed by aspiration and pellets were re-suspended in 80 µL ice-cold flow cytometry buffer and transferred in 96-well V-bottom microtiter plate (BD Biosciences, US). Samples were measured on a BD LSRFortessa™ flow cytometer (BD Biosciences, US). pHrodo™ Green fluorescence intensity, as an indicator of lysosomal localization of engulfed microparticles, was recorded. Flow cytometry data analysis was performed on using FlowJo™ software. The median fluorescence intensity values (MFI) of pHrodo™ Green signal from singlet-gated HUVEC cells was used as readout. Data analysis was performed using MS Excel and GraphPad Prism software for EC₅₀ calculation. The fusion protein FP278 promoted efferocytosis of platelet-derived microparticles by endothelial cells in a concentration-dependent manner as shown in FIG. 12. The promotion of uptake was concentration-dependent and was also observed in other types of endothelial cells (not shown).

### Example 5: Technical Properties of MFG-E8-HSA Fusion Proteins

### 5.1 Surface Plasmon Resonance (SPR) Binding Analysis of Fusion Protein FP330 to FcRn

A direct binding assay was performed to characterize the binding of the fusion protein FP330 (EGF-HSA-C1-C2; SEQ ID NO: 42) to FcRn. Kinetic binding affinity constants (KD) were measured on captured protein using recombinant human FcRn as analyte. Measurements were conducted on a BIAcore® T200 (GE Healthcare, Glattbrugg, Switzerland) at room temperature and at pH 5.8 and 7.4, respectively. For affinity measurements, the proteins were diluted in 10 mM NaP, 150 mM NaCl, 0.05% Tween 20, pH5.8 and immobilized on the flow cells of a CM5 research grade sensor chip (GE Healthcare, ref BR-1000-14) using standard procedure according to the manufacturer’s recommendation (GE Healthcare). To serve as reference, one flow cell was blank immobilized. Binding data were acquired by subsequent injection of analyte dilutions in series on the reference and measuring flow cell. Zero concentration samples (running buffer only) were included to allow double referencing during data evaluation. For data evaluation, doubled referenced sensorgrams were used and dissociation constants (KD) analyzed.

The fusion protein FP330 binds to FcRn at pH 5.8 with an affinity of 1380 nM, whereas there was no binding observed at pH 7.4 (See Table 5 above). These results are in good agreement with wild type HSA (1000-2000 nM, at pH 5.8, data not shown).

### 5.2 Differential Scanning Calorimetry (DSC) of MFG-E8 and Variants

The thermal stability of engineered MFG-E8 protein variant FP278 (EGF-HSA-C1-C2-His tag; SEQ ID NO: 44) was measured using differential scanning calorimetry. Measurements were carried out on a differential scanning micro calorimeter (Nano DSC, TA instruments). The cell volume was 0.5ml and the heating rate was 1° C./min. The protein was used at a concentration of 1 mg/ml in PBS (pH 7.4). The molar heat capacity of the protein was estimated by comparison with duplicate samples containing identical buffer from which the protein had been omitted. The partial molar heat capacities and melting curves were analysed using standard procedure. Thermograms were baseline corrected and concentration normalized. Two melting events were observed, first Tm was at 50° C., the second Tm at 64° C.

### 5.3 Aggregation Propensity and Solubility Measurements of MFG-E8 Variants

Firstly, the aggregation propensity of MFG-E8 variant protein FP278 (EGF-HSA-C1-C2-His tag; SEQ ID NO: 44) was measured by dynamic light scattering (DLS, Wyatt). Dynamic light scattering was applied to measure the translational diffusion coefficients of FP278 in solution by quantifying dynamic fluctuations in scattered light. Protein variant size distributions without fractionation, providing polydispersity estimates as well as hydrodynamic radii were measured at a concentration of 1 mg/ml. Hydrodynamic radii of the fusion protein FP278 were determined with a DynaPro™ plate reader (Wyatt Technology Europe GmbH, Dernbach, Germany) combined with the software DYNAMICS (version 7.1.0.25, Wyatt). 50 µL of the undiluted and filtered (0.22 µm PVDF-Filter (Millex® Syringe-driven Filter Unit, Millipore, Billerica, US)) protein solution was measured in a 384-well plate (384 round well plate, Polystyrol, Thermo Scientific, Langenselbold, Germany). Higher molecular weight aggregates of the protein sample could not be identified. The hydrodynamic radius of the protein was around 5-6 nm, indicating a monomeric protein in solution.

Secondly, concentration dependent hydrodynamic radius measurements of fusion protein FP278 were performed to estimate the solubility of the protein. Protein concentrations up to 22 mg/ml were applied. Hydrodynamic radii were determined as described above. Upon increasing concentration of the fusion protein FP278, no increase of the radius (5-7 nm) could be observed, whereas dynamic light scattering measurement of wtMFG-E8 (SEQ ID NO: 1) failed due to high aggregation at concentrations of around 0.2 mg/ml.

### Example 6: Optimization of MFG-E8 Fusion Proteins

Mass spectrometry (MS) was used to investigate the fusion protein FP330 (EGF-HSA-C1-C2) to generate a panel of variant MFG-E8 based fusion proteins optimized for improved expression and yield. A panel of variant proteins was generated with linkers of varying size and structure, for example, linkers comprising GS between the EGF and HSA domains and/or multiples of GS or G4S between the HSA and C1 domains. In addition, amino acid modifications (depicted as HSA* in Table 7) comprising deletions or substitutions were included in some of the variants. The panel of variant fusion proteins is summarized in Table 7 below.

TABLE 7

Summary of variant fusion proteins

| Variant | Domains | Amino acid modification¹ | Linker | SEQ ID NO: |
|---|---|---|---|---|
| wtMFG-E8 | EGF-C1-C2 | | | 1 |
| FP330 | EGF-GS-HSA-linker-C1-C2 | | (G₂S)₄ linker (SEQ ID 62) | 42 |
| FP278 | EGF-GS-HSA-linker-C1-C2-His tag | | (G₂S)₄ linker (SEQ ID 62) | 44 |
| FP811 | EGF-GS-HSA*-linker-C1-C2 | Deletion: G632 - L633 | G₄S (SEQ ID NO: 64) | 54 |
| FP010 | EGF-GS-HSA*-linker-C1-C2 | Deletion: G632 - L633 | (G₄S)₂ (SEQ ID NO: 65) | 56 |
| FP816 | EGF-HSA-C1-C2 | | | 58 |
| FP138 | EGF-GS-HSA*-linker-C1-C2 | Deletion: G632 - L633 | (G₂S)₄ linker (SEQ ID 62) | 52 |
| FP284 | EGF-GS-HSA*-linker-C1-C2 | Substitution L633V | (G₂S)₄ linker (SEQ ID 62) | 50 |
| FP776 | EGF-HSA*-C1-C2 | Deletion: A626 - L633 | | 48 |
| FP068 | EGF-HSA*-C1-C2 | Deletion: G632 - L633 | | 46 |

¹ Position of amino acid modification is numbered according to SEQ ID NO: 42 (FP330)

### Example 7: Variant MFG-E8 Fusion Proteins; Expression and Purification

Methods for generation of fusion proteins in HEK cell lines are described in Example 2. For expression in a proprietary CHO cell line, nucleic acids coding for MFG-E8 variants were synthesized at Geneart (LifeTechnologies) and cloned into a mammalian expression vector using restriction enzyme-ligation based cloning techniques. The resulting plasmids were transfected into CHO-S cells (Thermo). In brief, for transient expression of the fusion proteins, the expression vector was transfected into suspension-adapted CHO-S cells using ExpifectamineCHO transfecting agent (Thermo). Typically, 400 ml of cells in suspension at a density of 6 Mio cells per ml was transfected with DNA containing 400 µg of expression vector encoding the engineered protein. The recombinant expression vector was then introduced into the host cells for further secretion for seven days in culture medium (ExpiCHO expression media, supplemented with ExpiCHO feed and enhancer reagent (Thermo)).

As can be seen from the expression data shown in Table 8, the variant fusion proteins FP068 (SEQ ID NO: 46) and FP776 (SEQ ID NO: 48) showed an approximate two-fold improvement in expression over the fusion protein FP330 (SEQ ID NO: 42).

TABLE 8

Expression of variant fusion proteins in HEK and CHO* cell lines

| Protein | Expression post HSA capture (mg/l) |
|---|---|
| FP330 | 11 |
| FP138 | 10 |
| FP816 | 9 |
| FP068* | 18 |
| FP776* | 21 |
| FP284 | 10 |
| FP811 | 8 |
| FP010 | 10 |

* indicates fusion protein produced in a CHO cell line

Further therapeutic fusion proteins have been obtained according to the methods described Example 1. For example, expression levels (mg/I) obtained after full purification process (capture and polishing) are 4.3 for Seq ID 80 and 8.4 for Seq ID 82.

### Example 8: Characterization of Variant Fusion Proteins

The effect of the variant fusion proteins on efferocytosis was determined by performing efferocytosis assays as described in Example 3.

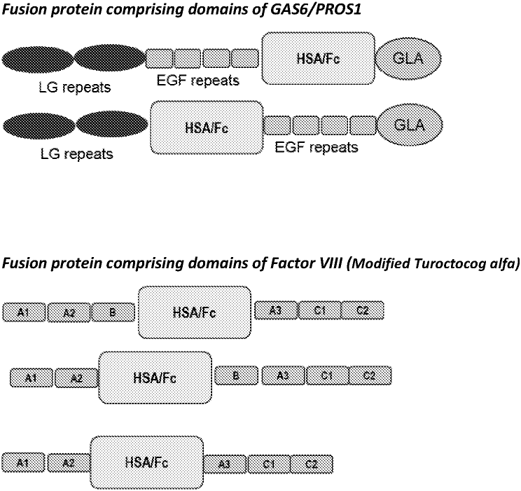

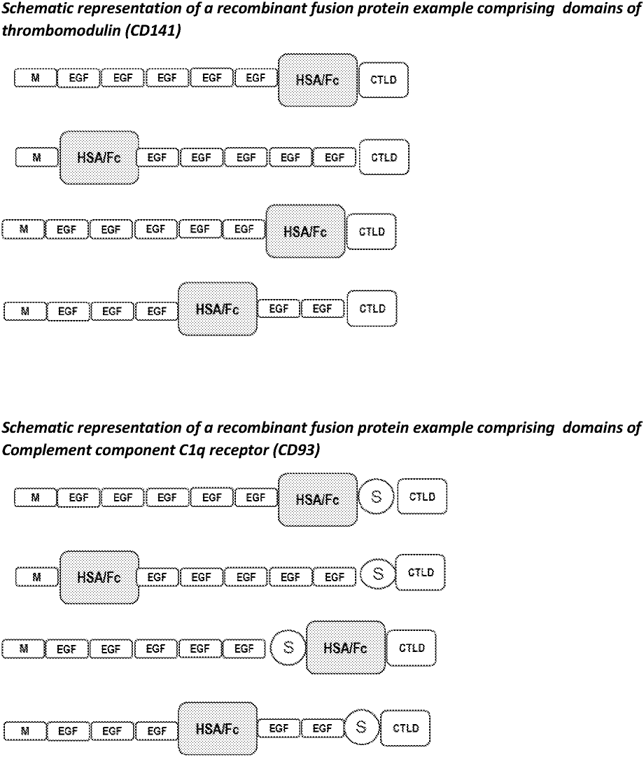

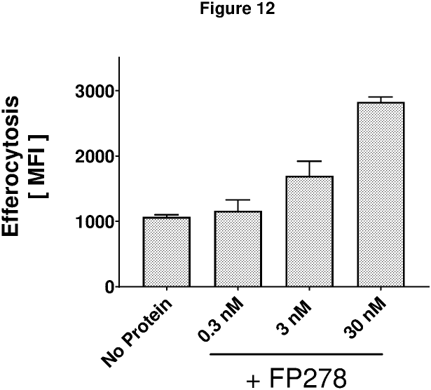

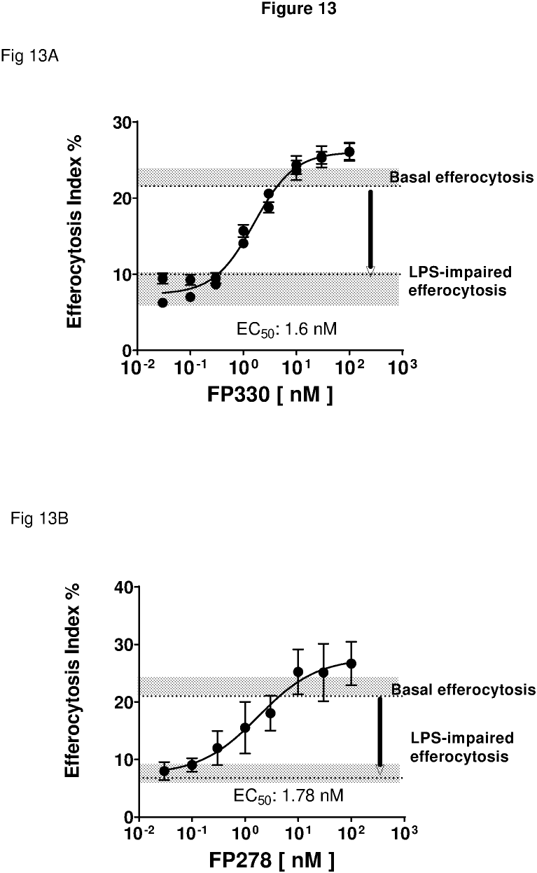

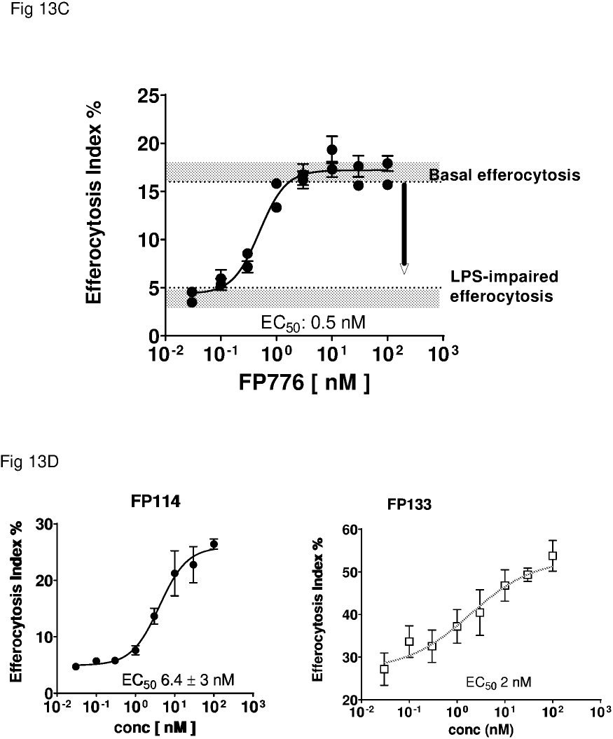

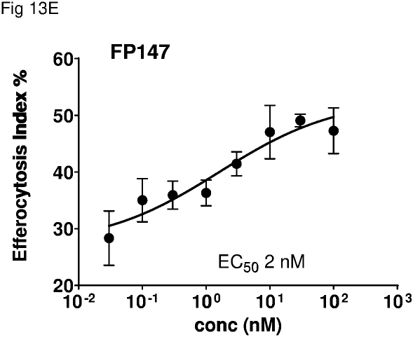

In a first assay, the effect of the variant fusion proteins in a human macrophage-neutrophil efferocytosis assay was determined according to the method described in Section 3.3 above. M0 macrophages were incubated with the fusion protein FP330 (EGF-HSA-C1-C2; SEQ ID No: 42) or variants FP278 (EGF-HSA-C1-C2-His tag; SEQ ID No: 44) or FP776 (EGF-HSA-C1-C2; SEQ ID No: 48) for 30 min. As shown in FIGS. 13, the fusion proteins FP330, FP278 and FP776 can rescue endotoxin (lipopolysaccharide (LPS))-impaired efferocytosis of dying neutrophils by human macrophages. Increasing concentrations of the fusion proteins FP330 (EC₅₀ = 1.6 nM; FIG. 13A), FP278 (EC₅₀ = 1.78 nM; FIG. 13B) and FP776 (EC₅₀ = 0.5 nM; FIG. 13C) led to rescue of impaired efferocytosis caused by the addition of LPS and even promoted efferocytosis once base levels had been reached.

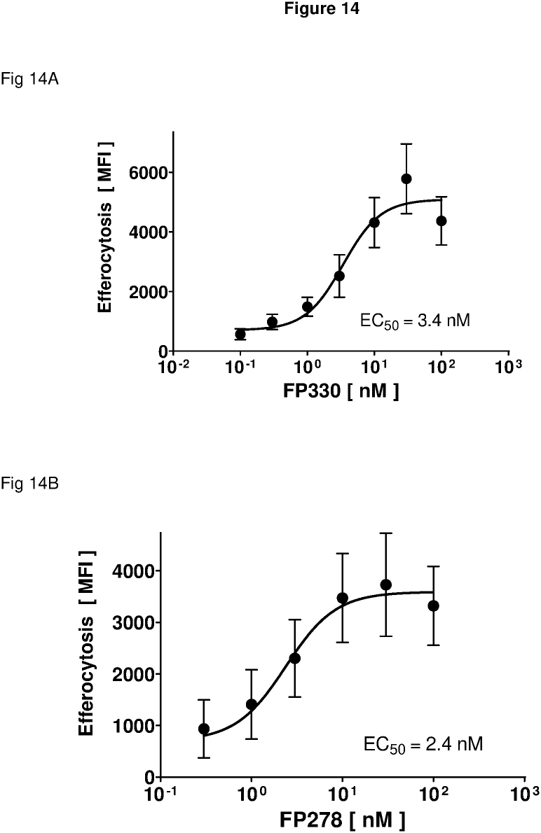

The fusion proteins FP330, FP278 and FP776 were further characterized in a human endothelial (HUVEC) cell- Jurkat cell efferocytosis assay according to the method described in Section 3.4 above. The effect of the fusion proteins FP330, FP278 and FP776 on the promotion of efferocytosis of dying Jurkat cells by HUVEC endothelial cells is shown in FIGS. 14. The internalization of pHrodo-labelled dying human Jurkat T cells by HUVECs was potently promoted by increasing concentrations of FP330 (EC₅₀ = 3.4 nM; FIG. 14A), FP278 (EC₅₀ = 2.4 nM; FIG. 14B) and FP776 (EC₅₀ = 3 nM; FIG. 14C). These results demonstrate that endothelial cells are armed by the fusion proteins to become efficient phagocytes of dying cells.

### Example 9: Protection of Mice From AKI and AKI-Triggered Acute Organ Response

### 9.1 Acute Kidney Injury Model

Female C57BL/6 mice (18-22 g) were purchased from Charles River (France) and housed in a temperature-controlled facility in filter-top-protected cages with 12-h light/dark cycles. Animals were handled in strict adherence to Swiss federal laws and the NIH Principles of Laboratory Animal Care. The therapeutic fusion protein under test was administered either intraperitonealy (i.p.) or intravenously (i.v.) two hours before surgery. Buprenorphine (Indivior Schweiz AG) was applied sub-cutaneously (s.c.) at a dose of 0.1 mg/kg 60 to 30 minutes before the surgery. The inhalation anesthesia with isoflurane was induced in a narcotic chamber (3.5-5 Vol. %, carrier gas: oxygen) for 5 min before surgery. During surgery, the animal was maintained under anesthesia via a face mask with 1-2 Vol% isoflurane /oxygen, the gas flow rate was 0.8- 1.2 I/min. The skin of the abdomen was shaved and disinfected with Betaseptic (Mundipharma, France). Animals were placed on a homeothermic blanket (Rothacher- Switzerland) with a homeothermic monitor system (PhysiTemp, US- Physitemp Instruments LLC, US) and covered by sterile gauze. The body temperature was monitored throughout the surgery by a rectal probe (Physitemp Instruments LLC, US) and controlled to allow a body temperature of 36.5-37.5° C. All animals including SHAM controls underwent unilateral nephrectomy of the right kidney: Following mid-line incision / laparotomy, abdominal content was retracted to the left to expose the right kidney. The right ureter and renal blood vessels were disconnected and ligated, the right kidney was then removed. For animals that underwent AKI, abdominal content was positioned to the right on sterile gauze and the left renal artery and vein were dissected to allow clamping for ischemia induction. A micro-aneurysm clamp (B Braun, Switzerland) was used to clamp the renal pedicle (artery and vein together using one clamp) to block blood flow to the kidney and to induce renal ischemia. Successful ischemia was confirmed by color change of the kidney from red to dark purple, which occurred in a few seconds. Following the ischemia induction (35-38 minutes), the micro-aneurysm clamp was removed. Warm sterile saline (~2 ml, 37° C.) was used for washing the abdominal contents to rehydrate tissues before closure of the wound. After the wash, an additional 1 ml of sterile saline was added i.p. as fluid replacement. When starting the reperfusion, the wound was closed in two layers (muscle and the skin, separately). The animals were then maintained under red warm lamp until fully recovered. Buprenorphine was administered again 1 h and 4 h after the surgery at a dose of 0.1 mg/kg and was also included into drinking water (9.091 µg/mL). After 24 h animals were euthanized for analysis.

### 9.2 Administration of Therapeutic Fusion Proteins

The therapeutic fusion proteins FP330 (EGF-HSA-C1-C2; SEQ ID No: 42), FP278 (EGF-HSA-C1-C2-His tag; SEQ ID No: 44) and FP776 (EGF-HSA-C1-C2; SEQ ID No: 48) were tested in the AKI model as described above at the doses set out in Table 9 below. For the studies to detect serum markers and qPCR marker expression, fusion protein FP278 was administered 2 hours before surgery. FP330 and FP776 were dosed i.v. 30 min before ischemia reperfusion injury onset. For the study to measure contrast agent uptake by magnetic resonance imaging, the fusion protein FP776 was dosed prophylactically 30 min before AKI induction at 1.26 mg/kg or dosed therapeutically 5 h post induction of ischemia reperfusion injury at 2 mg/kg i.v.

TABLE 9

Dosing of therapeutic fusion proteins

| Fusion protein | Dose (mg/kg) | Route of Administration |
|---|---|---|
| FP278 | 0.16 | i.p. |
| | 0.50 | |
| FP330 | 0.20 | i.v. |
| | 0.50 | |
| | 1.50 | |
| FP776 | 0.20 | i.v. |
| | 0.75 | |
| | 1.26 | |
| | 2.00 | |

### 9.3 Readouts/Analysis for AKI Protection Serum Markers

Serum samples were taken 24 h post ischemia reperfusion induction and analyzed for serum creatinine and blood urea nitrogen (BUN) content using a Hitachi M40 clinic analyzer according to manufacturer’s instruction (Axonlab, Switzerland).

### qPCR Marker Expression in Organs

Organs (kidney, liver, lung and heart) were harvested 24 h after AKI induction and were cut in 1 cm pieces and stored in RNA Later buffer (Thermo Fisher Scientific Inc, US) at 4° C. overnight. Organ pieces were transferred to RLT buffer (RNeasy Mini Kit, Qiagen, DE) containing 134 mM Beta-mercaptoethanol (Merck, DE) in Lysing Matrix D tubes (MP Biomedicals FR) and homogenized using the FastPrep-24 Instrument (MP Biomedicals). Heart fibrous tissue was subsequently digested with proteinase K (RNeasy Mini Kit), while kidney, liver and lung lysates were directly centrifuged for 3 min at full speed in a microcentrifuge (Eppendorf, DE). Supernatants were transferred onto a QIAshredder spin column (Qiagen, DE) and centrifuged for 2 min. RNA extraction of the flow-throughs was performed according to the RNeasy Mini Kit Manual, including DNase digestion. RNA concentration was measured with a Nano Drop 1000 device (Thermo Fisher Scientific Inc). 2 µg RNA per sample was reverse transcribed according to the High-Capacity cDNA Reverse Transcription Kit Manual (Thermo Fisher Scientific Inc) using a SimpliAmp Thermocycler (Applied Biosystems, US). cDNA was combined with Nuclease free water (Thermo Fisher Scientific Inc), TaqMan probe (TaqMan Gene Expression Assay (FAM), Thermo Fisher Scientific Inc) and TaqMan Gene Expression Master Mix (Thermo Fisher Scientific Inc) in a 384-well microplate (MicroAmp Optical 384-Well Reaction Plate, Thermo Fisher Scientific Inc). qPCR was performed on the ViiA 7 Real-Time PCR System (Applied Biosystems, US). Settings were 1: 2 min, 50° C.; 2: 10 min, 95° C.; 3: 15 s, 95° C.; 4: 1 min, 60° C. Steps 3 and 4 were repeated for 45 cycles. Data analysis was performed using the ViiA 7 Software, qPCR data analysis software were performed using MS Excel and GraphPad Prism software.

### Contrast Agent Uptake by the Liver as Measured by Magnetic Resonance Imaging (MRI)

The methods for performing the MRI were adapted from a publication by Egger et al (Egger et al., (2015) J Magn Reson Imaging, 41: 829-840). Experiments were performed on a 7-T Bruker Biospec MRI system (Bruker Biospin, Ettlingen, Germany). During MRI signal acquisitions, mice were placed in a supine position in a Plexiglas cradle. Body temperature was kept at 37±1° C. using a heating pad. Following a short period of induction, anesthesia was maintained with approx. 1.4% isoflurane in a mixture of O₂/N₂O (1:2), administered via a nose cone. All measurements were performed on spontaneously breathing animals; neither cardiac nor respiratory triggering was applied.

After placing a mouse in the scanner, scout fast images were acquired for localization purposes. Perfusion analyses were performed using an intravascular agent containing superparamagnetic iron oxide (SPIO) nanoparticles (Endorem®, Guerbet, France). Endorem® was injected intravenously as a bolus for 1.2 s into animals with AKI (at 24 h post disease induction) or after Sham operation (animals post 24 h nephrectomy). A first bolus was administered during 1.2 s, in conjunction with the sequential acquisition of echo-planar images at a resolution of 400 ms/image. Following the acquisition of 25 baseline images, a second bolus was injected during 1.2 s and a further 575 images were acquired after the bolus, resulting in a total of 600 images acquired in 4 min. The superparamagnetic contrast agent induced local changes in susceptibility which resulted in a signal attenuation proportional to the perfusion of the kidney. For a series of images, signal intensities were assessed on regions-of-interest (ROIs) located in the cortex/outer stripe of outer medulla. Position, shape, and size of the ROIs were carefully chosen in order to ensure that they covered approximately the same region, despite movements of the kidney caused by respiration. The mean signal intensities for the pre-injection images provided baseline intensities (S(0)). Perfusion indexes were determined from the mean values of the following ratios (Rosen et al., (1990) Magn Reson Med., 14: 249-265):

$$\text{-In} \left[\text{S} \left(\text{t}\right) \text{/S} \left(\text{0}\right)\right] ∼ \text{TE} \text{.} \text{V} \text{.} \text{cT} \left(\text{t}\right)$$

where TE is the echo time, V the blood volume, and cT the concentration of contrast agent.

The SPIO nanoparticles used in the study have a mean diameter of about 150 nm and are taken up by Kupffer cells in the liver. Therefore, in addition to kidney perfusion, MRI also allowed the uptake of the nanoparticles in the liver to be monitored, by detecting the contrast change assessed in ROIs placed in the liver.

### 9.4 Results

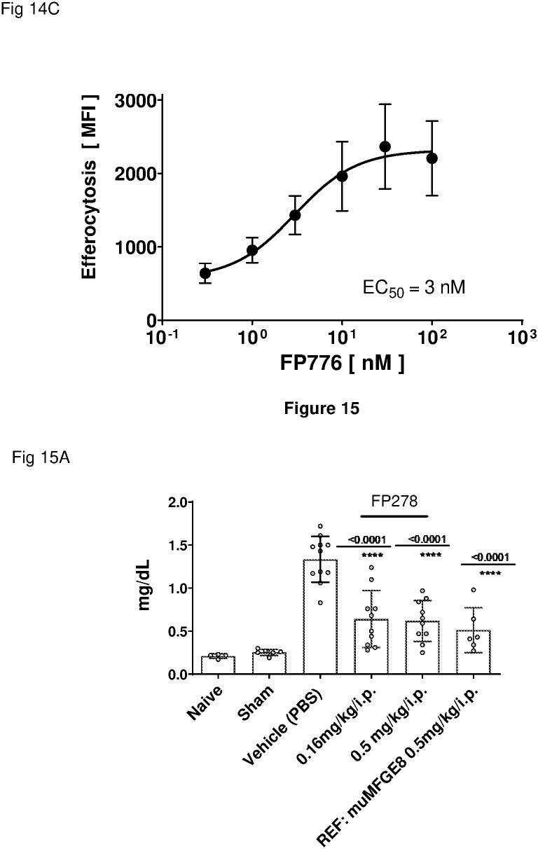

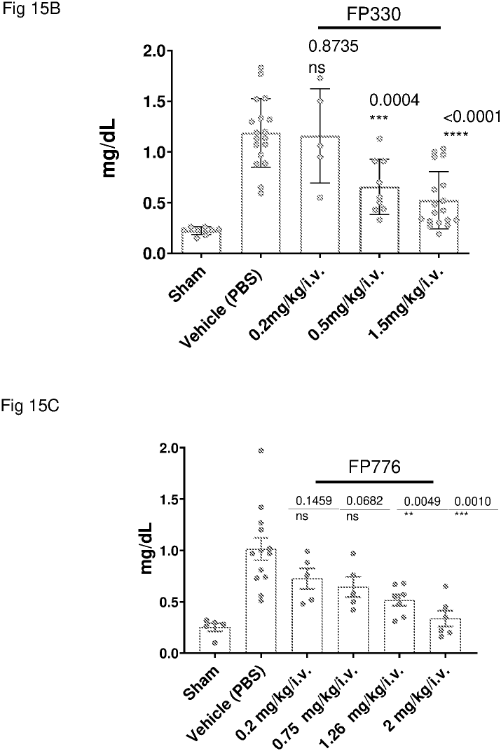

As shown in FIGS. 15, the fusion proteins FP330 (EGF-HSA-C1-C2; SEQ ID No: 42), FP278 (EGF-HSA-C1-C2-His tag; SEQ ID No: 44) and FP776 (EGF-HSA-C1-C2; SEQ ID No: 48) protected kidney function in this model of acute kidney injury (AKI) when administered either i.p. (FP278) or i.v. (FP330 and FP776). This protection is reflected by the block of serum creatinine rise (sCr). FIG. 15A shows that the fusion protein FP278 at both doses tested reduced serum creatinine levels significantly (p<0.0001) compared to vehicle treated animals and as effectively as murine MFG-E8. As shown in FIG. 15B, fusion protein FP330 protected kidney function in a dose dependent manner and likewise for fusion protein FP776 (FIG. 15C), where serum creatinine levels were also blocked in a dose dependent manner.

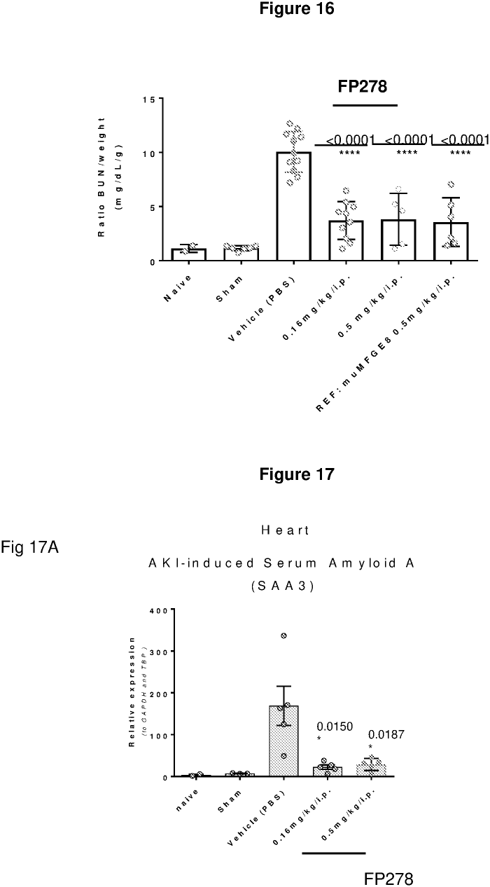

Impaired kidney function is also reflected in blood urea nitrogen (BUN) levels in the mice tested and the effect of the fusion protein FP278 on BUN levels is shown in FIG. 16.

In summary, as shown in FIGS. 15 and 16, the fusion proteins FP278, FP330 and FP776 potently protected against a raise of these markers used to clinically diagnose kidney failure. The observed efficacy was confirmed by histology (not shown).

Furthermore, as shown in FIGS. 17 a single dose of the fusion protein FP278 protects distant organs from acute phase response elicited by AKI. AKI induces a plethora of mRNA responses measurable by qPCR in lysates of distant highly perfused organs such as the spleen, lung liver heart and brain. Typical mRNAs induced selected damage (NGAL, KIM-1), induction of chemokines (not shown) or induction of acute phase response protein induction such as serum amyloid A (SAA). FIGS. 17A and 17B exemplify such AKI-induced response (serum amyloid A (SAA)) in the murine heart and lung which was potently blocked and returned to SHAM levels after a single injection of the fusion protein.

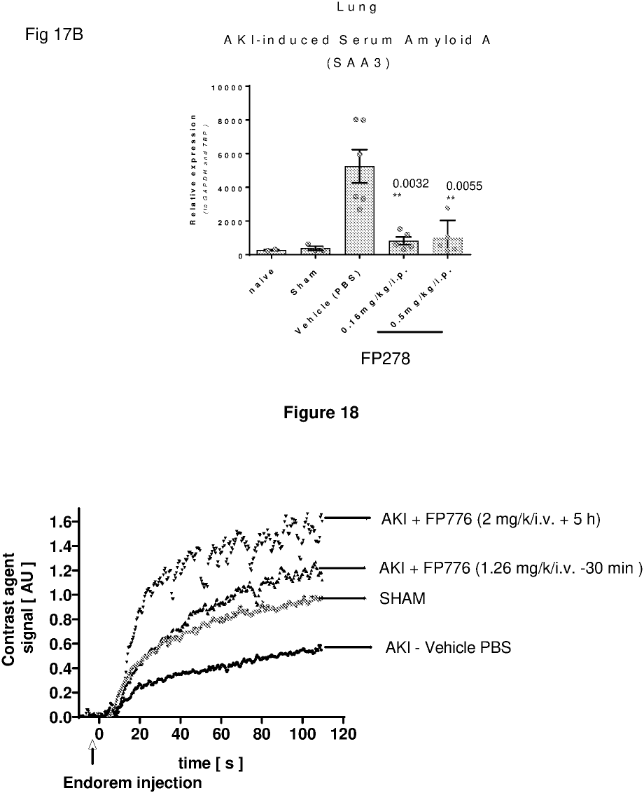

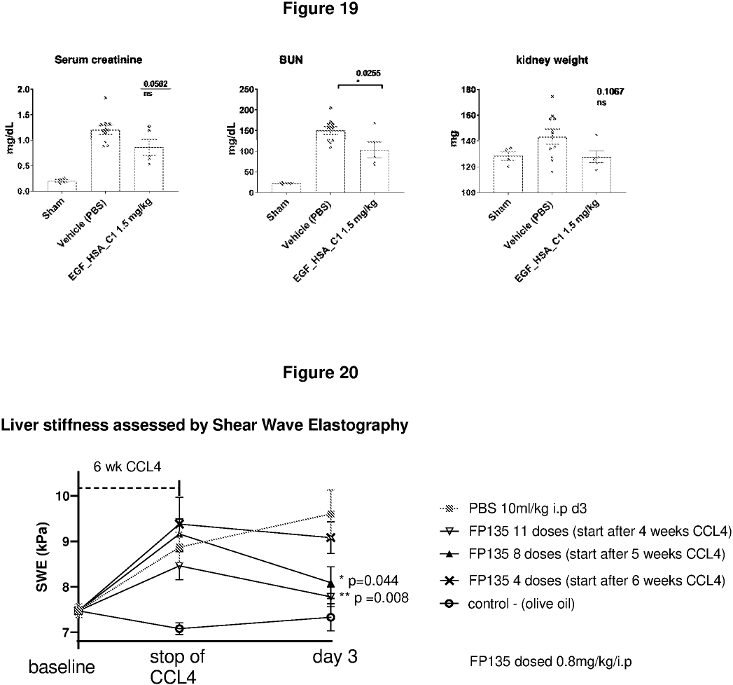

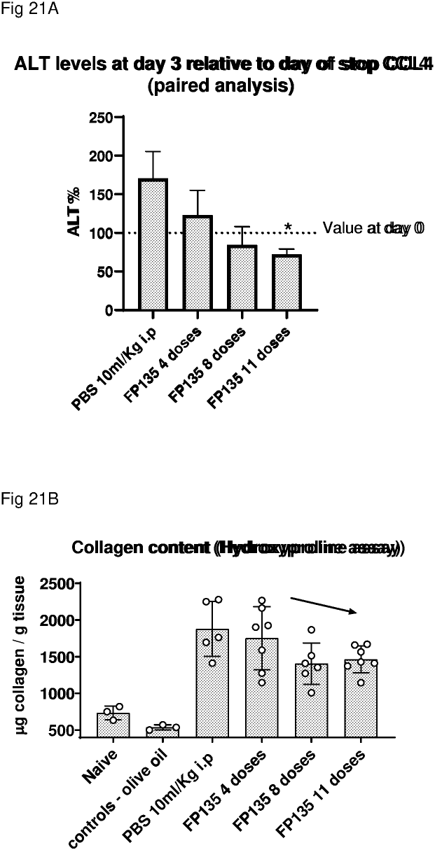

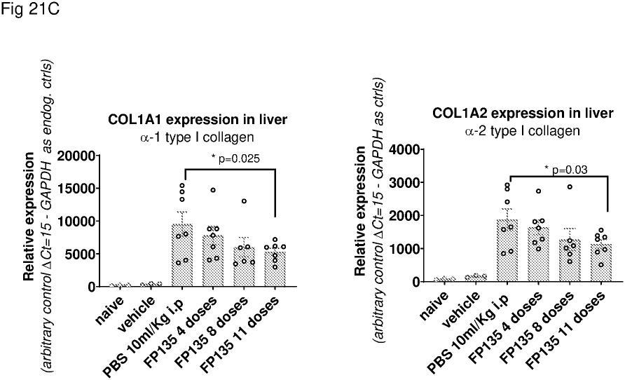

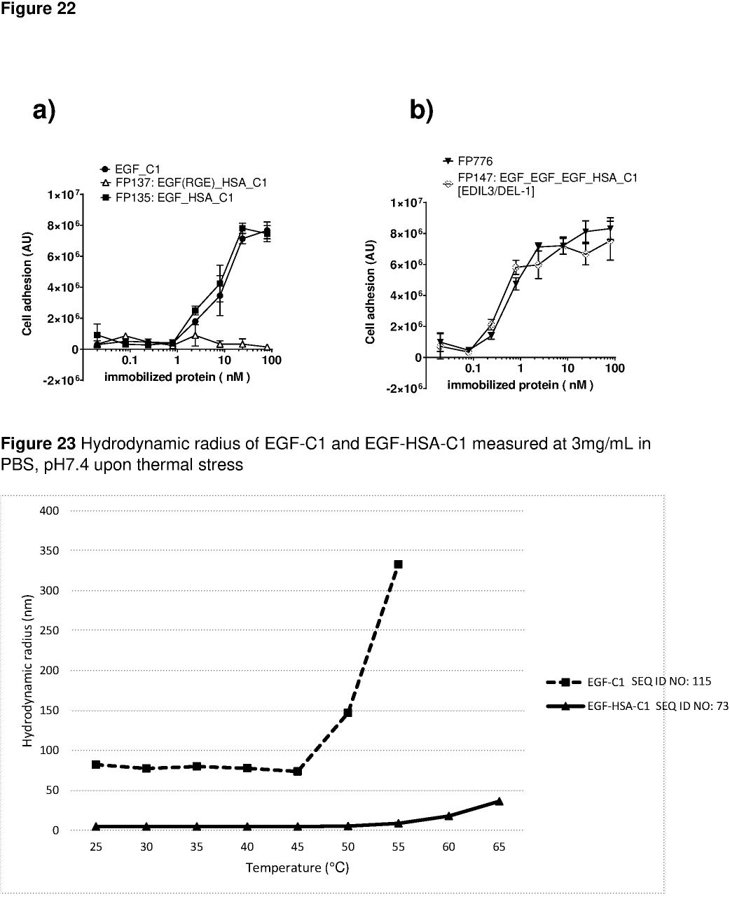

The uptake of the SPIO contrast agent Endorem® by the liver over time is shown in FIG. 18. Animals with AKI showed significantly reduced uptake of the contrast agent by the liver (target = Kupffer cells) compared to Sham animals. FP776 treatment (dosed prophylactically at 1.26 mg/kg, -30 min before AKI induction, or dosed therapeutically at 2 mg/kg, +5 h post ischemia reperfusion injury induction) protected from the loss of contrast agent accumulation in the liver of AKI mice. These results suggest that in this mouse model, AKI triggers a significant impairment of endogenous Kupffer cell-mediated clearance of particulate and that AKI causes microvascular disturbance which impacts on the accumulation of iron particle contrast agent in the liver. Treatment with fusion protein FP776 protected from loss of clearance and from microvascular disturbance, and even boosted the uptake of the contrast agent at both doses tested, when compared to sham animals.

### Examples 10: Characterization of MFG-E8-HSA Engineered Proteins

### 10.2 αV Integrin Adhesion Assay

Fusion proteins were diluted in phosphate buffered saline (PBS) pH 7.4 and 50 µL of the indicated concentration was immobilized by adsorption (96 well plate, Nunc Maxisorb) overnight. The plates were subsequently treated with PBS containing 3% fatty acid free bovine serum albumin (BSA) at RT for 1.5h. αvβ3 integrin- expressing lymphoma cells (ATCC-TIB-48 BW5147.G.1.4, ATCC, US) were cultivated in RPMI 1640 supplemented with GlutaMax, 25 mM HEPES, 10% FBS, Pen/Strep, 1 mM NaPyruvate, 50 µM β-Mercaptoethanol. Cells were labelled with 3 µg/mL 2′,7′-bis-(2carboxyethyl)-5-(and-6)-carboxyfluorescein, acetoxymethyl ester (BCECF AM) (Thermo Fisher Scientific Inc, US) for 30 min. BW5147.G.1.4 cells were resuspended in adhesion buffer (TBS, 0.5% BSA, 1 mM MnCI2, pH 7.4) and 50000 cells/well were allowed to adhere at RT for 40 min. Non-adherent cells were removed by manual washes with adhesion buffer. Fluorescence of adherent cells was quantified using an Envision^{TM} 2103 multilabel plate reader, Perkin Elmer, US. Data analysis was performed using MS Excel and GraphPad Prism software.

Adhesion of BW5147.G.1.4 cells to immobilized EGF-like domain containing fusion proteins. This finding suggests that under the tested experimental conditions, the RGD loop in EGF-like domain fused to HSA of MFG-E8 or EDIL3/DEL-1 based fusion proteins is accessible and allows interaction with cellular av integrins.

Taken together, these data demonstrate that fusion proteins of the present disclosure bind to cellular integrins, support integrin-dependent cell adhesion and indicate that in proteins with a HSA domain insert retain functionality.

### 10.3 Human Macrophage-Neutrophil Efferocytosis Assay

Human peripheral blood mononuclear cells (PBMCs) were isolated from buffy coat by means of Ficoll gradient centrifugation (Ficoll®-Paque PLUS, GE Healthcare, Sweden) followed by negative selection of monocytes using a Stemcell isolation kit (Stemcell 19059, Vancouver, Canada). Monocytes were differentiated to “M0” macrophages using recombinant human M-CSF 40 ng/mL (Macrophage Colony Stimulating Factor, R&D Systems, US) in RPMI 1640 containing 25 mM HEPES, 10 % FBS, Pen/Strep, 1 mM NaPyr, 50 µM β-Merc for 5 days. One day prior to efferocytosis, macrophages were labeled with PKH26 using the Red Fluorescent Dye Linker kit (Sigma MINI26, US). Cells were resuspended in RPMI 1640 containing 25 mM HEPES, 10% FBS, Pen/Strep, 1 mM NaPyr, 50 µM β-Merc and seeded into black 96-well plates (Corning,US) at 40000 cells/well and allowed to adhere for 20 h.

Neutrophils: Human neutrophils were isolated from buffy coats by dextran sedimentation in combination with a Ficoll™ density gradient as follows: Plasma of the buffy coat was removed by centrifugation of the diluted buffy coat. Cellular harvest was diluted in 1% dextran (from Leuconostoc spp. MW 450.000-650.000; Sigma, US) and allowed to sediment on ice for 2030 min. Leukocytes from supernatant were harvested and on a Ficoll™-Paque layer (GE Healthcare Sweden). After centrifugation the pellet was harvested and remaining erythrocytes were lysed using red blood cell (RBC) lysis buffer (BioConcept, Switzerland). Neutrophils were washed once in medium (RPMI 1640+GlutaMax containing 25 mM HEPES, 10% FBS, Pen/Strep, 0.1 mM NaPyr, 50 uM b-Merc) and kept overnight at 15° C. Apoptosis/cell death was induced by treatment of neutrophils with 1 µg/mL Superfas Ligand (Enzo Life Sciences, Lausanne, Switzerland) at 37° C. for 3 h. Neutrophils were stained with both Hoechst 33342 (Life technologies, US) for 25 min and with DRAQ5 (eBioscience, UK, diluted 1:2000) at 37° C. in the dark for 5 min.

### Efferocytosis Assay

M0 macrophages were incubated with the fusion proteins for 30 min. Apoptotic labelled neutrophils were added at a ratio of M0/neutrophil 1:4. Efferocytosis of apoptotic neutrophils by macrophages was visualized taking advantage of the fluorescence intensity increase of DRAQ5 upon localization of neutrophils in the pH-low lysosomal compartment of M0 macrophages. Efferocytosis was quantified using an ImageXpress Micro XLS wide field high-content analysis system (Molecular DEVICES. CA, US). Macrophages were identified via PKH26 fluorescence. The efferocytosis index (El, displayed as %) was calculated as the ratio of macrophages containing at least one ingested apoptotic neutrophil (DRAQ5high) event to the total number of macrophages. Data analysis was performed using MS Excel and GraphPad Prism software.

The effect of the fusion protein FP114 and FP133 (MFG-E8 derived EGF-HSA-C1 SEQ ID NO: xxx) on the rescue and promotion of efferocytosis of dying neutrophils by LPS treated human macrophages is shown in FIG. 13D. The fusion proteins increase internalization of pHrodo-labelled dying human neutrophils into macrophages over the already high efferocytosis capacity of M0 macrophages. In FIG. 13E it is shown that recombinant fusion protein FP147 (EDIL/DEL-1 derived EGF_EGF_EGF_HSA_C1) can rescue endotoxin (lipopolysaccharide)-impaired efferocytosis of dying neutrophils by human macrophages. Overall the data show the surprising finding that C2-trunctated MFGE8 or EDIL3/DEL-1 derived fusion proteins promote efferocytosis with low nM efficacy in vitro.

### Example 11: Protection of Mice From AKI

### 11.1 Acute Kidney Injury Model

Female C57BL/6 mice (18-22 g) were purchased from Charles River (France) and housed in a temperature-controlled facility in filter-top-protected cages with 12-h light/dark cycles. Animals were handled in strict adherence to Swiss federal laws and the NIH Principles of Laboratory Animal Care. The therapeutic fusion protein under test was administered either intraperitonealy (i.p.) or intravenously (i.v.) two hours before surgery. Buprenorphine (Indivior Schweiz AG) was applied sub-cutaneously (s.c.) at a dose of 0.1 mg/kg 60 to 30 minutes before the surgery. The inhalation anesthesia with isoflurane was induced in a narcotic chamber (3.5-5 Vol. %, carrier gas: oxygen) for 5 min before surgery. During surgery, the animal was maintained under anesthesia via a face mask with 1-2 Vol% isoflurane /oxygen, the gas flow rate was 0.8- 1.2 I/min. The skin of the abdomen was shaved and disinfected with Betaseptic (Mundipharma, France). Animals were placed on a homeothermic blanket (Rothacher-Switzerland) with a homeothermic monitor system (PhysiTemp, US- Physitemp Instruments LLC, US) and covered by sterile gauze. The body temperature was monitored throughout the surgery by a rectal probe (Physitemp Instruments LLC, US) and controlled to allow a body temperature of 36.5-37.5° C. All animals including SHAM controls underwent unilateral nephrectomy of the right kidney: Following mid-line incision / laparotomy, abdominal content was retracted to the left to expose the right kidney. The right ureter and renal blood vessels were disconnected and ligated, the right kidney was then removed. For animals that underwent AKI, abdominal content was positioned to the right on sterile gauze and the left renal artery and vein were dissected to allow clamping for ischemia induction. A micro-aneurysm clamp (B Braun, Switzerland) was used to clamp the renal pedicle (artery and vein together using one clamp) to block blood flow to the kidney and to induce renal ischemia. Successful ischemia was confirmed by color change of the kidney from red to dark purple, which occurred in a few seconds. Following the ischemia induction (35-38 minutes), the micro-aneurysm clamp was removed. Warm sterile saline (~2 ml, 37° C.) was used for washing the abdominal contents to rehydrate tissues before closure of the wound. After the wash, an additional 1 ml of sterile saline was added i.p. as fluid replacement. When starting the reperfusion, the wound was closed in two layers (muscle and the skin, separately). The animals were then maintained under red warm lamp until fully recovered. Buprenorphine was administered again 1 h and 4 h after the surgery at a dose of 0.1 mg/kg and was also included into drinking water (9.091 µg/mL). After 24 h animals were euthanized for analysis. The therapeutic fusion proteins FP135 (EGF-HSA-C1; SEQ ID No: x) was tested in the AKI model was dosed at 1.5 mg/kg i.v. 30 min before ischemia reperfusion injury onset. Serum samples were taken 24 h post ischemia reperfusion induction and analyzed for serum creatinine and blood urea nitrogen (BUN) content using a Hitachi M40 clinic analyzer according to manufacturer’s instruction (Axonlab, Switzerland).

### Examples 12: EGF_HSA_C1 Protects in Liver Fibrosis Model (CCL4 Model)

Liver fibrosis is a wound healing response to various types of insults. If it progresses, it can lead to liver cirrhosis and later, to hepatocellular carcinoma (HCC). Common causes of liver fibrosis in industrialized countries are alcohol abuse, viral hepatitis infections, and metabolic syndromes due to obesity, insulin resistance and diabetes.

Prolonged insult results in inflammation and the deposition of extracellular matrix (ECM) proteins by myofibroblast-like cells which are basically activated hepatic stellate cells (HSC). These cells produce alpha smooth muscle actin (αSMA) and deposit collagens type I and III, as well as producing matrix metalloproteinases (MMPs) and tissue inhibitors (TIMPs). As the disease becomes chronic, the composition of the ECM changes from collagens type IV and VI, glycoproteins and proteoglycans into collagens type I and III and fibronectin.

The liver is able to regenerate if the injury is not severe, whereby neighboring adult hepatocytes are capable of replacing apoptotic or necrotic cells. Resolution of fibrosis occurs when the activated HSC undergo apoptosis or revert into a more quiescent phenotype.

There are several in vivo models available that attempt to mimic various aspects of the disease. The liver fibrosis model needs to be able to mirror various pathological and molecular features of the human disease, as well as being easy to set up and with good reproducibility. Chemical-induced fibrosis models are the closest to these ideal characteristics with one such being the carbon tetrachloride (CCl₄) liver fibrosis model in rodents. Upon repeated intraperitoneal injection of this hepatoxin, a liver fibrosis develops that demonstrates a good likeness to human liver fibrosis. Further, withdrawal of the insult results in resolution of fibrosis and thus the model is reversible.

In the first phase, the CYP2E1 enzyme metabolizes CCl₄ to give the trichloromethyl free radical that contributes to an acute phase reaction characterized by damage of lipid membranes and internal organelles of hepatocytes ultimately leading to necrosis. Acute CC14-mediated liver fibrosis is then characterized by activation of Kupffer cells and induction of an inflammatory response, resulting in secretion of cytokines, chemokines and other proinflammatory factors. This in turn attracts and activates monocytes, neutrophils and lymphocytes, which further contributes to liver necrosis followed by a strong regenerative response resulting in substantial proliferation of hepatocytes and nonparenchymal liver cells around 48 hours after the first CCl₄ application. Histological fibrosis and scarring fibers appear 2 to 3 weeks later in a second phase of disease. A third phase with extensive fibrosis and massive hepatic fat accumulation and increased serum levels of triglycerides and AST can be observed after 4 to 6 weeks of CCl₄ injury. Complete resolution of CCl₄-induced liver fibrosis in mice is observed normally within several weeks after withdrawal of the CCl₄ toxin. An drug with the property to accelerate resolution of fibrosis would be of particular relevance for patients with established diseases. E.g. patients with NASH (non-alcoholic steatohepatitis) chronic kideny disease or sclerodermawho have established fibrosis the demonstration of resolution of fibrosis could become a major primary clinical endpoint and may enable not only to stop disease but also to restore organ function. (Yanguaset al 2016 . Experimental models of liver fibrosis. Arch Toxicol. 2016; 90: 1025-1048. doi: 10.1007/s00204-015-1543-4.)

### CCL4 Liver Fibrosis Model

### Disease Induction

CCl₄ was injected intraperitoneally 3 times perweek during 6 weeks in 8-12 week old male BALB/c mice at a dose of 500 µl/kg freshly diluted in olive oil. Netherlands). CCl₄ was given for a total of 6 weeks to induce liver fibrosis. Treatment with EGF_HSA_C1 (FP135) was initiated either after 4 weeks or 5 weeks or 6 weeks of CCL4 treatment. EGF_HSA_C1 (FP135) was applied at 0.8 mg/kg 3 times weekly intraperitoneally until termination of the experiment (3 days after cessation of CCL4).

### Readouts

Liver enzymes such as ALT (alanine transaminase) and AST (aspartate transaminase) were measured as an assessment of liver damage in serum samples obtained at stop of CCL4 (day 0) and after 3 days at termination of the experiment. ALT and AST were analyzed using a Hitachi M40 clinic analyzer according to manufacturer’s instruction (Axonlab, Switzerland).

To quantify the content of collagen in the livers of animals, a hydroxyproline assay was performed according to manufacturer’s instructions using the Total collagen assay (QuickZyme Biosciences, The Netherlands). The expression of collagen genes COL1A1 and COL1A2 by qPCR was performed as described in section 9.3.

Sonoelastography was used as a reliable and reproducible non-invasive method to assess liver elasticity (stiffness) and has been shown to positively correlate with the liver fibrosis (Li, R., Ren, X., Yan, F. et al. Liver fibrosis detection and staging: a comparative study of T1ρ MR imaging and 2D real-time shear-wave elastography. Abdom Radiol 43, 1713-1722 (2018).

https://doi.org/10.1007/s00261-017-1381-3). Further, this technique is used in the clinic and can help to better translate the outcome of preclinical data to the human liver disease with fibrosis. Liver stiffness was been determined usingultrasound-based shear wave elastography (SWE) assessment: SWE was performed with an Aixplorer® device (Supersonic Imagine, Aix-en-Provence, France). For the acquisitions, mice were anesthetized with isoflurane (~1.5%) and positioned on a heating pad. The ultrasound probe (model SL25-15, SuperSonic Imagine, bandwidth 25 MHz, number of elements 256) was attached to a support and approached to the liver for the assessments. The probe allowed sufficient penetration of the waves for both B-mode and SWE acquisitions.

To minimize movement artefacts due to breathing, elastograms were acquired at expiration. Three elastograms were acquired per mouse and time point. The mean stiffness was then extracted from the three elastograms. The ultrasound examination lasted for approximately 5 min.

### Example 13 Generation of C2-Truncated MFG-E8 (EGF-C1) and HSA Fusion (EGF-HSA-C1) Expression and Purification

Methods for generation of the proteins disclosed herein are described below.

DNA was synthesized at GeneArt (Regensburg, Germany) and cloned into a mammalian expression vector using restriction enzyme-ligation based cloning techniques. The resulting plasmid was transfected into HEK293T cells for transient expression of proteins. In brief, vectors were transfected into suspension-adapted HEK293T cells using Polyethylenimine (PEI; Cat# 24765 Polysciences, Inc.). Typically, 100 ml of cells in suspension at a density of 1-2 Mio cells per ml were transfected with DNA containing 100 µg of expression vector encoding the protein of interest. The recombinant expression vectors were then introduced into the host cells and the construct produced by further culturing of the cells for a period of 7 days to allow for secretion into the culture medium (HEK, serum-fee medium) supplemented with 0.1% pluronic acid, 4 mM glutamine, and 0.25 µg/ml antibiotic.

The produced constructs were then purified from cell-free supernatant, using immobilized metal ion affinity chromatography (IMAC) or anti-HSA capture chromatography.

When his-tagged protein was captured by IMAC, filtered conditioned media was mixed with IMAC resin (GE Healthcare), equilibrated with 20 mM NaPO4, 0.5Mn NaCl, 20 mM Imidazole, pH7.0. The resin was washed three times with 15 column volumes of 20 mM NaPO4, 0.5Mn NaCl, 20 mM Imidazole, pH7.0 before the protein was eluted with 10 column volumes elution buffer (20 mM NaPO4, 0.5Mn NaCl, 500 mM Imidazole, pH7.0).

When protein was captured by anti-HSA chromatography, filtered conditioned media was mixed with anti-HSA resin (Capture Select Human Albumin affinity matrix, Thermo), equilibrated with PBS, pH7.4. The resin was washed three times with 15 column volumes of PBS, pH7.4 before the protein was eluted with 10 column volumes elution buffer (50 mM citrate, 90 mM NaCl, pH 2.5) and pH neutralized using 1 M TRIS pH10.0.

Finally, eluted fractions were polished by using size exclusion chromatography (HiPrep Superdex 200, 16/60, GE Healthcare Life Sciences).

Aggregation content was followed over the purification process by analytical size exclusion chromatography (Superdex 200 Increase 3.2/300 GL, GE Healthcare Life Sciences).

Aggregation level after capture step and expression yield after purification of C2 truncated MFG-E8 and HSA fusion are shown in Table 10. HSA fusion of C2-truncated MFG-E8 shows at least 40-fold improvement in expression over C2-truncated MFG-E8. Moreover, HSA fusion of C2-truncated MFG-E8 shows at least 4-times less aggregation compare to C2-truncated MFG-E8. These data suggest HSA fusion of C2-truncated MFG-E8 exhibits better production properties compare to C2-truncated MFG-E8. By consequence, HSA fusion seems to have better developability for usage as drug.

TABLE 10

Aggregation level after capture step and expression yield after purification of EGF-C1 and EGF-HSA-C1 proteins

| | SEQ ID | Aggregation after capture step (%) | Expression yield after capture and polishing (mg/L) |
|---|---|---|---|
| EGF_C1 | 115 | 46.7 | 0.275 |
| EGF_HSA_C1 | 73 | 10.8 | 11.575 |

### Example 14: Dynamic Light Scattering (DLS) of C2-Truncated MFG-E8 (EGF-C1) and HSA Fusion (EGF-HSA-C1)

The aggregation propensity of C2-truncated MFG-E8 and HSA fusion was measured by dynamic light scattering (DLS, Wyatt). Dynamic light scattering was applied to measure the translational diffusion coefficients of protein in solution by quantifying dynamic fluctuations in scattered light. As an indicator of aggregation formation, hydrodynamic radius was measured upon thermal stress at a concentration of 3 mg/ml, using a DynaPro^{TM} plate reader (Wyatt Technology Europe GmbH, Dernbach, Germany) combined with the software DYNAMICS (version 7.1.0.25, Wyatt). Protein solution was measured in a 384-well plate (384 round well plate, Polystyrol, Thermo Scientific, Langenselbold, Germany).

As showed FIG. 23, C2 truncated MFG-E8 shows an overall higher hydrodynamic radius compare to HSA fusion (5 nm vs 80 nm at 25° C.). Moreover, C2-truncated MFG-E8 shows strong increase of hydrodynamic radius starting at 45° C., indicating a strong aggregation formation, whereas HSA fusion retains the same hydrodynamic radius until at least 55° C. These data suggest HSA fusion of C2-truncated MFG-E8 is more stable and exhibits better biophysical properties compare to C2-truncated MFG-E8. By consequence, HSA fusion seems to have better developability for usage as drug.

Taken together, these data demonstrate that fusion proteins of the present disclosure, e.g. with a HSA domain insert, are functional and efficacious and therefore may be used as therapeutics.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A therapeutic fusion protein for enhancing efferocytosis comprising an integrin binding domain, a phosphatidylserine (PS) binding domain and a solubilizing domain, wherein the solubilizing domain is inserted between the integrin binding domain and the PS binding domain, and wherein the PS binding domain is a truncated variant.

2. The fusion protein of claim 1, wherein the PS binding domain is a truncated variant of at least one PS binding domain listed in Table 2.

3. The fusion protein of claim 1 or claim 2, wherein the PS binding domain is a truncated variant of the PS binding motif of MFG-E8 or of EDIL3.

4. The fusion protein of claim 3, wherein the PS binding domain is a truncated variant of the PS binding motif of MFG-E8.

5. The fusion protein of claim 4, wherein the PS binding domain is a discoidin domain.

6. The fusion protein of any one of the preceding claims, wherein the PS binding domain is a C1 domain.

7. The fusion protein of any one of the preceding claims, wherein the PS binding domain does not comprise a C2 domain.

8. A fusion protein for enhancing efferocytosis comprising an integrin binding domain, a phosphatidylserine (PS) binding domain and a solubilizing domain, wherein the solubilizing domain is inserted between the integrin binding domain and the PS binding domain, and wherein the PS binding domain is a C1 domain.

9. The fusion protein of any one of the preceding claims, wherein the integrin binding domain binds to one or more intergins.

10. The fusion protein of claim 9, wherein the integrin binding domain binds to αvβ3 and/or αvβ5 and/or α8β1 integrin.

11. The fusion protein of any one of the preceding claims, wherein the integrin binding domain comprises a Arginine-Glycine-Aspartic acid (RGD) motif.

12. The fusion protein of any one of the preceding claims, wherein the solubilizing domain is linked directly to the integrin binding domain, to the PS binding domain or to both domains.

13. The fusion protein of any one of the preceding claims, wherein the solubilizing domain is linked indirectly to the integrin binding domain and/or the PS binding domain by a linker.

14. The fusion protein of any one of the preceding claims, wherein the integrin binding domain has an amino acid sequence of SEQ ID NO: 2, or at least 90% sequence identity thereto.

15. A therapeutic fusion protein comprising MFG-E8 and a solubilizing domain, wherein the MFG-E8 comprises from N-terminal to C-terminal: an EGF-like domain, a C1 domain or a C2 domain, and comprises a sequence from wild-type human MFG-E8 (SEQ ID NO: 1) or a functional variant thereof.

16. The fusion protein of claim 17, wherein the solubilizing domain is inserted between the EGF-like domain and the C1 or C2 domain.

17. The fusion protein of any one of the preceding claims, wherein the solubilizing domain is HSA, HSA D3 or Fc-IgG, or a functional variant thereof.

18. The fusion protein of any one of the preceding claims wherein the solubilizing domain comprises human serum albumin (HSA), or a functional variant thereof.

19. The fusion protein of any one of preceding claims for use in the treatment or prevention of an inflammatory disorder or inflammatory organ injury in an individual in need thereof, wherein the inflammatory disorder or inflammatory organ injury is acute kidney injury, acute respiratory distress syndrome, acute liver injury, sepsis, myocardial infarction, stroke, burns, traumatic injury and inflammatory and organ injuries resulting from ischemia/reperfusion.

20. The fusion protein of any one of preceding claims for use in the treatment or, prevention, or amelioration of inhibiting or slowing blood coagulation, microbiome treatment, Inflammatory bowel disease (IBD), fatty acid uptake and/or decreasing gastric motility, microthrombi-dependent disorders, atherosclerosis, cardiac remodeling, tissue fibrosis, acute liver injury, chronic liver diseases, non-alcoholic steatohepatitis (NASH), vascular diseases, age-related vascular disorders, intestinal diseases, sepsis, bone disorders, cancer, Thalassemia, pancreatitis, hepatitis, endocarditis, pneumonia, acute lung injury, osteoarthritis, periodontitis, tissue trauma-induced inflammation, colitis, diabetes, hemorrhagic shock, transplant rejection, radiation-induced damage, splenomegaly, sepsis-induced AKI or multi-organ failure, acute burns, adult and pediatric respiratory distress syndrome, wound healing, tendon repair and neurological diseases.

21. The fusion protein for use according to claim 19 or claim 20, wherein the fusion protein is administered in combination with another therapeutic agent, wherein the therapeutic agent is an immunosuppressive agent, an immunomodulating agent, an anti-inflammatory agent, an antioxidant, an anti-infective agent, a cytotoxic agent or an anti-cancer agent.
